SEQUENCE LISTING (133 sequences)

SEQ ID NO: 1 - protein, 8 aa, synthetic construct
  REGION 1..8: note = Linker 1
GGGSGGGG

SEQ ID NO: 2 - protein, 5 aa, synthetic construct
  REGION 1..5: note = Cysteine-Containing Domain ("Linker 2")
GGCGG

SEQ ID NO: 3 - protein, 28 aa, synthetic construct
  REGION 1..28: note = E-Coil Heterodimer-Promoting Domain
EVAALEKEVAALEKEVAALEKEVAALEK

SEQ ID NO: 4 - protein, 28 aa, synthetic construct
  REGION 1..28: note = K-Coil Heterodimer-Promoting Domain
KVAALKEKVAALKEKVAALKEKVAALKE

SEQ ID NO: 5 - protein, 10 aa, synthetic construct
  REGION 1..10: note = Cysteine-Containing Linker Peptide ("Linker 3")
DKTHTCPPCP

SEQ ID NO: 6 - protein, 219 aa, Homo sapiens
  REGION 1..218: note = MISC_FEATURE - Human Wild-Type IgG CH2-CH3 Domain
APAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 217 aa, synthetic construct
  REGION 1..217: note = "Knob-Bearing" CH2-CH3 Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 217 aa, synthetic construct
  REGION 1..217: note = "Hole-Bearing" IgG CH2-CH3 Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 9 - protein, 98 aa, Homo sapiens
  REGION 1..98: note = MISC_FEATURE - Human IgG1 CH1 Domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 15 aa, Homo sapiens
  REGION 1..15: note = MISC_FEATURE - Human IgG1 Hinge Domain
EPKSCDKTHTCPPCP

SEQ ID NO: 11 - protein, 6 aa, Homo sapiens
  REGION 1..6: note = MISC_FEATURE - Cysteine-Containing Portion of Human CL Domain
FNRGEC

SEQ ID NO: 12 - protein, 6 aa, Homo sapiens
  REGION 1..6: note = MISC_FEATURE - Cysteine-Containing Portion of Human IgG Hinge Domain
VEPKSC

SEQ ID NO: 13 - protein, 107 aa, Homo sapiens [sequence omitted]

SEQ ID NO: 14 - protein, 104 aa, Homo sapiens
  REGION 1..104: note = MISC_FEATURE - Human IgG1 CL Lambda2 Domain
QPKAAPSVTLFPPSSEELQANKATLVCLISDFYPGAVTVAWKADSSPVKAGVETTPSKQSNNKYAASSYLSLTPEQWKSHRSYSCQVTHEGSTVEKTVAPTECS

SEQ ID NO: 15 - protein, 5 aa, synthetic construct
  REGION 1..5: note = Peptide Linker
APSSS

SEQ ID NO: 16 - protein, 8 aa, synthetic construct
  REGION 1..8: note = Peptide Linker
APSSSPME

SEQ ID NO: 17 - protein, 107 aa, Mus musculus
  REGION 1..107: note = MISC_FEATURE - OKT3 Anti-CD3 Antibody Light Chain Variable Domain
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 18 - protein, 192 aa, Mus musculus
  REGION 1..192: note = MISC_FEATURE - OKT3 Anti-CD3 Antibody Heavy Chain Variable Domain
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGDTTGSSVTLGCLVKGYFPEPVTLTWNSGSLSSGVHTFPAVLQSDLYTLSSSVTVTSS

SEQ ID NO: 19 - protein, 106 aa, Mus musculus
  REGION 1..106: note = MISC_FEATURE - M291 Anti-CD3 Antibody Light Chain Variable Domain
DIVLTQSPAIMSASPGEKVTMTCSASSSVSYMNWYQQKSGTSPKRWTYDTSKLASGVPARFSGSGSGTSYSLTISSMEAEDADTYYCQQWSSNPPTFGSGTKLEIK

SEQ ID NO: 20 - protein, 120 aa, Mus musculus
  REGION 1..120: note = MISC_FEATURE - M291 Anti-CD3 Antibody Heavy Chain Variable Domain
QVQLQQSGAELARPGASVKMSCKASGYTFISYTMHWVKQRPGQGLEWIGYINPRSGYTHYNQKLKDKATLTADKSSSSAYMQLSSLTSEDSAVYYCARSAYYDYDGFAYWGQGTLVTVSA

SEQ ID NO: 21 - protein, 130 aa, Mus musculus
  REGION 1..130: note = MISC_FEATURE - YTH12.5 Anti-CD3 Antibody Light Chain Variable Domain
MGWSCIILFLVATATGVHSDIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 22 - protein, 138 aa, Mus musculus
  REGION 1..138: note = MISC_FEATURE - YTH12.5 Anti-CD3 Antibody Heavy Chain Variable Domain
MGWSCIILFLVATATGVHSEVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSS

SEQ ID NO: 23 - protein, 106 aa, synthetic construct
  REGION 1..106: note = Humanized Anti-CD3 "mAb 1" Light Chain Variable Domain Variant 1
DIQMTQSPSSLSASVGDRVTITCSASSSVSYMNWYQQKPGKAPKRLIYDSSKLASGVPSRFSGSGSGTEFTLTISSLQPEDFATYYCQQWSRNPPTFGGGTKVEIK

SEQ ID NO: 24 - protein, 106 aa, synthetic construct
  REGION 1..106: note = Humanized Anti-CD3 "mAb 1" Light Chain Variable Domain Variant 2
DVVMTQSPAIMSAFPGEKVTITCSASSSVSYMNWYQQKPGKAPKRWIYDSSKLASGVPSRFSGSGSGTEFTLTISSLQPEDFATYYCQQWSRNPPTFGGGTKVEIK

SEQ ID NO: 25 - protein, 121 aa, synthetic construct
  REGION 1..121: note = Humanized Anti-CD3 "mAb 1" Heavy Chain Variable Domain
QVQLVQSGAEVKKPGASVKVSCKASGYTFTRSTMHWVRQAPGQGLEWIGYINPSSAYTNYNQKFKDRVTITADKSTSTAYMELSSLRSEDTAVYYCASPQVHYDYNGFPYWGQGTLVTVSS

SEQ ID NO: 26 - protein, 110 aa, synthetic construct
  REGION 1..110: note = Humanized Anti-CD3 "mAb 2" Light Chain Variable Domain
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 27 - protein, 125 aa, synthetic construct
  REGION 1..125: note = Humanized Anti-CD3 "mAb 2" Heavy Chain Variable Domain
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 28 - protein, 125 aa, synthetic construct
  REGION 1..125: note = Humanized Anti-CD3 "mAb 2" Heavy Chain Variable Domain D65G Variant
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 29 - protein, 112 aa, Mus musculus
  REGION 1..112: note = MISC_FEATURE - OKT8 Anti-CD8 Antibody Light Chain Variable Domain
DIVMTQSPASLAVSLGQRATISCRASESVDSYDNSLMHWYQQKPGQPPKVLIYLASNLESGVPARFSGSGSRTDFTLTIDPVEADDAATYYCQQNNEDPYTFGGGTKLEIKR

SEQ ID NO: 30 - protein, 120 aa, Mus musculus
  REGION 1..120: note = MISC_FEATURE - OKT8 Anti-CD8 Antibody Heavy Chain Variable Domain
QVQLLESGPELLKPGASVKMSCKASGYTFTDYNMHWVKQSHGKSLEWIGYIYPYTGGTGYNQKFKNKATLTVDSSSSTAYMELRSLTSEDSAVYYCARNFRYTYWYFDVWGQGTTVTVSS

SEQ ID NO: 31 - protein, 106 aa, Mus musculus
  REGION 1..106: note = MISC_FEATURE - TRX2 Anti-CD8 Antibody Light Chain Variable Domain
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 32 - protein, 121 aa, Mus musculus
  REGION 1..121: note = MISC_FEATURE - TRX2 Anti-CD8 Antibody Heavy Chain Variable Domain
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 33 - protein, 110 aa, Mus musculus
  REGION 1..110: note = MISC_FEATURE - A32 Anti-HIV gp120 Antibody Light Chain Variable Domain
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVL

SEQ ID NO: 34 - protein, 123 aa, Mus musculus
  REGION 1..123: note = MISC_FEATURE - A32 Anti-HIV gp120 Antibody Heavy Chain Variable Domain
QVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTMVTVSS

SEQ ID NO: 35 - protein, 113 aa, Mus musculus
  REGION 1..113: note = MISC_FEATURE - 7B2 Anti-HIV gp41 Antibody Light Chain Variable Domain
DIVMTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVEIK

SEQ ID NO: 36 - protein, 126 aa, Mus musculus
  REGION 1..126: note = MISC_FEATURE - 7B2 Anti-HIV gp41 Antibody Heavy Chain Variable Domain
QVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSS

SEQ ID NO: 37 - protein, 106 aa, Mus musculus
  REGION 1..106: note = MISC_FEATURE - Palivizumab Anti-RSV Glycoprotein F Antibody Light Chain Variable Domain
DIQMTQSPSTLSASVGDRVTITCRASQSVGYMHWYQQKPGKAPKLLIYDTSKLASGVPSRFSGSGSGTEFTLTISSLQPDDFATYYCFQGSGYPFTFGGGTKLEIK

SEQ ID NO: 38 - protein, 120 aa, Mus musculus
  REGION 1..120: note = MISC_FEATURE - Palivizumab Anti-RSV Glycoprotein F Antibody Heavy Chain Variable Domain
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 39 - protein, 107 aa, synthetic construct
  REGION 1..107: note = "BRCA84D-5VL" Humanized Anti-B7-H3 Antibody Light Chain Variable Domain
DIQLTQSPSFLSASVGDRVTITCKASQNVDTNVAWYQQKPGQAPKALIYSASYRYSGVPSRFSGSGSGTDFTLTISSLQPEDFATYYCQQYNNYPFTFGQGTKLEIK

SEQ ID NO: 40 - protein, 122 aa, synthetic construct
  REGION 1..122: note = "BRCA84D-2VH" Humanized Anti-B7-H3 Antibody Heavy Chain Variable Domain
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 41 - protein, 107 aa, synthetic construct
  REGION 1..107: note = "BRCA69D" Humanized Anti-B7-H3 Antibody Light Chain Variable Domain
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIK

SEQ ID NO: 42 - protein, 120 aa, synthetic construct
  REGION 1..120: note = "BRCA69D" Humanized Anti-B7-H3 Antibody Heavy Chain Variable Domain
QVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSS

SEQ ID NO: 43 - protein, 108 aa, synthetic construct
  REGION 1..108: note = "PRCA157" Humanized Anti-B7-H3 Antibody Light Chain Variable Domain
DIQMTQSPASLSVSVGETVTITCRASESIYSYLAWYQQKQGKSPQLLVYNTKTLPEGVPSRFSGSGSGTQFSLKINSLQPEDFGRYYCQHHYGTPPWTFGGGTNLEIK

SEQ ID NO: 44 - protein, 117 aa, synthetic construct
  REGION 1..117: note = "PRCA157" Humanized Anti-B7-H3 Antibody Heavy Chain Variable Domain
EVQQVESGGDLVKPGGSLKLSCAASGFTFSSYGMSWVRQTPDKRLEWVATINSGGSNTYYPDSLKGRFTISRDNAKNTLYLQMRSLKSEDTAMYYCARHDGGAMDYWGQGTSVTVSS

SEQ ID NO: 45 - protein, 107 aa, synthetic construct
  REGION 1..107: note = Humanized Anti-A33 Antibody Light Chain Variable Domain
QIVLTQSPAIMSASPGERVTMTCSARSSISFMYWYQQKPGSSPRLLIYDTSNLASGVPVRFSGSGSGTSYSLTISRMEAEDAATYYCQQWSSYPLTFGSGTKLELKR

SEQ ID NO: 46 - protein, 119 aa, synthetic construct
  REGION 1..119: note = Humanized Anti-A33 Antibody Heavy Chain Variable Domain
QVQLQQSGPELVKPGASVKISCKASGYTFSGSWMNWVKQRPGQGLEWIGRIYPGDGETNYNGKFKDKATLTADKSSTTAYMELSSLTSVDSAVYFCARIYGNNVYFDVWGAGTTVTVSS

SEQ ID NO: 47 - protein, 107 aa, synthetic construct
  REGION 1..107: note = Humanized Anti-5T4 Antibody "mAb 1" Light Chain Variable Domain
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIK

SEQ ID NO: 48 - protein, 118 aa, synthetic construct
  REGION 1..118: note = Humanized Anti-5T4 Antibody "mAb 1" Heavy Chain Variable Domain
QVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSS

SEQ ID NO: 49 - protein, 112 aa, synthetic construct
  REGION 1..112: note = Humanized Anti-5T4 Antibody "mAb 2" Light Chain Variable Domain
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 50 - protein, 127 aa, synthetic construct
  REGION 1..127: note = Humanized Anti-5T4 Antibody "mAb 2" Heavy Chain Variable Domain
QVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSS

SEQ ID NO: 51 - protein, 112 aa, synthetic construct
  REGION 1..112: note = Humanized Anti-ROR1 mAb 1 Antibody Light Chain Variable Domain
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVL

SEQ ID NO: 52 - protein, 121 aa, synthetic construct
  REGION 1..121: note = Humanized Anti-ROR1 mAb 1 Antibody Heavy Chain Variable Domain
QEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFDIWGQGTTVTVSS

SEQ ID NO: 53 - protein, 107 aa, Mus musculus
  REGION 1..107: note = MISC_FEATURE - 2A2 Anti-ROR1 Antibody Light Chain Variable Domain
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 54 - protein, 118 aa, Mus musculus
  REGION 1..118: note = MISC_FEATURE - 2A2 Anti-ROR1 Antibody Heavy Chain Variable Domain
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 55 - protein, 110 aa, Mus musculus
  REGION 1..110: note = MISC_FEATURE - R11 Anti-ROR1 Antibody Light Chain Variable Domain
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 56 - protein, 116 aa, Mus musculus
  REGION 1..116: note = MISC_FEATURE - R11 Anti-ROR1 Antibody Heavy Chain Variable Domain
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 57 - protein, 112 aa, Mus musculus
  REGION 1..112: note = MISC_FEATURE - R12 Anti-ROR1 Antibody Light Chain Variable Domain
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 58 - protein, 121 aa, Mus musculus
  REGION 1..121: note = MISC_FEATURE - R12 Anti-ROR1 Antibody Heavy Chain Variable Domain
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 59 - protein, 504 aa, synthetic construct
  REGION 1..504: note = First Polypeptide Chain of Exemplary B7-H3 mAb 1/ CD3 mAb 2 / CD8 mAb 1 Tri-Specific Binding Molecule (VL(B7-H3 mAb 1)-VH(CD3 mAb 2)-E-Coil-(CH2-CH3))
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 272 aa, synthetic construct
  REGION 1..272: note = Second Polypeptide Chain of Exemplary B7-H3 mAb 1/ CD3 mAb 2 / CD8 mAb 1 Tri-Specific Binding Molecule (VL(CD3 mAb 2)-VH(B7-H3 mAb 1)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSSGGCGGGKVAALKEKVAALKEKVAALKEKVAALKE

SEQ ID NO: 61 - protein, 448 aa, synthetic construct
  REGION 1..448: note = Third Polypeptide Chain of Exemplary B7-H3 mAb 1/ CD3 mAb 2 / CD8 mAb 1 Tri-Specific Binding Molecule (Heavy Chain CD8 mAb 1)
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 62 - protein, 214 aa, synthetic construct
  REGION 1..214: note = Fourth Polypeptide Chain of Exemplary B7-H3 mAb 1/ CD3 mAb 2 / CD8 mAb 1 Tri-Specific Binding Molecule (Light Chain CD8 mAb 1)
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 504 aa, synthetic construct; identical to SEQ ID NO: 59
  REGION 1..504: note = First Polypeptide Chain of Exemplary B7-H3 mAb 1/ CD3 mAb 2 / CD8 mAb 2 Tri-Specific Binding Molecule (VL(B7-H3 mAb 1)-VH(CD3 mAb 2)-E-Coil-(CH2-CH3))

SEQ ID NO: 64 - protein, 272 aa, synthetic construct; identical to SEQ ID NO: 60
  REGION 1..272: note = Second Polypeptide Chain of Exemplary B7-H3 mAb 1/ CD3 mAb 2 / CD8 mAb 2 Tri-Specific Binding Molecule (VL(CD3 mAb 2)-VH(B7-H3 mAb 1)-K-Coil)

SEQ ID NO: 65 - protein, 451 aa, synthetic construct
  REGION 1..451: note = Third Polypeptide Chain of Exemplary B7-H3 mAb 1/ CD3 mAb 2 / CD8 mAb 2 Tri-Specific Binding Molecule (Heavy Chain CD8 mAb 2)
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 66 - protein, 213 aa, synthetic construct
  REGION 1..213: note = Fourth Polypeptide Chain of Exemplary B7-H3 mAb 1/ CD3 mAb 2 / CD8 mAb 2 Tri-Specific Binding Molecule (Light Chain CD8 mAb 2)
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 504 aa, synthetic construct
  REGION 1..504: note = First Polypeptide Chain of Exemplary CD3 mAb 2 / CD8 mAb 1 / B7-H3 mAb 1 Tri-Specific Binding Molecule (VL(CD3 mAb 2)-VH(CD8 mAb 1)-E-Coil-(CH2-CH3))
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELRGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 270 aa, synthetic construct
  REGION 1..270: note = Second Polypeptide Chain of Exemplary CD3 mAb 2 / CD8 mAb 1 / B7-H3 mAb 1 Tri-Specific Binding Molecule (VL(CD8 mAb 1)-VH(CD3 mAb 2)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSGGCGGGKVAALKEKVAALKEKVAALKEKVAALKE

SEQ ID NO: 69 - protein, 450 aa, synthetic construct
  REGION 1..450: note = Third Polypeptide Chain of Exemplary CD3 mAb 2 / CD8 mAb 1 / B7-H3 mAb 1 Tri-Specific Binding Molecule Heavy Chain B7-H3 mAb 1)
QVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 70 - protein, 214 aa, synthetic construct
  REGION 1..214: note = Fourth Polypeptide Chain of Exemplary CD3 mAb 2 / CD8 mAb 1 / B7-H3 mAb 1 Tri-Specific Binding Molecule (Light Chain B7-H3 mAb 1)
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 499 aa, synthetic construct
  REGION 1..499: note = First Polypeptide Chain of Exemplary B7-H3 mAb 1/ CD8 mAb 1 / CD3 mAb 2 Tri-Specific Binding Molecule (VL(B7-H3 mAb 1)-VH(CD8 mAb 1)-E-Coil-(CH2-CH3))
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELRGGGSGGGGQVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 267 aa, synthetic construct
  REGION 1..267: note = Second Polypeptide Chain of Exemplary B7-H3 mAb 1/ CD8 mAb 1 / CD3 mAb 2 Tri-Specific Binding Molecule (VL(CD8 mAb 1)-VH(B7-H3 mAb1)-K-Coil)
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIKGGGSGGGGEVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSGGCGGGKVAALKEKVAALKEKVAALKEKVAALKE

SEQ ID NO: 73 - protein, 455 aa, synthetic construct
  REGION 1..455: note = Third Polypeptide Chain of Exemplary B7-H3 mAb 1/ CD8 mAb 1 / CD3 mAb 2 Tri-Specific Binding Molecule (Heavy Chain CD3 mAb 2)
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 74 - protein, 217 aa, synthetic construct
  REGION 1..217: note = Fourth Polypeptide Chain of Exemplary B7-H3 mAb 1/ CD8 mAb 1 / CD3 mAb 2 Tri-Specific Binding Molecule (Light Chain CD3 mAb 2)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 509 aa, synthetic construct
  REGION 1..509: note = First Polypeptide Chain of Exemplary 5T4 mAb 2 / CD3 mAb 2 / CD8 mAb 1 Tri--Specific Binding Molecule (VL(5T4 mAb 2)-VH(CD3 mAb 2)-E-Coil-(CH2-CH3))
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 279 aa, synthetic construct
  REGION 1..279: note = Second Polypeptide Chain of Exemplary 5T4 mAb 2 / CD3 mAb 2 / CD8 mAb 1 Tri--Specific Binding Molecule (VL(CD3 mAb 2)-VH(5T4 mAb 2)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSSGGCGGGKVAALKEKVAALKEKVAALKEKVAALKE

SEQ ID NO: 77 - protein, 448 aa, synthetic construct; identical to SEQ ID NO: 61
  REGION 1..448: note = Third Polypeptide Chain of Exemplary 5T4 mAb 2 / CD3 mAb 2 / CD8 mAb 1 Tri--Specific Binding Molecule (Heavy Chain CD8 mAb 1)

SEQ ID NO: 78 - protein, 214 aa, synthetic construct; identical to SEQ ID NO: 62
  REGION 1..214: note = Fourth Polypeptide Chain of Exemplary 5T4 mAb 2 / CD3 mAb 2 / CD8 mAb 1 Tri--Specific Binding Molecule (Light Chain CD8 mAb 1)

SEQ ID NO: 79 - protein, 509 aa, synthetic construct; identical to SEQ ID NO: 75
  REGION 1..509: note = First Polypeptide Chain of Exemplary 5T4 mAb 2 / CD3 mAb 2 / CD8 mAb 2 Tri--Specific Binding Molecule (VL(5T4 mAb 2)-VH(CD3 mAb 2)-E-Coil-(CH2-CH3))

SEQ ID NO: 80 - protein, 279 aa, synthetic construct; identical to SEQ ID NO: 76
  REGION 1..279: note = Second Polypeptide Chain of Exemplary 5T4 mAb 2 / CD3 mAb 2 / CD8 mAb 2 Tri--Specific Binding Molecule (VL(CD3 mAb 2)-VH(5T4 mAb 2)-K-Coil)

SEQ ID NO: 81 - protein, 451 aa, synthetic construct; identical to SEQ ID NO: 65
  REGION 1..451: note = Third Polypeptide Chain of Exemplary 5T4 mAb 2 / CD3 mAb 2 / CD8 mAb 2 Tri--Specific Binding Molecule (Heavy Chain CD8 mAb 2)

SEQ ID NO: 82 - protein, 213 aa, synthetic construct; identical to SEQ ID NO: 66
  REGION 1..213: note = Fourth Polypeptide Chain of Exemplary 5T4 mAb 2 / CD3 mAb 2 / CD8 mAb 2 Tri--Specific Binding Molecule (Light Chain CD8 mAb 2)

SEQ ID NO: 83 - protein, 510 aa, synthetic construct
  REGION 1..510: note = First Polypeptide Chain of Exemplary ROR1 mAb 1 / CD3 mAb 2 / CD8 mAb 1 Tri--Specific Binding Molecule (VL(ROR1 mAb 1)-VH(CD3 mAb 2)-E-Coil-(CH2-CH3))
QLVLTQSPSASASLGSSVKLTCTLSSGHKTDTIDWYQQQPGKAPRYLMKLEGSGSYNKGSGVPDRFGSGSSSGADRYLTISSLQSEDEADYYCGTDYPGNYLFGGGTQLTVLGGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 273 aa, synthetic construct
  REGION 1..273: note = Second Polypeptide Chain of Exemplary ROR1 mAb 1 / CD3 mAb 2 / CD8 mAb 1 Tri--Specific Binding Molecule (VL(CD3 mAb 2)-VH(ROR1 mAb 1)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQEQLVESGGGLVQPGGSLRLSCAASGFTFSDYYMSWVRQAPGKGLEWVATIYPSSGKTYYADSVKGRFTISSDNAKNSLYLQMNSLRAEDTAVYYCARDSYADDAALFDIWGQGTTVTVSSGGCGGGKVAALKEKVAALKEKVAALKEKVAALKE

SEQ ID NO: 85 - protein, 447 aa, synthetic construct
  REGION 1..447: note = Third Polypeptide Chain of Exemplary ROR1 mAb 1 / CD3 mAb 2 / CD8 mAb 1 Tri--Specific Binding Molecule (Heavy Chain CD8 mAb 1)
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 86 - protein, 214 aa, synthetic construct; identical to SEQ ID NO: 62
  REGION 1..214: note = Fourth Polypeptide Chain of Exemplary ROR1 mAb 1 / CD3 mAb 2 / CD8 mAb 1 Tri--Specific Binding Molecule (Light Chain CD8 mAb 1)

SEQ ID NO: 87 - protein, 510 aa, synthetic construct; identical to SEQ ID NO: 83
  REGION 1..510: note = First Polypeptide Chain of Exemplary ROR1 mAb 1 / CD3 mAb 2 / CD8 mAb 2 Tri--Specific Binding Molecule (VL(ROR1 mAb 1)-VH(CD3 mAb 2)-E-Coil-(CH2-CH3))

SEQ ID NO: 88 - protein, 273 aa, synthetic construct; identical to SEQ ID NO: 84
  REGION 1..273: note = Second Polypeptide Chain of Exemplary ROR1 mAb 1 / CD3 mAb 2 / CD8 mAb 2 Tri--Specific Binding Molecule (VL(CD3 mAb 2)-VH(ROR1 mAb 1)-K-Coil)

SEQ ID NO: 89 - protein, 451 aa, synthetic construct; identical to SEQ ID NO: 65
  REGION 1..451: note = Third Polypeptide Chain of Exemplary ROR1 mAb 1 / CD3 mAb 2 / CD8 mAb 2 Tri--Specific Binding Molecule (Heavy Chain CD8 mAb 2)

SEQ ID NO: 90 - protein, 213 aa, synthetic construct; identical to SEQ ID NO: 66
  REGION 1..213: note = Fourth Polypeptide Chain of Exemplary ROR1 mAb 1 / CD3 mAb 2 / CD8 mAb 2 Tri--Specific Binding Molecule (Light Chain CD8 mAb 2)

SEQ ID NO: 91 - protein, 509 aa, synthetic construct
  REGION 1..509: note = First Polypeptide Chain of Exemplary HIV mAb 1 / CD3 mAb 2 / CD8 mAb 1 Tri--Specific Binding Molecule (VL(HIV mAb 1)-VH(CD3 mAb 2)-E-Coil-(CH2-CH3))
DIVMTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 277 aa, synthetic construct
  REGION 1..277: note = Second Polypeptide Chain of Exemplary HIV mAb 1 / CD3 mAb 2 / CD8 mAb 1 Tri--Specific Binding Molecule (VL(CD3 mAb 2)-VH(HIV mAb 1)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSSASTKGKVAACKEKVAALKEKVAALKEKVAALKE

SEQ ID NO: 93 - protein, 447 aa, synthetic construct; identical to SEQ ID NO: 85
  REGION 1..447: note = Third Polypeptide Chain of Exemplary HIV mAb 1 / CD3 mAb 2 / CD8 mAb 1 Tri--Specific Binding Molecule (Heavy Chain CD8 mAb 1)

SEQ ID NO: 94 - protein, 214 aa, synthetic construct; identical to SEQ ID NO: 62
  REGION 1..214: note = Fourth Polypeptide Chain of Exemplary HIV mAb 1 / CD3 mAb 2 / CD8 mAb 1 Tri--Specific Binding Molecule (Light Chain CD8 mAb 1)

SEQ ID NO: 95 - protein, 506 aa, synthetic construct
  REGION 1..506: note = First Polypeptide Chain of Exemplary HIV mAb 2 / CD3 mAb 2 / CD8 mAb 1 Tri--Specific Binding Molecule (VL(HIV mAb 2)-VH(CD3 mAb 2)-E-Coil-(CH2-CH3))
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVLGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 274 aa, synthetic construct
  REGION 1..274: note = Second Polypeptide Chain of Exemplary HIV mAb 2 / CD3 mAb 2 / CD8 mAb 1 Tri--Specific Binding Molecule (VL(CD3 mAb 2)-VH(HIV mAb 2)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLQESGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTLVTVSSASTKGKVAACKEKVAALKEKVAALKEKVAALKE

SEQ ID NO: 97 - protein, 447 aa, synthetic construct; identical to SEQ ID NO: 85
  REGION 1..447: note = Third Polypeptide Chain of Exemplary HIV mAb 2 / CD3 mAb 2 / CD8 mAb 1 Tri--Specific Binding Molecule (Heavy Chain CD8 mAb 1)

SEQ ID NO: 98 - protein, 214 aa, synthetic construct; identical to SEQ ID NO: 62
  REGION 1..214: note = Fourth Polypeptide Chain of Exemplary HIV mAb 2 / CD3 mAb 2 / CD8 mAb 1 Tri--Specific Binding Molecule (Light Chain CD8 mAb 1)

SEQ ID NO: 99 - protein, 110 aa, synthetic construct; identical to SEQ ID NO: 26
  REGION 1..110: note = Variable Light Chain Domain of Anti-Human CD3 mAb 2 Low

SEQ ID NO: 100 - protein, 125 aa, synthetic construct
  REGION 1..125: note = Variable Heavy Chain Domain of Anti-Human CD3 mAb 2 Low
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSS

SEQ ID NO: 101 - protein, 110 aa, synthetic construct; identical to SEQ ID NO: 26
  REGION 1..110: note = Variable Light Chain Domain of Anti-Human CD3 mAb 2 Fast

SEQ ID NO: 102 - protein, 125 aa, synthetic construct
  REGION 1..125: note = Variable Heavy Chain Domain of Anti-Human CD3 mAb 2 Fast
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSS

SEQ ID NO: 103 - protein, 504 aa, synthetic construct
  REGION 1..504: note = First Polypeptide Chain of Exemplary 5T4 mAb 1 / CD3 mAb 2 / CD8 mAb 1 Tri--Specific Binding Molecule (VL(5T4 mAb 1)-VH(CD3 mAb 2)-E-Coil-(CH2-CH3))
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 270 aa, synthetic construct
  REGION 1..270: note = Second Polypeptide Chain of Exemplary 5T4 mAb 1 / CD3 mAb 2 / CD8 mAb 1 Tri--Specific Binding Molecule (VL(CD3 mAb 2)-VH(5T4 mAb 1)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSSGGCGGGKVAALKEKVAALKEKVAALKEKVAALKE

SEQ ID NO: 105 - protein, 448 aa, synthetic construct; identical to SEQ ID NO: 61
  REGION 1..448: note = Third Polypeptide Chain of Exemplary 5T4 mAb 1 / CD3 mAb 2 / CD8 mAb 1 Tri--Specific Binding Molecule (Heavy Chain CD8 mAb 1)

SEQ ID NO: 106 - protein, 214 aa, synthetic construct; identical to SEQ ID NO: 62
  REGION 1..214: note = Fourth Polypeptide Chain of Exemplary 5T4 mAb 1 / CD3 mAb 2 / CD8 mAb 1 Tri--Specific Binding Molecule (Light Chain CD8 mAb 1)

SEQ ID NO: 107 - protein, 503 aa, synthetic construct
  REGION 1..503: note = First Polypeptide Chain of Exemplary 5T4 mAb 1 / CD3 mAb 2 Low / CD8 mAb 1 Tri--Specific Binding Molecule (VL(5T4 mAb 1)-VH(CD3 mAb 2 Low)-E-Coil-(CH2-CH3))
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVTWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 269 aa, synthetic construct
  REGION 1..269: note = Second Polypeptide Chain of Exemplary 5T4 mAb 1 / CD3 mAb 2 Low / CD8 mAb 1 Tri--Specific Binding Molecule (VL(CD3 mAb 2 Low)-VH(5T4 mAb 1)-K-Coil)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSFWMHWVRQAPGQGLEWMGRIDPNRGGTEYNEKAKSRVTMTADKSTSTAYMELSSLRSEDTAVYYCAGGNPYYPMDYWGQGTTVTVSSASTKGKVAACKEKVAALKEKVAALKEKVAALKE

SEQ ID NO: 109 - protein, 448 aa, synthetic construct; identical to SEQ ID NO: 61
  REGION 1..448: note = Third Polypeptide Chain of Exemplary 5T4 mAb 1 / CD3 mAb 2 Low / CD8 mAb 1 Tri--Specific Binding Molecule (Heavy Chain CD8 mAb 1)

SEQ ID NO: 110 - protein, 214 aa, synthetic construct; identical to SEQ ID NO: 62
  REGION 1..214: note = Fourth Polypeptide Chain of Exemplary 5T4 mAb 1 / CD3 mAb 2 Low / CD8 mAb 1 Tri--Specific Binding Molecule (Light Chain CD8 mAb 1)

SEQ ID NO: 111 - protein, 503 aa, synthetic construct
  REGION 1..503: note = First Polypeptide Chain of Exemplary 5T4 mAb 1 / CD3 mAb 2 Fast / CD8 mAb 1 Tri--Specific Binding Molecule (VL(5T4 mAb 1)-VH(CD3 mAb 2 Fast)-E-Coil-(CH2-CH3))
DIQMTQSPSSLSASVGDRVTITCRASQGISNYLAWFQQKPGKAPKSLIYRANRLQSGVPSRFSGSGSGTDFTLTISSLQPEDVATYYCLQYDDFPWTFGQGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHKNFGNSYVTWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 269 aa, synthetic construct; identical to SEQ ID NO: 108
  REGION 1..269: note = Second Polypeptide Chain of Exemplary 5T4 mAb 1 / CD3 mAb 2 Fast / CD8 mAb 1 Tri--Specific Binding Molecule (VL(CD3 mAb 2 Fast)-VH(5T4 mAb 1)-K-Coil)

SEQ ID NO: 113 - protein, 448 aa, synthetic construct; identical to SEQ ID NO: 61
  REGION 1..448: note = Third Polypeptide Chain of Exemplary 5T4 mAb 1 / CD3 mAb 2 Fast / CD8 mAb 1 Tri--Specific Binding Molecule (Heavy Chain CD8 mAb 1)

SEQ ID NO: 114 - protein, 214 aa, synthetic construct; identical to SEQ ID NO: 62
  REGION 1..214: note = Fourth Polypeptide Chain of Exemplary 5T4 mAb 1 / CD3 mAb 2 Fast / CD8 mAb 1 Tri--Specific Binding Molecule (Light Chain CD8 mAb 1)

SEQ ID NO: 115 - protein, 28 aa, synthetic construct
  REGION 1..28: note = Cysteine-Containing E-Coil Heterodimer-Promoting Domain
EVAACEKEVAALEKEVAALEKEVAALEK

SEQ ID NO: 116 - protein, 28 aa, synthetic construct
  REGION 1..28: note = Cysteine-Containing K-Coil Heterodimer-Promoting Domain
KVAACKEKVAALKEKVAALKEKVAALKE

SEQ ID NO: 117 - protein, 12 aa, synthetic construct
  REGION 1..12: note = Anti-ROR1 mAb 1 Light Chain Variable Domain CDRL1
TLSSGHKTDTID

SEQ ID NO: 118 - protein, 7 aa, synthetic construct
  REGION 1..7: note = Anti-ROR1 mAb 1 Light Chain Variable Domain CDRL2
LEGSGSY

SEQ ID NO: 119 - protein, 9 aa, synthetic construct
  REGION 1..9: note = Anti-ROR1 mAb 1 Light Chain Variable Domain CDRL3
GTDYPGNYL

SEQ ID NO: 120 - protein, 10 aa, synthetic construct
  REGION 1..10: note = Anti-ROR1 mAb 1 Heavy Chain Variable Domain CDRH1
GFTFSDYYMS

SEQ ID NO: 121 - protein, 17 aa, synthetic construct
  REGION 1..17: note = Anti-ROR1 mAb 1 Heavy Chain Variable Domain CDRH2
TIYPSSGKTYYADSVKG

SEQ ID NO: 122 - protein, 12 aa, synthetic construct
  REGION 1..12: note = Anti-ROR1 mAb 1 Heavy Chain Variable Domain CDRH3
DSYADDAALFDI

SEQ ID NO: 123 - protein, 46 aa, Streptococcus dysgalactiae
  REGION 1..46: note = MISC_FEATURE - Albumin-Binding Domain 3 (ABD3) of Protein G of Streptococcus Strain G148
LAEAKVLANRELDKYGVSDYYKNLIDNAKSAEGVKALIDEILAALP

SEQ ID NO: 124 - protein, 46 aa, synthetic construct
  REGION 1..46: note = Variant Deimmunized Albumin-Binding Domain 3 (ABD3) of Protein G of Streptococcus Strain G148
LAEAKVLANRELDKYGVSDYYKNAANNAKTVEGVKALIAEILAALP

SEQ ID NO: 125 - protein, 46 aa, synthetic construct
  REGION 1..46: note = Variant Deimmunized Albumin-Binding Domain 3 (ABD3) of Protein G of Streptococcus Strain G148
LAEAKVLANRELDKYGVSDYYKNLISNAKSVEGVKALIAEILAALP

SEQ ID NO: 126 - protein, 4 aa, synthetic construct
  REGION 1..4: note = Peptide Linker
GGGS

SEQ ID NO: 127 - protein, 6 aa, synthetic construct
  REGION 1..6: note = Cysteine-Containing Domain
AEPKSC

SEQ ID NO: 128 - protein, 7 aa, synthetic construct
  REGION 1..7: note = Cysteine-Containing Variant of Portion of Human CL Domain
GFNRGEC

SEQ ID NO: 129 - protein, 15 aa, synthetic construct
  REGION 1..15: note = Alternative Cysteine-Containing Linker Peptide ("Linker 3")
EPKSCDKTHTCPPCP

SEQ ID NO: 130 - protein, 16 aa, synthetic construct
  REGION 1..16: note = Alternative Cysteine-Containing Linker Peptide ("Linker 3")
LEPKSSDKTHTCPPCP

SEQ ID NO: 131 - protein, 5 aa, synthetic construct
  REGION 1..5: note = Peptide Linker
ASTKG

SEQ ID NO: 132 - protein, 6 aa, synthetic construct
  REGION 1..6: note = Peptide Linker
LEPKSS

SEQ ID NO: 133 - protein, 7 aa, synthetic construct
  REGION 1..7: note = Variant of Cysteine-Containing Portion of Human IgG Hinge Domain
GVEPKSC

(19) United States
(12) Patent Application Publication
(10) Pub. No.: US 2024/0182559 A1
(43) Pub. Date: Jun. 6, 2024

(54) MULTl-CHAIN POLYPEPTIDE-CONTAINING TRI-SPECIFIC BINDING MOLECULES THAT SPECIFICALLY BIND TO MULTIPLE CANCER ANTIGENS

(71) Applicant: MACROGENICS, INC., Rockville, MD (US)

(72) Inventors: Leslie S. JOHNSON, Rockville, MD (US); Ling HUANG, Rockville, MD (US); Gurunadh Reddy CHICHILI, Rockville, MD (US); Kalpana SHAH, Rockville, MD (US); Chia-Ying Kao LAM, Rockville, MD (US); Stephen James BURKE, Rockville, MD (US); Liqin LIU, Rockville, MD (US); Paul A. MOORE, Rockville, MD (US); Ezio BONVINI, Rockville, MD (US); Bhaswati BARAT, Rockville, MD (US)

(21) Appl. No.: 18/484,921

(22) Filed: Oct. 11, 2023

Related U.S. Application Data
(63) Continuation of application No. 16/818,893, filed on Mar. 13, 2020, now Pat. No. 11,820,818.
Application No. 16/818,893 is a division of application No. 15/313,741, filed on Nov. 23, 2016, now Pat. No. 10,647,768.
(60) Provisional application No. 62/107,824, filed on Jan. 26, 2015.
(60) Provisional application No. 62/008,229, filed on Jun. 5, 2014.
(60) Provisional application No. 62/004,571, filed on May 29, 2014.

(51) Int. Cl. C07K 16/28; C07K 16/30

(52) U.S. Cl. CPC C07K 16/28; C07K 16/2803; C07K 16/2809; C07K 16/2815; C07K 16/2827; C07K 16/283; C07K 16/2878; C07K 16/30; C07K 2317/31; C07K 2317/524; C07K 2317/526; C07K 2317/55; C07K 2317/56; C07K 2317/565; C07K 2317/622; C07K 2317/626; C07K 2317/73; C07K 2317/92; C07K 2318/20; C07K 2319/00

(57) ABSTRACT

The present invention relates to Tri-Specific Binding Molecules, which are multi-chain polypeptide molecules that possess three Binding Domains and are thus capable of mediating coordinated binding to three epitopes. The Binding Domains may be selected such that the Tri-Specific Binding Molecules are capable of binding to any three different epitopes. Such epitopes may be epitopes of the same antigen or epitopes of two or three different antigens. In a preferred embodiment, one of such epitopes will be capable of binding to CD3, the second of such epitopes will be capable of binding to CD8, and the third of such epitopes will be capable of binding to an epitope of a Disease-Associated Antigen. The invention also provides a novel ROR1-binding antibody, as well as derivatives thereof and uses for such compositions.

### CROSS-REFERENCE TO RELATED APPLICATIONS

This application is a continuation of U.S. Ser. No. 16/818,893 (filed Mar. 13, 2020) which is a division of Ser. No. 15/313,741 (filed Nov. 23, 2016) patented as U.S. Pat. No. 10,647,768. Ser. No. 15/313,741 is a National stage entry of PCT/US15/33076 (filed May 29, 2015, expired) claims priority to U.S. Patent Applications No. 62/008,229 (filed Jun. 5, 2014; expired), 62/004,571 (filed May 29, 2014; expired), and 62/107,824 (filed Jan. 26, 2015, expired), each of which applications is herein incorporated by reference in its entirety.

### REFERENCE TO SEQUENCE LISTING

The application contains a Sequence Listing which has been submitted electronically in .XML format and is hereby incorporated by reference in its entirety. Said .XML copy, created on Oct. 11, 2023, is named “0260-0001US3 Sequence Listing.xml” and is 200,177 bytes in size. The sequence listing contained in this .XML file is part of the specification and is hereby incorporated by reference herein in its entirety.

### BACKGROUND OF THE INVENTION

### Field of the Invention

The present invention relates to Tri-Specific Binding Molecules, which are multi-chain polypeptide molecules that possess three Binding Domains and are thus capable of mediating coordinated binding to three epitopes. The Binding Domains may be selected such that the Tri-Specific Binding Molecules are capable of binding to any three different epitopes. Such epitopes may be epitopes of the same antigen or epitopes of two or three different antigens. In a preferred embodiment, one of such epitopes will be capable of binding to CD3, the second of such epitopes will be capable of binding to CD8, and the third of such epitopes will be capable of binding to an epitope of a Disease-Associated Antigen. The invention also provides a novel ROR1-binding antibody, as well as derivatives thereof and uses for such compositions.

### Description of Related Art

### I. The Mammalian Immune System

The mammalian immune system serves as a defense against a variety of conditions, including, e.g., injury, infection and neoplasia. The efficiency with which humans and other mammals develop an immunological response to pathogens, foreign substances and cancer antigens rests on two characteristics: the exquisite specificity of the immune response for antigen recognition, and the immunological memory that allows for faster and more vigorous responses upon re-activation with the same antigen (Portolés, P. et al. (2009) “The TCR/CD3 Complex: Opening the Gate to Successful Vaccination,” Current Pharmaceutical Design 15:3290-3300; Guy, C. S. et al. (2009) “Organization of Proximal Signal Initiation at the TCR:CD3 Complex,” Immunol Rev. 232(1):7-21).

The mammalian immune system is mediated by two separate but interrelated systems: the cellular and humoral immune systems. Generally speaking, the humoral system is mediated by soluble products (antibodies or immunoglobulins) that have the ability to combine with and neutralize products recognized by the system as being foreign to the body. In contrast, the cellular immune system involves the mobilization of certain cells, termed “T cells,” that serve a variety of therapeutic roles. T cells are lymphocytes that are derived from the thymus and circulate between the tissues, lymphatic system and the circulatory system. In response to the presence and recognition of foreign structures (antigens), T cells become “activated” to initiate an immune response. In many instances these foreign antigens are expressed on host cells as a result of neoplasia or infection. Although T cells do not themselves secrete antibodies, they are usually required for antibody secretion by the second class of lymphocytes, B cells (which derive from bone marrow). Critically, T cells exhibit extraordinary immunological specificity so as to be capable of discerning one antigen from another). Two types of T cells, “T helper cells” and “cytotoxic T cells,” are of particular relevance.

T helper cells are characterized by their expression of the glycoprotein, CD4 (i.e., they are “CD4+”). CD4⁺ T cells are the essential organizers of most mammalian immune and autoimmune responses (Dong, C. et al. (2003) “Immune Regulation by Novel Costimulatory Molecules,” Immunolog. Res. 28(1):39-48). The activation of CD4⁺ T cells has been found to be mediated through co-stimulatory interactions between an antigen:major histocompability class II (MHC II) molecule complex that is arrayed on the surface of an Antigen Presenting Cell (such as a B cell, a macrophage or a dendritic cell) and a complex of two molecules, the T Cell Receptor (“TCR”) and a CD3 cell-surface receptor ligand, that are arrayed on surface of a naive CD4⁺ T cell. Activated T helper cells are capable of proliferating into Th1 cells that are capable of mediating an inflammatory response to the target cell.

Cytotoxic T cells are characterized by their expression of CD8 (i.e., they are “CD8+” as well as CD3⁺). The activation of CD8⁺ T cells has been found to be mediated through co-stimulatory interactions between an antigen:major histocompability class I (MHC I) molecule complex that is arrayed on the surface of a target cell and a complex of CD8 and the T Cell Receptor, that are arrayed on surface of the CD8⁺ T cell. Unlike MHC II molecules, which are expressed by only certain immune system cells, MHC I molecules are very widely expressed. Thus, cytotoxic T cells are capable of binding to a wide variety of cell types. Activated cytotoxic T cells mediate cell killing through their release of the cytotoxins perforin, granzymes, and granulysin. Through the action of perforin, granzymes enter the cytoplasm of the target cell and their serine protease function triggers the caspase cascade, which is a series of cysteine proteases that eventually lead to apoptosis (programmed cell death) of targeted cells.

The T cell receptor (“TCR”) is a covalently linked heterodimer of α and β chains (“TCRαρ”). These chains are class I membrane polypeptides of 259 (α) and 296 (β) amino acids in length. The CD3 molecule is a T cell co-receptor composed of five distinct polypeptide chains (a CD3 γ chain, a CD3 δ chain, two CD3 ε chains and two zeta chains). The individual polypeptide chains associate to form a complex of three dimers (εγ, εδ, ζζ) (Wucherpfennig, K. W. et al. (2010) “Structural Biology Of The T Cell Receptor: Insights into Receptor Assembly, Ligand Recognition, And Initiation of Signaling,” Cold Spring Harb. Perspect. Biol. 2(4):a005140; pages 1-14; Chetty, R. et al. (1994) “CD3: Structure, Function And The Role Of Immunostaining In Clinical Practice,” J. Pathol. 173:303-307; Guy, C. S. et al. (2009) “Organization of Proximal Signal Initiation at the TCR:CD3 Complex,” Immunol Rev. 232(1):7-21; Call, M. E. et al. (2007) “Common Themes In The Assembly And Architecture Of Activating Immune Receptors,” Nat. Rev. Immunol. 7:841-850; Weiss, A. (1993) “T Cell Antigen Receptor Signal Transduction: A Tale Of Tails And Cytoplasmic Protein-Tyrosine Kinases,” Cell 73:209-212). The CD3 complex associates with TCR in order to generate an activation signal in T lymphocytes. In the absence of CD3, TCRs do not assemble properly and are degraded (Thomas, S. et al. (2010) “Molecular Immunology Lessons From Therapeutic T Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 February; 24(2):133-139).

The TCR and CD3 complex, along with the CD3 (chain zeta chain (also known as T cell receptor T3 zeta chain or CD247) comprise the TCR complex (van der Merwe, P. A. etc. (epub Dec. 3, 2010) “Mechanisms For T Cell Receptor Triggering,” Nat. Rev. Immunol. 11:47-55; Wucherpfennig, K. W. et al. (2010) “Structural Biology of the T cell Receptor: Insights into Receptor Assembly, Ligand Recognition, and Initiation of Signaling,” Cold Spring Harb. Perspect. Biol. 2:a005140). The complex is particularly significant since it contains a large number (ten) of immunoreceptor tyrosine-based activation motifs (ITAMs).

Two interactions are required for T cell activation (Viglietta, V. et al. (2007) “Modulating Co-Stimulation,” Neurotherapeutics 4:666-675; Korman, A. J. et al. (2007) “Checkpoint Blockade in Cancer Immunotherapy,” Adv. Immunol. 90:297-339). In the first interaction, a Cell must display the relevant target antigen bound to the cell's Major Histocompatibility Complex so that it can bind to the T cell Receptor (“TCR”) of a naive T lymphocyte. In the second interaction, a ligand of the Cell must bind to a co-receptor of the T lymphocyte (Dong, C. et al. (2003) “Immune Regulation by Novel Costimulatory Molecules,” Immunolog. Res. 28(1):39-48; Lindley, P. S. et al. (2009) “The Clinical Utility Of Inhibiting CD28-Mediated Costimulation,” Immunol. Rev. 229:307-321). T cells experiencing both stimulatory signals are then capable of responding to cytokines (such as Interleukin-2 and Interleukin-12). In the absence of both co-stimulatory signals during TCR engagement, T cells enter a functionally unresponsive state, referred to as clonal anergy (Khawli, L. A. et al. (2008) “Cytokine, Chemokine, and Co-Stimulatory Fusion Proteins for the Immunotherapy of Solid Tumors,” Exper. Pharmacol. 181:291-328). In pathologic states, T cells are the key players of various organ-specific autoimmune diseases, such as type I diabetes, rheumatoid arthritis, and multiple sclerosis (Dong, C. et al. (2003) “Immune Regulation by Novel Costimulatory Molecules,” Immunolog. Res. 28(1):39-48). E

The need for two signals to activate T cells such that they achieve an adaptive immune response is believed to provide a mechanism for avoiding responses to self-antigens that may be present on an Antigen Presenting Cell at locations in the system where it can be recognized by a T cell. Where contact of a T cell with a Cell results in the generation of only one of two required signals, the T cell does not become activated and an adaptive immune response does not occur.

### II. Antibodies and Other Epitope-Binding Molecules

### A. Antibodies

“Antibodies” are immunoglobulin molecules capable of specific binding to a target, such as a carbohydrate, polynucleotide, lipid, polypeptide, etc., through at least one antigen recognition site, located in the Variable Domain of the immunoglobulin molecule. As used herein, the term encompasses not only intact polyclonal or monoclonal antibodies, but also mutants thereof, naturally occurring variants, fusion proteins comprising an antibody portion with an antigen recognition site of the required specificity, humanized antibodies, and chimeric antibodies, and any other modified configuration of the immunoglobulin molecule that comprises an antigen recognition site of the required specificity. Throughout this application, the numbering of amino acid residues of the light and heavy chains of antibodies is according to the EU index as in Kabat et al. (1992) SEQUENCES OF PROTEINS OF IMMUNOLOGICAL INTEREST, National Institutes of Health Publication No. 91-3242. As used herein, an “antigen-binding fragment of an antibody” is a portion of an antibody that possesses an at least one antigen recognition site. As used herein, the term encompasses fragments (e.g., Fab, Fab′, F(ab′)₂ Fv), and single-chain molecules (e.g., scFv).

Natural antibodies (such as IgG antibodies) are composed of two Light Chains complexed with two Heavy Chains. Each Light Chain contains a Variable Domain (VL) and a Constant Domain (CL). Each heavy chain contains a Variable Domain (VH), three Constant Domains (CH1, CH2 and CH3), and a Hinge Domain located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N”) portion of each chain includes a variable region of about 100 to 110 or more amino acids primarily responsible for antigen recognition. The carboxy-terminal (“C”) portion of each chain defines a constant region, with light chains having a single Constant Domain and heavy chains usually having three Constant Domains and a hinge region. Thus, the structure of the light chains of an IgG molecule is n-VL-CL-c and the structure of the IgG heavy chains is n-VH-CH1-H-CH2-CH3-c (where H is the hinge region, and n and c represent, respectively, the N-terminus and the C-terminus of the polypeptide).

The ability of an intact, unmodified antibody (e.g., an IgG antibody) to bind an epitope of an antigen depends upon the presence of Variable Domains on the immunoglobulin light and heavy chains (i.e., the VL Domain and VH Domain, respectively). Interaction of an antibody Light Chain and an antibody heavy chain and, in particular, interaction of its VL and VH Domains forms one of the epitope-binding sites of the antibody. The variable regions of an IgG molecule consist of the complementarity determining regions (CDR), which contain the residues in contact with epitope, and non-CDR segments, referred to as framework segments (FR), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of an antibody Light Chain are herein respectively designated CDR_{L}1 Domain, CDR_{L}2 Domain, and CDR_{L}3 Domain. Similarly, polypeptides that are (or may serve as) the first, second and third CDR of an antibody heavy chain are herein respectively designated CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to an specific epitope regardless of whether such protein is an antibody having light and heavy chains or a diabody or a single-chain binding molecule (e.g., an scFv, a BiTe, etc.), or is another type of protein. In contrast to such antibodies, the scFv construct comprises a VL and VH Domain of an antibody contained in a single polypeptide chain wherein the Domains are separated by a flexible linker of sufficient length to allow self-assembly of the two Domains into a functional epitope-binding site. Where self-assembly of the VL and VH Domains is rendered impossible due to a linker of insufficient length (less than about 12 amino acid residues), two of the scFv constructs may interact with one another other to form a bivalent molecule in which the VL of one chain associates with the VH of the other (reviewed in Marvin et al. (2005) “Recombinant Approaches To IgG-Like Bispecific Antibodies,” Acta Pharmacol. Sin. 26:649-658).

In addition to their known uses in diagnostics, antibodies have been shown to be useful as therapeutic agents. The last few decades have seen a revival of interest in the therapeutic potential of antibodies, and antibodies have become one of the leading classes of biotechnology-derived drugs (Chan, C. E. et al. (2009) “The Use Of Antibodies In The Treatment Of Infectious Diseases,” Singapore Med. J. 50(7):663-666). Nearly 200 antibody-based drugs have been approved for use or are under development.

The term “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single epitope (or antigenic site). The term “monoclonal antibody” encompasses not only intact monoclonal antibodies and full-length monoclonal antibodies, but also fragments thereof (such as Fab, Fab′, F(ab′)₂ Fv), single-chain (scFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody.” Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125).

In general, cells should be kept intact and preferably viable when used as immunogens. Intact cells may allow antigens to be better detected than ruptured cells by the immunized animal. Use of denaturing or harsh adjuvants, e.g., Freud's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate a chimeric antibody, a humanized antibody, or a caninized antibody, or to improve the affinity, or other characteristics of the antibody. The general principle in humanizing an antibody involves retaining the basic sequence of the antigen-binding portion of the antibody, while swapping the non-human remainder of the antibody with human antibody sequences. There are four general steps to humanize a monoclonal antibody. These are: (1) determining the nucleotide and predicted amino acid sequence of the starting antibody light and heavy variable Domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

The epitope-binding domain of such antibodies may comprise either complete Variable Domains fused onto Constant Domains or only the complementarity determining regions (CDRs) grafted onto appropriate framework regions in the Variable Domains. Antigen-binding sites may be wild-type or modified by one or more amino acid substitutions. This eliminates the constant region as an immunogen in human individuals, but the possibility of an immune response to the foreign variable region remains (LoBuglio, A. F. et al. (1989) “Mouse Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable regions as well so as to reshape them as closely as possible to human form. It is known that the variable regions of both heavy and light chains contain three complementarity determining regions (CDRs) which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable regions can be “reshaped” or “humanized” by grafting CDRs derived from non-human antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Natl. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, or six) which differ in sequence relative to the original antibody.

### B. Bi-Specific Antibodies, Multi-Specific Diabodies and DART™ Diabodies

Natural antibodies are capable of binding to only one epitope species (i.e., they are “mono-specific”), although they may be able to bind multiple copies of that species (i.e., they may exhibit bi-valency or multi-valency). A wide variety of recombinant bi-specific antibody formats have been developed (see, e.g., PCT Publication Nos. WO 2008/003116, WO 2009/132876, WO 2008/003103, WO 2007/146968, WO 2007/146968, WO 2009/018386, WO 2012/009544, WO 2013/070565), most of which use linker peptides either to fuse the antibody core (IgA, IgD, IgE, IgG or IgM) to a further binding protein (e.g., scFv, VL VH, etc.) to, or within, the antibody core, or to fuse multiple antibody portions or to fuse (e.g. two Fab fragments or scFv) to a Heterodimerization-Promoting Domain such as the CH2-CH3 Domain or alternative polypeptides (WO 2005/070966, WO 2006/107786A WO 2006/107617A, WO 2007/046893). Typically, such approaches involve compromises and trade-offs. For example, PCT Publications Nos. WO 2013/174873, WO 2011/133886 and WO 2010/136172 disclose that the use of linkers may cause problems in therapeutic settings, and teaches a tri-specific antibody in which the CL and CH1 Domains are switched from their respective natural positions and the VL and VH Domains have been diversified (WO 2008/027236; WO 2010/108127) to allow them to bind to more than one antigen. Thus, the molecules disclosed in these documents trade binding specificity for the ability to bind additional antigen species. PCT Publications Nos. WO 2013/163427 and WO 2013/119903 disclose modifying the CH2 Domain to contain a fusion protein adduct comprising a binding domain. The document notes that the CH2 Domain likely plays only a minimal role in mediating effector function. PCT Publications Nos. WO 2010/028797, WO2010028796 and WO 2010/028795 disclose recombinant antibodies whose Fc Domains have been replaced with additional VL and VH Domains, so as to form tri-valent binding molecules. PCT Publications Nos. WO 2003/025018 and WO2003012069 disclose recombinant diabodies whose individual chains contain scFv domains. PCT Publications No. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publications Nos. WO 2014/022540, WO 2013/003652, WO 2012/162583, WO 2012/156430, WO 2011/086091, WO 2007/075270, WO 1998/002463, WO 1992/022583 and WO 1991/003493 disclose adding additional Binding Domains or functional groups to an antibody or an antibody portion (e.g., adding a diabody to the antibody's Light Chain, or adding additional VL and VH Domains to the antibody's light and heavy chains, or adding a heterologous fusion protein or chaining multiple Fab Domains to one another). Thus, the molecules disclosed in these documents trade native antibody structure for the ability to bind additional antigen species.

The art has additionally noted the capability to produce diabodies that differ from such natural antibodies in being capable of binding two or more different epitope species (i.e., exhibiting bi-specificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Wu, A. et al. (2001) “Multimerization OfA Chimeric Anti-CD20 Single-chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T-Cell Engaging Antibodies For Cancer Therapy,” Cancer Res. 69(12):4941-4944).

The design of a diabody is based on the structure of single-chain Variable Domain fragments (scFv). Such molecules are made by linking light and/or Heavy Chain Variable Domains to one another via a short linking peptide. Bird et al. (1988) (“Single-Chain Antigen-Binding Proteins,” Science 242:423-426) describes an example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

U.S. Pat. No. 7,585,952 and United States Patent Publication No. 2010-0173978 concern scFv molecules that are immunospecific for ErbB2. Bi-specific T cell engagers (“BiTEs”), a type of scFv molecule has been described (WO 05/061547; Baeuerle, P et al. (2008) “BiTE: A New Class Of Antibodies That Recruit T Cells,” Drugs of the Future 33: 137-147; Bargou, et al. 2008) “Tumor Regression in Cancer Patients by Very Low Doses of a T Cell-Engaging Antibody,” Science 321: 974-977). Such molecules are composed of a single polypeptide chain molecule having two Antigen-Binding Domains, one of which immunospecifically binds to a CD3 epitope and the second of which immunospecifically binds to an antigen present on the surface of a target cell.

The provision of non-mono-specific diabodies provides a significant advantage: the capacity to co-ligate and co-localize cells that express different epitopes. Bivalent diabodies thus have wide-ranging applications including therapy and immunodiagnosis. Bi-valency allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligating of differing cells, for example, the cross-linking of cytotoxic T cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

Diabody epitope-binding domains may be directed to a surface determinant of any immune effector cell such as CD3, CD16, CD32, CD64, etc., which are expressed on T lymphocytes, Natural Killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bispecific Antibody Conjugates In Therapeutics,”wAdv. Drug. Deliv. Rev. 55:171-197).

For example, U.S. Pat. No. 6,171,586, concerns the production of bi-specific antibodies by proteolytically cleaving two antibodies to obtain their F(ab′)₂ fragments, reducing such fragments under conditions for preventing intermolecular disulfide bond formation, and then mixing the fragments to generate the bi-specific antibody). U.S. Pat. Nos. 6,551,592; 6,994,853 and 8,277,806 and PCT Publications Nos. WO 2012/156430, WO 2002/020039, WO 2000/018806 and WO 1998/003670 concern the production of tri-specific antibodies capable of simultaneously binding to T cells and other antigens on a tumor cell, and, via the Fc portion of the bi-specific antibody, to the Fc receptor of cells possessing such a receptor. PCT Publications Nos. WO 2000/018806, WO 1998/003670 and WO 2006/072152 concern the production of tri-specific antibodies capable of simultaneously binding to T cells and other antigens. United States Patent Publication No. 2008-0057054 discloses bi-specific conjugates specific for a binding element against amyloid beta oligomers and a binding element against transmembrane protein telencephalin. United States Patent Publication No. 2010-0291112 concerns bi-specific and tri-specific single-chain Fv molecules that specifically bind to a one (or two) tumor antigen(s) and an effector cell antigen (such as CD3, CD16 CD32, CD64, etc.).

PCT Publication Nos. WO 1999/042597 and WO 1998/006749 disclose antibody derivatives that comprise human Major Histocompatibility Complex binding domains, with or without bound MHC binding peptides. PCT Publication No. WO 02/072141 concerns multi-specific binding molecules whose on-rates (rates at which they bind to target molecules) and off-rates (rates at which they release target molecules) differ so as to preferentially bind to one target compared to their binding to the other such target molecule. Tri-specific molecules, for example molecules having a monovalent first portion which is an Anti-CD3 or anti-CD28 antibody, and a second portion comprising a divalent immune function exerting moiety which immunospecifically binds to one or more target ligands on a target diseased cell or immune cell.

U.S. Pat. No. 7,695,936 and Patent Publication 2007/0196363 concern bi-specific antibodies that are formed from the heavy chains of two antibodies, one of which possess a protuberance engineered into its heavy chain and the second of which possess a complementary cavity engineered into its heavy chain. The presence of such complementary “knobs” and “holes” is taught to preferentially form bi-specific hetero-antibodies (having one heavy chain of each such antibody) relative to mono-specific homo-antibodies that contain two heavy chains of the same antibody. Various bi-specific hetero-antibodies are proposed, including those that are immunospecific for CD3 and a tumor cell antigen. Various tri-specific hetero-antibodies are also proposed, including some that are immunospecific for CD3, CD8 and CD37 (a transmembrane protein expressed predominantly on B cells that is involved the regulation of T cell proliferation (Robak, T. et al. (2014) “Anti-CD37 Antibodies For Chronic Lymphocytic Leukemia,” Expert Opin. Biol. Ther. 14(5):651-661), however, no mechanism for their production and no disclosure of their structure is provided.

PCT Publication WO2012-162561 concerns bi-specific, tetravalent binding molecules that comprise two polypeptides, each of which comprises two diabody structures, separated by an intervening CH2-CH3 Domain. The document also concerns tetravalent binding molecules composed of four polypeptide chains in which two of the polypeptide chains contain variable light and variable heavy Domains for two antigens, and in which the other two polypeptide chains contain the complementary variable heavy and variable light Domains for the antigens and a terminal CH2-CH3 Domain. The bi-specific, tetravalent binding molecules form through the association of their respective CH2-CH3 Domains. In the four polypeptide chain construct, the “light” chains are not covalently bound to the heavy chains, thus leading to instability (see, Lu, D. et al. (2005) “A Fully Human Recombinant IgG-Iike Bispecific Antibody To Both The Epidermal Growth Factor Receptor And The Insulin-Like Growth Factor Receptor For Enhanced Antitumor Activity,” J. Biol. Chem. 280(20):19665-19672). The document discloses a third construct in which the chains are altered to provide such covalent bonding, but at the cost of eliminating their bi-specificity (i.e., the molecules are mono-specific). Molecules having specificity for CD2, CD3, CD4, CD8, CD161, a chemokine receptor, CD95, CCR5, etc. are disclosed. A bi-specific molecule capable of binding to both CD3 and CD8 is not disclosed.

However, the above advantages come at salient cost. The formation of such non-mono-specific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-mono-specific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bi-specific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-mono-specific diabodies, termed DARTs™ (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; and Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449). Such diabodies comprise two or more covalently complexed polypeptides and involve engineering one or more cysteine residues into each of the employed polypeptide species that permit disulfide bonds to form and thereby covalently bond two polypeptide chains. For example, the addition of a cysteine residue to the C-terminus of such constructs has been shown to allow disulfide bonding between the polypeptide chains, stabilizing the resulting heterodimer without interfering with the binding characteristics of the bivalent molecule.

There are many DART™ embodiments. Each of the two polypeptides of the simplest DART™ embodiment comprises three Domains (FIG. 1A). The first polypeptide comprises: (i) a first domain that comprises a binding region of a Light Chain Variable Domain of the a first immunoglobulin (VL1), (ii) a second domain that comprises a binding region of a Heavy Chain Variable Domain of a second immunoglobulin (VH2), and (iii) a third domain that contains a cysteine residue (or a Cysteine-Containing Domain) and a Heterodimerization-Promoting Domain that serves to promote heterodimerization with the second polypeptide chain (FIG. 1B). The cysteine residue (or a Cysteine-Containing Domain) of the third domain serves to promote the covalent bonding of the first polypeptide chain to the second polypeptide chain of the diabody. The second polypeptide contains: (i) a complementary first domain (a VL2-containing Domain), (ii) a complementary second domain (a VH1-containing Domain) and (iii) a third domain that contains a cysteine residue (or a Cysteine-Containing Domain) and, optionally, a complementary Heterodimerization-Promoting Domain that complexes with the Heterodimerization-Promoting Domain of the first polypeptide chain in order to promote heterodimerization with the first polypeptide chain. The cysteine residue (or a Cysteine-Containing Domain) of the third domain of the second polypeptide chain serves to promote the covalent bonding of the second polypeptide chain to the first polypeptide chain of the diabody. Such molecules are stable, potent and have the ability to simultaneously bind two or more antigens. They are able to promote re-directed T cell mediated killing of cells expressing target antigens.

In one embodiment, the third domains of the first and second polypeptides each contain a cysteine residue, which serves to bind the polypeptides together via a disulfide bond. The third domain of one or both of the polypeptides may additionally possesses the sequence of a CH2-CH3 Domain, such that complexing of the diabody polypeptides forms an Fc Domain that is capable of binding to the Fc receptor of cells (such as B lymphocytes, dendritic cells, Natural Killer cells, macrophages, neutrophils, eosinophils, basophils and mast cells) (FIGS. 2A-2B).

Many variations of such molecules have been described (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538). These Fc-bearing DARTs may comprise three polypeptide chains (e.g., FIG. 2B). The first polypeptide chain of such a diabody contains three domains: (i) a VL1-containing Domain, (ii) a VH2-containing Domain and (iii) a domain containing a cysteine residue (or a Cysteine-Containing Domain) and a Heterodimerization-Promoting Domain, and (iv) a cysteine residue (or a Cysteine-Containing Domain and a CH2-CH3 Domain. The second polypeptide chain of such DART™ contains: (i) a VL2-containing Domain, (ii) a VH1-containing Domain and (iii) a Domain that contains a cysteine residue (or a Cysteine-Containing Domain) and a Heterodimerization-Promoting Domain that promotes heterodimerization with the first polypeptide chain. The cysteine residue (or a Cysteine-Containing Domain) of the third domain of the second polypeptide chain serves to promote the covalent bonding of the second polypeptide chain to the first polypeptide chain of the diabody. The third polypeptide of such DART™ comprises a cysteine residue (or a Cysteine-Containing Domain) and a CH2-CH3 Domain. Thus, the first and second polypeptide chains of such DART™ associate together to form a VL1/VH1 binding site that is capable of binding to the epitope, as well as a VL2/VH2 binding site that is capable of binding to the second epitope. The first and second polypeptides are bonded to one another through a disulfide bond involving cysteine residues in their respective third domains. Notably, the first and third polypeptide chains complex with one another to form an Fc Domain that is stabilized via a disulfide bond. Such diabodies have enhanced potency. Such Fc-bearing DARTs™ may have either of two orientations (Table 1):

TABLE 1

| | | |
|---|---|---|
| First | 3^{rd} Chain | NH₂—CH2—CH3—COOH |
| Orien- | 1^{st} Chain | NH₂-VL1-VH2-Cys-Heterodimer- |
| tation | | Promoting Domain-CH2—CH3—COOH |
| | 2^{nd} Chain | NH₂-VL2-VH1-Cys-Heterodimer- |
| | | Promoting Domain-COOH |
| Second | 3^{rd} Chain | NH₂—CH2—CH3—COOH |
| Orien- | 1^{st} Chain | NH₂—CH2—CH3-VL1-VH2-Cys- |
| tation | | Heterodimer-Promoting Domain-COOH |
| | 2^{nd} Chain | NH₂-VL2-VH1-Cys-Heterodimer- |
| | | Promoting Domain-COOH |

Even more complex DART™ diabodies, termed Ig-DART™ (FIGS. 3A-3B) and Fc-DART™ diabodies (FIG. 3C) have been described (WO 2012/018687). Fc-DARTs™ have four polypeptide chains. The first and third polypeptide chains of such a diabody contain three Domains: (i) a VL1-containing Domain, (ii) a VH2-containing Domain and (iii) a Domain containing a CH2-CH3 sequence. The second and fourth polypeptide of the Fc-DART™ contain: (i) a VL2-containing Domain, (ii) a VH1-containing Domain and (iii) a Domain that promotes heterodimerization and covalent bonding with the Fc-DART's™ first polypeptide chain. The third and fourth, and the first and second polypeptide chains may be the same or different so as to permit tetravalent binding that is either mono-specific, bi-specific or tetra-specific. Such more complex DART™ molecules also possess Cysteine-Containing Domains which function to form a covalently bonded complex. Fc-DART™ diabodies contain CH1 and CL Domains.

Alternative constructs are known in the art for applications where a tetravalent molecule is desirable but an Fc is not required including, but not limited to, tetravalent tandem antibodies, also referred to as “TandAbs” (see, e.g. United States Patent Publications Nos. 2005-0079170, 2007-0031436, 2010-0099853, 2011-020667 2013-0189263; European Patent Publication Nos. EP 1078004, EP 2371866, EP 2361936 and EP 1293514; PCT Publications Nos. WO 1999/057150, WO 2003/025018, and WO 2013/013700) which are formed by the homo-dimerization of two identical chains each possessing a VH1, VL2, VH2, and VL2 Domain.

### III. Re-Directed Killing

As discussed above, interactions between CD8, the MHC I and the T Cell Receptor lead to the activation of cytotoxic T cells and their ability to kill nearby cells. Bi-specific diabodies that bind to CD3 and to a tumor antigen may be used to co-localize cytotoxic CD8+ T cells to the tumor cells, achieving a “re-directed killing” of such cells (WO 2010/080538, WO 2012/018687, WO/2012/162068, US 2010/0174053; US 2013/0295121).

However, efforts to treat cancer or infectious disease by co-localizing CD3⁺ T cells to the locus of tumor or pathogen cells have not been fully successful. Antibodies that target CD3 bind to both CD3⁺ CD8⁺ cytotoxic T cells and to CD3⁺ CD4⁺ T helper cells, leading to the activation of both such cells. The cytokines produced by activated CD3+CD4+T helper cells, however, contribute to severe side-effects, e.g., life-threatening cytokine storms (Ferran, C. et al. (1990) “Cytokine-Related Syndrome Following Injection Of Anti-CD3 Monoclonal Antibody: Further Evidence For Transient In Vivo T Cell Activation,” Eur. J. Immunol. 20:509-515). Additionally, such anti-CD3 antibodies bind to other cell types, including CD3⁺ CD4⁻ CD8⁻ double negative T cells, etc. which express cytokines upon activation (Johansson, Martina et al. (2003) “A Unique Population of Extrathymically Derived αβTCR⁺ CD4⁻ CD8⁻ T Cells with Regulatory Functions Dominates the Mouse Female Genital Tract,” J. Immunol. 170:1659-1666; Blank, C. et al. (2003) “Absence of Programmed Death Receptor 1 Alters Thymic Development and Enhances Generation of CD4/CD8 Double-Negative TCR-Transgenic T Cells,” J. Immunol. 171:4574-4581; McIntyre, M. S. F. et al. (2011) “Consequences Of Double Negative Regulatory T Cell And Antigen Presenting Cell Interaction On Immune Response Suppression,” Intl. Immunopharmacol. 11:597-603), and which suppress the cytotoxicity mediated by CD3⁺ CD8⁺ T cells (Hillhouse, E. E. (2013) “A Comprehensive Review Of The Phenotype And Function Of Antigen-Specific Immunoregulatory Double Negative T Cells,” J. Autoimmun. 40:58-65).

It has been proposed that cytokine production associated with the administration of antibodies that target CD3 could be avoided using bi-specific antibodies that target CD8 and the tumor antigen (Michalk, I. et al. (2014) “Characterization of a Novel Single-Chain Bispecific Antibody for Retargeting of T Cells to Tumor Cells via the TCR Co-Receptor CD8,” PLOS One 9(4):e95517, pages 1-8). Anti-CD8 antibodies have therefore been studied to determine whether they would be capable of inducing effector function when used alone. Clement, M. et al. reported that six of seven anti-human CD8 antibodies tested failed to activate CD8⁺ T cells, but that such activation could be achieved using very high concentrations (10-100 μg/mL) of the anti-human CD8 antibody “OKT8” (Clement, M. et al. (2011) “Anti-CD8 Antibodies Can Trigger CD8⁺ T Cell Effector Function In The Absence Of TCR Engagement And Improve Peptide-MHCI Tetramer Staining,” J. Immunol. 187(2):654-663). Cooperative binding to two CD8 molecules was required for such an effect, since OKT8 F(ab)′₂ fragments were found to be able to mediate the effect, whereas OKT8 Fab were found to be incapable of doing so.

Thus, despite such studies, the cytokine-mediated toxicity attending to the use of anti-CD4 or anti-CD8 antibodies has not been fully understood. Studies have revealed that the cytokine toxicity seen upon administration of anti-CD3 antibody is not eliminated by depleting CD3⁺ CD4⁺ T cells or by deleting CD3⁺ CD8⁺ T cells. Thus, both CD3⁺ CD4⁺ T cells and CD3⁺ CD8⁺ T cells contribute to the toxic effects of anti-CD3 antibodies, and relatively few cells are required to mediate the full effect (Finck, B. K. et al. (1992) “The Role Of T-Cell Subsets In The Response To Anti-CD3 Monoclonal Antibodies,” Clin Immunol Immunopathol. 1992 December; 65(3):234-41).

Moreover, a bi-specific antibody that targets CD8 and a tumor antigen is not specific for CD3⁺ CD8⁺ T cells and tumor cells, but rather is specific only for CD8⁺ cells and tumor cells. In particular, the CD3⁻ CD8⁺ subset of Natural Killer (NK) cells would be targeted by such an antibody. Such cells, which represent a majority of NK cells are potent producers of cytokines and their activation would likely contribute to a cytokine storm. CD3⁻ CD8⁺ NK cells are the primary source of IFN-γ in HIV-1-infected chimpanzees (Rodriquez, A. R. et al. (2007) “Influence Of Interleukin-15 On CD8+ Natural Killer Cells In Human Immunodeficiency Virus Type 1-Infected Chimpanzees,” J. Gen. Virol. 88:641-651).

Consequently, despite all prior advances, a need remains for improved compositions capable of more vigorously directing the body's immune system to attack cancer cells or pathogen-infected cells, especially at lower therapeutic concentrations. As described in detail below, the present invention addresses this need by providing Tri-Specific Binding Molecules that bind to: (1) an epitope of CD3, (2) an epitope of CD8, and (3) an epitope of a Disease-Associated Antigen that is expressed on a target cell (especially a cancer cell, or a pathogen-infected cell) and mediate coordinated binding of cytotoxic T cells to cells presenting the Disease-Associated Antigen.

### SUMMARY OF THE INVENTION

The present invention relates to Tri-Specific Binding Molecules, which are multi-chain polypeptide molecules that possess three Binding Domains and are thus capable of mediating coordinated binding to three epitopes. The Binding Domains may be selected such that the Tri-Specific Binding Molecules are capable of binding to any three different epitopes. Such epitopes may be epitopes of the same antigen or epitopes of two or three different antigens. The invention also provides a novel ROR1-binding antibody, as well as derivatives thereof and uses for such compositions.

The present invention particularly relates to the embodiment of such Tri-Specific Binding Molecules in which the three epitopes are selected such that one or two of such epitopes are epitope(s) of an immune system cell, and especially, a cytotoxic lymphocyte immune system cell (CTL), and in which the remaining epitope(s) are epitope(s) of a Disease-Associated Antigen. Such particularly preferred Tri-Specific Binding Molecules are capable of localizing a cytotoxic lymphocyte cell to a cell that expresses a Disease-Associated Antigen, and of thereby facilitating the killing of cells that express the Disease-Associated Antigen. The Disease-Associated Antigen may be a cancer antigen, or may be an antigen that is characteristic of a pathogen (e.g., bacterial, fungal, viral or protozoan) infection. More particularly, the invention relates to such Tri-Specific Binding Molecules that are capable of mediating coordinated binding to: (1) an epitope of CD3, (2) an epitope of CD8, and (3) an epitope of a Disease-Associated Antigen. By binding to CD3 and CD8, and to the Disease-Associated Antigen, such molecules co-localize cytotoxic T cells to cells presenting the Disease-Associated Antigen, leading to the activation of such T cells and the initiation of a cytotoxic response against cells expressing the Disease-Associated Antigen.

In detail, the invention provides a Tri-Specific Binding Molecule capable of immunospecifically binding to three different epitopes, wherein the binding molecule comprises four different polypeptide chains covalently complexed together and comprises:
(I) an Antigen-Binding Domain I that is capable of immunospecifically binding to an Epitope I present on a first antigen, and an Antigen-Binding Domain II that is capable of immunospecifically binding to an Epitope II present on a second antigen, wherein the Antigen-Binding Domain I and the Antigen-Binding Domain II are both Diabody-Type Binding Domains;
(II) a Non-Diabody-Type Antigen-Binding Domain III that is capable of immunospecifically binding to an Epitope III present on a third antigen; and
(III) an Fc Domain that is formed by the association of two CH2-CH3 Domains to one another.
wherein the first, second and third antigens are the same antigen, or are independently the same or different from another of the antigens.

The invention particularly concerns the embodiment of such Tri-Specific Binding Molecule, wherein one of Epitope I, Epitope II or Epitope III is an epitope of a cellular receptor.

The invention additionally concerns the embodiments of such Tri-Specific Binding Molecules, wherein one of Epitope I, Epitope II or Epitope III is an epitope of a Disease-Associated Antigen (and especially wherein the Disease-Associated Antigen is a cancer antigen that is arrayed on the surface of a cancer cell, or is a pathogen antigen that is arrayed on the surface of a pathogen or pathogen-infected cell).

The invention additionally concerns the embodiments of such Tri-Specific Binding Molecules, wherein the Fc Domain is capable of binding to an Fc Receptor arrayed on the surface of a cell.

The invention especially concerns the embodiments of such Tri-Specific Binding Molecules, wherein one of Epitope I, Epitope II or Epitope III is an epitope of CD3, a second of Epitope I, Epitope II or Epitope III is an epitope of CD8, and the third of Epitope I, Epitope II or Epitope III is an epitope of the Disease-Associated Antigen, and wherein the Antigen-Binding Domains I, II and III of the Tri-Specific Binding Molecules mediate coordinated binding of a cytotoxic T cell and a cell expressing the Disease-Associated Antigen. The invention particularly concerns the embodiments of such Tri-Specific Binding Molecules, wherein the CD3, the CD8 are arrayed on the surface of a T cell and wherein the Disease-Associated Antigen is arrayed on the surface of a cancer cell, pathogen or pathogen-infected cell, and wherein the immunospecific binding is sufficient to co-localize the CD3 and the CD8, and the Disease-Associated Antigen, thereby facilitating the activation of the CD8-arraying T cell against the Disease-Associated Antigen-arraying cell.

The invention additionally concerns the embodiments of above-described Tri-Specific Binding Molecules, wherein the Non-Diabody-Type Binding Domain III comprises the Fab-Type Binding Domain (VL_{III}/VH_{III}) that is capable of immunospecifically binding to the Epitope III, wherein the molecule comprises:
(A) a first polypeptide chain:
   (I) that comprises in the N-terminus to C-terminus direction:
      (1) a Light Chain Variable Domain of an immunoglobulin capable of binding to a first of the three epitopes (VL_{I});
      (2) a Heavy Chain Variable Domain of an immunoglobulin capable of binding to a second of the three epitopes (VH_{II});
      (3) (a) a first Cysteine-Containing Domain; and a Heterodimer-Promoting Domain; or
         (b) a Cysteine-Containing Heterodimer-Promoting Domain;
      (5) a second Cysteine-Containing Domain; and
      (6) CH2 and CH3 Domains of an IgG;
   or
   (II) that comprises in the N-terminus to C-terminus direction:
      (1) a first Cysteine-Containing Domain;
      (2) CH2 and CH3 Domains of an IgG;
      (3) a Light Chain Variable Domain of an immunoglobulin capable of binding to a first of the three epitopes (VL_{I});
      (4) a Heavy Chain Variable Domain of an immunoglobulin capable of binding to a second of the three epitopes (VHr);
      (5) (a) a second Cysteine-Containing Domain; and a Heterodimer-Promoting Domain; or
         (b) a Cysteine-Containing Heterodimer-Promoting Domain;
(B) a second polypeptide chain that comprises, in the N-terminus to C-terminus direction:
   (1) a Light Chain Variable Domain of an immunoglobulin capable of binding to the second of the three epitopes (VL_{I});
   (2) a Heavy Chain Variable Domain of an immunoglobulin capable of binding to the first of the three epitopes (VH_{I});
   (3) (a) a first Cysteine-Containing Domain; and a Heterodimer-Promoting Domain; or
      (b) a Cysteine-Containing Heterodimer-Promoting Domain;
      wherein the Heterodimer-Promoting Domain of the second polypeptide chain is complementary to the Heterodimer-Promoting Domain of the first polypeptide chain;
(C) a third polypeptide chain that comprises, in the N-terminus to C-terminus direction:
   (1) a Heavy Chain Variable Domain of an immunoglobulin capable of binding to a third of the three epitopes (VH_{III}); and
   (2) a CH1 Domain, a Cysteine-Containing Hinge Domain, and a CH2-CH3 Domain of an IgG;
and
(D) a fourth polypeptide chain that comprises, in the N-terminus to C-terminus direction:
   (1) a Light Chain Variable Domain of an immunoglobulin capable of binding to the third of the three epitopes (VL_{III}); and
   (2) a Cysteine-Containing Light Chain Constant Domain (CL);
wherein:
(i) the VL_{I} and VH_{I} Domains associate to form a Domain capable of binding the Epitope I;
(ii) the VL_{II} and VH_{II} Domains associate to form a Domain capable of binding the Epitope II;
(iii) the VL_{III} and VH_{III} Domains associate to form a Domain capable of binding the Epitope III;
(iv) the CH2-CH3 Domain of the first polypeptide chain and the CH2-CH3 Domain of the third polypeptide chain associate to form an Fc Domain;
(v) the first and second polypeptide chains are covalently bonded to one another;
(vi) the first and third polypeptide chains are covalently bonded to one another; and
(vii) the third and fourth polypeptide chains are covalently bonded to one another.

The invention additionally concerns the embodiments of above-described Tri-Specific Binding Molecules, wherein:
(A) the Heterodimer-Promoting Domain is an E-coil and the complementary Heterodimer-Promoting Domain is a K-coil; or
(B) the Heterodimer-Promoting Domain is a K-coil and the complementary Heterodimer-Promoting Domain is an E-coil.

The invention additionally concerns the embodiments of above-described Tri-Specific Binding Molecules, wherein:
(A) the CH2-CH3 Domains of the first and third polypeptide chains each have the sequence of SEQ ID NO:6, such that the Fc Domain formed from their association exhibits normal FcγR-mediated effector function; or
(B) the CH2-CH3 Domain of the first and third polypeptide chains comprise at least one amino acid substitution, relative to the sequence of SEQ ID NO:6, such that the Fc Domain formed from their association exhibits altered FcγR-mediated effector function.

The invention additionally concerns the embodiments of above-described Tri-Specific Binding Molecules, wherein the CH2-CH3 Domain of the first and third polypeptide chains differ from one another and have an amino acid sequence selected from the group consisting of SEQ ID NO:7 and SEQ ID NO:8.

The invention additionally concerns the embodiments of above-described Tri-Specific Binding Molecules, wherein:
(A) the Epitope I, Epitope II and Epitope III are, respectively, an epitope of CD3, an epitope of CD8 and an epitope of the Disease-Associated Antigen;
(B) the Epitope I, Epitope II and Epitope III are, respectively, an epitope of CD3, an epitope of the Disease-Associated Antigen and an epitope of CD8;
(C) the Epitope I, Epitope II and Epitope III are, respectively, an epitope of CD8, an epitope of CD3, and an epitope of the Disease-Associated Antigen;
(D) the Epitope I, Epitope II and Epitope III are, respectively, an epitope of CD8, an epitope of the Disease-Associated Antigen and an epitope of CD3;
(E) the Epitope I, Epitope II and Epitope III are, respectively, an epitope of the Disease-Associated Antigen, an epitope of CD3, and an epitope of CD8; or
(F) the Epitope I, Epitope II and Epitope III are, respectively, an epitope of the Disease-Associated Antigen, an epitope of CD8, and an epitope of CD3.

The invention additionally concerns the embodiments of above-described Tri-Specific Binding Molecules, wherein:
(A) the epitope of CD3 is a CD3 epitope recognized by antibodyOKT3, M291, YTH12.5, CD3 mAb 1 or CD3 mAb 2; or
(B) the epitope of CD8 is a CD8 epitope recognized by antibody TRX2 or OKT8.

The invention additionally concerns a pharmaceutical composition that comprises the above-described Tri-Specific Binding Molecule and a pharmaceutically acceptable carrier, excipient or diluent.

The invention additionally concerns a method of treating cancer which comprises administering an effective amount of the above-described pharmaceutical composition to an individual in need thereof, wherein the Disease-Associated Antigen is the cancer antigen.

The invention additionally concerns a method of treating a disease-associated with the presence of a pathogen which comprises administering an effective amount of the pharmaceutical composition of claim 15 to an individual in need thereof, wherein the Disease-Associated Antigen is the pathogen antigen.

The invention additionally concerns an anti-ROR1 antibody, or ROR1-binding fragment, wherein the antibody comprises:
(A) a Light Chain Variable Domain that comprises a CDR_{L}1 having the sequence of SEQ ID NO:117, a CDR_{L}2 having the sequence of SEQ ID NO:118, and a CDR_{L}3 having the sequence of SEQ ID NO:119; and
(B) a Heavy Chain Variable Domain that comprises a CDR_{H}1 having the sequence of SEQ ID NO:120, a CDR_{H}2 having the sequence of SEQ ID NO:121, and a CDR_{H}3 having the sequence of SEQ ID NO:122.

The invention additionally concerns the embodiments of such anti-ROR1 antibody or ROR1-binding fragment thereof, wherein the antibody has a Light Chain Variable Domain having the sequence of SEQ ID NO:51. The invention additionally concerns the embodiments of such anti-ROR1 antibodies or ROR1-binding fragments thereof, wherein the antibody has a Heavy Chain Variable Domain having the sequence of SEQ ID NO:52, or both a Light Chain Variable Domain having the sequence of SEQ ID NO:51 and a Heavy Chain Variable Domain having the sequence of SEQ ID NO:52.

The invention additionally concerns a diabody, BiTe or single-chain antibody that comprises the ROR1 binding fragment of any of such claims anti-ROR1 antibodies.

The invention additionally concerns a pharmaceutical composition that comprises any of the above-described anti-ROR1 antibodies or ROR1-binding fragments thereof and a pharmaceutically acceptable carrier, excipient or diluent. The invention additionally concerns a method of treating cancer which comprises administering an effective amount of such a pharmaceutical composition to an individual in need thereof.

### BRIEF DESCRIPTION OF THE FIGURES

FIGS. 1A-1B show diagrammatic representation of the Domains of DART™ diabodies. FIG. 1A shows a diagrammatic representation of the Domains of a basic DART™ diabody. FIG. 1B provides a schematic of a covalently bonded diabody composed of two polypeptide chains, each having a Heterodimer-Promoting Domain VL and VH domains that recognize the same epitope are shown using the same shading.

FIGS. 2A-2B provide a schematic of covalently bonded diabodies composed of two polypeptide chains, each having a CH2 and CH3 Domain (FIG. 2A) or in which only one has a CH2 and CH3 Domain (FIG. 2B), such that the associated chains form an Fc Domain that comprises all or part of a naturally occurring Fc Domain. VL and VH domains that recognize the same epitope are shown using the same shading.

FIGS. 3A-3C provide schematics showing tetravalent diabodies composed of two pairs of polypeptide chains. The pairs are different, thus resulting in a bi-specific molecule that is bivalent with respect to each of two epitopes, in which one is an epitope of DR5 and the other is an epitope of a molecule present on the surface of an effector cell. One polypeptide of each pair possesses a CH2 and CH3 Domain, such that the associated chains form an Fc Domain that comprises all or part of a naturally occurring Fc Domain. VL and VH domains that recognize the same epitope are shown using the same shading. Only one pair of epitopes (shown with the same shading) is capable of binding to DR5. FIG. 3A shows an Ig diabody. FIG. 3B shows an Ig diabody, which contains E-coil and K-coil heterodimer-promoting domains. FIG. 3C, shows an Fc-DART™ diabody that contains antibody CH1 and CL domains. The notation “VL1” and “VH1” denote respectively, the Variable Light Chain Domain and Variable Heavy Chain Domain that bind the “first” epitope. Similarly, the notation “VL2” and “VH2” denote respectively, the Variable Light Chain Domain and Variable Heavy Chain Domain that bind the “second” epitope.

FIGS. 4A-4L provide a diagrammatic representation of the Domains of preferred Tri-Specific Binding Molecules. FIGS. 4A and 4B, respectively, illustrate schematically the Domains of preferred Tri-Specific Binding Molecules in which the Tri-Specific Binding Molecule's Non-Diabody-Type Binding Domain is a Fab-Type Binding Domain or a T cell Receptor Binding Domain. FIGS. 4C and 4D, respectively, illustrate schematically the Domains of preferred Tri-Specific Binding Molecules having different Domain orientations in which the Non-Diabody-Type Binding Domain is a Fab-Type Binding Domain or a T Cell Receptor-Type Binding Domain. FIGS. 4E-4J depict similar molecules having three polypeptide chains. The molecule may possess Hinge and CL Domains (FIGS. 4E, 4H) or may contain an alternative linker peptide (FIG. 4F, 4I). FIGS. 4K-4L depict similar molecules having five polypeptide chains.

FIGS. 5A-5D show the ability of B7-H3 mAb 1/CD3 mAb 2/CD8 mAb 1 Tri-Specific Binding Molecule to bind to A498 target cells (FIG. 5A), and JIMT-1 target cells (FIG. 5B), CD5+/CD4− gated PBMCs (FIG. 5C) and CD5+/CD4+ gated PBMCs (FIG. 5D).

FIGS. 6A-6C demonstrate the ability of the Tri-Specific Binding Molecules of the present invention to mediate the re-directed killing of target cells. FIG. 6A shows the results of a luciferase assay of cell lysis of JIMT-1 cells. FIG. 6B shows the results of an LDH assay of cytotoxicity of JIMT-1 cells. FIG. 6C shows the results of an LDH assay of cytotoxicity of A498 cells.

FIGS. 7A-7D demonstrate the ability of the Tri-Specific Binding Molecules of the present invention to mediate T cell activation upon incubation with JIMT-1 cells (FIG. 7A: CD4/CD69 T cells; FIG. 7B: CD4/CD25 T cells; FIG. 7C: CD8/CD69 T cells; FIG. 7D: CD8/CD25 T cells).

FIGS. 8A-8D demonstrate the ability of the Tri-Specific Binding Molecules of the present invention to mediate T cell activation upon incubation with A498 cells (FIG. 8A: CD4/CD69 T cells; FIG. 8B: CD4/CD25 T cells; FIG. 8C: CD8/CD69 T cells; FIG. 8D: CD8/CD25 T cells).

FIGS. 9A-9B show the CD5⁺ CD4⁺ gated (FIG. 9A) or CD5⁺ CD4⁻ gated (FIG. 9B) cell populations of human PMBC as a function of increasing concentration of B7-H3 mAb 1/CD3 mAb 2/CD8 mAb 1 Tri-Specific Binding Molecules or B7-H3 mAb 1/CD3 mAb 2/CD8 mAb 2 Tri-Specific Binding Molecules. B7-H3×CD3 DARTs™ (with and without Fc Domain) were used as controls.

FIGS. 10A-10C show the effect of different CD8 Binding Domains on the cytotoxicity of a B7-H3 mAb 1/CD3 mAb 2/CD8 Tri-Specific Binding Molecule.

FIGS. 11A-11C demonstrate the ability to modulate the binding of the Tri-Specific Binding Molecules of the present invention by selecting Site A, Site B or Site C for the CD3 Binding Domain. The employed Tri-Specific Binding Molecules were capable of immunospecifically binding to the Disease-Associated Antigen, B7-H3. Cytotoxicity is measured using a luciferase assay.

FIGS. 12A-12C demonstrate the effect of positional selection (Site A, Site B or Site C) on the cytotoxicity mediated by the Tri-Specific Binding Molecules of the present invention using an LDH assay.

FIGS. 13A-13E show the effect of positional variation on cytotoxicity using a B7-H3 mAb 1/CD3 mAb 2/CD8 mAb 1 Tri-Specific Binding Molecule, a CD3 mAb 2/CD8 mAb 1/B7-H3 mAb 1 Tri-Specific Binding Molecule and a B7-H3 mAb 1/CD8 mAb 1/CD3 mAb 2 Tri-Specific Binding Molecule. A B7-H3×CD3 DART™ with Fc Domain was used as a control.

FIGS. 14A-14B, placement of the CD3 Binding Domain at Site C, greatly diminished binding to both the CD5⁺ CD4⁺ cells (FIG. 14A) and the CD5⁺CD4⁻ cells (FIG. 14B).

FIGS. 15A-15B show the CD5⁺ CD4⁺ gated (FIG. 15A) or CD5⁺ CD4⁻ gated (FIG. 15B) cell populations of human PMBC as a function of increasing concentration of 5T4 mAb 2/CD3 mAb 2/CD8 mAb 1 Tri-Specific Binding Molecules or 5T4 mAb2/CD3 mAb 2/CD8 mAb 2 Tri-Specific Binding Molecules. 5T4×CD3 DARTs™ (with and without Fc Domain) were used as controls.

FIGS. 16A-16C show that the observed effect of positional variation on cytotoxicity was not dependent on the employed CD8 Binding Domain.

FIGS. 17A-17C demonstrate the ability of Tri-Specific Binding Molecules of the present invention to mediate the re-directed killing of target cells expressing ROR1.

FIGS. 18A-18C show the ability of ability of HIV mAb 1/CD3 mAb 2/CD8 mAb 1 and HIV mAb 2/CD3 mAb 2/CD8 mAb 1 Tri-Specific Binding Molecules to bind to soluble, immobilized gp140 protein (FIG. 18A), human CD3 (FIG. 18B) and both gp140 protein and human CD3 (FIG. 18C).

FIGS. 19A-19C show the ability of ability of HIV mAb 1/CD3 mAb 2/CD8 mAb 1 and HIV mAb 2/CD3 mAb 2/CD8 mAb 1 Tri-Specific Binding Molecules to bind to HIV env-expressing HEK293/D375 cells in contrast to a control Tri-Specific Binding Molecule (FIG. 19C).

FIGS. 20A-20B show the ability of ability of HIV mAb 1/CD3 mAb 2/CD8 mAb 1 and HIV mAb 2/CD3 mAb 2/CD8 mAb 1 Tri-Specific Binding Molecules to bind to exhibit specific binding to the CD5+/CD5− cell population of human PBMCs.

FIGS. 21A-21F show the cytotoxic activity mediated by the HIV mAb 1/CD3 mAb 2/CD8 mAb 1 or HIV mAb 2/CD3 mAb 2/CD8 mAb 1 Tri-Specific Binding Molecule on Jurkat cells in the presence or absence of tetracycline (FIGS. 21A-21B; FIGS. 21C-21D). FIGS. 21E-21F show the cytotoxic activity of a control anti-RSV antibody (Palivizumab; RSV mAb 1) Tri-Specific Binding Molecule.

FIGS. 22A-22B show the percentage of live HIV env-expressing Jurkat 522 FY cells at one day and 2 days after incubation with purified pan T cells and HIV mAb 1/CD3 mAb 2/CD8 mAb 1 or HIV mAb 2/CD3 mAb 2/CD8 mAb 1 Tri-Specific Binding Molecules.

FIGS. 23A-23C show the results of an assessment of the CTL activity of the HIV mAb 1/CD3 mAb 2/CD8 mAb 1 Tri-Specific Binding Molecule on HIV env-expressing Jurkat 522 FY cells using CD4+, CD8+ or pan T cells.

FIGS. 24A-24C show the results of an assessment of the CTL activity of the HIV mAb 2/CD3 mAb 2/CD8 mAb 1 Tri-Specific Binding Molecule on HIV env-expressing Jurkat 522 FY cells using CD4+, CD8+ or pan T cells.

FIGS. 25A-25C show the kinetics of binding for DART™ molecules having the CD3 mAb 2 Binding Domain (FIG. 25A), and its CD3 mAb 2 Low (FIG. 25B) and CD3 mAb 2 Fast (FIG. 25C) affinity variants.

FIGS. 26A-26B show the CD5⁺ CD4⁺ gated (FIG. 26A) or CD5⁺ CD4⁻ gated (FIG. 26B) cell populations of human PMBC as a function of increasing concentration of the 5T4 mAb 1/CD3 mAb 2/CD8 mAb 1 Tri-Specific Binding Molecule, the 5T4 mAb 1/CD3 mAb 2 Low/CD8 mAb 1 Tri-Specific Binding Molecule and the 5T4 mAb 1/CD3 mAb 2 Fast/CD8 mAb 1 Tri-Specific Binding Molecule. 5T4×CD3 DARTs™ (with wild-type, Low and Fast CD3 specificities were used as controls.

FIGS. 27A-27C show the effect of the CD3 mAb variants (CD3 mAb 2 Low and CD3 mAb 2 Fast) on the cytotoxicity of a 5T4 mAb 1/CD3 mAb 2/CD8 mAb 1 Tri-Specific Binding Molecule using an LDH assay.

FIGS. 28A-28F demonstrate the level of IFN-γ (FIG. 28A), TNF-α (FIG. 28B), IL-10 (FIG. 28C), IL-6 (FIG. 28D), IL-4 (FIG. 28E), and IL-2 (FIG. 28F) released from PBMCs from Donor 1 in the presence of increasing concentrations of 5T4 mAb 1/CD3 mAb 2/CD8 mAb 1 Tri-Specific Binding Molecule, 5T4 mAb 1/CD3 mAb 2 Low/CD8 mAb 1 Tri-Specific Binding Molecule and 5T4 mAb 1/CD3 mAb 2 Fast/CD8 mAb 1 Tri-Specific Binding Molecule.

FIGS. 29A-29F demonstrate the level of IFN-γ (FIG. 29A), TNF-α (FIG. 29B), IL-10 (FIG. 29C), IL-6 (FIG. 29D), IL-4 (FIG. 29E), and IL-2 (FIG. 29F) released from PBMCs from Donor 2 in the presence of increasing concentrations of 5T4 mAb 1/CD3 mAb 2/CD8 mAb 1 Tri-Specific Binding Molecule, 5T4 mAb 1/CD3 mAb 2 Low/CD8 mAb 1 Tri-Specific Binding Molecule and 5T4 mAb 1/CD3 mAb 2 Fast/CD8 mAb 1 Tri-Specific Binding Molecule.

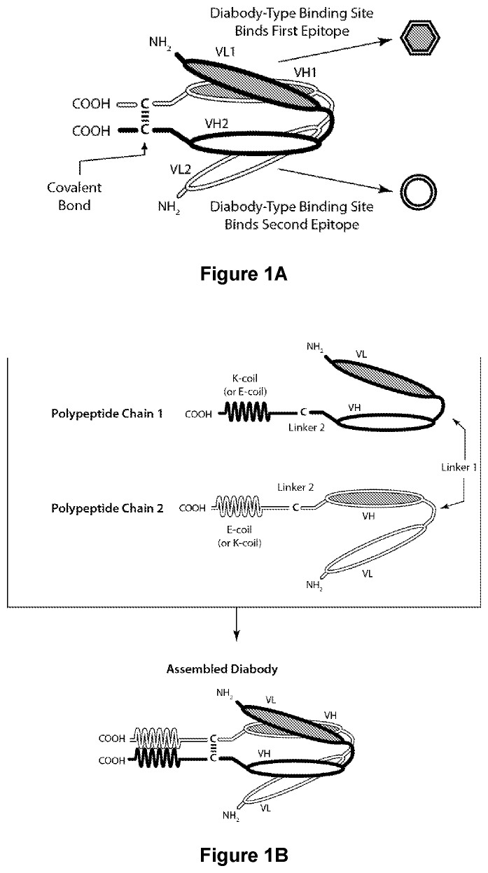

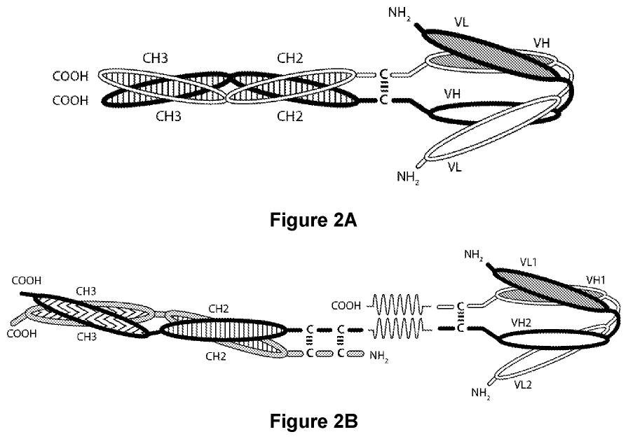

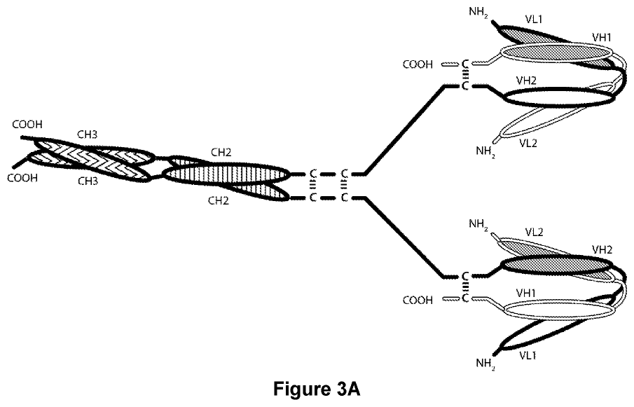

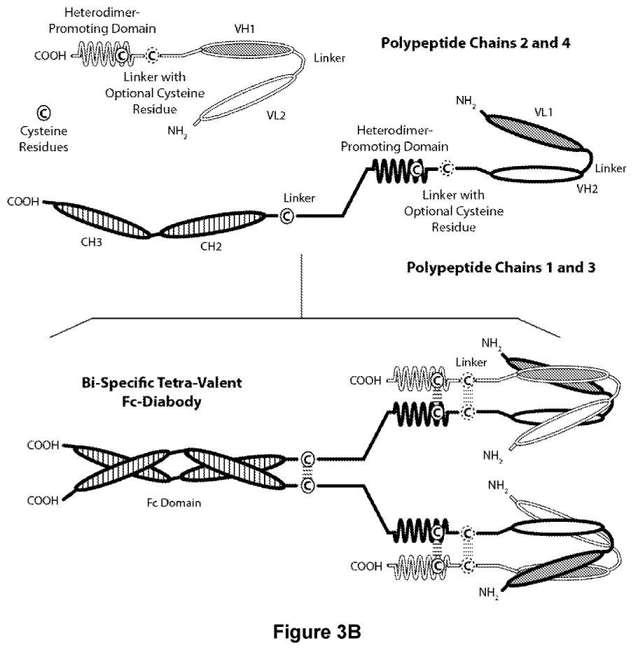

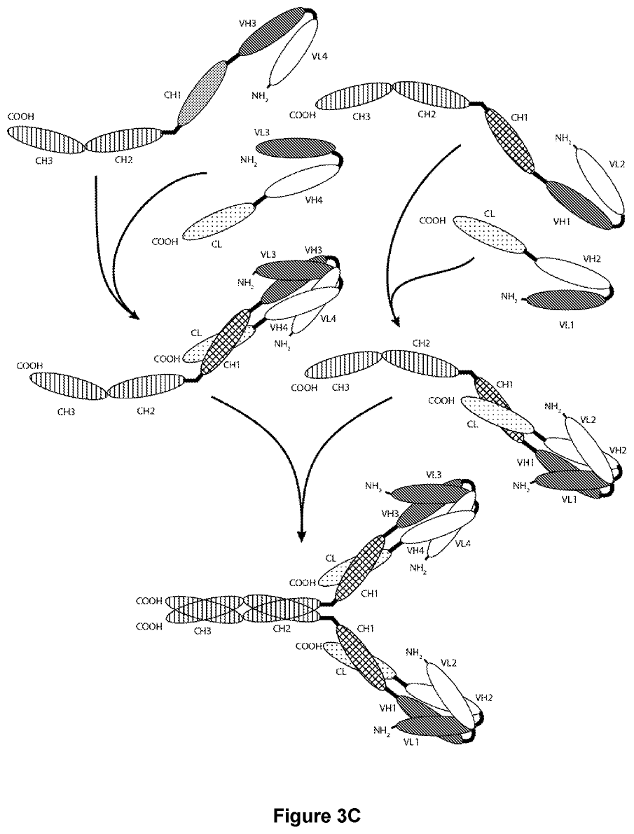

### DETAILED DESCRIPTION OF THE INVENTION

The present invention relates to Tri-Specific Binding Molecules, which are multi-chain polypeptide molecules that possess three Binding Domains and are thus capable of mediating coordinated binding to three epitopes. The Binding Domains may be selected such that the Tri-Specific Binding Molecules are capable of binding to any three different epitopes. Such epitopes may be epitopes of the same antigen or epitopes of two or three different antigens. The invention also provides a novel ROR1-binding antibody, as well as derivatives thereof and uses for such compositions.

### I. General Techniques and General Definitions

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, MOLECULAR CLONING: A LABORATORY MANUAL, Third Edition (Sambrook et al. Eds., 2001) Cold Spring Harbor Press, Cold Spring Harbor, NY; OLIGONUCLEOTIDE SYNTHESIS: METHODS AND APPLICATIONS (Methods in Molecular Biology), Herdewijn, P., Ed., Humana Press, Totowa, NJ; OLIGONUCLEOTIDE SYNTHESIS (Gait, M. J., Ed., 1984); METHODS IN MOLECULAR BIOLOGY, Humana Press, Totowa, NJ; CELL BIOLOGY: A LABORATORY NOTEBOOK (Cellis, J. E., Ed., 1998) Academic Press, New York, NY; ANIMAL CELL CULTURE (Freshney, R. I., Ed., 1987); INTRODUCTION TO CELL AND TISSUE CULTURE (Mather, J. P. and Roberts, P. E., Eds., 1998) Plenum Press, New York, NY; CELL AND TISSUE CULTURE: LABORATORY PROCEDURES (Doyle, A. et al., Eds., 1993-8) John Wiley and Sons, Hoboken, NJ; METHODS IN ENZYMOLOGY (Academic Press, Inc.) New York, NY; WEIR'S HANDBOOK OF EXPERIMENTAL IMMUNOLOGY (Herzenberg, L. A. et al. Eds. 1997) Wiley-Blackwell Publishers, New York, NY; GENE TRANSFER VECTORS FOR MAMMALIAN CELLS (Miller, J. M. et al. Eds., 1987) Cold Spring Harbor Press, Cold Spring Harbor, NY; CURRENT PROTOCOLS IN MOLECULAR BIOLOGY (Ausubel, F. M. et al., Eds., 1987) Greene Pub. Associates, New York, NY; PCR: THE POLYMERASE CHAIN REACTION, (Mullis, K. et al., Eds., 1994) Birkhäuser, Boston MA; CURRENT PROTOCOLS IN IMMUNOLOGY (Coligan, J. E. et al., eds., 1991) John Wiley and Sons, Hoboken, NJ; SHORT PROTOCOLS IN MOLECULAR BIOLOGY (John Wiley and Sons, 1999) Hoboken, NJ; IMMUNOBIOLOGY 7 (Janeway, C. A. et al. 2007) Garland Science, London, UK; Antibodies (P. Finch, 1997) Stride Publications, Devoran, UK; ANTIBODIES: A PRACTICAL APPROACH (D. Catty., ed., 1989) Oxford University Press, USA, New York NY); MONOCLONAL ANTIBODIES: A PRACTICAL APPROACH (Shepherd, P. et al. Eds., 2000) Oxford University Press, USA, New York NY; USING ANTIBODIES: A LABORATORY MANUAL (Harlow, E. et al. Eds., 1998) Cold Spring Harbor Laboratory Press, Cold Spring Harbor, NY; THE ANTIBODIES (Zanetti, M. et al. Eds. 1995) Harwood Academic Publishers, London, UK); and DEVITA, HELLMAN, AND ROSENBERG'S CANCER: PRINCIPLES & PRACTICE OF ONCOLOGY, EIGHTH EDITION, DeVita, V. et al. Eds. 2008, Lippincott Williams & Wilkins, Philadelphia, PA.

### II. Preferred Tri-Specific Binding Molecules of the Present Invention

### A. Binding Capabilities

The preferred Tri-Specific Binding Molecules of the present invention are able to coordinately and simultaneously bind to three different epitopes. Such preferred Tri-Specific Binding Molecules of the present invention comprise:
(I) a “Binding Domain I” that is capable of immunospecifically binding to an “Epitope I” present on a first antigen, and a “Binding Domain II” that is capable of immunospecifically binding to an “Epitope II” present on a second antigen, wherein said Binding Domain I and said Binding Domain II are both “Diabody-Type Binding Domains;”
(II) a “Non-Diabody-Type” “Binding Domain III” that is capable of immunospecifically binding to an “Epitope III” present on a third antigen; and
(III) an Fc Domain that is formed by the complexing of two CH2-CH3 Domains to one another.

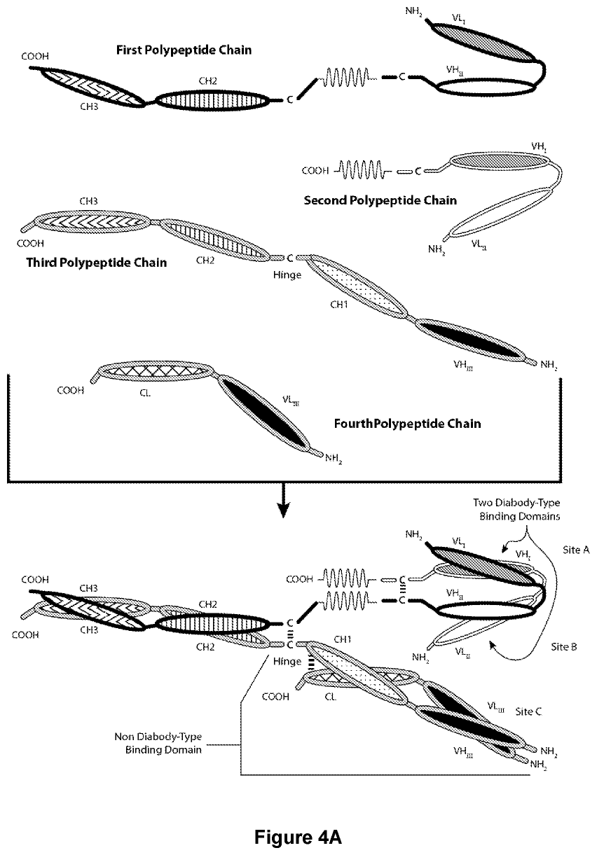

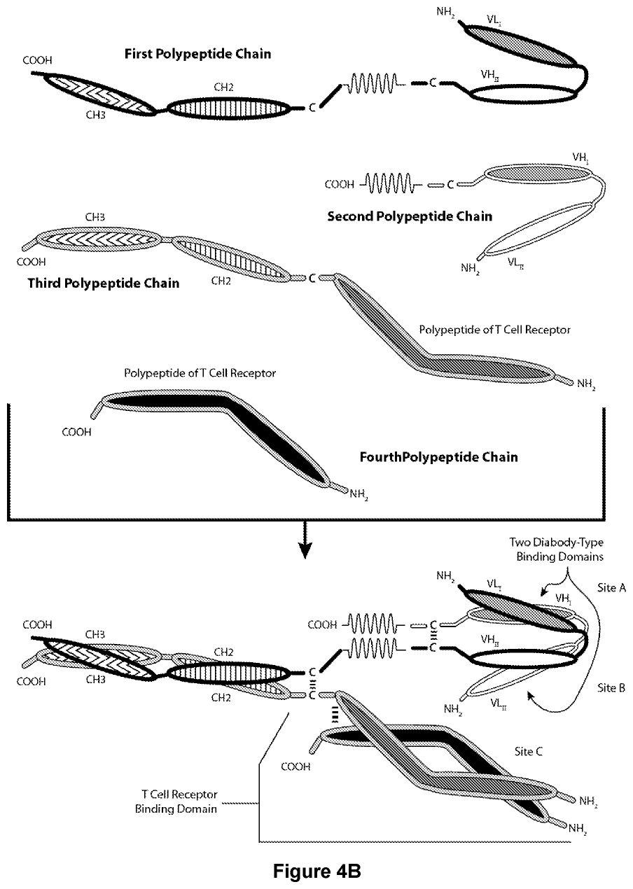

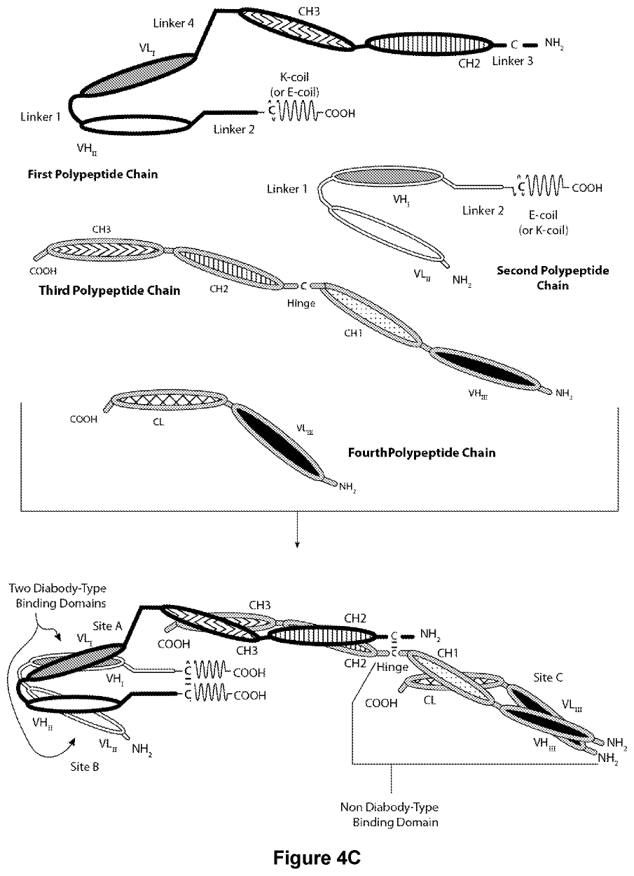

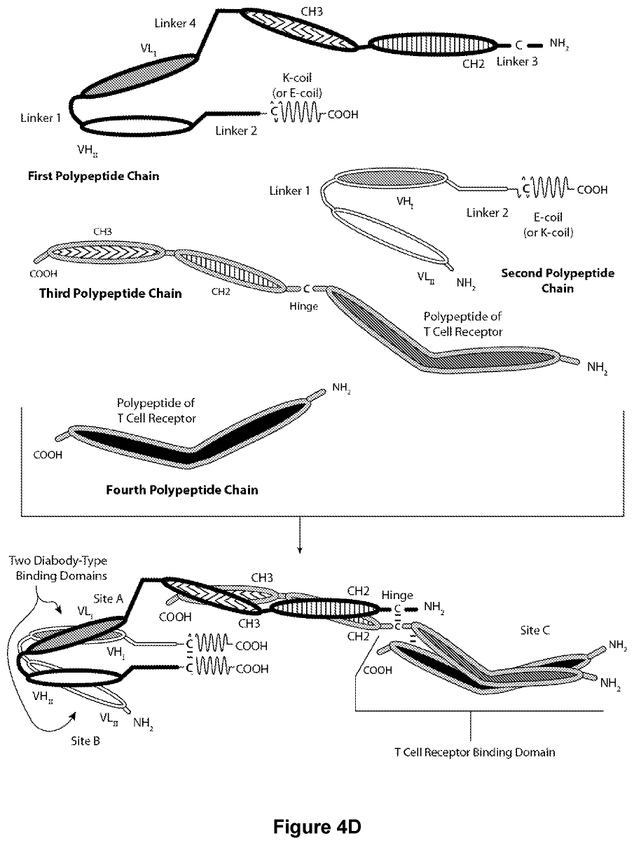

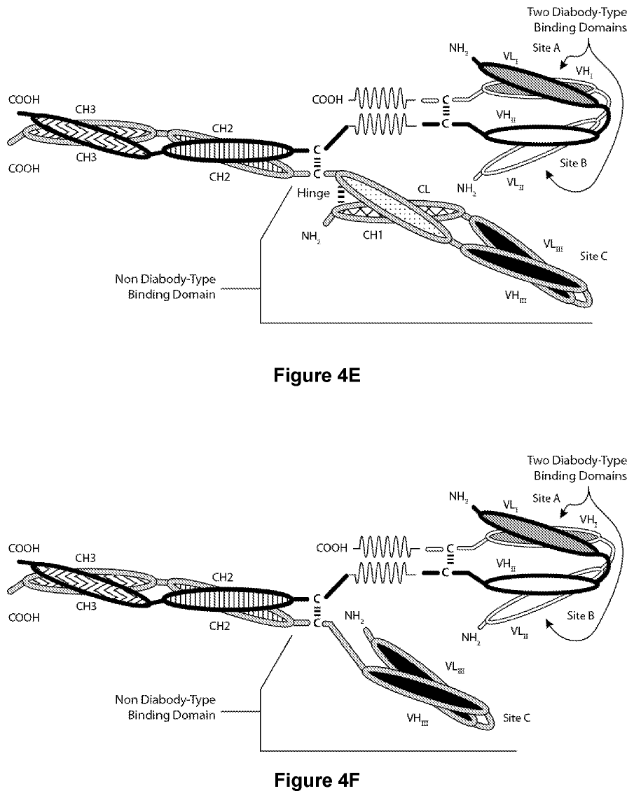

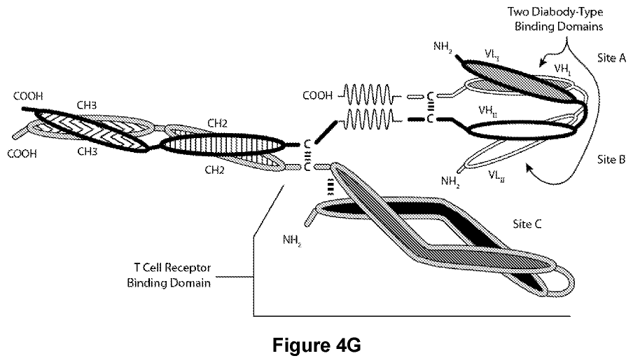

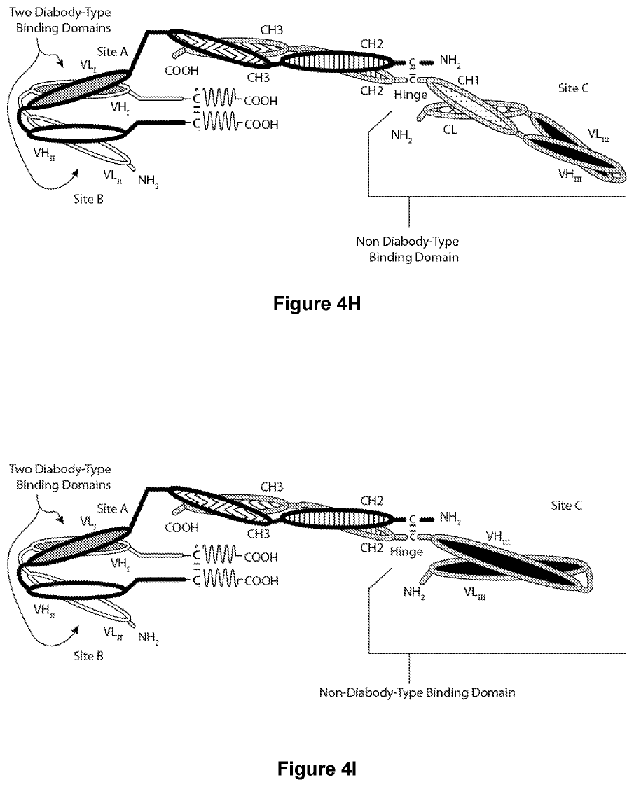

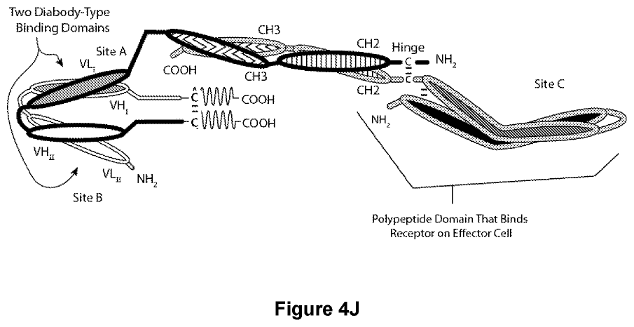

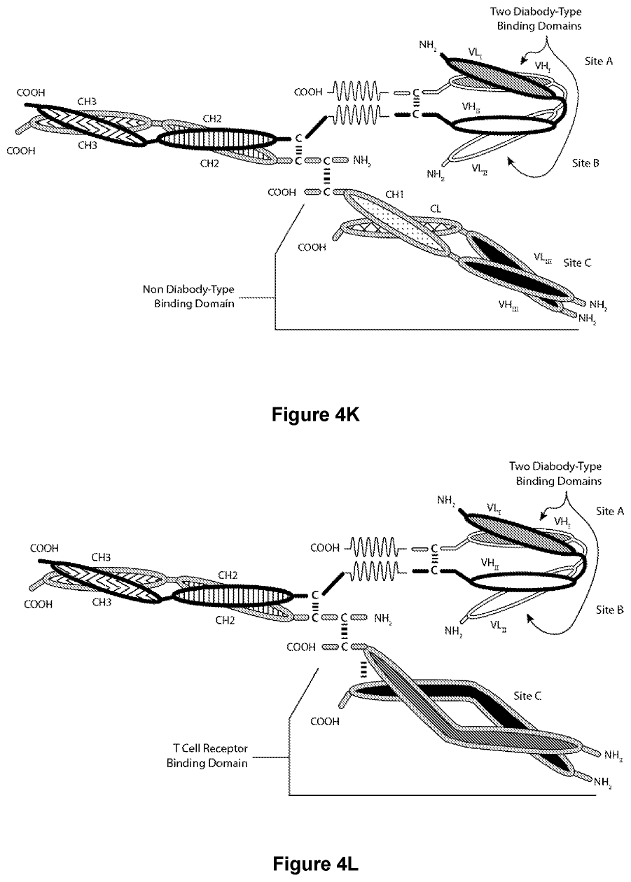

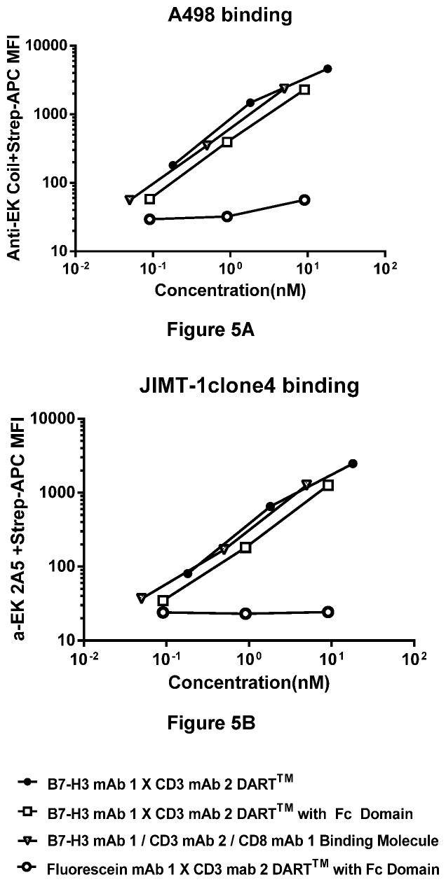

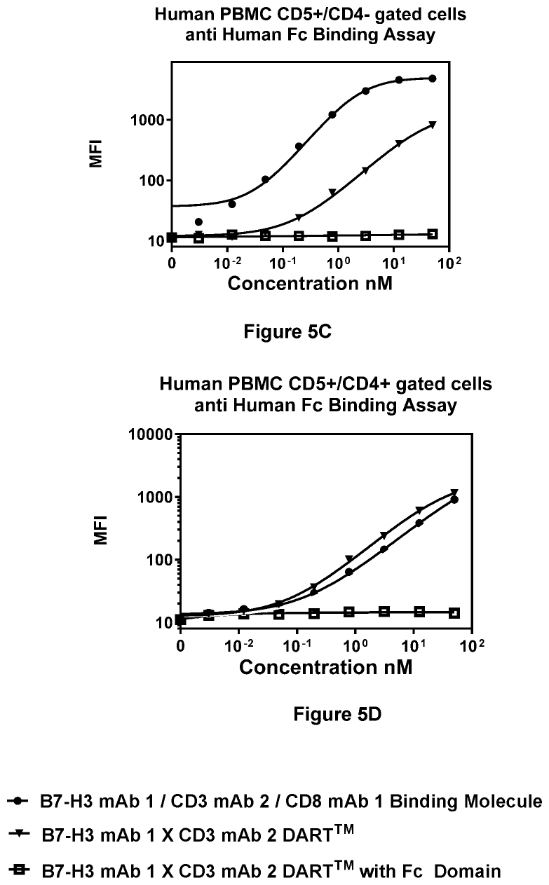

Typically, the Tri-Specific Binding Molecules of the present invention will comprise four different polypeptide chains, each having an amino terminus and a carboxyl terminus (see FIG. 4A-4D, FIG. 5A and FIG. 5B), however, the molecules may comprise fewer or greater numbers of polypeptide chains by fusing such polypeptide chains to one another (e.g., via a peptide bond) or by dividing such polypeptide chains to form additional polypeptide chains. FIGS. 4E-4J illustrate this aspect of the present invention by schematically depicting such molecules having three polypeptide chains. FIG. 4K-4L illustrate this aspect of the present invention by schematically depicting molecules having five polypeptide chains.

Although such Tri-Specific Binding Molecules are particularly preferred, the invention additionally specifically contemplates Tri-Specific Binding Molecules that comprise any combination of Binding Domains sufficient to produce a molecule having three binding specificities, of which two are binding specificities directed against Cancer Antigens, and one is a binding specificity directed against an Effector Cell Antigen. Thus, for example, the invention contemplates: a Tri-Specific Binding Molecule that comprises three Fab-Type Binding Domains, a Tri-Specific Binding Molecule that comprises one bivalent, bi-specific antibody domain (formed for example, by complexing two different light chains and two different heavy chains) and one Fab-Type Binding Domain, a Tri-Specific Binding Molecule that comprises two bivalent, bi-specific antibody domains (formed for example, by complexing four different light chains and two different heavy chains), but in which one of antibodiy domains has been rendered inactive, etc.

The terms “polypeptide,” “polypeptide chain,” and “peptide” are used interchangeably herein to refer to polymers of amino acids of any length, but especially lengths greater than 3, 5, 10, 15, 20 or 25 amino acid residues, in which two, and more preferably all, amino acid residues are joined via an amide (peptide) bond (—NH—C(O)—). The polymer may however be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The polypeptides of this invention can occur as single-chains or as complexed chains.

A “Diabody-Type Binding Domain” is the Epitope-Binding Domain of a diabody, and especially, a DART® diabody. The terms “diabody” and “DART® diabody” has been discussed above, and refers to a molecule that comprises at least two polypeptide chains that preferably complex with one another through a covalent interaction to form at least two epitope binding sites, which may recognize the same or different epitopes. Two of the polypeptide chains of a diabody or DART® diabody each comprise immunoglobulin Light Chain Variable Region and an immunoglobulin Heavy Chain Variable Region, but these regions do not interact to form an epitope binding site (i.e., they are not mutually “complementary”). Rather, the immunoglobulin Heavy Chain Variable Region of one (e.g., the first) of the diabody, or DART® diabody, chains interacts with the immunoglobulin Light Chain Variable Region of a different (e.g., the second) diabody or, DART® diabody, polypeptide chain to form an epitope binding site. Similarly, the immunoglobulin Light Chain Variable Region of one (e.g., the first) of the diabody, or DART® diabody, polypeptide chains interacts with the immunoglobulin Heavy Chain Variable Region of a different (e.g., the second) diabody, or DART® diabody, polypeptide chain to form an epitope binding site. DART® diabody molecules are disclosed in United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538; WO 2006/113665, WO 2008/157379 and Moore, P. A. et al. (2011) “Application Of Dual Affinity Retargeting Molecules To Achieve Optimal Redirected T-Cell Killing Of B-Cell Lymphoma,” Blood 117(17):4542-4551; Veri, M. C. et al. (2010) “Therapeutic Control Of B Cell Activation Via Recruitment Of Fcgamma Receptor IIb (CD32B) Inhibitory Function With A Novel Bi-specific Antibody Scaffold,” Arthritis Rheum. 62(7):1933-1943; and Johnson, S. et al. (2010) “Effector Cell Recruitment With Novel Fv-Based Dual-Affinity Re-Targeting Protein Leads To Potent Tumor Cytolysis And in vivo B-Cell Depletion,” J. Mol. Biol. 399(3):436-449.

Binding Domain III is preferably a “Non-Diabody-Type” Binding Domain, which is intended to denote that Binding Domain III does not have the structure of a Diabody-Type Binding Domain. Preferably, Binding Domain III is a Non-Diabody-Type Binding Domain that is a Fab-Type Binding Domain or a Receptor-Type Binding Domain. As used herein, the term “Fab-Type Binding Domain” refers to an epitope-Binding Domain that is formed by the interaction of the VL Domain of an immunoglobulin Light Chain and a complementing VH Domain of an immunoglobulin heavy chain. Fab-Type Binding Domains differ from Diabody-Type Binding Domain in that the two polypeptide chains that form a Fab-Type Binding Domain comprise only a single epitope-Binding Domain, whereas the two polypeptide chains that form a Diabody-Type Binding Domain comprise at least two epitope-Binding Domains. Thus, as used herein Fab-Type Binding Domains are distinct from Diabody-Type Binding Domain. As used herein, the term “Receptor-Type Binding Domain” refers to an epitope-binding domain of a cellular receptor that is formed by the interaction of two polypeptides. Receptor-Type Binding Domains are exemplified herein by reference to a T Cell Receptor-Type Binding Domain, which is formed from the interaction of a Variable Domain of a T Cell Receptor alpha chain and a Variable Domain of a T Cell Receptor beta chain. Such T Cell Receptor Binding Domains recognize peptides displayed in the context of MHC and are thus capable of recognizing intracellular epitopes. Although the invention is illustrated with regard to such Receptor-Type Binding Domains, it will be appreciated that Receptor-Type Binding Domains other than T Cell Receptor-Type Binding Domains may be employed, and are encompassed by the present invention. Other examples of receptors having Receptor-Type Binding Domains include the IL-2 receptor, the IL-4 receptor, the IL-7 receptor, the IL-9 receptor, the IL-15 receptor, the IL-21 the insulin receptor, and thymic stromal lymphopoietin.

The Tri-Specific Binding Molecules of the present invention are thus distinguished from tetravalent binding molecules, such as those produced from the dimerization of a bivalent antibody, and preferably possess three and not four Binding Domains. As discussed below, the trispecific molecules of the present invention may possess additional binding domains (such as an Albumin-Binding Domain, an FcγR-Binding Domain, etc.). Such additional Binding Domains are not intended to be considered or counted as being one of the three Binding Domains of the Tri-Specific Binding Molecules of the present invention.

As used herein, the terms “association” or “associating,” with regard to polypeptides (e.g., one diabody polypeptide to another, an immunoglobulin Light Chain to an immunoglobulin heavy chain, one CH2-CH3 Domain to another CH2-CH3 Domain, etc.) is intended to denote a non-covalent combining of the polypeptides. The terms “complexes” or “complexing” are intended to denote a covalent combining of the polypeptides.

As used herein, Binding Domains of the Tri-Specific Binding Molecules of the invention are said to mediate “coordinated binding” if at least two of its Binding Domains and preferably all of its Binding Domains, are capable of concurrently being bound to their respective recognized epitopes or binding ligand. Such binding may be simultaneous. However, one aspect of the present invention relates to modifying the “on” and/or “off” rates with which such Binding Domains bind to their recognized epitopes. As used here, the “on rate” of binding is a measure of the affinity with which such Binding Domains recognize and initiate binding to their recognized epitopes. In contrast, the “off rate” of binding is a measure of the degree of stability of the Binding Domain:epitope complex. The “on” and/or “off” rates of binding can be modified by altering the amino acid sequence of the CDRs of a Binding Domain. As discussed below, independent of any CDR modifications, the extent of coordinated binding of the molecules of the present invention may be modulated by changing the configuration of the their Binding Domains so that a particular Binding Domain (i.e., a VLx/VHx Domain) is present as Binding Domain III or as an internal or external Diabody-Type Binding Domain relative to Binding Domain III (discussed in detail below).

The on- and off-rates of the Binding Domains of the Tri-Specific Binding Molecules of the present invention can be readily measured by methods well-known in the art, for example by Biacore® analysis (Jason-Moller, L. et al. (2006) “Overview Of Biacore Systems And Their Applications,” Curr. Protoc. Protein Sci. Chapter 19:Unit 19.13; Swanson, S. J. (2005) “Characterization Of An Immune Response,” Dev. Biol. (Basel). 122:95-101; Buijs, J. et al. (2005) “SPR-MS In Functional Proteomics,” Brief Funct. Genomic Proteomic. 4(1):39-47; Karlsson, R. et al. (2004) “SPR For Molecular Interaction Analysis: A Review Of Emerging Application Areas,” J. Mol. Recognit. 17(3):151-161; Van Regenmortel, M. H. (2003) “Improving The Quality Of BIACORE-Based Affinity Measurements,” Dev. Biol. (Basel) 112:141-151; Malmqvist, M. (1999) “BIACORE: An Affinity Biosensor System For Characterization Of Biomolecular Interactions,” Biochem. Soc. Trans. 27(2):335-340; Malmqvist, M. et al. (1997) “Biomolecular Interaction Analysis: Affinity Biosensor Technologies For Functional Analysis Of Proteins,” Curr. Opin. Chem. Biol. 1(3):378-383; Fivash, M. et al. (1998) “Biacore For Macromolecular Interaction,” Curr. Opin. Biotechnol. 9(1):97-101; Malmborg, A. C. et al. (1995) “Biacore As A Tool In Antibody Engineering,” J. Immunol. Methods. 183(1):7-13). The on- and off-rates of the Binding Domains of the Tri-Specific Binding Molecules of the present invention can be readily altered by random or directed mutagenesis of nucleic acid molecules that encode such Binding Domains, followed by the routine screening of recovered nucleic acid molecules for their ability to encode mutated proteins that exhibit such altered binding kinetics.

The Binding Domains of the Tri-Sprecific Binding Molecules of the present invention bind to epitopes in an “immunospecific” manner. As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternative epitopes. For example, an antibody that immunospecifically binds to a viral epitope is an antibody that binds this viral epitope with greater affinity, avidity, more readily, and/or with greater duration than it immunospecifically binds to other viral epitopes or non-viral epitopes. It is also understood by reading this definition that, for example, an antibody (or moiety or epitope) that immunospecifically binds to a first target may or may not specifically or preferentially bind to a second target. As such, “specific binding” does not necessarily require (although it can include) exclusive binding. Generally, but not necessarily, reference to binding means “specific” binding. Two molecules are said to be capable of binding to one another in a “physiospecific” manner, if such binding exhibits the specificity with which receptors bind to their respective ligands.

Thus, in their simplest embodiment, the preferred binding molecules of the present invention are at least tri-specific—being capable of mediating coordinated binding to three different epitopes. Significantly, such molecules have at least three “sites” that are capable of binding antigen: an “external” Diabody-Type Binding Domain that is located furthest from Binding Domain III, an “internal” Diabody-Type Binding Domain that is located nearest to Binding Domain III, and Binding Domain III itself. The positions of such Domains are respectively designated as Site A, Site B and Site C (FIGS. 4A-4D).

The Binding Domains that bind to Epitopes I, II and III are selected to be different from one another. However, Epitopes I, II and III may be epitopes of the same antigen, of two different antigens, or of three different antigens. Thus the Tri-Specific Binding Molecules of the present invention may be capable of coordinately binding 1, 2, or 3 different antigen molecules. The Tri-Specific Binding Molecules of the present invention may be employed with respect to any possible epitope and any possible antigen. For example, the Tri-Specific Binding Molecules of the present invention may have 1, 2, or 3 Binding Domains that bind to an epitope of an effector cell (e.g., CD2, CD3, CD16, CD19, CD20, CD22, CD32B, CD64, the B cell Receptor (BCR), the T cell Receptor (TCR), and the NKG2D Receptor), or to an epitope of a cytotoxic T cell (e.g., CD8 present on cytotoxic T cells), or to an epitope of a Disease-Associated Antigen, or any combination of such potential Binding Domains.

As used herein, a “Disease-Associated Antigen” is an antigen that is characteristically expressed on a “pathogen-infected” cell or on a “cancer cell,” but characteristically not expressed on a normal cell.

As used herein, the term “pathogen-infected” cell refers to a cell that has been infected by a bacterium (e.g., E. coli, C. difficile, Salmonella thyphimurium, Pseudomonas aeruginosa, Vibrio cholerae, Neisseria gonorrhoeae, Helicobacter pylori, Hemophilus influenzae, Shigella dysenteriae, Staphylococcus aureus, Mycobacterium tuberculosis and Streptococcus pneumonia, etc.), a fungus (e.g., Candida, Aspergillus, Cryptococcus, Coccidioides, Histoplasma, Pneumocystis, Stachybotrys, etc.), a protozoan (Amoebozoa, Excavata, Chromalveolata, Entamoeba, Plasmodium, Giardia, Trypanosoma, Coccidia, Besnoitia, Dicrocoelium, Leishmania, etc.) or a virus (and especially an adenovirus, an adeno-associated virus, a B virus (macacine herpesvirus I), a BK virus, a bunyavirus, a chikungunya virus, a cocksackie virus, a coronavirus, a cytomegalovirus, an eastern equine encephalitis virus, an ebola virus, an enterovirus, an Epstein-Barr virus, a hantavirus, a hepatitis A virus, a hepatitis B virus, a hepatitis C virus, a hepatitis D virus, a hepatitis E virus, a herpes simplex virus 1, a herpes simplex virus 2, a human foamy virus, a human herpes virus 3, a human herpes virus 5, a human herpes virus 6, a human herpes virus 7, a human immunodeficiency virus, a human papillomavirus, a human P-lymphotropic virus, a human T cell leukemia virus I, a human T cell leukemia virus II, an influenza virus, a JC virus, a JEV, a Kaposi's sarcoma-associated herpesvirus, a Lassa virus, a lymphocytic choriomenengitis virus, a Marburg virus, a measles virus, a mumps virus, a Nipah virus, a norovirus, a Norwalk virus, an orthoreovirus, a parainfluenza virus, a parvovirus, a poliovirus, a rabies virus, a reovirus, a respiratory syncytial virus, rhinovirus, a Rift Valley fever virus, a rotavirus, rubella virus, a smallpox virus, a St Louis encephalitis virus, a variola major virus, a variola minor virus, a vericella-zoster virus, a West Nile virus, a western equine encephalitis virus, or a yellow fever virus).

As used herein, the term “cancer cell” refers to a malignant cell of: an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, or a uterine cancer.

Examples of antigens that are characteristically expressed by cancer cells include a “cancer antigen” such as a breast cancer antigen, an ovarian cancer antigen, a prostate cancer antigen, a cervical cancer antigen, a pancreatic carcinoma antigen, a lung cancer antigen, a bladder cancer antigen, a colon cancer antigen, a testicular cancer antigen, a glioblastoma cancer antigen, an antigen associated with a B cell malignancy, an antigen associated with multiple myeloma, an antigen associated with non-Hodgkins lymphoma, or an antigen associated with chronic lymphocytic leukemia. Exemplary antigens that are characteristically expressed by cancer cells include the antigens: colon cancer antigen 19.9; gastric cancer mucin antigen 4.2; colorectal carcinoma antigen A33 (Almqvist, Y. 2006, Nucl Med Biol. November; 33(8):991-998); ADAM-9 (United States Patent Publication No. 2006/0172350; PCT Publication No. WO 06/084075; AFP oncofetal antigen-alpha-fetoprotein (Malaguarnera, G. et al. (2010) “Serum markers of hepatocellular carcinoma,” Dig. Dis. Sci. 55(10):2744-2755); ALCAM (PCT Publication No. WO 03/093443); BAGE (Bodey, B. 2002 Expert Opin Biol Ther. 2(6):577-84); beta-catenin (Prange W. et al. 2003 J Pathol. 201(2):250-9); CA125 (Bast, R. C. Jr. et al. 2005 Int J Gynecol Cancer 15 Suppl 3:274-81); Carboxypeptidase M (United States Patent Publication No. 2006/0166291); B1 (Egloff, A. M. et al. 2006, Cancer Res. 66(1):6-9); CD5 (Calin, G. A. et al. 2006 Semin Oncol. 33(2):167-73; CD19 (Troussard, X. et al. 1998 Hematol Cell Ther. 40(4):139-48); CD20 (Thomas, D. A. et al. 2006 Hematol Oncol Clin North Am. 20(5):1125-36); CD20 (Cang, S. et al. (2012) “Novel CD20 Monoclonal Antibodies For Lymphoma Therapy,” J. Hematol. Oncol. 5:64 pp. 1-9); CD22 (Kreitman, R. J. 2006 AAPS J. 18; 8(3):E532-51); CD23 (Rosati, S. et al. 2005 Curr Top Microbiol Immunol. 5; 294:91-107); CD25 (Troussard, X. et al. 1998 Hematol Cell Ther. 40(4):139-48); CD27 (Bataille, R. 2006 Haematologica 91(9):1234-40); CD28 (Bataille, R. 2006 Haematologica 91(9):1234-40); CD30 (Muta, H. et al. (2013) “CD30: From Basic Research To Cancer Therapy,” Immunol. Res. 57(1-3):151-158); CD33 (Walter, R. B. et al. (2012) “Acute myeloid leukemia stem cells and CD33-targeted immunotherapy,” Blood 119(26):6198-6208); CD36 (Ge, Y. 2005 Lab Hematol. 11(1):31-7); CD40/CD154 (Messmer, D. et al. 2005 Ann N Y Acad Sci. 1062:51-60); CD45 (Jurcic, J. G. 2005 Curr Oncol Rep. 7(5):339-46); CD56 (Bataille, R. 2006 Haematologica 91(9):1234-40); CD46 (U.S. Pat. No. 7,148,038; PCT Publication No. WO 03/032814; Russell, S. et al. (2004) “CD46: A Complement Regulator And Pathogen Receptor That Mediates Links Between Innate And Acquired Immune Function,” Tissue Antigens 64(2):111-118); CD52 (Hoelzer, D. et al. (2013) “Targeted therapy with monoclonal antibodies in acute lymphoblastic leukemia,” Curr. Opin. Oncol. 25(6):701-706); CD79a/CD79b (Troussard, X. et al. 1998 Hematol Cell Ther. 40(4):139-48; Chu, P. G. et al. 2001 Appl Immunohistochem Mol Morphol. 9(2):97-106); CD103 (Troussard, X. et al. 1998 Hematol Cell Ther. 40(4):139-48); CD317 (Palma, G. et al. (2012) “Plasmacytoids Dendritic Cells Are A Therapeutic Target In Anticancer Immunity,” Biochim. Biophys. Acta. 1826(2):407-414; CDK4 (Lee, Y. M. et al. 2006 Cell Cycle 5(18):2110-4); CEA (carcinoembryonic antigen; Mathelin, C. 2006 Gynecol Obstet Fertil. 34(7-8):638-46; Tellez-Avila, F. I. et al. 2005 Rev Invest Clin. 57(6):814-9); CEACAM5 and CEACAM6 (PCT Publication No. WO 2011/034660; Zheng, C. et al. (2011) “A Novel Anti-CEACAM5 Monoclonal Antibody, CC4, Suppresses Colorectal Tumor Growth and Enhances NK Cells-Mediated Tumor Immunity,” PLoS One 6(6):e21146, pp. 1-11); CO17-1A (Adkins, J. C. et al. (1998) “Edrecolomab (Monoclonal Antibody 17-1A),” Drugs 56(4):619-626; CO-43 (blood group Leb) and CO-514 (blood group Lea) (Garratty, G. (1995) “Blood Group Antigens As Tumor Markers, Parasitic Bacterial/Viral Receptors, And Their Association With Immunologically Important Proteins,” Immunol. Invest. 24(1-2):213-232; CTLA-1 and CTLA-4 (Peggs, K. S. et al. 2006 Curr Opin Immunol. 18(2):206-13); Cytokeratin 8 (PCT Publication No. WO 03/024191); antigen D1.1 (Dao, T. et al. (2009) “Identification Of A Human Cyclin D1-Derived Peptide That Induces Human Cytotoxic CD4 T Cells,” PLoS One. 4(8):e6730); DR5 (Abdulghani, J. et al. (2010) “TRAIL Receptor Signaling And Therapeutics,” Expert Opin. Ther. Targets 14(10):1091-1108; Andera, L. (2009) “Signaling Activated By The Death Receptors Of The TNFR Family,” Biomed. Pap. Med. Fac. Univ. Palacky Olomouc Czech. Repub. 153(3):173-180; Carlo-Stella, C. et al. (2007) “Targeting TRAIL Agonistic Receptors for Cancer Therapy,” Clin, Cancer 13(8):2313-2317; Chaudhari, B. R. et al. (2006) “Following the TRAIL to Apoptosis,” Immunologic Res. 35(3):249-262); E1 series (blood group B); EGF-R (epidermal growth factor receptor; Adenis, A. et al. 2003 Bull Cancer. 90 Spec No:S228-32); Ephrin receptors (and in particular EphA2 (U.S. Pat. No. 7,569,672; PCT Publication No. WO 06/084226); Erb (ErbB1; ErbB3; ErbB4; Zhou, H. et al. 2002 Oncogene 21(57):8732-40; Rimon, E. et al. 2004 Int J Oncol. 24(5):1325-38); lung adenocarcinoma antigen F3 (Greulich, H. et al. (2012) “Functional analysis of receptor tyrosine kinase mutations in lung cancer identifies oncogenic extracellular domain mutations of ERBB2,” Proc. Natl. Acad. Sci. (U.S.A.) 109(36):14476-14481); antigen FC10.2 (Loveless, W. et al. (1990) “Developmental Patterning Of The Carbohydrate Antigen FC10.2 During Early Embryogenesis In The Chick,” Development 108(1):97-106); GAGE (GAGE-1; GAGE-2; Akcakanat, A. et al. 2006 Int J Cancer. 118(1):123-8); GD2/GD3/GD49/GM2/GM3 (Livingston, P. O. et al. 2005 Cancer Immunol Immunother. 54(10):1018-25); GICA 19-9 (Herlyn et al. (1982) “Monoclonal Antibody Detection Of A Circulating Tumor-Associated Antigen. I. Presence Of Antigen In Sera Of Patients With Colorectal, Gastric, And Pancreatic Carcinoma,” J. Clin. Immunol. 2:135-140); gp37 (human leukemia T cell antigen ((Bhattacharya-Chatterjee et al. (1988) “Idiotype Vaccines Against Human T Cell Leukemia. II. Generation And Characterization Of A Monoclonal Idiotype Cascade (Ab1, Ab2, and Ab3),” J. Immunol. 141:1398-1403); gp75 (melanoma antigen) (Vijayasardahl et al. (1990) “The Melanoma Antigen Gp75 Is The Human Homologue Of The Mouse B (Brown) Locus Gene Product,” J. Exp. Med. 171(4):1375-1380); gp100 (Lotem, M. et al. 2006 J Immunother. 29(6):616-27); HER-2/neu (Kumar, Pal S et al. 2006 Semin Oncol. 33(4):386-91); human B-lymphoma antigen-CD20 (Reff et al. (1994) “Depletion Of B Cells In Vivo By A Chimeric Mouse Human Monoclonal Antibody To CD20,” Blood 83:435-445); human milk fat globule antigen; human papillomavirus-E6/human papillomavirus-E7 (DiMaio, D. et al. 2006 Adv Virus Res. 66:125-59; HMW-MAA (high molecular weight melanoma antigen) (Natali et al. (1987) “Immunohistochemical Detection Of Antigen In Human Primary And Metastatic Melanomas By The Monoclonal Antibody 140.240 And Its Possible Prognostic Significance,” Cancer 59:55-63; Mittelman et al. (1990) “Active Specific Immunotherapy In Patients With Melanoma. A Clinical Trial With Mouse Antiidiotypic Monoclonal Antibodies Elicited With Syngeneic Anti-High-Molecular-Weight-Melanoma-Associated Antigen Monoclonal Antibodies,” J. Clin. Invest. 86:2136-2144); I antigen (differentiation antigen) (Feizi (1985) “Demonstration By Monoclonal Antibodies That Carbohydrate Structures Of Glycoproteins And Glycolipids Are Onco-Developmental Antigens,” Nature 314:53-57) such as I(Ma) as found in gastric adenocarcinomas; Integrin Alpha-V-Beta-6 Integrinβ6 (ITGB6) (PCT Publication No. WO 03/087340); JAM-3 (PCT Publication No. WO 06/084078); Interleukin-13 Receptor α2 (IL13Rα2) (Bodhinayake, I. et al. (2014) “Targeting A Heterogeneous Tumor: The Promise Of The Interleukin-13 Receptor α2,” Neurosurgery 75(2):N18-9); JAM-3 (PCT Publication No. WO 06/084078); KID3 (PCT Publication No. WO 05/028498); KID3 (PCT Publication No. WO 05/028498); KID31 (PCT Publication No. WO 06/076584); KID31 (PCT Publication No. WO 06/076584); KS 1/4 pan-carcinoma antigen (Perez et al. (1989) “Isolation And Characterization Of A cDNA Encoding The Ks1/4 Epithelial Carcinoma Marker,” J. Immunol. 142:3662-3667; Moller et al. (1991) “Bispecific-Monoclonal-Antibody-Directed Lysis Of Ovarian Carcinoma Cells By Activated Human T Lymphocytes,” Cancer Immunol. Immunother. 33(4):210-216; Ragupathi, G. 2005 Cancer Treat Res. 123:157-80); KS 1/4 pan-carcinoma antigen (Perez et al. (1989) “Isolation And Characterization Of A cDNA Encoding The Ks1/4 Epithelial Carcinoma Marker,” J. Immunol. 142:3662-3667; Moller et al. (1991) “Bispecific-Monoclonal-Antibody-Directed Lysis Of Ovarian Carcinoma Cells By Activated Human T Lymphocytes,” Cancer Immunol. Immunother. 33(4):210-216; Ragupathi, G. 2005 Cancer Treat Res. 123:157-80); KSA (17-1A) (Ragupathi, G. 2005 Cancer Treat Res. 123:157-80); human lung carcinoma antigens L6 and L20 (Hellstrom et al. (1986) “Monoclonal Mouse Antibodies Raised Against Human Lung Carcinoma,” Cancer Res. 46:3917-3923); LEA (Velizquez-Mirquez, N. et al. (2012) “Sialyl Lewis x expression in cervical scrapes of premalignant lesions,” J. Biosci. 37(6):999-1004); LUCA-2 (United States Patent Publication No. 2006/0172349; PCT Publication No. WO 06/083852); M1:22:25:8, M18, M39 (Cambier, L. et al. (2012) “M19 Modulates Skeletal Muscle Differentiation And Insulin Secretion In Pancreatic B-Cells Through Modulation Of Respiratory Chain Activity,” PLoS One 7(2):e31815; Pui, C. H. et al. (1991) “Characterization of childhood acute leukemia with multiple myeloid and lymphoid markers at diagnosis and at relapse,” Blood 78(5):1327-1337); MAGE (MAGE-1; MAGE-3; (Bodey, B. 2002 Expert Opin Biol Ther. 2(6):577-84); MART (Kounalakis, N. et al. 2005 Curr Oncol Rep. 7(5):377-82; Myl, MUC-1 (Mathelin, C. 2006 Gynecol Obstet Fertil. 34(7-8):638-46); MUM-1 (Castelli, C. et al. 2000 J Cell Physiol. 182(3):323-31); N-acetylglucosaminyltransferase (Dennis, J. W. 1999 Biochim Biophys Acta. 6; 1473(1):21-34); neoglycoprotein (Legendre, H. et al. (2004) “Prognostic Stratification Of Dukes B Colon Cancer By A Neoglycoprotein,” Int. J. Oncol. 25(2):269-276); NS-10; OFA-1 and OFA-2 (Takahashi, M. (1984) “A Study On Clinical Significance Of Oncofetal Antigen-1 In Gynecologic Tumors,” Nihon Sanka Fujinka Gakkai Zasshi. 36(12):2613-2618); Oncostatin M (Oncostatin Receptor Beta) (U.S. Pat. No. 7,572,896; PCT Publication No. WO 06/084092); p15 (Gil, J. et al. 2006 Nat Rev Mol Cell Biol. 7(9):667-77); PSA (prostate specific antigen; Cracco, C. M. et al. 2005 Minerva Urol Nefrol. 57(4):301-11); PSMA (Ragupathi, G. 2005 Cancer Treat Res. 123:157-80); PEMA (polymorphic epithelial mucin antigen) (Chu, N.J. et al. (2015) “Nonviral Oncogenic Antigens and the Inflammatory Signals Driving Early Cancer Development as Targets for Cancer Immunoprevention,” Clin. Cancer Res. 21(7):1549-1557); PIPA (U.S. Pat. No. 7,405,061; PCT Publication No. WO 04/043239); prostatic acid phosphate (Tailor et al. (1990) “Nucleotide Sequence Of Human Prostatic Acid Phosphatase Determined From A Full-Length cDNA Clone,” Nucl. Acids Res. 18(16):4928); R24 (Zhou, M. et al. (2008) “Constitutive Overexpression Of A Novel 21 Kda Protein By Hodgkin Lymphoma And Aggressive Non-Hodgkin Lymphomas,” Mol. Cancer 7:12); ROR1 (U.S. Pat. No. 5,843,749); Rabbani, H. et al. (2010) “Expression Of ROR1 In Patients With Renal Cancer—A Potential Diagnostic Marker,” Iran Biomed. J. 14(3):77-82); sphingolipids (Hakomori, S. (1998) “Cancer-Associated Glycosphingolipid Antigens: Their Structure, Organization, And Function,” Acta Anat. (Basel) 161(1-4):79-90; SSEA-1, SSEA-3 and SSEA-4 (Muramatsu, T. et al. (2004) “Carbohydrate Antigens Expressed On Stem Cells And Early Embryonic Cells,” Glycoconj. J. 21(1-2):41-45); sTn (Holmberg, L. A. 2001 Expert Opin Biol Ther. 1(5):881-91); T cell receptor derived peptide (Edelson (1998) “Cutaneous T-Cell Lymphoma: A Model For Selective Immunotherapy,” Cancer J Sci Am. 4:62-71); T₅A₇(Hogg, R. J. et al. (1991) “A monoclonal antibody exhibiting reactivity with both X-hapten- and lactose-bearing glycolipids,” Tissue Antigens 37(1):33-38); TAG-72 (Yokota et al. (1992) “Rapid Tumor Penetration Of A Single-Chain Fv And Comparison With Other Immunoglobulin Forms,” Cancer Res. 52:3402-3408); TL5 (blood group A) (Gooi, H. C. et al. (1983) “Monoclonal antibody reactive with the human epidermal-growth-factor receptor recognizes the blood-group-A antigen,” Biosci. Rep. 3(11):1045-1052); TNF-receptor (TNF-α receptor, TNF-β receptor; or TNF-γ receptor (van Horssen, R. et al. 2006 Oncologist. 11(4):397-408; Gardnerova, M. et al. 2000 Curr Drug Targets. 1(4):327-64); TRA-1-85 (blood group H) (Williams, B. P. et al. (1988) “Biochemical and genetic analysis of the OKa blood group antigen,” Immunogenetics 27(5):322-329); Transferrin Receptor (U.S. Pat. No. 7,572,895; PCT Publication No. WO 05/121179); TSTA tumor-specific transplantation antigen (Hellstrom et al. (1985) “Monoclonal Antibodies To Cell Surface Antigens Shared By Chemically Induced Mouse Bladder Carcinomas,” Cancer. Res. 45:2210-2188); VEGF-R (O'Dwyer. P. J. 2006 Oncologist. 11(9):992-8); and Y hapten, Le^{y} (Durrant, L. G. et al. (1989) “Development Of An ELISA To Detect Early Local Relapse Of Colorectal Cancer,” Br. J. Cancer 60(4):533-537).

Exemplary antibodies that immunospecifically bind to an epitope of a Disease-Associated Antigen that may be used to provide the Variable Light Chain Domains, Variable Heavy Chain Domains, Antibody Light Chains or Antibody Heavy Chains of the Tri-Specific Binding Molecules of the present invention are presented in Table 2.

TABLE 2

| Antibody | Disease-Associated | |
|---|---|---|
| Name | Antigen | Therapeutic Target Application |
| 3F8 | Gd2 | Neuroblastoma |
| 8H9 | B7-H3 | Neuroblastoma, Sarcoma, |
| | | Metastatic Brain Cancers |
| Abagovomab | CA-125 | Ovarian Cancer |
| Abciximab | CD41 | Platelet Aggregation Inhibitor |
| Actoxumab | Clostridium | Clostridium Difficile Infection |
| | Difficile | |
| Adalimumab | TNF-A | Rheumatoid Arthritis, Crohn's |
| | | Disease, Plaque Psoriasis, Psoriatic |
| | | Arthritis, Ankylosing Spondylitis, |
| | | Juvenile Idiopathic Arthritis, |
| | | Hemolytic Disease Of The Newborn |
| Adecatumumab | Epcam | Prostate And Breast Cancer |
| Aducanumab | Beta-Amyloid | Alzheimer's Disease |
| Afelimomab | TNF-A | Sepsis |
| Afutuzumab | CD20 | Lymphoma |
| Alacizumab | VEGFR2 | Cancer |
| Ald518 | Il-6 | Rheumatoid Arthritis |
| Alemtuzumab | CD52 | Multiple Sclerosis |
| Alirocumab | NARP-1 | Hypercholesterolemia |
| Altumomab | CEA | Colorectal Cancer |
| Amatuximab | Mesothelin | Cancer |
| Anatumomab | TAG-72 | Non-Small Cell Lung Carcinoma |
| Mafenatox | | |
| Anifrolumab | Interferon A/B Receptor | Systemic Lupus Erythematosus |
| Anrukinzumab | IL-13 | Cancer |
| Apolizumab | HLA-DR | Hematological Cancers |
| Arcitumomab | CEA | Gastrointestinal Cancer |
| Aselizumab | L-Selectin (CD62L) | Severely Injured Patients |
| Atinumab | RTN4 | Cancer |
| Atlizumab | IL-6 Receptor | Rheumatoid Arthritis |
| Atorolimumab | Rhesus Factor | Hemolytic Disease Of The Newborn |
| Bapineuzumab | Beta-Amyloid | Alzheimer's Disease |
| Basiliximab | CD25 | Prevention Of Organ Transplant |
| | | Rejections |
| Bavituximab | Phosphatidylserine | Cancer, Viral Infections |
| Bectumomab | CD22 | Non-Hodgkin's Lymphoma (Detection) |
| Belimumab | BAFF | Non-Hodgkin Lymphoma |
| Benralizumab | CD125 | Asthma |
| Bertilimumab | CCL11 (Eotaxin-1) | Severe Allergic Disorders |
| Besilesomab | CEA-Related Antigen | Inflammatory Lesions And Metastases |
| | | (Detection) |
| Bevacizumab | VEGF-A | Metastatic Cancer, Retinopathy Of |
| | | Prematurity |
| Bezlotoxumab | Clostridium difficile | Clostridium difficile Infection |
| Biciromab | Fibrin II, Beta Chain | Thromboembolism (Diagnosis) |
| Bimagrumab | ACVR2B | Myostatin Inhibitor |
| Bivatuzumab | CD44 V6 | Squamous Cell Carcinoma |
| Blinatumomab | CD19 | Cancer |
| Blosozumab | SOST | Osteoporosis |
| Brentuximab | CD30 (TNFRSF8) | Hematologic Cancers |
| Briakinumab | IL-12, IL-23 | Psoriasis, Rheumatoid Arthritis, |
| | | Inflammatory Bowel Diseases, |
| | | Multiple Sclerosis |
| Brodalumab | IL-17 | Inflammatory Diseases |
| Canakinumab | IL-1 | Rheumatoid Arthritis |
| Cantuzumab | Mucin Canag | Colorectal Cancer |
| Mertansine | | |
| Cantuzumab | MUC1 | Cancers |
| Caplacizumab | VWF | Cancers |
| Capromab | Prostatic Carcinoma | Prostate Cancer (Detection) |
| | Cells | |
| Carlumab | MCP-1 | Oncology/Immune Indications |
| Catumaxomab | Epcam, CD3 | Ovarian Cancer, Malignant Ascites, |
| | | Gastric Cancer |
| Cc49 | Tag-72 | Tumor Detection |
| Certolizumab | TNF-A | Crohn's Disease |
| Cetuximab | EGFR | Metastatic Colorectal Cancer And |
| | | Head And Neck Cancer |
| Ch.14.18 | Undetermined | Neuroblastoma |
| Citatuzumab | Epcam | Ovarian Cancer And Other Solid |
| | | Tumors |
| Cixutumumab | IGF-1 Receptor | Solid Tumors |
| Clazakizumab | Oryctolagus Cuniculus | Rheumatoid Arthritis |
| Clivatuzumab | MUC1 | Pancreatic Cancer |
| Conatumumab | TRAIL-R2 | Cancer |
| Concizumab | TFPI | Bleeding |
| Crenezumab | 1-40-B-Amyloid | Alzheimer's Disease |
| Cr6261 | Influenza A | Infectious Disease/Influenza A |
| | Hemagglutinin | |
| Dacetuzumab | CD40 | Hematologic Cancers |
| Daclizumab | CD25 | Prevention Of Organ Transplant |
| | | Rejections |
| Dalotuzumab | Insulin-Like Growth | Cancer |
| | Factor I Receptor | |
| Daratumumab | CD38 | Cancer |
| Demcizumab | DLL4 | Cancer |
| Denosumab | RANKL | Osteoporosis, Bone Metastases |
| Detumomab | B-Lymphoma Cell | Lymphoma |
| Dorlimomab | Undetermined | Cancer |
| Aritox | | |
| Drozitumab | DR5 | Cancer |
| Duligotumab | HER3 | Cancer |
| Dupilumab | IL4 | Atopic Diseases |
| Dusigitumab | ILGF2 | Cancer |
| Ecromeximab | GD3 Ganglioside | Malignant Melanoma |
| Eculizumab | C5 | Paroxysmal Nocturnal Hemoglobinuria |
| Edobacomab | Endotoxin | Sepsis Caused By Gram-Negative Bacteria |
| Edrecolomab | Epcam | Colorectal Carcinoma |
| Efalizumab | LFA-1 (CD11a) | Psoriasis (Blocks T Cell Migration) |
| Efungumab | Hsp90 | Invasive Candida Infection |
| Eldelumab | Interferon-Gamma- | Crohn's Disease, Ulcerative Colitis |
| | Induced Protein | |
| Elotuzumab | SLAMF7 | Multiple Myeloma |
| Elsilimomab | IL-6 | Cancer |
| Enavatuzumab | TWEAK Receptor | Cancer |
| Enlimomab | ICAM-1 (CD54) | Cancer |
| Enokizumab | IL9 | Asthma |
| Enoticumab | DLL4 | Cancer |
| Ensituximab | 5AC | Cancer |
| Epitumomab | Episialin | Cancer |
| Cituxetan | | |
| Epratuzumab | CD22 | Cancer, SLE |
| Erlizumab | ITGB2 (CD18) | Heart Attack, Stroke, Traumatic Shock |
| Ertumaxomab | HER2/Neu, CD3 | Breast Cancer |
| Etaracizumab | Integrin Aᵥβ₃ | Melanoma, Prostate Cancer, Ovarian Cancer |
| Etrolizumab | Integrin A₇ B₇ | Inflammatory Bowel Disease |
| Evolocumab | PCSK9 | Hypocholesterolemia |
| Exbivirumab | Hepatitis B Surface | Hepatitis B |
| | Antigen | |
| Fanolesomab | CD15 | Appendicitis (Diagnosis) |
| Faralimomab | Interferon Receptor | Cancer |
| Farletuzumab | Folate Receptor 1 | Ovarian Cancer |
| Fasinumab^{[51]} | HNGF | Cancer |
| Fbta05 | CD20 | Chronic Lymphocytic Leukaemia |
| Felvizumab | Respiratory Syncytial | Respiratory Syncytial Virus Infection |
| | Virus | |
| Fezakinumab | IL-22 | Rheumatoid Arthritis, Psoriasis |
| Ficlatuzumab | HGF | Cancer |
| Figitumumab | IGF-1 Receptor | Adrenocortical Carcinoma, Non-Small |
| | | Cell Lung Carcinoma |
| Flanvotumab | TYRP1 | Melanoma |
| | (Glycoprotein 75) | |
| Fontolizumab | IFN-γ | Crohn's Disease |
| Foravirumab | Rabies Virus | Rabies (Prophylaxis) |
| | Glycoprotein | |
| Fresolimumab | TGF-B | Idiopathic Pulmonary Fibrosis, Focal |
| | | Segmental Glomerulosclerosis, Cancer |
| Fulranumab | NGF | Pain |
| Futuximab | EGFR | Cancer |
| Galiximab | CD80 | B Cell Lymphoma |
| Ganitumab | IGF-I | Cancer |
| Gantenerumab | Beta-Amyloid | Alzheimer's Disease |
| Gavilimomab | CD147 (Basigin) | Graft-Versus-Host Disease |
| Gemtuzumab | CD33 | Acute Myelogenous Leukemia |
| Ozogamicin | | |
| Gevokizumab | IL-1β | Diabetes |
| Girentuximab | Carbonic Anhydrase 9 | Clear Cell Renal Cell Carcinoma^{[64]} |
| | (CA-IX) | |
| Glembatumumab | GPNMB | Melanoma, Breast Cancer |
| Vedotin | | |
| Golimumab | TNF-A | Rheumatoid Arthritis, Psoriatic Arthritis, |
| | | Ankylosing Spondylitis |
| Gomiliximab | CD23 (Ige Receptor) | Allergic Asthma |
| Guselkumab | IL13 | Psoriasis |
| Ibritumomab | CD20 | Non-Hodgkin's Lymphoma |
| Tiuxetan | | |
| Icrucumab | VEGFR-1 | Cancer |
| Igovomab | CA-125 | Ovarian Cancer (Diagnosis) |
| Imab362 | Cldn1 8.2 | Gastrointestinal Adenocarcinomas And |
| | | Pancreatic Tumor |
| Imgatuzumab | EGFR | Cancer |
| Inclacumab | Selectin P | Cancer |
| Indatuximab | SDC1 | Cancer |
| Ravtansine | | |
| Infliximab | TNF-A | Rheumatoid Arthritis, Ankylosing |
| | | Spondylitis, Psoriatic Arthritis, Psoriasis, |
| | | Crohn's Disease, Ulcerative Colitis |
| Intetumumab | CD51 | Solid Tumors (Prostate Cancer, Melanoma) |
| Inolimomab | CD25 (A Chain Of IL-2 | Graft-Versus-Host Disease |
| | Receptor) | |
| Inotuzumab | CD22 | Cancer |
| Ozogamicin | | |
| Ipilimumab | CD152 | Melanoma |
| Iratumumab | CD30 (TNFRSF8) | Hodgkin's Lymphoma |
| Itolizumab | CD6 | Cancer |
| Ixekizumab | IL-17A | Autoimmune Diseases |
| Keliximab | CD4 | Chronic Asthma |
| Labetuzumab | CEA | Colorectal Cancer |
| Lambrolizumab | PDCD1 | Antineoplastic Agent |
| Lampalizumab | CFD | Cancer |
| Lebrikizumab | IL-13 | Asthma |
| Lemalesomab | NCA-90 | Diagnostic Agent |
| | (Granulocyte Antigen) | |
| Lerdelimumab | TGF Beta 2 | Reduction Of Scarring After Glaucoma |
| | | Surgery |
| Lexatumumab | TRAIL-R2 | Cancer |
| Libivirumab | Hepatitis B Surface | Hepatitis B |
| | Antigen | |
| Ligelizumab | IGHE | Cancer |
| Lintuzumab | CD33 | Cancer |
| Lirilumab | KIR2D | Cancer |
| Lodelcizumab | PCSK9 | Hypercholesterolemia |
| Lorvotuzumab | CD56 | Cancer |
| Lucatumumab | CD40 | Multiple Myeloma, Non-Hodgkin's |
| | | Lymphoma, Hodgkin's Lymphoma |
| Lumiliximab | CD23 | Chronic Lymphocytic Leukemia |
| Mapatumumab | TRAIL-R1 | Cancer |
| Margetuximab | Ch4d5 | Cancer |
| Mavrilimumab | GMCSF Receptor A- | Rheumatoid Arthritis |
| | Chain | |
| Matuzumab | EGFR | Colorectal, Lung And Stomach Cancer |
| Mepolizumab | IL-5 | Asthma And White Blood Cell Diseases |
| Metelimumab | TGF Beta 1 | Systemic Scleroderma |
| Milatuzumab | CD74 | Multiple Myeloma And Other Hematological |
| | | Malignancies |
| Minretumomab | TAG-72 | Cancer |
| Mitumomab | GD3 Ganglioside | Small Cell Lung Carcinoma |
| Mogamulizumab | CCR4 | Cancer |
| Morolimumab | Rhesus Factor | Cancer |
| Motavizumab | Respiratory Syncytial | Respiratory Syncytial Virus (Prevention) |
| | Virus | |
| Moxetumomab | CD22 | Cancer |
| Pasudotox | | |
| Muromonab-CD3 | CD3 | Prevention Of Organ Transplant Rejections |
| Nacolomab | C242 Antigen | Colorectal Cancer |
| Tafenatox | | |
| Namilumab | CSF2 | Cancer |
| Naptumomab | 5T4 | Non-Small Cell Lung Carcinoma, Renal Cell |
| Estafenatox | | Carcinoma |
| Narnatumab | RON | Cancer |
| Natalizumab | Integrin A4 | Multiple Sclerosis, Crohn's Disease |
| Nebacumab | Endotoxin | Sepsis |
| Necitumumab | EGFR | Non-Small Cell Lung Carcinoma |
| Nerelimomab | TNF-A | Cancer |
| Nesvacumab | Angiopoietin 2 | Cancer |
| Nimotuzumab | EGFR | Squamous Cell Carcinoma, Head And Neck |
| | | Cancer, Nasopharyngeal Cancer, Glioma |
| Nivolumab | Igg4 | Cancer |
| Nofetumomab | Undetermined | Cancer |
| Merpentan | | |
| Ocaratuzumab | CD20 | Cancer |
| Ocrelizumab | CD20 | Rheumatoid Arthritis, Lupus Erythematosus |
| Odulimomab | LFA-1 (CD11a) | Prevention Of Organ Transplant |
| | | Rejections, Immunological Diseases |
| Ofatumumab | CD20 | Chronic Lymphocytic Leukemia |
| Olaratumab | PDGF-R A | Cancer |
| Olokizumab | IL6 | Cancer |
| Onartuzumab | Human Scatter Factor | Cancer |
| | Receptor Kinase | |
| Ontuxizumab | TEM1 | Cancer |
| Oportuzumab | Epcam | Cancer |
| Monatox | | |
| Oregovomab | CA-125 | Ovarian Cancer |
| Orticumab | Oxldl | Cancer |
| Otlertuzumab | CD37 | Cancer |
| Oxelumab | OX-40 | Asthma |
| Ozanezumab | NOGO-A | ALS And Multiple Sclerosis |
| Ozoralizumab | TNF-A | Inflammation |
| Pagibaximab | Lipoteichoic Acid | Sepsis (Staphylococcus) |
| Palivizumab | F Protein Of | Respiratory Syncytial Virus (Prevention) |
| | Respiratory Syncytial | |
| | Virus | |
| Panitumumab | EGFR | Colorectal Cancer |
| Pankomab | Tumor Specific | Ovarian Cancer |
| | Glycosylation Of | |
| | MUC1 | |
| Panobacumab | Pseudomonas | Pseudomonas Aeruginosa Infection |
| | Aeruginosa | |
| Parsatuzumab | EGFL7 | Cancer |
| Pascolizumab | IL-4 | Asthma |
| Pateclizumab | LTA | TNF |
| Patritumab | HER3 | Cancer |
| Pemtumomab | MUC1 | Cancer |
| Perakizumab | IL17A | Arthritis |
| Pertuzumab | HER2/Neu | Cancer |
| Pexelizumab | C5 | Reduction Of Side-Effects Of |
| | | Cardiac Surgery |
| Pidilizumab | PD -1 | Cancer And Infectious Diseases |
| Pinatuzumab | CD22 | Cancer |
| Vedotin | | |
| Pintumomab | Adenocarcinoma | Adenocarcinoma |
| | Antigen | |
| Placulumab | Human TNF | Cancer |
| Polatuzumab | CD79B | Cancer |
| Vedotin | | |
| Ponezumab | Human Beta-Amyloid | Alzheimer's Disease |
| Pritoxaximab | E. Coli Shiga Toxin | Cancer |
| | Type-1 | |
| Pritumumab | Vimentin | Brain Cancer |
| Pro 140 | Ccr5 | HIV Infection |
| Quilizumab | IGHE | Cancer |
| Racotumomab | N-Glycolylneuraminic | Cancer |
| | Acid | |
| Radretumab | Fibronectin Extra | Cancer |
| | Domain-B | |
| Rafivirumab | Rabies Virus | Rabies (Prophylaxis) |
| | Glycoprotein | |
| Ramucirumab | VEGFR2 | Solid Tumors |
| Ranibizumab | VEGF-A | Macular Degeneration (Wet Form) |
| Raxibacumab | Anthrax Toxin, | Anthrax (Prophylaxis And Treatment) |
| | Protective Antigen | |
| Regavirumab | Cytomegalovirus | Cytomegalovirus Infection |
| | Glycoprotein B | |
| Reslizumab | IL-5 | Inflammations Of The Airways, Skin And |
| | | Gastrointestinal Tract |
| Rilotumumab | HGF | Solid Tumors |
| Rituximab | CD20 | Lymphomas, Leukemias, Some |
| | | Autoimmune Disorders |
| Robatumumab | IGF-1 Receptor | Cancer |
| Roledumab | RHD | Cancer |
| Romosozumab | Sclerostin | Osteoporosis |
| Rontalizumab | IFN-α | Systemic Lupus Erythematosus |
| Rovelizumab | CD11, CD18 | Haemorrhagic Shock |
| Ruplizumab | CD154 (CD40L) | Rheumatic Diseases |
| Samalizumab | CD200 | Cancer |
| Sarilumab | IL6 | Rheumatoid Arthritis, Ankylosing |
| | | Spondylitis |
| Satumomab | TAG-72 | Cancer |
| Pendetide | | |
| Secukinumab | IL-17A | Uveitis, Rheumatoid Arthritis Psoriasis |
| Seribantumab | ERBB3 | Cancer |
| Setoxaximab | E. Coli Shiga Toxin | Cancer |
| | Type-1 | |
| Sevirumab | Cytomegalovirus | Cytomegalovirus Infection |
| Sibrotuzumab | FAP | Cancer |
| Sgn-CD19a | CD19 | Acute Lymphoblastic Leukemia And B |
| | | Cell Non-Hodgkin Lymphoma |
| Sgn-CD33a | CD33 | Acute Myeloid Leukemia |
| Sifalimumab | IFN-A | SLE, Dermatomyositis, Polymyositis |
| Siltuximab | IL-6 | Cancer |
| Simtuzumab | LOXL2 | Fibrosis |
| Siplizumab | CD2 | Psoriasis, Graft-Versus-Host Disease |
| | | (Prevention) |
| Sirukumab | IL-6 | Rheumatoid Arthritis |
| Solanezumab | Beta-Amyloid | Alzheimer's Disease |
| Solitomab | Epcam | Cancer |
| Sonepcizumab | Sphingosine-1- | Choroidal And Retinal Neovascularization |
| | Phosphate | |
| Sontuzumab | Episialin | Cancer |
| Stamulumab | Myostatin | Muscular Dystrophy |
| Sulesomab | NCA-90 | Osteomyelitis |
| | (Granulocyte Antigen) | |
| Suvizumab | HIV-1 | Viral Infections |
| Tabalumab | BAFF | B Cell Cancers |
| Tacatuzumab | Alpha-Fetoprotein | Cancer |
| Tetraxetan | | |
| Tadocizumab | Integrin AIIBβ3 | Percutaneous Coronary Intervention |
| Tanezumab | NGF | Pain |
| Taplitumomab | CD19 | Cancer |
| Paptox | | |
| Tefibazumab | Clumping Factor A | Staphylococcus Aureus Infection |
| Telimomab | Undetermined | Cancer |
| Tenatumomab | Tenascin C | Cancer |
| Teneliximab | CD40 | Cancer |
| Teprotumumab | CD221 | Hematologic Tumors |
| Ticilimumab | CTLA-4 | Cancer |
| Tildrakizumab | IL23 | Immunologically Mediated |
| | | Inflammatory Disorders |
| Tigatuzumab | TRAIL-R2 | Cancer |
| Tnx-650 | Il-13 | Hodgkin's Lymphoma |
| Tocilizumab | IL-6 Receptor | Rheumatoid Arthritis |
| Toralizumab | CD154 (CD40L) | Rheumatoid Arthritis, Lupus Nephritis |
| Tositumomab | CD20 | Follicular Lymphoma |
| Tovetumab | CD140a | Cancer |
| Tralokinumab | IL-13 | Asthma |
| Trastuzumab | HER2/Neu | Breast Cancer |
| Trbs07 | Gd2 | Melanoma |
| Tremelimumab | CTLA-4 | Cancer |
| Tucotuzumab | Epcam | Cancer |
| Celmoleukin | | |
| Tuvirumab | Hepatitis B Virus | Chronic Hepatitis B |
| Ublituximab | MS4A1 | Cancer |
| Urelumab | 4-1BB | Cancer |
| Urtoxazumab | Escherichia Coli | Diarrhoea Caused By E. Coli |
| Ustekinumab | IL-12, IL-23 | Multiple Sclerosis, Psoriasis, Psoriatic |
| | | Arthritis |
| Vantictumab | Frizzled Receptor | Cancer |
| Vapaliximab | AOC3 (VAP-1) | Cancer |
| Vatelizumab | ITGA2 | Cancer |
| Vedolizumab | Integrin A4β7 | Crohn's Disease, Ulcerative Colitis |
| Veltuzumab | CD20 | Non-Hodgkin's Lymphoma |
| Vepalimomab | AOC3 (VAP-1) | Inflammation |
| Vesencumab | NRP1 | Cancer |
| Volociximab | Integrin A5β1 | Solid Tumors |
| Vorsetuzumab | CD70 | Cancer |
| Votumumab | Tumor Antigen | Colorectal Tumors |
| | CTAA16.88 | |
| Zalutumumab | EGFR | Squamous Cell Carcinoma Of The |
| | | Head And Neck |
| Zatuximab | HER1 | Cancer |
| Ziralimumab | CD147 | Cancer |
| Zolimomab | CD5 | Systemic Lupus Erythematosus, |
| Aritox | | Graft-Versus-Host Disease |

A Disease-Associated Antigen may be characteristically expressed in a pathogen-infected cell or in a cancer cells and processed and displayed on a cell surface in the context of an MHC complex, but not characteristically expressed in a normal cell. Antibodies that recognize such peptide fragments are known in the art or can be generated using well-known methods, including those described in WO 2002/014870.

The polypeptides of the Tri-Specific Binding Molecules of the present invention can be adapted to contain the Variable Light or Variable Heavy Domains (the case of the first and second polypeptide chains of such molecules) or the heavy or light chains (in the case of the third and fourth polypeptide chains of such molecules) of such antibodies. Thus, the above-described antibodies may be used to produce Tri-Specific Binding Molecules of the present invention whose Site A, Site B or Site C is capable of binding to an epitope of such Disease-Associated Antigens].

### B. Preferred Structural Attributes

Typically, the Tri-Specific Binding Molecules of the present molecules will comprise four different polypeptide chains, each having an amino terminus and a carboxyl terminus (see FIGS. 4A-4D), however, the molecules may comprise fewer or greater numbers of polypeptide chains by fusing such polypeptide chains to one another (e.g., via a peptide bond) or by dividing such polypeptide chains to form additional polypeptide chains, or by associating fewer or additional polypeptide chains via disulfide bonds. FIGS. 4E-4J illustrate this aspect of the present invention by schematically depicting such molecules having three polypeptide chains. FIGS. 4K-4L illustrate this aspect of the present invention by schematically depicting molecules having five polypeptide chains.

The various immunoglobulin Domains of such molecules may be derived from immunoglobulins of any isotype or allotype including, but not limited to, IgA, IgD, IgG, IgE and IgM. In preferred embodiments, as discussed below such immunoglobulins are derived from IgG immunoglobulins. In specific embodiments, the IgG isotype used is IgG1, however IgG of other isotypes (e.g., IgG2, IgG3 or IgG4 or an allotype thereof) may be employed. When an IgG4 Fc Domain is utilized, the present invention encompasses the introduction of a stabilizing mutation such as S228P, as numbered by the EU index as set forth in Kabat (Lu et al., (2008) “The Effect Of A Point Mutation On The Stability Of Igg4 As Monitored By Analytical Ultracentrifugation,” J. Pharmaceutical Sciences 97:960-969) to reduce the incidence of strand exchange. Other stabilizing mutations known in the art may be introduced into an IgG4 Fc Domain (Peters, P et al., (2012) “Engineering an Improved IgG4 Molecule with Reduced Disulfide Bond Heterogeneity and Increased Fab Domain Thermal Stability,” J. Biol. Chem., 287:24525-24533; PCT Patent Publication No: WO 2008/145142). Since the N297A, L234A, L235A and D265A substitutions abolish effector function, in circumstances in which effector function is desired, these substitutions would preferably not be employed.

FIGS. 4A-4D provide a diagrammatic representation of the Domains of preferred Tri-Specific Binding Molecules. FIGS. 4A and 4B, respectively, illustrate schematically the Domains of preferred Tri-Specific Binding Molecules in which the Non-Diabody-Type Binding Domain is a Fab-Type Binding Domain or a Receptor-Type Binding Domain. FIGS. 4C and 4D, respectively, illustrate schematically the Domains of preferred Tri-Specific Binding Molecules having different Domain orientations in which the Non-Diabody-Type Binding Domain is a Fab-Type Binding Domain or a Receptor-Type Binding Domain.

As indicated above, one of Epitope I, Epitope II or Epitope III that is bound by the Binding Domains of such exemplary preferred Tri-Specific Binding Molecules may be an epitope of a Disease-Associated Antigen. Most preferably, the Binding Domain of such exemplary preferred Tri-Specific Binding Molecule that binds to such epitope of a Disease-Associated Antigen is a Fab-Type Binding Domain. The polypeptides of such Tri-Specific Binding Molecules of the present invention can be adapted to contain the Variable Light or Variable Heavy Domains (the case of the first and second polypeptide chains of such molecules) or the heavy or light chains (in the case of the third and fourth polypeptide chains of such molecules). Thus, such antibodies may be used to produce Tri-Specific Binding Molecules of the present invention whose Site A, Site B or Site C is capable of binding to an epitope of such Disease-Associated Antigens.

### 1. Preferred First Polypeptide Chain

A first polypeptide chain of a preferred Tri-Specific Binding Molecule of the present invention will comprise a Variable Light Chain Domain capable of binding to Epitope I (VL_{I}), a Variable Heavy Chain Domain capable of binding to Epitope II (VH_{II}), a cysteine residue or Cysteine-Containing Domain and a Heterodimer-Promoting Domain and a CH2-CH3 Domain.

Since the Variable Light Chain and Variable Heavy Chain Domains of the first polypeptide are directed toward different epitopes, they cannot associate together to form a Binding Domain that is able to bind either Epitope I or Epitope II. The Variable Light Chain and Variable Heavy Chain Domains of the first polypeptide are spaced apart from one another by an intervening linker peptide that is sufficiently short as to substantially prevent the association of these Domains. An exemplary linker, termed “Linker 1,” has the sequence (SEQ ID NO:1): GGGSGGGG.

The Variable Heavy Chain Domain of the first polypeptide and the Heterodimer-Promoting Domain of that polypeptide are preferably spaced apart from one another by an intervening linker peptide that contains 1, 2, 3 or more cysteine residues. A preferred Cysteine-Containing Domain (“Linker 2”) has the sequence is SEQ ID NO:2: GGCGGG. Alternatively, or additionally, a Cysteine-Containing Heterodimer-Promoting Domain, as described below, may be used.

Thus, in some embodiments, one or more cysteine residues (or Cysteine-Containing Domain, such as a cysteine-containing peptide linker) will be incorporated into the first polypeptide chain (and/or into the second, third, fourth or further polypeptide chains of the Tri-Specific Binding Molecules of the present invention) in order to covalently bond two such polypeptide chains together, whereas in equivalent embodiments such cysteine residue(s) may be incorporated into a Heterodimer-Promoting Domain, or into another domain in order to achieve the same result.

The Heterodimer-Promoting Domain of the first polypeptide and the Heterodimer-Promoting Domain of the second polypeptide are coordinately selected. The Domains differ from one another and are designed to associate with one another so as to promote the association of the first and second polypeptide chains. For example, one of the Heterodimer-Promoting Domains will be engineered to have a negative charge at pH 7, while the other of the two polypeptide chains will be engineered to have a positive charge at pH 7. The presence of such charged Domains promotes association between the first and second polypeptides, and thus fosters heterodimerization. It is immaterial which Heterodimer-Promoting Domains is provided to which chain, as long as the Domains employed on the first and second polypeptide chains differ so as to foster heterodimerization between such chains.

In a preferred embodiment, the Heterodimer-Promoting Domain of the first polypeptide chain is either an “E-coil” Domain (SEQ ID NO:3): EVAALEKEVAALEKEVAALEKEVAALEK, or a “K-coil” Domain (SEQ ID NO:4): KVAALKEKVAALKEKVAALKEKVAALKE. More preferably, the first polypeptide chain will possess an “E-coil” Domain. The first polypeptide chain may contain only a single such coil separator, or it may contain more than one such coil separators (e.g., two separators) and can be the same charge preferably of opposite charge.

In a preferred embodiment, the Heterodimer-Promoting Domain of the first polypeptide chain will comprise either four tandem “E-coil” helical domains (SEQ ID NO:3: EVAALEK-EVAALEK-EVAALEK-EVAALEK), whose glutamate residues will form a negative charge at pH 7, or four tandem “K-coil” domains (SEQ ID NO:4: KVAALKE-KVAALKE-KVAALKE-KVAALKE), whose lysine residues will form a positive charge at pH 7. The presence of such charged domains promotes association between the first and second polypeptide chains, and thus fosters heterodimerization. Especially preferred is a Heterodimer-Promoting Domain in which one of the four tandem “E-coil” helical domains of SEQ ID NO:3 or SEQ ID NO:4 has been modified to contain a cysteine residue: EVAACEK-EVAALEK-EVAALEK-EVAALEK (SEQ ID NO:115) or in which one of the four tandem “K-coil” helical domains of SEQ ID NO:4 has been modified to contain a cysteine residue: KVAACKE-KVAALKE-KVAALKE-KVAALKE (SEQ ID NO:116). Other E-coil and K-coil Domains that may be employed in accordance with the present invention are disclosed in: Woolfson, D. N. (2005) “The Design Of Coiled-Coil Structures And Assemblies,” Adv. Prot. Chem. 70:79-112, Straussman, R. et al. (2007) “Kinking the Coiled Coil—Negatively Charged Residues at the Coiled-coil Interface,” J. Molec. Biol. 366:1232-1242; Apostolovic, B. et al. (2008) “pH-Sensitivity of the E3/K3 Heterodimeric Coiled Coil,” Biomacromolecules 9:3173-3180; Arndt, K. M. et al. (2001) “Helix-stabilized Fv (hsFv) Antibody Fragments: Substituting the Constant Domains of a Fab Fragment for a Heterodimeric Coiled-coil Domain,” J. Molec. Biol. 312:221-228; Steinkruger, J. D. et al. (2012) “The d′-d-d′ Vertical Triad is Less Discriminating Than the a′-a-a′ Vertical Triad in the Antiparallel Coiled-coil Dimer Motif” J. Amer. Chem. Soc. 134(5):2626-2633; Ghosh, T. S. et al. (2009) “End-To-End And End-To-Middle Interhelical Interactions: New Classes Of Interacting Helix Pairs In Protein Structures,” Acta Crystallographica D65:1032-1041; Grigoryan, G. et al. (2008) “Structural Specificity In Coiled-Coil Interactions,” Curr. Opin. Struc. Biol. 18:477-483; Boucher, C. et al. (2010) “Protein Detection By Western Blot Via Coiled—Coil Interactions,” Analytical Biochemistry 399:138-140; Cachia, P. J. et al. (2004) “Synthetic Peptide Vaccine Development: Measurement Of Polyclonal Antibody Affinity And Cross-Reactivity Using A New Peptide Capture And Release System For Surface Plasmon Resonance Spectroscopy,” J. Mol. Recognit. 17:540-557; De Crescenzo, G. D. et al. (2003) “Real-Time Monitoring of the Interactions of Two-Stranded de novo Designed Coiled-Coils: Effect of Chain Length on the Kinetic and Thermodynamic Constants of Binding,” Biochemistry 42:1754-1763; Tripet, B. et al. (2002) “Kinetic Analysis of the Interactions between Troponin C and the C-terminal Troponin I Regulatory Region and Validation of a New Peptide Delivery Capture System used for Surface Plasmon Resonance,” J. Molec. Biol. 323:345-362; and Zeng, Y. et al. (2008) “A Ligand-Pseudoreceptor System Based On de novo Designed Peptides For The Generation Of Adenoviral Vectors With Altered Tropism,” J. Gene Med. 10:355-367.

Preferably, the employed Heterodimer-Promoting Domain and the CH2-CH3 Domain of the first polypeptide chain are spaced apart from one another by an intervening cysteine-containing linker peptide that provides improved stabilization to the Heterodimer-Promoting Domain. A preferred cysteine-containing linker peptide (“Linker 3”) has the amino acid sequence (SEQ ID NO:5): DKTHTCPPCP.

The amino acid sequence of a wild-type CH2-CH3 Domain is as follows (positioning is as in the EU index as in Kabat et al. (1992) SEQUENCES OF PROTEINS OF IMMUNOLOGICAL INTEREST, National Institutes of Health Publication No. 91-3242) (SEQ ID NO:6):

```
| CH2→
APELLGGPS VFLFPPKPKD TLMISRTPEV TCVVVDVSHE DPEVKFNWYV DGVEVHNAKT
231 240 250 260 270 280
                                                                                                      ←CH2 | CH3→
KPREEQYNST YRVVSVLTVL HQDWLNGKEY KCKVSNKALP APIEKTISKA K GQPREPQVY
290 300 310 320 330 340
TLPPSREEMT KNQVSLTCLV KGFYPSDIAV EWESNGQPEN NYKTTPPVLD SDGSFFLYSK
350 360 370 380 390 400
                                                                          ←CH3 |
LTVDKSRWQQ GNVFSCSVMH EALHNHYTQK SLSLSPGK
410 420 430 440
```

In some expression systems the C-terminal amino acid residue of the CH3 Domain may be post-translationally removed. Accordingly, the C-terminal residue of the CH3 Domain is an optional amino acid residue.

The CH2-CH3 Domain of the first polypeptide chain will preferably be modified to promote heterodimerization between the CH2-CH3 Domain of the third polypeptide chain (see below). For example, an amino acid substitution (preferably a substitution with an amino acid comprising a bulky side group forming a ‘knob’, e.g., tryptophan) can be introduced into the CH2 or CH3 Domain of the first polypeptide chain such that steric interference will prevent interaction with a similarly mutated Domain and will obligate the mutated Domain to pair with a Domain into which a complementary, or accommodating mutation has been engineered, i.e., ‘the hole’ (e.g., a substitution with glycine). Such sets of mutations can be engineered into any pair of polypeptides comprising the diabody molecule, and further, engineered into any portion of the polypeptides chains of said pair. Methods of protein engineering to favor heterodimerization over homodimerization are well-known in the art, in particular with respect to the engineering of immunoglobulin-like molecules, and are encompassed herein (see e.g., U.S. Pat. No. 7,695,936 and Patent Publication 2007/0196363, Ridgway et al. (1996) “‘Knobs-Into-Holes’ Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety. A preferred knob is created by modifying a native IgG Fc Domain to contain the modification T366W. A preferred hole is created by modifying a native IgG Fc Domain to contain the modification T366S, L368A and Y407V. To aid in purifying the Tri-Specific Binding Molecules of the present invention, the polypeptide chain containing the hole mutations additionally comprises a substitution at position 435 (H435R) to remove the Protein A binding site. Thus, homodimers of polypeptides containing the hole mutations will not bind to protein A, whereas the Tri-Specific Binding Molecules that form as a result of knob and hole containing heterodimers will retain its ability to bind protein A via the protein A binding site on the polypeptide chain containing the knob mutation.

The CH2-CH3 Domain of the first polypeptide chain will preferably be modified to reduce or abrogate binding of the Fc to Fc receptors. Such mutations are well-known in the art and include substitutions at positions 234, 235, 265 and 297 (see U.S. Pat. No. 5,624,821). Preferred substitutions include one or more of L234A and L235A, D265A and N297Q.

Preferably, therefore the CH2-CH3 Domain of the first polypeptide chain will have the “knob-bearing” sequence (SEQ ID NO:7):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGK

or the “hole-bearing” sequence with an H435R substitution to abrogate Protein A binding (SEQ ID NO:8):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGK

It is preferred that the first polypeptide chain will have a “knob-bearing” CH2-CH3 sequence, such as that of SEQ ID NO:7.

As will be recognized, a “hole-bearing” CH2-CH3 Domain (e.g., SEQ ID NO:8) could be employed in the first polypeptide chain, in which case, a “knob-bearing” CH2-CH3 Domain (e.g., SEQ ID NO:7) would be employed in the third polypeptide chain.

Thus, in sum, a preferred first polypeptide chain of a preferred Tri-Specific Binding Molecule of the present invention will comprise the Domains and linkers: (VL_{I} Domain)-(Linker 1)-(VH_{II} Domain)-(Cysteine-Containing Domain (Linker 2))-(E-coil Heterodimer-Promoting Domain)-(Linker 3)-(Knob-Bearing CH2-CH3 Domain) or (VL_{I} Domain)-(Linker 1)-(VH_{II} Domain)-(Linker 2)-(Cysteine-Containing E-coil Heterodimer-Promoting Domain)-(Linker 3)-(Knob-Bearing CH2-CH3 Domain or (VL_{I} Domain)-(Linker 1)-(VH_{III} Domain)-(Linker 2)-(Cysteine-Containing E-coil Heterodimer-Promoting Domain)-(Linker 3)-(Knob-Bearing CH2-CH3 Domain).

### 2. Preferred Second Polypeptide Chain

A second polypeptide chain of such preferred Tri-Specific Binding Molecules will comprise, in the N-terminal to C-terminal direction, a Variable Light Chain Domain capable of binding to Epitope II (VL_{II}), a Variable Heavy Chain Domain capable of binding to Epitope I (VH_{I}), a cysteine residue or Cysteine-Containing Domain and a Heterodimer-Promoting Domain.

Since the Variable Light Chain and Variable Heavy Chain Domains of the second polypeptide are directed toward different epitopes, they cannot associate together to form a Binding Domain that is able to bind either Epitope I or Epitope II. The Variable Light Chain and Variable Heavy Chain Domains of the second polypeptide are spaced apart from one another by an intervening linker peptide that is sufficiently short as to substantially prevent the association of these Domains. “Linker 1,” having the sequence (SEQ ID NO:1): GGGSGGGG is an exemplary linker for this purpose.

As in the case of the first polypeptide chain, the Variable Heavy Chain Domain of the second polypeptide and the Heterodimer-Promoting Domain of that polypeptide are preferably spaced apart from one another by an intervening Cysteine-Containing Domain that contains 1, 2, 3 or more cysteine residues. “Linker 2,” having the sequence (SEQ ID NO:2) GGCGGG is an exemplary linker for this purpose. Such cysteine residues can form disulfide bonds with cysteine residues in the cysteine-containing spacer peptide that separates the Variable Heavy Chain Domain of the first polypeptide and the Heterodimer-Promoting Domain of that polypeptide. Thus, the first and second polypeptides of the Tri-Specific Binding Molecules of the present invention are covalently bonded to one another. Alternatively, a Cysteine-Containing Heterodimer-Promoting Domain, as described above, may be used.

As discussed above, the Heterodimer-Promoting Domain of the second polypeptide chain is selected so as coordinate with the Heterodimer-Promoting Domain of the first polypeptide chain. Thus, in a preferred embodiment, the Heterodimer-Promoting Domain of the first polypeptide chain is either a “K-coil” Domain (e.g., SEQ ID NO:4 or SEQ ID NO:116) or an “E-coil” Domain (e.g., SEQ ID NO:3 or SEQ ID NO:115). Since the first polypeptide chain will preferably possess an “E-coil” Domain, the second polypeptide chain will preferably contain a “K-coil” Domain.

As the first and second polypeptide chains are polypeptide chains of a diabody, they are able to associate together to form a Domain I Binding Domain (VL_{A}/VH_{A}) that recognizes and immunospecifically binds to Epitope I, and a Domain II Binding Domain (VL_{B}/VH_{B}) that recognizes and immunospecifically binds to Epitope II.

Thus, in sum, a preferred second polypeptide chain of a preferred Tri-Specific Binding Molecule of the present invention will comprise the Domains and linkers: (VL_{II} Domain)-(Linker 1)-(VH_{I} Domain)-(Cysteine-Containing Domain (Linker 2))-(K-coil Heterodimer-Promoting Domain) or (VL_{II} Domain)-(Linker 1)-(VH_{I} Domain)-(Linker 2)-(Cysteine-Containing K-coil Heterodimer-Promoting Domain).

### 3. Preferred Third Polypeptide Chain

A third polypeptide chain of a preferred Tri-Specific Binding Molecule of the present invention is a polypeptide that comprises, in the N-terminal to C-terminal direction, a Binding Domain, a Cysteine-Containing Domain that may optionally comprise a CH1-Hinge Domain, and a CH2-CH3 Domain. The Binding Domain of the third polypeptide chain of a preferred Tri-Specific Binding Molecule of the present invention may be a Variable Heavy Chain Domain capable of binding to Epitope III (VH_{III}), in which case, the fourth polypeptide chain of the preferred Tri-Specific Binding Molecules of the present invention (discussed below) is a polypeptide that comprises a Variable Light Chain Domain capable of binding to Epitope III (VL_{III}), such that the Binding Domain is capable of immunospecific binding to an antigen possessing Epitope III. Alternatively, the Binding Domain of the third polypeptide chain of the preferred Tri-Specific Binding Molecules of the present invention may comprise a T Cell Receptor-Type Binding Domain, in which case, the fourth polypeptide chain of the preferred Tri-Specific Binding Molecules of the present invention (discussed below) is a polypeptide that comprises a complementary T Cell Receptor-Type Binding Domain, such that the interaction of two polypeptide chains forms a Binding Domain that is capable of physiospecific binding to an antigen molecule displayed in the MHC complex arrayed on the surface of a Cell. The third polypeptide chain may be isolated from naturally occurring antibodies. Alternatively, it may be constructed recombinantly. An exemplary CH1 Domain is a human IgG1 CH1 Domain having the amino acid sequence (SEQ ID NO:9):

ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKV

An exemplary Hinge Domain is a human IgG1 Hinge Domain having the amino acid sequence (SEQ ID NO:10): EPKSCDKTHTCPPCP. As will be recognized, the exemplary Hinge Domain comprises multiple cysteine residues (Elkabetz et al. (2005) “Cysteines In CH1 Underlie Retention Of Unassembled Ig Heavy Chains,” J. Biol. Chem. 280:14402-14412) that may participate in interchain covalent bonding. Alternatively, a different Cysteine-Containing Domain may be employed (e.g., a peptide having the amino acid sequence: VEPKSC (SEQ ID NO:12), AEPKSC (SEQ ID NO:127), GVEPKSC (SEQ ID NO:133) or GGCGGG (SEQ ID NO:2)).

Although a wild-type CH2-CH3 Domain may be employed, it is preferred, as described above, to employ a modified CH2-CH3 Domain that promotes heterodimerization with the CH2-CH3 Domain of the first polypeptide chain.

Preferably, therefore the CH2-CH3 Domain of the third polypeptide chain will be a “hole-bearing” CH2-CH3 Domain whose amino acid sequence is complementary to the “knob-bearing” CH2-CH3 Domain (SEQ ID NO:7) employed in the first polypeptide. As discussed above, the hole-bearing CH2-CH3 Domain preferably should comprise a substitution at position 435 (H435R) to remove the Protein A binding site. An exemplary “hole-bearing” CH2-CH3 Domain with the H435R substitution for the third polypeptide is SEQ ID NO:8.

As will be recognized, a “knob-bearing” CH2-CH3 Domain (e.g., SEQ ID NO:7) could be employed in the third polypeptide chain, in which case, a “hole-bearing” CH2-CH3 Domain (e.g., SEQ ID NO:8) would be employed in the first polypeptide chain.

In the embodiment in which the third (and fourth) polypeptide chains of the preferred Tri-Specific Binding Molecules of the present invention each comprise a polypeptide chain of a T cell Receptor-Type Binding Domain, which recognized antigen displayed on a cell surface in the context of class I MHC. Methods are well-known in the art to produce such T cell receptor-type binding domains (e.g., US2012/0294874A1).

Thus, in sum, a third polypeptide chain of the preferred Tri-Specific Binding Molecules of the present invention will comprise the Domains and linkers: (VH_{I} Domain)-(Cysteine-Containing Domain (optionally a CH1 Domain and/or a Hinge Domain)-(Hole-Bearing CH2-CH3 Domain), or (Receptor-Type Binding Domain; first or second polypeptide thereof)-(Cysteine-Containing Domain (optionally a CH1 Domain and/or a Hinge Domain)-(Hole-Bearing CH2-CH3 Domain).

### 4. Preferred Fourth Polypeptide Chain

A fourth polypeptide chain of the preferred Tri-Specific Binding Molecules of the present invention is either a polypeptide of a Receptor-Type Binding Domain (wherein the third and fourth polypeptide chains form a Receptor-Type Binding Domain), or more preferably, a polypeptide portion of a Light Chain of the above-indicated antibody that immunospecifically binds to Epitope III and/or which is complementary to the Binding Domain of the third polypeptide chain.

Thus, wherein the third and fourth polypeptides form a Fab-Type Binding Domain such fourth polypeptide chain comprises, in the N-terminal to C-terminal direction, a Variable Light Chain Domain capable of binding to Epitope III (VL_{III}), and a Cysteine-Containing Domain for promoting covalent bonding to the third polypeptide chain, or a Binding Domain and such Cysteine-Containing Domain for promoting covalent bonding to the third polypeptide chain. Such Cysteine-Containing Domain may be a CL Domain, or a cysteine-containing portion thereof, such as (SEQ ID NO:11) FNRGEC or (SEQ ID NO:128) GFNRGEC or a linker such as Linker 2 (having the sequence (SEQ ID NO:2) GGCGGG. An exemplary a Cysteine-Containing Domain that forms disulfide bonds with such Linker 2 comprises the amino acid sequence VEPKSC (SEQ ID NO:12) or a Hinge Domain.

The fourth polypeptide chain may be isolated from naturally occurring antibodies. Alternatively, it may be constructed recombinantly. An preferred CL Domain is a human IgG1 CL Kappa Domain having the amino acid sequence (SEQ ID NO:13):

RTVAAPSVFI FPPSDEQLKS GTASVVCLLN NFYPREAKVQ
WKVDNALQSG NSQESVTEQD SKDSTYSLSS TLTLSKADYE
KHKVYACEVT HQGLSSPVTK SFNRGEC

Alternatively, an exemplary CL Domain is a human IgG1 CL Lambda2 Domain having the amino acid sequence (SEQ ID NO:14):

QPKAAPSVTL FPPSSEELQA NKATLVCLIS DFYPGAVTVA
WKADSSPVKA GVETTPSKQS NNKYAASSYL SLTPEQWKSH
RSYSCQVTHE GSTVEKTVAP TECS

As will be noticed, the CL Domain, or other Cysteine-Containing Domain, of the fourth polypeptide chain comprises a cysteine residue that is able to covalently bond to a cysteine residue of the Cysteine-Containing Domain of the third polypeptide chain (e.g., a CH1 Domain) to thereby covalently complex the third and fourth polypeptide chains of the Tri-Specific Binding Molecules of the present invention to one another. Thus the third and fourth polypeptide chains are covalently bonded to one another.

Additionally, cysteine residues of the CH2-CH3 Domain of the first polypeptide chain can form disulfide bonds with cysteine residues of the CH2-CH3 Domain of the third polypeptide chain. Thus the first and third polypeptide chains are covalently bonded to one another.

Thus, in sum, a fourth polypeptide chain of the preferred Tri-Specific Binding Molecules of the present invention will comprise the Domains and linkers: (VL_{III} Domain)-(Cysteine-Containing Domain (optionally a CL Domain), or (Receptor-Type Binding Domain; first or second polypeptide thereof)-(Cysteine-Containing Domain (optionally a CL Domain).

### C. Alternative First Polypeptide Chain

In one embodiment, the orientations of the above-described Domains will be in the N-terminal to C-terminal direction. The present invention, however, also contemplates a variation thereof, wherein the orientations of the Domains of the first polypeptide chain are: NH₂-(Knob-Bearing CH3-CH2 Domain)-(VL_{I} Domain)-(Linker 1)-(VH_{II} Domain)-(Cysteine-Containing Domain Linker 2)-(E-coil Heterodimer-Promoting Domain). Preferably, a Cysteine-Containing Domain is present, N-terminal to such CH2-CH3 Domain. The sequence of an exemplary peptide is (SEQ ID NO:5): DKTHTCPPCP, however, alternative linkers may be employed, e.g., EPKSCDKTHTCPPCP (SEQ ID NO:129) or LEPKSSDKTHTCPPCP; SEQ ID NO:130). Preferably in this embodiment, the CH3 Domain is spaced apart from the VL_{I} Domain by an intervening peptide linker, such as one having the amino acid sequence of (SEQ ID NO:15): APSSS, and more preferably, the amino acid sequence (SEQ ID NO:16) APSSSPME, however, alternative linkers may be employed, e.g., ASTKG (SEQ ID NO:131), LEPKSS (SEQ ID NO:132), GGC or GGG.

### D. Albumin-Binding Domain

As disclosed in WO 2012/018687, in order to improve the in vivo pharmacokinetic properties of diabodies, a diabody may be modified to contain a polypeptide portion of a serum-binding protein at one or more of the termini of the diabody. Such considerations are also applicable to the Tri-Specific Binding Molecules of the present invention. Most preferably, when a polypeptide portion of a serum-binding protein is desired to be incorporated into the Tri-Specific Binding Molecules of the present invention, such polypeptide portion will be installed at the C-terminus of one of the polypeptide chains of the Tri-Specific Binding Molecule.

Albumin is the most abundant protein in plasma and has a half-life of 19 days in humans. Albumin possesses several small molecule binding sites that permit it to non-covalently bind to other proteins and thereby extend their serum half-lives. The Albumin-Binding Domain 3 (ABD3) of protein G of Streptococcus strain G148 consists of 46 amino acid residues forming a stable three-helix bundle and has broad albumin-binding specificity (Johansson, M. U. et al. (2002) “Structure, Specificity, And Mode Of Interaction For Bacterial Albumin-Binding Modules,” J. Biol. Chem. 277(10):8114-8120. Thus, a particularly preferred polypeptide portion of a serum-binding protein for improving the in vivo pharmacokinetic properties of a diabody is the Albumin-Binding Domain (ABD) from streptococcal protein G, and more preferably, the Albumin-Binding Domain 3 (ABD3) of protein G of Streptococcus strain G148 (SEQ ID NO:123): LAEAKVLANR ELDKYGVSDY YKNLIDNAKS AEGVKALIDE ILAALP.

As disclosed in WO 2012/162068 (herein incorporated by reference), “deimmunized” variants of SEQ ID NO:123 have the ability to attenuate or eliminate MHC class II binding. Based on combinational mutation results, the following combinations of substitutions are considered to be preferred substitutions for forming such a deimmunized Albumin-Binding Domain: 66S/70S+71A; 66S/70S+79A; 64A/65A/71A+66S; 64A/65A/71A+66D; 64A/65A/71A+66E; 64A/65A/79A+66S; 64A/65A/79A+66D; 64A/65A/79A+66E. Variant ABDs having the modifications L64A, I65A and D79A or the modifications N66S, T70S and D79A. Variant deimmunized ABD having the amino acid sequence:

(SEQ ID NO: 124)
LAEAKVLANR ELDKYGVSDY YKNA₆₄A₆₅NNAKT VEGVKALIA₇₉E
ILAALP,

or the amino acid sequence:

(SEQ ID NO: 125)
LAEAKVLANR ELDKYGVSDY YKNLIS₆₆NAKS₇₀ VEGVKALIA₇₉E
ILAALP,

are particularly preferred as such deimmunized Albumin-Binding Domains exhibit substantially wild-type binding while providing attenuated MHC class II binding. Although such Albumin-Binding Domains may be incorporated into any of the polypeptide chains of the Tri-Specific Binding Molecules of the present invention, it is preferred to position such Domain C-terminally to the E-coil (or K-coil) Domain of the first or third polypeptide chain (via a linker that intervenes between the E-coil (or K-coil) Domain and the Albumin-Binding Domain (which is preferably a deimmunized Albumin-Binding Domain)). A preferred sequence for such a linker is SEQ ID NO:126: GGGS.

### E. Functionality of the Fc Domain

In one embodiment, the CH2-CH3 Domain of the first polypeptide chain and the CH2-CH3 Domain of the third polypeptide will complex to form an Fc Domain that is substantially incapable of binding to an Fc receptor (i.e., binding at less than 10% the extent of a wild-type Fc Domain. Alternatively, the Fc Domain of such molecules will be capable of binding to the Fc receptor under physiological conditions, so that such Tri-Specific Binding Molecules will be tetra-specific, capable of mediating coordinated binding to four molecules (Epitope I, Epitope II and Epitope III, and an Fc receptor). Most preferably, such molecules capable of binding to the Fc receptor will additionally mediate Fc receptor-dependent effector function.

The invention also encompasses molecules comprising variant Fc Domains comprising one or more amino acid substitutions, insertions, or deletions relative to a comparable wild-type Fc Domain. Molecules comprising variant Fc Domains normally have altered phenotypes relative to molecules comprising wild-type Fc Domains The variant phenotype may be expressed as altered serum half-life, altered stability, altered susceptibility to cellular enzymes or altered effector function as assayed in an NK dependent or macrophage dependent assay. Fc Domain modifications identified as altering effector function are known in the art, including modifications that increase binding to activating receptors (e.g., FcγRIIA (CD16A) and reduce binding to inhibitory receptors (e.g., FcγRIIB (CD32B) (see, e.g., Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890). Exemplary variants of human IgG1 Fc Domains with reduced binding to CD32B and/or increased binding to CD16A contain F243L, R292P, Y300L, V305I or P296L substitutions. These amino acid substitutions may be present in a human IgG1 Fc Domain in any combination. In one embodiment, the human IgG1 Fc Domain variant contains a F243L, R292P and Y300L substitution. In another embodiment, the human IgG1 Fc Domain variant contains a F243L, R292P, Y300L, V305I and P296L substitution. In another embodiment, the human IgG1 Fc Domain variant contains an N297Q substitution, L234A and L235A substitutions or a D265A substitution, as these mutations abolish FcR binding.

III. Exemplification of the Tri-Specific Binding Molecules of the Present Invention: Tri-Specific Binding Molecules Having Binding Domains that Bind to Epitopes of CD3 and CD8 and to an Epitope of a Disease-Associated Antigen

As stated above, the present invention particularly relates to the embodiment of Tri-Specific Binding Molecules in which the three epitopes are selected such that one or two of such epitopes are epitope(s) of an immune system cell, and especially, a cytotoxic lymphocyte immune system cell (CTL), and in which the remaining epitope(s) are epitope(s) of a Disease-Associated Antigen. In a particularly preferred embodiment of such Tri-Specific Binding Molecule, the Binding Domains of such molecule are selected such that Epitope I, Epitope II or Epitope III is an epitope of CD3, a second of Epitope I, Epitope II or Epitope III is an epitope of CD8, and the third of Epitope I, Epitope II or Epitope III is an epitope of a Disease-Associated Antigen, wherein the Binding Domains I, II and III of such Tri-Specific Binding Molecules mediate coordinated binding of a cytotoxic T cell and a cell expressing the Disease-Associated Antigen. Such Tri-Specific Binding Molecules are capable of localizing a cytotoxic lymphocyte cell to a cell that expresses a Disease-Associated Antigen, and of thereby facilitate the killing of cells that express the Disease-Associated Antigen. The Disease-Associated Antigen may be a cancer antigen, or may be an antigen that is characteristic of a pathogen (e.g., bacterial, fungal, viral or protozoan) infection. More particularly, the invention relates to such Tri-Specific Binding Molecules that are capable of mediating coordinated binding to: (1) an epitope of CD3, (2) an epitope of CD8, and (3) an epitope of a Disease-Associated Antigen. By binding to CD3 and CD8, and to the Disease-Associated Antigen, such molecules co-localize cytotoxic T cells to cells presenting the Disease-Associated Antigen, leading to the activation of such T cells and the initiation of a cytotoxic response against cells expressing the Disease-Associated Antigen.

The heavy chains of an anti-CD3 or anti-CD8 antibody may be employed as the third polypeptide chain of such exemplary Tri-Specific Binding Molecules of the present invention. Likewise, the light chains of such antibodies may be employed as the fourth polypeptide chain of the Tri-Specific Binding Molecules of the present invention. Alternatively, the Light Chain Variable Domains and/or the Heavy Chain Variable Domains of such antibodies may be combined with other immunoglobulin constant regions to achieve such third and fourth polypeptide chains. Thus, such antibodies may be used to produce Tri-Specific Binding Molecules of the present invention whose Site C is capable of binding CD3 or CD8.

Similarly, such Variable Domains can be incorporated into the Variable Domain portions of the first and third polypeptide of the Tri-Specific Binding Molecules of the present invention so as to produce Tri-Specific Binding Molecules of the present invention whose Site A is capable of binding CD3 or CD8, or whose Site B is capable of binding CD3 or CD8.

### 1. Exemplary Anti-CD3 Antibodies

Any of the exemplary anti-CD3 or anti-CD8 antibodies provided below may be employed to make the CD3 or CD8 Binding Domains of the Tri-Specific Binding Molecules of the present invention.

### OKT3

OKT3 Light Chain Variable Domain (SEQ ID NO:17) (CDRs shown underlined):

QIVLTQSPAI MSASPGEKVT MTCSASSSVS YMNWYQQKSG
TSPKRWIYDT SKLASGVPAH FRGSGSGTSY SLTISGMEAE
DAATYYCQQW SSNPFTFGSG TKLEINR

OKT3 Heavy Chain Variable Domain (SEQ ID NO:18) (CDRs shown underlined):

QVQLQQSGAE LARPGASVKM SCKASGYTFT RYTMHWVKQR
PGQGLEWIGY INPSRGYTNY NQKFKDKATL TTDKSSSTAY
MQLSSLTSED SAVYYCARYY DDHYCLDYWG QGTTLTVSSA
KTTAPSVYPL APVCGDTTGS SVTLGCLVKG YFPEPVTLTW
NSGSLSSGVH TFPAVLQSDL YTLSSSVTVT SS
M291

### M291

M291 Light Chain Variable Domain (SEQ ID NO:19) (CDRs shown underlined):

DIVLTQSPAI MSASPGEKVT MTCSASSSVS YMNWYQQKSG
TSPKRWTYDT SKLASGVPAR FSGSGSGTSY SLTISSMEAE
DADTYYCQQW SSNPPTFGSG TKLEIK

M291 Heavy Chain Variable Domain (SEQ ID NO:20) (CDRs shown underlined):

QVQLQQSGAE LARPGASVKM SCKASGYTFI SYTMHWVKQR
PGQGLEWIGY INPRSGYTHY NQKLKDKATL TADKSSSSAY
MQLSSLTSED SAVYYCARSA YYDYDGFAYW GQGTLVTVSA

### YTH12.5

YTH12.5 Light Chain Variable Domain (SEQ ID NO:21) (CDRs shown underlined):

MGWSCIILFL VATATGVHSD IQLTQPNSVS TSLGSTVKLS
CTLSSGNIEN NYVHWYQLYE GRSPTTMIYD DDKRPDGVPD
RFSGSIDRSS NSAFLTIHNV AIEDEAIYFC HSYVSSFNVF
GGGTKLTVLR

YTH12.5 Heavy Chain Variable Domain (SEQ ID NO:22) (CDRs shown underlined):

MGWSCIILFL VATATGVHSE VQLLESGGGL VQPGGSLRLS
CAASGFTFSS FPMAWVRQAP GKGLEWVSTI STSGGRTYYR
DSVKGRFTIS RDNSKNTLYL QMNSLRAEDT AVYYCAKFRQ
YSGGFDYWGQ GTLVTVSS

### Humanized Anti-CD3 Antibody 1 (“CD3 mAb 1”) (US2014/0099318A1)

CD3 mAb 1 Light Chain Variable Domain (SEQ ID NO:23) Variant 1 (CDRs shown underlined):

DIQMTQSPSS LSASVGDRVT ITCSASSSVS YMNWYQQKPG
KAPKRLIYDS SKLASGVPSR FSGSGSGTEF TLTISSLQPE
DFATYYCQQW SRNPPTFGGG TKVEIK

CD3 mAb 1 Light Chain Variable Domain (SEQ ID NO:24) Variant 2 (CDRs shown underlined):

DVVMTQSPAI MSAFPGEKVT ITCSASSSVS YMNWYQQKPG
KAPKRWIYDS SKLASGVPSR FSGSGSGTEF TLTISSLQPE
DFATYYCQQW SRNPPTFGGG TKVEIK

CD3 mAb 1 Heavy Chain Variable Domain (SEQ ID NO:25) Variant 1 (CDRs shown underlined):

QVQLVQSGAE VKKPGASVKV SCKASGYTFT RSTMHWVRQA
PGQGLEWIGY INPSSAYTNY NQKFKDRVTI TADKSTSTAY
MELSSLRSED TAVYYCASPQ VHYDYNGFPY WGQGTLVTVS S

### Humanized Anti-CD3 Antibody 2 (“CD3 mAb 2”) (US2014/0099318A1)

CD3 mAb 2 Light Chain Variable Domain (SEQ ID NO:26) (CDRs shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

CD3 mAb 2 Heavy Chain Variable Domain (SEQ ID NO:27) (CDRs shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL VTVSS

CD3 mAb 2 Heavy Chain Variable Domain D65G Variant (SEQ ID NO:28) (CDRs shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL VTVSS

### 2. Exemplary Anti-CD8 Antibodies

### OKT8 (“CD8 mAb 1”)

OKT8 Light Chain Variable Domain (SEQ ID NO:29) (CDRs shown underlined):

DIVMTQSPAS LAVSLGQRAT ISCRASESVD SYDNSLMHWY
QQKPGQPPKV LIYLASNLES GVPARFSGSG SRTDFTLTID
PVEADDAATY YCQQNNEDPY TFGGGTKLEI KR

OKT8 Heavy Chain Variable Domain (SEQ ID NO:30) (CDRs shown underlined):

QVQLLESGPE LLKPGASVKM SCKASGYTFT DYNMHWVKQS
HGKSLEWIGY IYPYTGGTGY NQKFKNKATL TVDSSSSTAY
MELRSLTSED SAVYYCARNF RYTYWYFDVW GQGTTVTVSS

### TRX2 (“CD8 mAb 2”)

TRX2 Light Chain Variable Domain (SEQ ID NO:31) (CDRs shown underlined):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIK

TRX2 Heavy Chain Variable Domain (SEQ ID NO:32) (CDRs shown underlined):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS S

### 3. Exemplary Binding Domains That Bind to Epitopes of Disease-Associated Antigens

(a) HIV gp41

An illustrative Disease-Associated Antigen is HIV gp41. An exemplary gp41 antibody is 7B2 (“HIV mAb 1”).

Amino Acid Sequence of 7B2 Light Chain Variable Domain (SEQ ID NO:35):

DIVMTQSPDS LAVSPGERAT IHCKSSQTLL YSSNNRHSIA
WYQQRPGQPP KLLLYWASMR LSGVPDRFSG SGSGTDFTLT
INNLQAEDVA IYYCHQYSSH PPTFGHGTRV EIK

Amino Acid Sequence of 7B2 Heavy Chain Variable Domain (SEQ ID NO:36):

QVQLVQSGGG VFKPGGSLRL SCEASGFTFT EYYMTWVRQA
PGKGLEWLAY ISKNGEYSKY SPSSNGRFTI SRDNAKNSVF
LQLDRLSADD TAVYYCARAD GLTYFSELLQ YIFDLWGQGA RVTVSS

(b) HIV gp120

A second illustrative Disease-Associated Antigen is HIV gp120. An exemplary gp120 antibody is A32 (“HIV mAb 2”).

Amino Acid Sequence of A32 VL Light Chain Variable Domain (SEQ ID NO:33):

QSALTQPPSA SGSPGQSVTI SCTGTSSDVG GYNYVSWYQH
HPGKAPKLII SEVNNRPSGV PDRFSGSKSG NTASLTVSGL
QAEDEAEYYC SSYTDIHNFV FGGGTKLTVL

Amino Acid Sequence of A32 VH Heavy Chain Variable Domain (SEQ ID NO:34):

QVQLQESGPG LVKPSQTLSL SCTVSGGSSS SGAHYWSWIR
QYPGKGLEWI GYIHYSGNTY YNPSLKSRIT ISQHTSENQF
SLKLNSVTVA DTAVYYCARG TRLRTLRNAF DIWGQGTMVT VSS

(c) RSV glycoprotein F

A further illustrative Disease-Associated Antigen is RSV glycoprotein F. An exemplary anti-RSV glycoprotein F antibody is palivizumab (“RSV mAb 1”).

Amino Acid Sequence of Palivizumab Light Chain Variable Domain (SEQ ID NO:37):

DIQMTQSPST LSASVGDRVT ITCRASQSVG YMHWYQQKPG
KAPKLLIYDT SKLASGVPSR FSGSGSGTEF TLTISSLQPD
DFATYYCFQG SGYPFTFGGG TKLEIK

Amino Acid Sequence of palivizumab Heavy Chain Variable Domain (SEQ ID NO:38):

QVTLRESGPA LVKPTQTLTL TCTFSGFSLS TSGMSVGWIR
QPPGKALEWL ADIWWDDKKD YNPSLKSRLT ISKDTSKNQV
VLKVTNMDPA DTATYYCARS MITNWYFDVW GAGTTVTVSS

### (d) B7-H3

A particularly preferred illustrative Disease-Associated Antigen is B7-H3, which is expressed on a variety of cancer cells (e.g., neuroblastomas, gastric, ovarian and non-small cell lung cancers, etc.). B7-H3 protein expression has been immunohistologically detected in tumor cell lines (Chapoval, A. et al. (2001) “B7-H3: A Costimulatory Molecule For T Cell Activation and IFN-γ Production,” Nature Immunol. 2:269-274; Saatian, B. et al. (2004) “Expression Of Genes For B7-H3 And Other T Cell Ligands By Nasal Epithelial Cells During Differentiation And Activation,” Amer. J. Physiol. Lung Cell. Mol. Physiol. 287:L217-L225; Castriconi et al. (2004) “Identification Of 4Ig-B7-H3 As A Neuroblastoma-Associated Molecule That Exerts A Protective Role From An NK Cell-Mediated Lysis,” Proc. Natl. Acad. Sci. (U.S.A.) 101(34):12640-12645); Sun, M. et al. (2002) “Characterization of Mouse and Human B7-H3 Genes,” J. Immunol. 168:6294-6297). mRNA expression has been found in heart, kidney, testes, lung, liver, pancreas, prostate, colon, and osteoblast cells (Collins, M. et al. (2005) “The B7 Family Of Immune-Regulatory Ligands,” Genome Biol. 6:223.1-223.7). At the protein level, B7-H3 is found in human liver, lung, bladder, testis, prostate, breast, placenta, and lymphoid organs (Hofmeyer, K. et al. (2008) “The Contrasting Role Of B7-H3,” Proc. Natl. Acad. Sci. (U.S.A.) 105(30):10277-10278). Illustrative antibodies that bind to B7-H3 include humanized “BRCA84D,” “BRCA69D” and “PRCA157” (WO 2011/109400). Exemplary light and heavy variable chains have the following sequences (CDRs shown underlined):

Amino Acid Sequence of exemplary humanized BRCA84D-5VL Light Chain Variable Domain (SEQ ID NO:39):

DIQLTQSPSF LSASVGDRVT ITCKASQNVD TNVAWYQQKP
GQAPKALIYS ASYRYSGVPS RFSGSGSGTD FTLTISSLQP
EDFATYYCQQ YNNYPFTFGQ GTKLEIK

Amino Acid Sequence of exemplary humanized BRCA84D-2VH Heavy Chain Variable Domain (SEQ ID NO:40):

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCGRGR ENIYYGSRLD YWGQGTTVTV SS

Amino Acid Sequence of exemplary BRCA69D (“B7-H3 mAb 1”) Light Chain Variable Domain (SEQ ID NO:41):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIK

Amino Acid Sequence of exemplary BRCA69D (“B7-H3 mAb 1”) Heavy Chain Variable Domain (SEQ ID NO:42):

QVQLVQSGAE VKKPGASVKV SCKASGYTFT SYWMQWVRQA
PGQGLEWMGT IYPGDGDTRY TQKFKGRVTI TADKSTSTAY
MELSSLRSED TAVYYCARRG IPRLWYFDVW GQGTTVTVSS

Amino Acid Sequence of exemplary PRCA157 Light Chain Variable Domain (SEQ ID NO:43):

DIQMTQSPAS LSVSVGETVT ITCRASESIY SYLAWYQQKQ
GKSPQLLVYN TKTLPEGVPS RFSGSGSGTQ FSLKINSLQP
EDFGRYYCQH HYGTPPWTFG GGTNLEIK

Amino Acid Sequence of exemplary PRCA157 Heavy Chain Variable Domain (SEQ ID NO:44):

EVQQVESGGD LVKPGGSLKL SCAASGFTFS SYGMSWVRQT
PDKRLEWVAT INSGGSNTYY PDSLKGRFTI SRDNAKNTLY
LQMRSLKSED TAMYYCARHD GGAMDYWGQG TSVTVSS

### (e) A33 Tumor Antigen

The A33 tumor antigen is another illustrative Disease-Associated Antigen. The amino acid sequence of the Light Chain Variable Domain of an exemplary humanized anti-A33 antibody (“gpA33 mAb 1”) is (SEQ ID NO:45):

QIVLTQSPAI MSASPGERVT MTCSARSSIS FMYWYQQKPG
SSPRLLIYDT SNLASGVPVR FSGSGSGTSY SLTISRMEAE
DAATYYCQQW SSYPLTFGSG TKLELKR

The amino acid sequence of the Heavy Chain Variable Domain of such exemplary humanized anti-A33 (gpA33 mAb 1) antibody is (SEQ ID NO:46):

QVQLQQSGPE LVKPGASVKI SCKASGYTFS GSWMNWVKQR
PGQGLEWIGR IYPGDGETNY NGKFKDKATL TADKSSTTAY
MELSSLTSVD SAVYFCARIY GNNVYFDVWG AGTTVTVSS

### (f) 5T4 Tumor Antigen

The 5T4 tumor antigen is another illustrative Disease-Associated Antigen. The amino acid sequence of the Light Chain Variable Domain of an exemplary humanized anti-5T4 mAb 1 antibody (“5T4 mAb 1”) is (SEQ ID NO:47):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIK

The amino acid sequence of the Heavy Chain Variable Domain of such exemplary humanized 5T4 mAb 1 is (SEQ ID NO:48):

QVQLVQSGAE VKKPGASVKV SCKASGYTFT SFWMHWVRQA
PGQGLEWMGR IDPNRGGTEY NEKAKSRVTM TADKSTSTAY
MELSSLRSED TAVYYCAGGN PYYPMDYWGQ GTTVTVSS

The amino acid sequence of the Light Chain Variable Domain of a second exemplary humanized 5T4 mAb 1 antibody (“5T4 mAb 2”) is (SEQ ID NO:49):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IK

The amino acid sequence of the Heavy Chain Variable Domain of such second exemplary humanized 5T4 mAb 2 is (SEQ ID NO:50):

QVQLQQPGAE LVKPGASVKM SCKASGYTFT SYWITWVKQR
PGQGLEWIGD IYPGSGRANY NEKFKSKATL TVDTSSSTAY
MQLSSLTSED SAVYNCARYG PLFTTVVDPN SYAMDYWGQG
TSVTVSS

### (g) ROR1 Antigen

The ROR1 tumor antigen is another illustrative Disease-Associated Antigen. Exemplary anti-ROR1 antibodies include antibody 2A2 (WO 2010/124188), R11 (WO 2012/075158) and R12 (WO 2012/075158).

The amino acid sequence of the Light Chain Variable Domain of the 2A2 antibody is (SEQ ID NO:53):

DIVMTQSQKI MSTTVGDRVS ITCKASQNVD AAVAWYQQKP
GQSPKLLIYS ASNRYTGVPD RFTGSGSGTD FTLTISNMQS
EDLADYFCQQ YDIYPYTFGG GTKLEIK

The amino acid sequence of the Heavy Chain Variable Domain of the 2A2 antibody is (SEQ ID NO:54):

QVQLQQSGAE LVRPGASVTL SCKASGYTFS DYEMHWVIQT
PVHGLEWIGA IDPETGGTAY NQKFKGKAIL TADKSSSTAY
MELRSLTSED SAVYYCTGYY DYDSFTYWGQ GTLVTVSA

The amino acid sequence of the Light Chain Variable Domain of the R11 antibody is (SEQ ID NO:55):

ELVMTQTPSS TSGAVGGTVT INCQASQSID SNLAWFQQKP
GQPPTLLIYR ASNLASGVPS RFSGSRSGTE YTLTISGVQR
EDAATYYCLG GVGNVSYRTS FGGGTEVVVK

The amino acid sequence of the Heavy Chain Variable Domain of the R11 antibody is (SEQ ID NO:56):

QSVKESEGDL VTPAGNLTLT CTASGSDIND YPISWVRQAP
GKGLEWIGFI NSGGSTWYAS WVKGRFTISR TSTTVDLKMT
SLTTDDTATY FCARGYSTYY GDFNIWGPGT LVTISS

The amino acid sequence of the Light Chain Variable Domain of the R12 antibody is (SEQ ID NO:57):

ELVLTQSPSV SAALGSPAKI TCTLSSAHKT DTIDWYQQLQ
GEAPRYLMQV QSDGSYTKRP GVPDRFSGSS SGADRYLIIP
SVQADDEADY YCGADYIGGY VFGGGTQLTV TG

The amino acid sequence of the Heavy Chain Variable Domain of the R12 antibody is (SEQ ID NO:58):

QEQLVESGGR LVTPGGSLTL SCKASGFDFS AYYMSWVRQA
PGKGLEWIAT IYPSSGKTYY ATWVNGRFTI SSDNAQNTVD
LQMNSLTAAD RATYFCARDS YADDGALFNI WGPGTLVTIS S

One aspect of the present invention (discussed in detail below) is the provision of a more preferred humanized anti-ROR1 antibody (“ROR1 mAb 1”). This more preferred ROR1 mAb 1 has a Light Chain Variable Domain having the sequence (SEQ ID NO:51):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VL

The amino acid sequence of the Heavy Chain Variable Domain of such more preferred humanized ROR1 mAb 1 is (SEQ ID NO:52):

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCARDS YADDAALFDI WGQGTTVTVS S

### IV. Selection of Binding Site: Site A, Site B and Site C

As indicated above, the preferred Tri-Specific Binding Molecules of the present invention are at least tri-specific, having an “external” Diabody-Type Binding Domain (Site A) that is located furthest from a Binding Domain III, an “internal” Diabody-Type Binding Domain (Site B) that is located nearest to a Binding Domain III, and the Binding Domain III itself (Site C). As used herein, a description of a Tri-Specific Binding Molecule such as “X/YIZ” indicates that the X Binding Domain is at Site A, the Y Binding Domain is at Site B and the Z Binding Domain is at Site C. For example, the Tri-Specific Binding Molecule designation “B7-H3 mAb 1/CD3 mAb 2/CD8 mAb 1” indicates the B7-H3 mAb 1 Variable Domains occupy Site A of the Tri-Specific Binding Molecule, CD3 mAb 2 Variable Domains occupy Site B and CD8 mAb 1 Variable Domains occupy Site C of the Tri-Specific Binding Molecule.

The present invention thus permits choice as to which of such Sites is to be used to bind a particular desired epitope. One factor to guide such selection, particularly with Tri-Specific Binding Molecules that bind to CD3, CD8 and a Disease-Associated Antigen, involves a consideration of the effect and desirability of trogocytosis. “Trogocytosis” is a process through which a cell can acquire a portion of a cell membrane of a contacting cell (Masuda, S. et al. (2013) “Possible Implication Of Fc γ Receptor-Mediated Trogocytosis In Susceptibility To Systemic Autoimmune Disease,” Clin. Dev. Immunol. 2013: Article ID 345745, 6 pages); Dhainaut, M. et al. (2014) “Regulation of Immune Reactivity by Intercellular Transfer,” Front Immunol. 5:112; Ahmed, K. A. et al. (2011) “Mechanisms Of Cellular Communication Through Intercellular Protein Transfer,” J. Cell. Mol. Med. 15(7):1458-1473; Ahmed, K. A. et al. (2008) “Intercellular Trogocytosis Plays An Important Role In Modulation Of Immune Responses,” Cell. Mol. Immunol. 5(4):261-269; LeMaoult, J. et al. (2007) “Exchanges Of Membrane Patches (Trogocytosis) Split Theoretical And Actual Functions Of Immune Cells,” Hum. Immunol. 68(4):240-243; Caumartin. J. et al. (2006) “Intercellular Exchanges Of Membrane Patches (Trogocytosis) Highlight The Next Level Of Immune Plasticity,” Transpl. Immunol. 17(1):20-22).

The acquisition of cognate MHC class I ligands by trogocytosis induces cytotoxic T lymphocytes to become “Acquired Treg” cells that mediate the killing (“fratricide”) of other cytotoxic T cells, thereby contributing to the clearance of CD8⁺ cells (D'Acquisto, F. et al. (2011) “CD3⁺ CD4⁻ CD8⁻ (Double Negative) T Cells: Saviours Or Villains Of The Immune Response?” Biochem. Pharmacol. 82:333-340; Joly, E. et al. (2003) “What Is Trogocytosis And What Is Its Purpose?” Nat. Immunol. 4:815-; Hudrisier, D. et al. (2007) “Capture Of Target Cell Membrane Components Via Trogocytosis Is Triggered By A Selected Set Of Surface Molecules On T Or B Cells,” J. Immunol. 178:3637-3647).

A Tri-Specific Binding Molecule of the present invention that possesses a CD3 Binding Domain as its Site C position has the attributes of an anti-CD3 antibody, and likewise such a Tri-Specific Binding Molecule that possesses a CD8 Binding Domain as its Site C position has the attributes of an anti-CD8 antibody. It has been shown that a neutrophil, monocyte or macrophage having an Fc receptor that is bound to the Fc Domain of an anti-CD8 antibody (that has bound to a CD8 molecule of a T cell) is capable of transferring the antibody and the bound CD8 molecule from the T cell to itself via trogocytosis and of then rapidly internalizing the antibody; bystander molecules, such as TCR and CD3 may also be transferred in this process (Masuda, S. et al., (2013) “Possible Implication of Fcg Receptor-Mediated Trogocytosis in Susceptibility to Systemic Autoimmune Disease,” Clin. Develop. Immunol. 2013:Article ID 345745, 6 pages).

The structures of CD3 and CD8 differ in that CD3 lies close to the cell membrane, while CD8 extends further from the cell membrane. It is thus expected that Fc receptor trogocytosis of CD3 by an anti-CD3 antibody would be more efficient than Fc receptor trogocytosis of CD8 by an anti-CD8 antibody.

This phenomenon indicates that a Tri-Specific Binding Molecule of the present invention that binds to CD3, CD8 and a Disease-Associated Antigen whose CD3 Binding Domain is located on the Site C position will exhibit less cytotoxicity than an analogous Tri-Specific Binding Molecule in which the CD3 Binding Domain is located at Site A or at Site B. Thus, by electing to place the CD3 Binding Domain on the Site C position (as opposed to either of the Sites A or B), one may modulate the extent of cytotoxicity. Additionally, one may compound pharmaceutical compositions that contain a mixture of a “Site C” and a “Site A (or B)” CD3 in order to obtain a preferred extent of cytotoxicity.

### V. Anti-ROR1 mAb 1 Antibody

As indicated above, one aspect of the present application is the provision of highly preferred humanized anti-ROR1 antibody (“ROR1 mAb 1”) whose Light Chain Variable Domain has the amino acid sequence (SEQ ID NO:51):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VL

and whose Heavy Chain Variable Domain has the amino acid sequence (SEQ ID NO:52):

QEQLVESGGG LVQPGGSLRL SCAASGFTFS DYYMSWVRQA
PGKGLEWVAT IYPSSGKTYY ADSVKGRFTI SSDNAKNSLY
LQMNSLRAED TAVYYCARDS YADDAALFDI WGQGTTVTVS S

The sequences of CDR_{L}1, CDR_{L}2, and CDR_{L}3 of the Light Chain Variable Domain of such ROR1 mAb 1 antibody are:

Light Chain Variable Domain CDR_{L}1
(SEQ ID NO: 117): TLSSGHKTDTID
Light Chain Variable Domain CDR_{L}2
(SEQ ID NO: 118): LEGSGSY
Light Chain Variable Domain CDR_{L}3
(SEQ ID NO: 119): GTDYPGNYL

The sequences of CDR_{H}1, CDR_{H}2, and CDR_{H}3 of the Heavy Chain Variable Domain of such ROR1 mAb 1 antibody are:

Heavy Chain Variable Domain CDR_{H}1
(SEQ ID NO: 120): GFTFSDYYMS
Heavy Chain Variable Domain CDR_{H}2
(SEQ ID NO: 121): TIYPSSGKTYYADSVKG
Heavy Chain Variable Domain CDR_{H}3
(SEQ ID NO: 122): DSYADDAALFDI

The ROR1 mAb 1 antibody mediates increased cytotoxicity and is less immunogenic relative to prior art anti-ROR1 antibodies (e.g., anti-ROR1 antibody R12).

The invention encompasses not only such sequences, but also intact ROR1 mAb 1 antibody derivatives (including chimeric or humanized derivatives thereof) that possess 1, 2 or 3 of the CDRs of such Light Chain Variable Domain (SEQ ID NO:51, CDRs shown underlined) or 1, 2 or 3 of the CDRs of such Heavy Chain Variable Domain (SEQ ID NO:52; CDRs shown underlined), and which immunospecifically bind to ROR1. More preferably, such encompassed antibodies, chimeric antibodies and humanized antibodies will possess 1, 2 or 3 of the CDRs of such Light Chain Variable Domain (SEQ ID NO:51, CDRs shown underlined) and also 1, 2 or 3 of the CDRs of such Heavy Chain Variable Domain (SEQ ID NO:52; CDRs shown underlined), and will immunospecifically bind to ROR1. Most preferably, such encompassed antibodies, chimeric antibodies and humanized antibodies will possess all 3 of the CDRs of such Light Chain Variable Domain, and all 3 of the CDRs of such Heavy Chain Variable Domain and be capable of immunospecifically binding to ROR1.

The invention additionally encompasses fragments and derivatives of such encompassed ROR1 mAb 1 antibodies, including Fab, Fab′, F(ab′)₂ Fv), single-chain (ScFv), “BiTEs®,” “DART™” diabody molecules, mutants thereof, naturally occurring variants, and fusion proteins, all of which comprise 1, 2, or 3 of the Light Chain Variable Domain CDRs, or 1, 2, or 3 of the Heavy Chain Variable Domain CDRs, or 1, 2, or 3 of the Light Chain Variable Domain CDRs, and also 1, 2, or 3 of the Heavy Chain Variable Domain CDRs, and which are capable of immunospecifically binding to ROR1.

In a preferred embodiment, such ROR1 mAb 1 antibodies or their fragments or derivatives may have variant Fc Domains. Modification of the Fc Domain normally leads to an altered phenotype, for example altered serum half-life, altered stability, altered susceptibility to cellular enzymes or altered effector function. It may be desirable to modify the antibody of the invention with respect to effector function, so as to enhance the effectiveness of the antibody in treating cancer, for example. Reduction or elimination of effector function is desirable in certain cases, for example in the case of antibodies whose mechanism of action involves blocking or antagonism, but not killing of the cells bearing a target antigen. Increased effector function is generally desirable when directed to undesirable cells, such as tumor and foreign cells, where the FcγRs are expressed at low levels, for example, tumor specific B cells with low levels of FcγRIIB (e.g., non-Hodgkins lymphoma, CLL, and Burkitt's lymphoma). In said embodiments, molecules of the invention with conferred or enhanced effector function activity are useful for the treatment and/or prevention of a disease, disorder or infection where an enhanced efficacy of effector function activity is desired.

In certain embodiments, such ROR1 mAb 1 antibodies or their fragments or derivatives comprise one or more modifications to the amino acids of the Fc Domain, which reduce the affinity and avidity of the Fc Domain of such molecule for one or more FcγR receptors. In other embodiments, such ROR1 mAb 1 antibodies or their fragments or derivatives may comprise one or more modifications to the amino acids of the Fc Domain that increase the affinity and avidity of the Fc Domain of such molecule for one or more FcγR receptors. In other embodiments, the molecules comprise a variant Fc Domain wherein said variant confers or mediates increased ADCC activity and/or an increased binding to FcγRIIA, relative to a molecule comprising no Fc Domain or comprising a wild-type Fc Domain. In alternate embodiments, the molecules comprise a variant Fc Domain wherein said variant confers or mediates decreased ADCC activity (or other effector function) and/or an increased binding to FcγRIIB, relative to a molecule comprising no Fc Domain or comprising a wild-type Fc Domain.

In some embodiments, the invention encompasses such ROR1 mAb 1 antibodies or their fragments or derivatives that comprise a variant Fc Domain, which variant Fc Domain does not show a detectable binding to any FcγR, relative to a comparable molecule comprising the wild-type Fc Domain. In other embodiments, the invention encompasses such ROR1 mAb 1 antibodies or their fragments or derivatives that comprise a variant Fc Domain, which variant Fc Domain only binds a single FcγR, preferably one of FcγRIIA, FcγRIIB, or FcγRIIIA.

Such ROR1 mAb 1 antibodies or their fragments or derivatives may comprise altered affinities for an activating and/or inhibitory Fcγ receptor. In one embodiment, the antibody or molecule comprises a variant Fc Domain that has increased affinity for FcγRIIB and decreased affinity for FcγRIIIA and/or FcγRIIA, relative to a comparable molecule with a wild-type Fc Domain. In another embodiment, such ROR1 mAb 1 antibodies or their fragments or derivatives may comprise a variant Fc Domain that has decreased affinity for FcγRIIB and increased affinity for FcγRIIIA and/or FcγRIIA, relative to a comparable molecule with a wild-type Fc Domain. In yet another embodiment, such ROR1 mAb 1 antibodies or their fragments or derivatives comprise a variant Fc Domain that has decreased affinity for FcγRIIB and decreased affinity for FcγRIIIA and/or FcγRIIA, relative to a comparable molecule with a wild-type Fc Domain. In still another embodiment, such ROR1 mAb 1 antibodies or their fragments or derivatives may comprise a variant Fc Domain that has unchanged affinity for FcγRIIB and decreased (or increased) affinity for FcγRIIIA and/or FcγRIIA, relative to a comparable molecule with a wild-type Fc Domain.

In certain embodiments, the invention encompasses such ROR1 mAb 1 antibodies or their fragments or derivatives that comprise a variant Fc Domain with an altered affinity for FcγRIIIA and/or FcγRIIA such that the immunoglobulin has an enhanced effector function, e.g., ADCC. Non-limiting examples of effector cell functions include ADCC, antibody dependent cellular phagocytosis (ADCP), phagocytosis, opsonization, opsonophagocytosis, cell binding, rosetting, C1q binding, and CDC.

In a preferred embodiment, the alteration in affinity or effector function is at least 2-fold, preferably at least 4-fold, at least 5-fold, at least 6-fold, at least 7-fold, at least 8-fold, at least 9-fold, at least 10-fold, at least 50-fold, or at least 100-fold, relative to a comparable molecule comprising a wild-type Fc Domain. In other embodiments of the invention, the variant Fc Domain specifically binds one or more FcRs with at least 65%, preferably at least 70%, 75%, 80%, 85%, 90%, 95%, 100%, 125%, 150%, 175%, 200%, 225%, or 250% greater affinity relative to a molecule comprising a wild-type Fc Domain. Such measurements can be in vivo or in vitro assays, and in a preferred embodiment are in vitro assays such as ELISA or surface plasmon resonance assays.

In different embodiments, such ROR1 mAb 1 antibodies or their fragments or derivatives comprise a variant Fc Domain wherein said variant agonizes at least one activity of an FcγR receptor, or antagonizes at least one activity of an FcγR receptor. In a preferred embodiment, the molecules comprise a variant that agonizes (or antagonizes) one or more activities of FcγRIIB, for example, B cell receptor-mediated signaling, activation of B cells, B cell proliferation, antibody production, intracellular calcium influx of B cells, cell cycle progression, FcγRIIB-mediated inhibition of FcεRI signaling, phosphorylation of FcγRIIB, SHIP recruitment, SHIP phosphorylation and association with She, or activity of one or more downstream molecules (e.g., MAP kinase, INK, p38, or Akt) in the FcγRIIB signal transduction pathway. In another embodiment, the molecules comprise a variant that agonizes (or antagonizes) one or more activities of FcRI, for example, mast cell activation, calcium mobilization, degranulation, cytokine production, or serotonin release.

In certain embodiments, such ROR1 mAb 1 antibodies or their fragments comprise an Fc Domain-comprising domain from two or more IgG isotypes (e.g., IgG1, IgG2, IgG3 and IgG4). The various IgG isotypes exhibit differing physical and functional properties including serum half-life, complement-fixation, FcγR binding affinities and effector function activities (e.g. ADCC, CDC, etc.) due to differences in the amino acid sequences of their hinge and/or Fc Domains, for example as described in Flesch, B. K. and Neppert, J. (1999) “Functions Of The Fc Receptors For Immunoglobulin G,” J. Clin. Lab. Anal. 14:141-156; Chappel, M. S. et al. (1993) “Identification Of A Secondary Fc Gamma RI Binding Site Within A Genetically Engineered Human IgG Antibody,” J. Biol. Chem. 33:25124-25131; Chappel, M. S. et al. (1991) “Identification Of The Fc Gamma Receptor Class I Binding Site In Human IgG Through The Use Of Recombinant IgG1/IgG2 Hybrid And Point-Mutated Antibodies,” Proc. Natl. Acad. Sci. (U.S.A.) 88:9036-9040; Brüggemann, M. et al. (1987) “Comparison Of The Effector Functions Of Human Immunoglobulins Using A Matched Set Of Chimeric Antibodies,” J. Exp. Med 166:1351-1361. This type of variant Fc Domain may be used alone, or in combination with an amino acid modification, to affect Fc-mediated effector function and/or binding activity. In combination, the amino acid modification and IgG hinge/Fc Domain may display similar functionality (e.g., increased affinity for FcγRIIA) and may act additively or, more preferably, synergistically to modify the effector functionality in the molecule of the invention, relative to a molecule of the invention comprising a wild-type Fc Domain. In other embodiments, the amino acid modification and IgG Fc Domain may display opposite functionality (e.g., increased and decreased affinity for FcγRIIA, respectively) and may act to selectively temper or reduce a specific functionality in the molecule of the invention, relative to a molecule of the invention not comprising an Fc Domain or comprising a wild-type Fc Domain of the same isotype.

In a preferred specific embodiment, such ROR1 mAb 1 antibodies or their fragments comprise a variant Fc Domain, wherein said variant Fc Domain comprises at least one amino acid modification relative to a wild-type Fc Domain, such that the molecule has an altered affinity for an FcR, provided that said variant Fc Domain does not have a substitution at positions that make a direct contact with FcγR based on crystallographic and structural analysis of Fc-FcR interactions such as those disclosed by Sondermann, P. et al. (2000) “The 3.2-A Crystal Structure Of The Human IgG1 Fc Fragment-Fc GammaRIHI Complex,” Nature 406:267-273. Examples of positions within the Fc Domain that make a direct contact with FcγR are amino acid residues 234-239 (Hinge Domain), amino acid residues 265-269 (B/C loop), amino acid residues 297-299 (C′/E loop), and amino acid residues 327-332 (F/G loop). In some embodiments, the molecules of the invention comprise variant Fc Domains comprise modification of at least one residue that does not make a direct contact with an FcγR based on structural and crystallographic analysis, e.g., is not within the Fc-FcγR binding site.

Variant Fc Domains are well-known in the art, and any known Fc variant may be used in the present invention to confer or modify the effector function exhibited by such ROR1 mAb 1 antibodies or their fragments comprising an Fc Domain (or portion thereof) as functionally assayed, e.g., in an NK dependent or macrophage dependent assay. For example, Fc Domain variants identified as altering effector function are disclosed in PCT Publications No. WO 04/063351; WO 06/088494; WO 07/024249; WO 06/113665; WO 07/021841; WO 07/106707; WO 2008/140603, and any suitable variant disclosed therein may be used in the present molecules.

In certain embodiments, such ROR1 mAb 1 antibodies or their fragments comprise a variant Fc Domain, having one or more amino acid modifications in one or more sites, which modification(s) alter (relative to a wild-type Fc Domain) the Ratio of Affinities of the variant Fc Domain to an activating FcγR (such as FcγRIIA or FcγRIIIA) relative to an inhibiting FcγR (such as FcγRIIB):

$$Ratio of Affinities = \frac{Wild ‐ Type to Variant Change in Affinity to Fc γ {R}_{Activating}}{Wild ‐ Type to Variant Change in Affinity to Fc γ {R}_{Inhibiting}}$$

Where an Fc variant has a Ratio of Affinities greater than 1, the methods of the invention have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. Where an Fc variant has a Ratio of Affinities less than 1, the methods of the invention have particular use in providing a therapeutic or prophylactic treatment of a disease or disorder, or the amelioration of a symptom thereof, where a decreased efficacy of effector cell function mediated by FcγR is desired, e.g., autoimmune or inflammatory disorders. Table 3 lists exemplary single, double, triple, quadruple and quintuple mutations by whether their Ratio of Affinities is greater than or less than 1, and more information concerning these mutations may be found in PCT Publications No. WO 04/063351; WO 06/088494; WO 07/024249; WO 06/113665; WO 07/021841; WO 07/106707; WO 2008/140603.

TABLE 3
Exemplary Single and Multiple Mutations Listed by
Ratio of Affinities

| Ratio | Single | Double | Triple | Quadruple | Quintuple |
|---|---|---|---|---|---|
| >1 | F243L | F243L & | F243L, | L234F, F243L, | L235V, |
| | D270E | R292P | P247L & | R292P & Y300L | F243L, |
| | R292G | F243L & | N421K | L235I, F243L, | R292P, |
| | R292P | Y300L | F243L, | R292P & Y300L | Y300L & |
| | | F243L & | R292P & | L235Q, F243L, | P396L |
| | | P396L | Y300L | R292P & Y300L | L235P, |
| | | D270E & | F243L, | F243L, P247L, | F243L, |
| | | P396L | R292P & | D270E & N421K | R292P, |
| | | R292P & | V305I | F243L, R255L, | Y300L & |
| | | Y300L | F243L, | D270E & P396L | P396L |
| | | R292P & | R292P & | F243L, D270E, | F243L, |
| | | V305I | P396L | G316D & R416G | R292P, |
| | | R292P & | F243L, | F243L, D270E, | V305I, |
| | | P396L | Y300L & | K392T & P396L | Y300L & |
| | | Y300L & | P396L | F243L, D270E, | P396L |
| | | P396L | P247L, | P396L & Q419H | |
| | | P396L & | D270E & | F243L, R292P, | |
| | | Q419H | N421K | Y300L, & P396L | |
| | | | R255L, | F243L, R292P, | |
| | | | D270E & | V305I & P396L | |
| | | | P396L | P247L, D270E, | |
| | | | D270E, | Y300L & N421K | |
| | | | G316D & | R255L, D270E, | |
| | | | R416G | R292G & P396L | |
| | | | D270E, | R255L, D270E, | |
| | | | K392T & | Y300L & P396L | |
| | | | P396L | D270E, G316D, | |
| | | | D270E, | P396L & R416G | |
| | | | P396L & | | |
| | | | Q419H | | |
| | | | V284M, | | |
| | | | R292L | | |
| | | | & K370N | | |
| | | | R292P, | | |
| | | | Y300L & | | |
| | | | P396L | | |
| <1 | Y300L | F243L & | F243L, | | |
| | P396L | P396L | R292P & | | |
| | | P247L & | V305I | | |
| | | N421K | | | |
| | | R255L & | | | |
| | | P396L | | | |
| | | R292P & | | | |
| | | V305I | | | |
| | | K392T & | | | |
| | | P396L | | | |
| | | P396L & | | | |
| | | Q419H | | | |

In a specific embodiment, in variant Fe Domains, any amino acid modifications (e.g., substitutions) at any of positions 235, 240, 241, 243, 244, 247, 262, 263, 269, 298, 328, or 330 and preferably one or more of the following residues: A240, 1240, L241, L243, H244, N298, I328 or V330. In a different specific embodiment, in variant Fc Domains, any amino acid modifications (e.g., substitutions) at any of positions 268, 269, 270, 272, 276, 278, 283, 285, 286, 289, 292, 293, 301, 303, 305, 307, 309, 331, 333, 334, 335, 337, 338, 340, 360, 373, 376, 416, 419, 430, 434, 435, 437, 438 or 439 and preferably one or more of the following residues: H280, Q280, Y280, G290, S290, T290, Y290, N294, K295, P296, D298, N298, P298, V298, I300 or L300.

In a preferred embodiment, in variant Fc Domains that bind an FcγR with an altered affinity, any amino acid modifications (e.g., substitutions) at any of positions 255, 256, 258, 267, 268, 269, 270, 272, 276, 278, 280, 283, 285, 286, 289, 290, 292, 293, 294, 295, 296, 298, 300, 301, 303, 305, 307, 309, 312, 320, 322, 326, 329, 330, 332, 331, 333, 334, 335, 337, 338, 339, 340, 359, 360, 373, 376, 416, 419, 430, 434, 435, 437, 438 or 439. Preferably, the variant Fc Domain has any of the following residues: A256, N268, Q272, D286, Q286, S286, A290, S290, A298, M301, A312, E320, M320, Q320, R320, E322, A326, D326, E326, N326, S326, K330, T339, A333, A334, E334, H334, L334, M334, Q334, V334, K335, Q335, A359, A360 or A430.

In a different embodiment, in variant Fc Domains that bind an FcγR (via its Fc Domain) with a reduced affinity, any amino acid modifications (e.g., substitutions) at any of positions 252, 254, 265, 268, 269, 270, 278, 289, 292, 293, 294, 295, 296, 298, 300, 301, 303, 322, 324, 327, 329, 333, 335, 338, 340, 373, 376, 382, 388, 389, 414, 416, 419, 434, 435, 437, 438, or 439.

In a different embodiment, in variant Fc Domains that bind an FcγR (via its Fc Domain) with an enhanced affinity, any amino acid modifications (e.g., substitutions) at any of positions 280, 283, 285, 286, 290, 294, 295, 298, 300, 301, 305, 307, 309, 312, 315, 331, 333, 334, 337, 340, 360, 378, 398, or 430. In a different embodiment, in variant Fc Domains that binds FcγRIIA with an enhanced affinity, any of the following residues: A255, A256, A258, A267, A268, N268, A272, Q272, A276, A280, A283, A285, A286, D286, Q286, S286, A290, S290, M301, E320, M320, Q320, R320, E322, A326, D326, E326, S326, K330, A331, Q335, A337 or A430.

Preferred variants include one or more modifications at any of positions: 228, 230, 231, 232, 233, 234, 235, 239, 240, 241, 243, 244, 245, 247, 262, 263, 264, 265, 266, 271, 273, 275, 281, 284, 291, 296, 297, 298, 299, 302, 304, 305, 313, 323, 325, 326, 328, 330 or 332.

Particularly preferred variants include one or more modifications selected from groups A-AL:

| | |
|---|---|
| A | 228E, 228K, 228Y or 228G; |
| B | 230A, 230E, 230Y or 230G; |
| C | 231E, 231K, 231Y, 231P or 231G; |
| D | 232E, 232K, 232Y, 232G; |
| E | 233D; |
| F | 234I or 234F; |
| G | 235D, 235Q, 235P, 235I or 235V; |
| H | 239D, 239E, 239N or 239Q; |
| I | 240A, 240I, 240M or 240T; |
| J | 243R, 243, 243Y, 243L, 243Q, 243W, 243H or 243I; |
| K | 244H; |
| L | 245A; |
| M | 247G, 247V or 247L; |
| N | 262A, 262E, 262I, 262T, 262E or 262F; |
| O | 263A, 263I, 263M or 263T; |
| P | 264F, 264E, 264R, 264I, 264A, 264T or 264W; |
| Q | 265F, 265Y, 265H, 265I, 265L, 265T, 265V, 265N or 265Q; |
| R | 266A, 266I, 266M or 266T; |
| S | 271D, 271E, 271N, 271Q, 271K, 271R, 271S, 271T, 271H, |
| | 271A, 271V, 271L, 271I, 271F, 271M, 271Y, 271W or 271G; |
| T | 273I; |
| U | 275L or 275W; |
| V | 281D, 281K, 281Y or 281P; |
| W | 284E, 284N, 284T, 284L, 284Y or 284M; |
| X | 291D, 291E, 291Q, 291T, 291H, 291I or 291G; |
| Y | 299A, 299D, 299E, 299F, 299G, 299H, 299I, 299K, 299L, |
| | 299M, 299N, 299P, 299Q, 299R, 299S, 299V, 299W or 299Y; |
| Z | 302I; |
| AA | 304D, 304N, 304T, 304H or 304L |
| AB | 305I; |
| AC | 313F; |
| AD | 323I; |
| AE | 325A, 325D, 325E, 325G, 325H, 325I, 325L, 325K, 325R, |
| | 325S, 325F, 325M, 325T, 325V, 325Y, 325W or 325P; |
| AF | 328D, 328Q, 328K, 328R, 328S, 328T, 328V, 328I, 328Y, |
| | 328W, 328P, 328G, 328A, 328E, 328F, 328H, 328M or 328N; |
| AG | 330L, 330Y, 330I or 330V; |
| AH | 332A, 332D, 332E, 332H, 332N, 332Q, 332T, 332K, 332R, |
| | 332S, 332V, 332L, 332F, 332M, 332W, 332P, 332G or 332Y; |
| | and |
| AI | 336E, 336K or 336Y |

Still more particularly preferred variants include one or more modifications selected from groups 1-105:

| Group | Variant |
|---|---|
| 1 | A330L/I332E |
| 2 | D265F/N297E/I332E |
| 3 | D265Y/N297D/I332E |
| 4 | D265Y/N297D/T299L/ |
| | I332E |
| 5 | F241E/F243Q/V262T/ |
| | V264F |
| 6 | F241E/F243Q/V262T/ |
| | V264E/I332E |
| 7 | F241E/F243R/V262E/ |
| | V264R |
| 8 | F241E/F243R/V262E/ |
| | V264R/I332E |
| 9 | F241E/F243Y/V262T/ |
| | V264R |
| 10 | F241E/F243Y/V262T/ |
| | V264R/I332E |
| 11 | F241L/F243L/V262I/ |
| | V264I |
| 12 | F241L/V262I |
| 13 | F241R/F243Q/V262T/ |
| | V264R |
| 14 | F241R/F243Q/V262T/ |
| | V264R/I332E |
| 15 | F241W/F243W/V262A/ |
| | V264A |
| 16 | F241Y/F243Y/V262T/ |
| | V264T |
| 17 | F241Y/F243Y/V262T/ |
| | V264T/N297D/I332E |
| 18 | F243L/V262I/V264W |
| 19 | P243L/V264I |
| 20 | L328D/I332E |
| 21 | L328E/I332E |
| 22 | L328H/I332E |
| 23 | L328I/I332E |
| 24 | L328M/I332E |
| 25 | L328N/I332E |
| 26 | L328Q/I332E |
| 27 | L328T/I332E |
| 28 | L328V/I332E |
| 29 | N297D/A330Y/I332E |
| 30 | N297D/I332E |
| 31 | N297D/I332E/S239D/ |
| | A330L |
| 32 | N297D/S298A/A330Y/I332E |
| 33 | N297D/T299L/I332E |
| 34 | N297D/T299F/I332E/ |
| | N297D/T299H/I332E |
| 35 | N297D/T299I/I332E |
| 36 | N297D/T299L/I332E |
| 37 | N297D/T299V/I332E |
| 38 | N297E/I332E |
| 39 | N297S/I332E |
| 40 | P230A/E233D/I332E |
| 41 | P244H/P245A/P247V |
| 42 | S239D/A330L/I332E |
| 43 | S239D/A330Y/I332E |
| 44 | S239D/A330Y/I332E/ |
| | K326E |
| 45 | S239D/A330Y/I332E/ |
| | K326T |
| 46 | S239D/A330Y/I332E/ |
| | L234I |
| 47 | S239D/A330Y/I332E/ |
| | L235D |
| 48 | S239D/A330Y/I332E/ |
| | V240I |
| 49 | S239D/A330Y/I332E/ |
| | V264T |
| 50 | S239D/A330Y/I332E/ |
| | V266I |
| 51 | S239D/D265F/N297D/ |
| | I332E |
| 52 | S239D/D265H/N297D/ |
| | I332E |
| 53 | S239D/D265I/N297D/ |
| | I332E |
| 54 | S239D/D265L/N297D/ |
| | I332E |
| 55 | S239D/D265T/N297D/ |
| | I332E |
| 56 | S239D/D265V/N297D/ |
| | I332E |
| 57 | S239D/D265Y/N297D/ |
| | I332E |
| 58 | S239D/I332D |
| 59 | S239D/I332E |
| 60 | S239D/I332E/A330I |
| 61 | S239D/I332N |
| 62 | S239D/I332Q |
| 63 | S239D/N297D/I332E |
| 64 | S239D/N297D/I332E/ |
| | A330Y |
| 65 | S239D/N297D/I332E/ |
| | A330Y/F241S/F243H/ |
| | V262T/V264T |
| 66 | S239D/N297D/I332E/ |
| | K326E |
| 67 | S239D/N297D/I332E/ |
| | L235D |
| 68 | S239D/S298A/I332E |
| 69 | S239D/V264I/A330L/ |
| | I332E |
| 70 | S239D/V264I/I332E |
| 71 | S239D/V264I/S298A/ |
| | I332E |
| 72 | S239E/D265N |
| 73 | S239E/D265Q |
| 74 | S239E/I332D |
| 75 | S239E/I332E |
| 76 | S239E/I332N |
| 77 | S239E/I332Q |
| 78 | S239E/N297D/I332E |
| 79 | S239E/V264I/A330Y/I332E |
| 80 | S239E/V264I/I332E |
| 81 | S239E/V264I/S298A/ |
| | A330Y/I332E |
| 82 | S239N/A330L/I332E |
| 83 | S239N/A330Y/I332E |
| 84 | S239N/I332D |
| 85 | S239N/I332E |
| 86 | S239N/I332N |
| 87 | S239N/I332Q |
| 88 | S239N1S298A/I332E |
| 89 | S239Q/I332D |
| 90 | S239Q/I332E |
| 91 | S239Q/I332N |
| 92 | S239Q/I332Q |
| 93 | S239Q/V264I/I332E |
| 94 | S298A/I332E |
| 95 | V264E/N297D/I332E |
| 96 | V264I/A330L/I332E |
| 97 | V264I/A330Y/I332E |
| 98 | V264I/I332E |
| 99 | V264I/S298A/I332E |
| 100 | Y296D/N297D/I332E |
| 101 | Y296E/N297D/I332E |
| 102 | Y296H/N297D/I332E |
| 103 | Y296N/N297D/I332E |
| 104 | Y296Q/N297I/I332E |
| 105 | Y296T/N297D/I332E |

In one embodiment, such ROR1 mAb 1 antibodies or their fragments will comprise a variant Fc Domain having at least one modification in the Fc Domain. In certain embodiments, the variant Fc Domain comprises at least one substitution selected from the group consisting of L235V, F243L, R292P, Y300L, V305I, and P396L, wherein said numbering is that of the EU index as in Kabat. In a specific embodiment, the variant Fc Domain comprises:
(A) at least one substitution selected from the group consisting of F243L, R292P, Y300L, V305I, and P396L;
(B) at least two substitutions selected from the group consisting of:
   (1) F243L and P396L;
   (2) F243L and R292P; and
   (3) R292P and V305I;
(C) at least three substitutions selected from the group consisting of:
   (1) F243L, R292P and Y300L;
   (2) F243L, R292P and V305I;
   (3) F243L, R292P and P396L; and
   (4) R292P, V305I and P396L;
(D) at least four substitutions selected from the group consisting of:
   (1) F243L, R292P, Y300L and P396L; and
   (2) F243L, R292P, V305I and P396L; or
(E) at least the five substitutions selected from the group consisting of:
   (1) F243L, R292P, Y300L, V305I and P396L; and
   (2) L235V, F243L, R292P, Y300L and P396L.

In another specific embodiment, the variant Fc Domain comprises substitutions of:
(A) F243L, R292P, and Y300L;
(B) L235V, F243L, R292P, Y300L, and P396L; or
(C) F243L, R292P, Y300L, V305I, and P396L.

In other embodiments, such ROR1 mAb 1 antibodies or their fragments may possess any Fc variant known in the art, such as those disclosed in Jefferis, R. et al. (2002) “Interaction Sites On Human IgG-Fc For FcgammaR: Current Models,” Immunol. Lett. 82:57-65; Presta, L. G. et al. (2002) “Engineering Therapeutic Antibodies For Improved Function,” Biochem. Soc. Trans. 30:487-90; Idusogie, E. E. et al. (2001) “Engineered Antibodies With Increased Activity To Recruit Complement,” J. Immunol. 166:2571-75; Shields, R. L. et al. (2001) “High Resolution Mapping Of The Binding Site On Human IgG1 For Fc Gamma RI, Fc Gamma RII, Fc Gamma RII, And FcRn And Design Of IgG1 Variants With Improved Binding To The Fc gamma R,” J. Biol. Chem. 276:6591-6604; Idusogie, E. E. et al. (2000) “Mapping Of The C1q Binding Site On Rituxan, A Chimeric Antibody With A Human IgG Fc,” J. Immunol. 164:4178-84; Reddy, M. P. et al. (2000) “Elimination Of Fc Receptor-Dependent Effector Functions Of A Modified IgG4 Monoclonal Antibody To Human CD4,” J. Immunol. 164:1925-1933; Xu, D. et al. (2000) “In Vitro Characterization of Five Humanized OKT3 Effector Function Variant Antibodies,” Cell. Immunol. 200:16-26; Armour, K. L. et al. (1999) “Recombinant human IgG Molecules Lacking Fcgamma Receptor I Binding And Monocyte Triggering Activities,” Eur. J. Immunol. 29:2613-24; Jefferis, R. et al. (1996) “Modulation Of Fc(Gamma)R And Human Complement Activation By IgG3-Core Oligosaccharide Interactions,” Immunol. Lett. 54:101-04; Lund, J. et al. (1996) “Multiple Interactions Of IgG With Its Core Oligosaccharide Can Modulate Recognition By Complement And Human Fc Gamma Receptor I And Influence The Synthesis Of Its Oligosaccharide Chains,” J. Immunol. 157:4963-4969; Hutchins et al. (1995) “Improved Biodistribution, Tumor Targeting, And Reduced Immunogenicity In Mice With A Gamma 4 Variant Of Campath-1H,” Proc. Natl. Acad. Sci. (U.S.A.) 92:11980-84; Jefferis, R. et al. (1995) “Recognition Sites On Human IgG For Fc Gamma Receptors: The Role Of Glycosylation,” Immunol. Lett. 44:111-17; Lund, J. et al. (1995) “Oligosaccharide-Protein Interactions In IgG Can Modulate Recognition By Fc Gamma Receptors,” FASEB J. 9:115-19; Alegre, M. L. et al. (1994) “A Non-Activating “Humanized” Anti-CD3 Monoclonal Antibody Retains Immunosuppressive Properties In Vivo,” Transplantation 57:1537-1543; Lund et al. (1992) “Multiple Binding Sites On The CH2 Domain Of IgG For Mouse Fc Gamma R11,” Mol. Immunol. 29:53-59; Lund et al. (1991) “Human Fc Gamma RI And Fc Gamma RII Interact With Distinct But Overlapping Sites On Human IgG,” J. Immunol. 147:2657-2662; Duncan, A. R. et al. (1988) “Localization Of The Binding Site For The Human High-Affinity Fc Receptor On IgG,” Nature 332:563-564; U.S. Pat. Nos. 5,624,821; 5,885,573; 6,194,551; 7,276,586; and 7,317,091; and PCT Publications WO 00/42072 and PCT WO 99/58572.

In some embodiments, such ROR1 mAb 1 antibodies or their fragments may further comprise one or more glycosylation sites, so that one or more carbohydrate moieties are covalently attached to the molecule. Preferably, such ROR1 mAb 1 antibodies or their fragments with one or more glycosylation sites and/or one or more modifications in the Fc Domain confer or have an enhanced antibody mediated effector function, e.g., enhanced ADCC activity, compared to the unmodified ROR1 mAb 1 antibodies or fragment. In some embodiments, the invention further comprises such ROR1 mAb 1 antibodies or their fragments comprising one or more modifications of amino acids that are directly or indirectly known to interact with a carbohydrate moiety of the Fc Domain, including but not limited to amino acids at positions 241, 243, 244, 245, 245, 249, 256, 258, 260, 262, 264, 265, 296, 299, and 301. Amino acids that directly or indirectly interact with a carbohydrate moiety of an Fc Domain are known in the art, see, e.g., Jefferis, R. et al. (1995) “Recognition Sites On Human IgG For Fc Gamma Receptors: The Role Of Glycosylation,” Immunol. Lett. 44:111-17.

In another embodiment, the invention encompasses such ROR1 mAb 1 antibodies or their fragments that have been modified by introducing one or more glycosylation sites into one or more sites of the molecules, preferably without altering the functionality of the molecules, e.g., binding activity to target antigen or FcγR. Glycosylation sites may be introduced into the variable and/or constant region of the molecules of the invention. As used herein, “glycosylation sites” include any specific amino acid sequence in an antibody to which an oligosaccharide (i.e., carbohydrates containing two or more simple sugars linked together) will specifically and covalently attach. Oligosaccharide side chains are typically linked to the backbone of an antibody via either N- or O-linkages. N-linked glycosylation refers to the attachment of an oligosaccharide moiety to the side chain of an asparagine residue. O-linked glycosylation refers to the attachment of an oligosaccharide moiety to a hydroxyamino acid, e.g., serine, threonine. Such ROR1 mAb 1 antibodies or their fragments may comprise one or more glycosylation sites, including N-linked and 0-linked glycosylation sites. Any glycosylation site for N-linked or O-linked glycosylation known in the art may be used in accordance with the instant invention. An exemplary N-linked glycosylation site is the amino acid sequence: Asn-X-Thr/Ser, wherein X may be any amino acid and Thr/Ser indicates a threonine or a serine. Such a site or sites may be introduced into a molecule of the invention using methods well-known in the art to which this invention pertains (see for example, IN VITRO MUTAGENESIS, RECOMBINANT DNA: A SHORT COURSE, J. D. Watson, et al. W. H. Freeman and Company, New York, 1983, chapter 8, pp. 106-116, which is incorporated herein by reference in its entirety. An exemplary method for introducing a glycosylation site into such ROR1 mAb 1 antibodies or their fragments may comprise: modifying or mutating an amino acid sequence of the molecule so that the desired Asn-X-Thr/Ser sequence is obtained, or expressing a ROR1 mAb 1 antibodies encoding nucleic acid molecule having such a sequence.

In some embodiments, the invention encompasses methods of modifying the carbohydrate content of such ROR1 mAb 1 antibodies or their fragments by adding or deleting a glycosylation site. Methods for modifying the carbohydrate content of antibodies (and molecules comprising antibody domains, e.g., Fc Domain) are well-known in the art and encompassed within the invention, see, e.g., U.S. Pat. No. 6,218,149; EP 0 359 096 Bi; U.S. Publication No. US 2002/0028486; WO 03/035835; U.S. Publication No. 2003/0115614; U.S. Pat. Nos. 6,218,149; 6,472,511; all of which are incorporated herein by reference in their entirety. In other embodiments, the invention encompasses methods of modifying the carbohydrate content of such ROR1 mAb 1 antibodies or their fragments by deleting one or more endogenous carbohydrate moieties of the molecule. In a specific embodiment, the invention encompasses shifting the glycosylation site of the Fc Domain of an antibody, by modifying positions adjacent to 297. In a specific embodiment, the invention encompasses modifying position 296 so that position 296 and not position 297 is glycosylated.

Effector function can be modified by techniques such as those described in PCT Publications No. WO 04/063351; WO 06/088494; WO 07/024249; WO 06/113665; WO 07/021841; WO 07/106707; WO 2008/140603, or by other means. For example, cysteine residue(s) may be introduced in the Fc Domain, thereby allowing interchain disulfide bond formation in this region, resulting in the generation of a homodimeric antibody that may have improved internalization capability and/or increased complement-mediated cell killing and ADCC. See Caron, P. C. et al. (1992) “Engineered Humanized Dimeric Forms Of IgG Are More Effective Antibodies,” J. Exp. Med. 176:1191-1195; Shopes, B. (1992) “A Genetically Engineered Human IgG Mutant With Enhanced Cytolytic Activity,” J. Immunol. 148(9):2918-2922. Homodimeric antibodies with enhanced antitumor activity may also be prepared using heterobifunctional cross-linkers as described in Wolff, E. A. et al. (1993) “Monoclonal Antibody Homodimers: Enhanced Antitumor Activity In Nude Mice,” Cancer Research 53:2560-2565. Alternatively, an antibody can be engineered which has dual Fc Domains and may thereby have enhanced complement lysis and ADCC capabilities (Stevenson, G. T. et al. (1989) “A Chimeric Antibody With Dual Fc Domains (bisFabFc) Prepared By Manipulations At The IgG Hinge,” Anti-Cancer Drug Design 3:219-230.

The fact that a single amino acid alteration of a CDR residue can result in loss of functional binding (Rudikoff, S. etc. (1982) “Single Amino Acid Substitution Altering Antigen-Binding Specificity,” Proc. Natl. Acad. Sci. (USA) 79(6):1979-1983) provides a means for systematically identifying alternative functional CDR sequences. In one preferred method for obtaining such variant CDRs, a polynucleotide encoding the CDR is mutagenized (for example via random mutagenesis or by a site-directed method (e.g., polymerase chain-mediated amplification with primers that encode the mutated locus)) to produce a CDR having a substituted amino acid residue. By comparing the identity of the relevant residue in the original (functional) CDR sequence to the identity of the substituted (non-functional) variant CDR sequence, the BLOSUM62.iij substitution score for that substitution can be identified. The BLOSUM system provides a matrix of amino acid substitutions created by analyzing a database of sequences for trusted alignments (Eddy, S. R. (2004) “Where Did The BLOSUM62 Alignment Score Matrix Come From?,” Nature Biotech. 22(8):1035-1036; Henikoff, J. G. (1992) “Amino acid substitution matrices from protein blocks,” Proc. Natl. Acad. Sci. (USA) 89:10915-10919; Karlin, S. et al. (1990) “Methods For Assessing The Statistical Significance Of Molecular Sequence Features By Using General Scoring Schemes,” Proc. Natl. Acad. Sci. (USA) 87:2264-2268; Altschul, S. F. (1991) “Amino Acid Substitution Matrices From An Information Theoretic Perspective,” J. Mol. Biol. 219, 555-565. Currently, the most advanced BLOSUM database is the BLOSUM62 database (BLOSUM62.iij). Table 4 presents the BLOSUM62.iij substitution scores (the higher the score the more conservative the substitution and thus the more likely the substitution will not affect function). If an antigen-binding fragment comprising the resultant CDR fails to bind to ROR1, for example, then the BLOSUM62.iij substitution score is deemed to be insufficiently conservative, and a new candidate substitution is selected and produced having a higher substitution score. Thus, for example, if the original residue was glutamate (E), and the non-functional substitute residue was histidine (H), then the BLOSUM62.iij substitution score will be 0, and more conservative changes (such as to aspartate, asparagine, glutamine, or lysine) are preferred.

TABLE 4

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | +4 | −1 | −2 | −2 | 0 | −1 | −1 | 0 | −2 | −1 | −1 | −1 | −1 | −2 | −1 | +1 | 0 | −3 | −2 | 0 |
| R | −1 | +5 | 0 | −2 | −3 | +1 | 0 | −2 | 0 | −3 | −2 | +2 | −1 | −3 | −2 | −1 | −1 | −3 | −2 | −3 |
| N | −2 | 0 | +6 | +1 | −3 | 0 | 0 | 0 | +1 | −3 | −3 | 0 | −2 | −3 | −2 | +1 | 0 | −4 | −2 | −3 |
| D | −2 | −2 | +1 | +6 | −3 | 0 | +2 | −1 | −1 | −3 | −4 | −1 | −3 | −3 | −1 | 0 | −1 | −4 | −3 | −3 |
| C | 0 | −3 | −3 | −3 | +9 | −3 | −4 | −3 | −3 | −1 | −1 | −3 | −1 | −2 | −3 | −1 | −1 | −2 | −2 | −1 |
| Q | −1 | +1 | 0 | 0 | −3 | +5 | +2 | −2 | 0 | −3 | −2 | +1 | 0 | −3 | −1 | 0 | −1 | −2 | −1 | −2 |
| E | −1 | 0 | 0 | +2 | −4 | +2 | +5 | −2 | 0 | −3 | −3 | +1 | −2 | −3 | −1 | 0 | −1 | −3 | −2 | −2 |
| G | 0 | −2 | 0 | −1 | −3 | −2 | −2 | +6 | −2 | −4 | −4 | −2 | −3 | −3 | −2 | 0 | −2 | −2 | −3 | −3 |
| H | −2 | 0 | +1 | −1 | −3 | 0 | 0 | −2 | +8 | −3 | −3 | −1 | −2 | −1 | −2 | −1 | −2 | −2 | +2 | −3 |
| I | −1 | −3 | −3 | −3 | −1 | −3 | −3 | −4 | −3 | +4 | +2 | −3 | +1 | 0 | −3 | −2 | −1 | −3 | −1 | +3 |
| L | −1 | −2 | −3 | −4 | −1 | −2 | −3 | −4 | −3 | +2 | +4 | −2 | +2 | 0 | −3 | −2 | −1 | −2 | −1 | +1 |
| K | −1 | +2 | 0 | −1 | −3 | +1 | +1 | −2 | −1 | −3 | −2 | +5 | −1 | −3 | −1 | 0 | −1 | −3 | −2 | −2 |
| M | −1 | −1 | −2 | −3 | −1 | 0 | −2 | −3 | −2 | +1 | +2 | −1 | +5 | 0 | −2 | −1 | −1 | −1 | −1 | +1 |
| F | −2 | −3 | −3 | −3 | −2 | −3 | −3 | −3 | −1 | 0 | 0 | −3 | 0 | +6 | −4 | −2 | −2 | +1 | +3 | −1 |
| P | −1 | −2 | −2 | −1 | −3 | −1 | −1 | −2 | −2 | −3 | −3 | −1 | −2 | −4 | +7 | −1 | −1 | −4 | −3 | −2 |
| S | +1 | −1 | +1 | 0 | −1 | 0 | 0 | 0 | −1 | −2 | −2 | 0 | −1 | −2 | −1 | +4 | +1 | −3 | −2 | −2 |
| T | 0 | −1 | 0 | −1 | −1 | −1 | −1 | −2 | −2 | −1 | −1 | −1 | −1 | −2 | −1 | +1 | +5 | −2 | −2 | 0 |
| W | −3 | −3 | −4 | −4 | −2 | −2 | −3 | −2 | −2 | −3 | −2 | −3 | −1 | +1 | −4 | −3 | −2 | +11 | +2 | −3 |
| Y | −2 | −2 | −2 | −3 | −2 | −1 | −2 | −3 | +2 | −1 | −1 | −2 | −1 | +3 | −3 | −2 | −2 | +2 | +7 | −1 |
| V | 0 | −3 | −3 | −3 | −1 | −2 | −2 | −3 | −3 | +3 | +1 | −2 | +1 | −1 | −2 | −2 | 0 | −3 | −1 | +4 |

The invention thus contemplates the use of random mutagenesis to identify improved CDRs. Phage display technology can alternatively be used to increase (or decrease) CDR affinity. This technology, referred to as affinity maturation, employs mutagenesis or “CDR walking” and re-selection uses the target antigen or an antigenic fragment thereof to identify antibodies having CDRs that bind with higher (or lower) affinity to the antigen when compared with the initial or parental antibody (See, e.g. Glaser et al. (1992) J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased (or decreased) binding affinity for the antigen can be screened by contacting the immobilized mutants with labeled antigen. Any screening method known in the art can be used to identify mutant antibodies with increased or decreased affinity to the antigen (e.g., ELISA) (See Wu et al. 1998, Proc. Natl. Acad. Sci. (U.S.A.) 95:6037; Yelton et al., 1995, J. Immunology 155:1994). CDR walking which randomizes the Light Chain may be used possible (see, Schier et al., 1996, J. Mol. Bio. 263:551).

Methods for accomplishing such affinity maturation are described for example in: Krause, J. C. et al. (2011) “An Insertion Mutation That Distorts Antibody Binding Site Architecture Enhances Function Of A Human Antibody,” MBio. 2(1) pii: e00345-10. doi: 10.1128/mBio.00345-10; Kuan, C. T. et al. (2010) “Affinity-Matured Anti-Glycoprotein NMB Recombinant Immunotoxins Targeting Malignant Gliomas And Melanomas,” Int. J. Cancer 10.1002/ijc.25645; Hackel, B. J. et al. (2010) “Stability And CDR Composition Biases Enrich Binder Functionality Landscapes,” J. Mol. Biol. 401(1):84-96; Montgomery, D. L. et al. (2009) “Affinity Maturation And Characterization Of A Human Monoclonal Antibody Against HIV-1 gp41,” MAbs 1(5):462-474; Gustchina, E. et al. (2009) “Affinity Maturation By Targeted Diversification Of The CDR-H2 Loop Of A Monoclonal Fab Derived From A Synthetic Naïve Human Antibody Library And Directed Against The Internal Trimeric Coiled-Coil Of Gp41 Yields A Set Of Fabs With Improved HIV-1 Neutralization Potency And Breadth,” Virology 393(1):112-119; Finlay, W. J. et al. (2009) “Affinity Maturation Of A Humanized Rat Antibody For Anti-RAGE Therapy: Comprehensive Mutagenesis Reveals A High Level Of Mutational Plasticity Both Inside And Outside The Complementarity-Determining Regions,” J. Mol. Biol. 388(3):541-558; Bostrom, J. et al. (2009) “Improving Antibody Binding Affinity And Specificity For Therapeutic Development,” Methods Mol. Biol. 525:353-376; Steidl, S. et al. (2008) “In Vitro Affinity Maturation Of Human GM-CSF Antibodies By Targeted CDR-Diversification,” Mol. Immunol. 46(1):135-144; and Barderas, R. et al. (2008) “Affinity maturation of antibodies assisted by in silico modeling,” Proc. Natl. Acad. Sci. (USA) 105(26):9029-9034. As an example, multi-well plates may be coated with a selected ROR1 mAb 1 antibody (e.g., 100 ng/well in carbonate buffer at room temperature for 2 hrs) and subsequently incubated with soluble ROR1 added at a dilution of 1/10 and incubated at room temperature for 16 hours or diluted to a concentration of 50 ng/ml in PBS-T-BSA (0.05 ml added to each well and incubated for at least 2 h at room temperature). The plate is then washed and dilutions of recombinant antibodies starting at 0.5 μg/ml in PBS-T-BSA are then added and incubated for 1 hour at room temp. Binding of recombinant antibodies to the captured antigen is then measured using, for example, an anti-human IgG-HRP conjugate and TMB substrate. After stopping color development using dilute sulfuric acid, the plate is read at 450 nM and higher affinity antibodies identified (see, e.g., U.S. Pat. No. 7,351,803).

### VI. Pharmaceutical Compositions

In one embodiment, the present invention includes pharmaceutical compositions for the treatment of a cancer or disease that is characterized by the presence of a Disease-Associated Antigen. Such compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a modified diabody of the present invention, or a combination of such agents and a pharmaceutically acceptable carrier. Preferably, compositions of the invention comprise a prophylactically or therapeutically effective amount of one or more molecules of the invention and a pharmaceutically acceptable carrier. The invention also encompasses pharmaceutical compositions comprising such modified diabodies and a second therapeutic antibody that is specific for a particular disease antigen, and a pharmaceutically acceptable carrier.

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) infected cells or other diseased cells, decreasing symptoms resulting from the disease, increasing the quality of life of those suffering from the disease, decreasing the dose of other medications required to treat the disease, delaying the progression of the disease, and/or prolonging survival of companion animal recipients.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans (see, e.g., REMINGTON: THE SCIENCE AND PRACTICE OF PHARMACY (2012) Allen, Loyd V., Jr. (Ed.) 22^{nd} Edition, Pharmaceutical Press, London UK). The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers containing a modified diabody of the present invention, alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises one or more molecules of the invention. In another embodiment, a kit further comprises one or more other prophylactic or therapeutic agents useful for the treatment of cancer or a disease characterized by the presence of a Disease-Associated Antigen, in one or more containers. In another embodiment, a kit further comprises one or more antibodies or diabodies that bind one or more Disease-Associated Antigens. In certain embodiments, the other prophylactic or therapeutic agent is a chemotherapeutic. In other embodiments, the prophylactic or therapeutic agent is a biological or hormonal therapeutic.

### VII. Methods of Producing the Tri-Specific Binding Molecules of the Present Invention

The Tri-Specific Tri-Specific Binding Molecules of the present invention are most preferably produced through the recombinant expression of nucleic acid molecules that encode such polypeptides, as is well-known in the art.

Polypeptides of the invention may be conveniently prepared using solid-phase peptide synthesis (Merrifield, B. (1986) “Solid Phase Synthesis,” Science 232(4748):341-347; Houghten, R. A. (1985) “General Method For The Rapid Solid-Phase Synthesis Of Large Numbers Of Peptides: Specificity Of Antigen-Antibody Interaction At The Level Of Individual Amino Acids,” Proc. Natl. Acad. Sci. (U.S.A.) 82(15):5131-5135; Ganesan, A. (2006) “Solid-Phase Synthesis In The Twenty-First Century,” Mini Rev. Med. Chem. 6(1):3-10).

In an alternative, antibodies may be made recombinantly and expressed using any method known in the art. Antibodies may be made recombinantly by first isolating the antibodies made from host animals, obtaining the gene sequence, and using the gene sequence to express the antibody recombinantly in host cells (e.g., CHO cells). Another method that may be employed is to express the antibody sequence in plants (e.g., tobacco) or transgenic milk. Suitable methods for expressing antibodies recombinantly in plants or milk have been disclosed (see, for example, Peeters et al. (2001) “Production Of Antibodies And Antibody Fragments In Plants,” Vaccine 19:2756; Lonberg, N. et al. (1995) “Human Antibodies From Transgenic Mice,” Int. Rev. Immunol 13:65-93; and Pollock et al. (1999) “Transgenic Milk As A Method For The Production Of Recombinant Antibodies,” J. Immunol Methods 231:147-157). Suitable methods for making derivatives of antibodies, e.g., chimeric, humanized, single-chain, etc. are known in the art. In another alternative, antibodies may be made recombinantly by phage display technology (see, for example, U.S. Pat. Nos. 5,565,332; 5,580,717; 5,733,743; 6,265,150; and Winter, G. et al. (1994) “Making Antibodies By Phage Display Technology,” Annu. Rev. Immunol. 12.433-455).

The antibodies or protein of interest may be subjected to sequencing by Edman degradation, which is well-known to those of skill in the art. The peptide information generated from mass spectrometry or Edman degradation can be used to design probes or primers that are used to clone the protein of interest.

An alternative method of cloning the protein of interest is by “panning” using purified proteins or portions thereof for cells expressing the antibody or protein of interest. The “panning” procedure may be conducted by obtaining a cDNA library from tissues or cells that express or over-express the desired cDNAs in a second cell type, and screening the transfected cells of the second cell type for a specific binding to the desired protein. Detailed descriptions of the methods used in cloning mammalian genes coding for cell-surface proteins by “panning” can be found in the art (see, for example, Aruffo, A. et al. (1987) “Molecular Cloning Of A CD28 cDNA By A High-Efficiency COS Cell Expression System,” Proc. Natl. Acad. Sci. (U.S.A.) 84:8573-8577 and Stephan, J. et al. (1999) “Selective Cloning Of Cell Surface Proteins Involved In Organ Development: Epithelial Glycoprotein Is Involved In Normal Epithelial Differentiation,” Endocrinol. 140:5841-5854).

cDNAs encoding antibodies, and other peptide agonists, antagonists and modulators can be obtained by reverse transcribing the mRNAs from a particular cell type according to standard methods in the art. Specifically, mRNA can be isolated using various lytic enzymes or chemical solutions according to the procedures set forth in Sambrook et al. supra or extracted by commercially available nucleic-acid-binding resins following the accompanying instructions provided by manufacturers (e.g., Qiagen, Invitrogen, Promega). The synthesized cDNAs are then introduced into an expression vector to produce the antibody or protein of interest in cells of a second type. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, and cosmids.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Any host cells capable of over-expressing heterologous DNAs can be used for the purpose of isolating the genes encoding the antibody, polypeptide or protein of interest. Non-limiting examples of suitable mammalian host cells include but are not limited to COS, HeLa, and CHO cells. Preferably, the host cells express the cDNAs at a level of about 5-fold higher, more preferably 10-fold higher, more preferably 20-fold higher, more preferably 50-fold higher, more preferably 100-fold higher than that of the corresponding endogenous antibody or protein of interest, if present, in the host cells. Screening the host cells for a specific binding to a desired protein is preferably effected by an immunoassay or FACS. A cell over-expressing the antibody or protein of interest can be identified in this way.

Various techniques are also available which may now be employed to produce mutant peptide agonists, antagonists, and modulators which encodes for additions, deletions, or changes in amino acid sequence of the resultant protein relative to the parent peptide agonist, antagonist or modulator molecule.

The invention includes modifications to the Tri-Specific Binding Molecules of the invention that do not significantly affect their properties and variants that have enhanced or decreased activity. Modification of polypeptides is routine practice in the art. Examples of modified polypeptides include polypeptides with conservative substitutions of amino acid residues, one or more deletions or additions of amino acids which do not significantly deleteriously change the functional activity, or use of chemical analogs. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tyrosine. These polypeptides also include glycosylated and non-glycosylated polypeptides, as well as polypeptides with other post translational modifications, such as, for example, glycosylation with different sugars, acetylation, and phosphorylation. Preferably, the amino acid substitutions would be conservative, i.e., the substituted amino acid would possess similar chemical properties as that of the original amino acid. Such conservative substitutions are known in the art, and examples have been provided above. Amino acid modifications can range from changing or modifying one or more amino acids to complete redesign of a region, such as the Variable Domain. Changes in the Variable Domain can alter binding affinity and/or specificity. Other methods of modification include using coupling techniques known in the art, including, but not limited to, enzymatic means, oxidative substitution and chelation. Modifications can be used, for example, for attachment of labels for immunoassay, such as the attachment of radioactive moieties for radioimmunoassay. Modified polypeptides are made using established procedures in the art and can be screened using standard assays known in the art.

The invention also encompasses fusion proteins comprising one or more fragments or regions from the polypeptides and antibodies of this invention. In one embodiment, a fusion polypeptide is provided that comprises at least 10 contiguous amino acids of variable Light Chain region and at least 10 amino acids of variable heavy chain region. In another embodiment, the fusion polypeptide contains a heterologous immunoglobulin constant region. In another embodiment, the fusion polypeptide contains a Light Chain Variable Domain and a Heavy Chain Variable Domain of an antibody produced from a publicly-deposited hybridoma. For purposes of this invention, an antibody fusion protein contains one or more polypeptide Domains that specifically bind to a desired viral epitope or a desired activating receptor of an immune effector cell or a protein present on the surface of an immune effector cell that expresses such an activating receptor and another amino acid sequence to which it is not attached in the native molecule, for example, a heterologous sequence or a homologous sequence from another region.

The invention includes polypeptides comprising an amino acid sequence of the antibodies of this invention. The polypeptides of this invention can be made by procedures known in the art. The polypeptides can be produced by proteolytic or other degradation of the antibodies, by recombinant methods (i.e., single or fusion polypeptides) as described above or by chemical synthesis. Polypeptides of the antibodies, especially shorter polypeptides up to about 50 amino acids, are conveniently made by chemical synthesis. Methods of chemical synthesis are known in the art and are commercially available. For example, such a polypeptide could be produced by an automated polypeptide synthesizer employing the solid-phase method.

### VIII. Uses of the Compositions of the Invention

The present invention encompasses compositions, including pharmaceutical compositions, comprising the Tri-Specific Binding Molecules of the invention, polypeptides derived from such molecules, polynucleotides comprising sequences encoding such molecules or polypeptides, and other agents as described herein.

The Tri-Specific Binding Molecules of the present invention have the ability to coordinately bind to three epitopes, and thus have substantial use in diagnostics, chemical separation, and therapeutics involving such epitopes. For example, such molecules may be used as a reagent in a sandwich immunoassay.

In the embodiment in which such Tri-Specific Binding Molecules bind to an epitope of a Disease-Associated Antigen, such molecules may be used to treat the disease or condition associated with or characterized by the expression of such Disease-Associated Antigen. Thus, without limitation, pharmaceutical compositions comprising such molecules may be employed in the diagnosis or treatment of cancer, and diseases caused by a pathogen (e.g., bacterial, fungal, viral or protozoan) infection.

### IX. Methods of Administration

The compositions of the present invention may be provided for the treatment, prophylaxis, and amelioration of one or more symptoms associated with a disease, disorder or infection by administering to a subject an effective amount of a pharmaceutical composition of the invention. In a preferred aspect, such compositions are substantially purified (i.e., substantially free from substances that limit its effect or produce undesired side-effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering the Tri-Specific Binding Molecules of the present invention include, but are not limited to, parenteral administration (e.g., intradermal, intramuscular, intraperitoneal, intravenous and subcutaneous), epidural, and mucosal (e.g., intranasal and oral routes). In a specific embodiment, the molecules of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the Tri-Specific Binding Molecules of the present invention may be packaged in a hermetically sealed container such as an ampoule or sachette indicating the quantity of such molecules. In one embodiment, the Tri-Specific Binding Molecules of the present invention are supplied as a dry sterilized lyophilized powder or water free concentrate in a hermetically sealed container and can be reconstituted, e.g., with water or saline to the appropriate concentration for administration to a subject. Preferably, the Tri-Specific Binding Molecules of the present invention are supplied as a dry sterile lyophilized powder in a hermetically sealed container at a unit dosage of at least 5 μg, more preferably at least 10 μg, at least 15 μg, at least 25 μg, at least 50 μg, at least 100 μg, or at least 200 μg.

The lyophilized Tri-Specific Binding Molecules of the present invention should be stored at between 2 and 8° C. in their original container and the molecules should be administered within 12 hours, preferably within 6 hours, within 5 hours, within 3 hours, or within 1 hour after being reconstituted. In an alternative embodiment, the Tri-Specific Binding Molecules of the present invention are supplied in liquid form in a hermetically sealed container indicating the quantity and concentration of the molecule, fusion protein, or conjugated molecule. Preferably, the liquid form of the Tri-Specific Binding Molecules of the present invention is supplied in a hermetically sealed container in which the molecules are present at a concentration of least 1 μg/ml, more preferably at least 2.5 μg/ml, at least 5 μg/ml, at least 10 μg/ml, at least 50 μg/ml, or at least 100 μg/ml.

As used herein, an “effective amount” of a pharmaceutical composition, in one embodiment, is an amount sufficient to effect beneficial or desired results including, without limitation, clinical results such as decreasing symptoms resulting from the disease attenuating a symptom of infection (e.g., viral load, fever, pain, sepsis, etc.) or a symptom of cancer (e.g., the proliferation, of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

An effective amount can be administered in one or more administrations. For purposes of this invention, an effective amount of drug, compound, or pharmaceutical composition is an amount sufficient to reduce the proliferation of (or the effect of) viral presence and to reduce and/or delay the development of the viral disease, either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art. Typical dosages for antibody administration comprise one or more unit doses between 0.1- to 100 mg/kg/body weight.

The amount of the Tri-Specific Binding Molecule of the present invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For the Tri-Specific Binding Molecules of the present invention, the dosage administered to a patient is typically at least about 0.01 μg/kg/day, at least about 0.05 μg/kg/day, at least about 0.1 μg/kg/day, at least about 0.2 μg/kg/day, at least about 0.5 μg/kg/day, at least about 1 μg/kg/day, at least about 2 μg/kg/day, at least about 5 μg/kg/day, at least about 10 μg/kg/day, at least about 20 μg/kg/day, at least about 50 μg/kg/day, at least about 0.1 mg/kg/day, or more of the subject's body weight

Preferably, the dosage administered to a patient is between about 0.01 g/kg/day and about 0.1 mg/kg/day, more preferably, between about 0.01 μg/kg/day and about 50 μg/kg/day, more preferably, between about 0.01 μg/kg/day and about 50 g/kg/day, more preferably, between about 0.01 μg/kg/day and about 10 μg/kg/day, more preferably, between about 0.01 μg/kg/day and about 1 μg/kg/day, more preferably, between about 0.01 μg/kg/day and about 0.5 μg/kg/day, and more preferably, between about 0.01 μg/kg/day and about 0.1 μg/kg/day of the subject's body weight. The dosage and frequency of administration of the Tri-Specific Binding Molecules of the invention may be reduced or altered by enhancing uptake and tissue penetration of the Tri-Specific Binding Molecules by modifications such as, for example, lipidation.

In another embodiment, the patient is administered a treatment regimen comprising one or more doses of such prophylactically or therapeutically effective amount of the Tri-Specific Binding Molecules encompassed by the invention, wherein the treatment regimen is administered over 2 days, 3 days, 4 days, 5 days, 6 days or 7 days. In certain embodiments, the treatment regimen comprises intermittently administering doses of the prophylactically or therapeutically effective amount of the Tri-Specific Binding Molecules encompassed by the invention (for example, administering a dose on day 1, day 2, day 3 and day 4 of a given week and not administering doses of the prophylactically or therapeutically effective amount of the Tri-Specific Binding Molecules encompassed by the invention on day 5, day 6 and day 7 of the same week). Typically, there are 1, 2, 3, 4, 5, or more courses of treatment. Each course may be the same regimen or a different regimen.

In another embodiment, the administered dose escalates over the first quarter, first half or first two-thirds or three-quarters of the regimen(s) (e.g., over the first, second, or third regimens of a 4 course treatment) until the daily prophylactically or therapeutically effective amount of the Tri-Specific Binding Molecules encompassed by the invention is achieved.

In one embodiment, the dosage of the Tri-Specific Binding Molecules of the present invention administered to a patient may be calculated for use as a single agent therapy. In another embodiment the Tri-Specific Binding Molecules of the present invention are used in combination with other therapeutic compositions and the dosage administered to a patient are lower than when such Tri-Specific Binding Molecules are used as a single agent therapy.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

In another embodiment, the compositions can be delivered in a vesicle, in particular a liposome (See Langer (1990) “New Methods Of Drug Delivery,” Science 249:1527-1533); Treat et al., in Liposomes in the Therapy of Infectious Disease and Cancer, Lopez-Berestein and Fidler (eds.), Liss, New York, pp. 353-365 (1989); Lopez-Berestein, ibid., pp. 3 17-327; see generally ibid.).

In yet another embodiment, the compositions can be delivered in a controlled release or sustained release system. Any technique known to one of skill in the art can be used to produce sustained release formulations comprising one or more molecules of the invention. See, e.g., U.S. Pat. No. 4,526,938; PCT publication WO 91/05548; PCT publication WO 96/20698; Ning et al. (1996) “Intratumoral Radioimmunotherapy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety. In one embodiment, a pump may be used in a controlled release system (See Langer, supra; Sefton, (1987) “Implantable Pumps,” CRC Crit. Rev. Biomed. Eng. 14:201-240; Buchwald et al. (1980) “Long-Term, Continuous Intravenous Heparin Administration By An Implantable Infusion Pump In Ambulatory Patients With Recurrent Venous Thrombosis,” Surgery 88:507-516; and Saudek et al. (1989) “A Preliminary Trial Of The Programmable Implantable Medication System For Insulin Delivery,” N. Engl. J. Med. 321:574-579). In another embodiment, polymeric materials can be used to achieve controlled release of antibodies (see e.g., MEDICAL APPLICATIONS OF CONTROLLED RELEASE, Langer and Wise (eds.), CRC Pres., Boca Raton, Florida (1974); CONTROLLED DRUG BIOAVAILABILITY, DRUG PRODUCT DESIGN AND PERFORMANCE, Smolen and Ball (eds.), Wiley, New York (1984); Levy et al. (1985) “Inhibition Of Calcification Of Bioprosthetic Heart Valves By Local Controlled-Release Diphosphonate,” Science 228:190-192; During et al. (1989) “Controlled Release Of Dopamine From A Polymeric Brain Implant: In Vivo Characterization,” Ann. Neurol. 25:351-356; Howard et al. (1989) “Intracerebral Drug Delivery In Rats With Lesion-Induced Memory Deficits,” J. Neurosurg. 7(1):105-112); U.S. Pat. Nos. 5,679,377; 5,916,597; 5,912,015; 5,989,463; 5,128,326; PCT Publication No. WO 99/15154; and PCT Publication No. WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In yet another embodiment, a controlled release system can be placed in proximity of the therapeutic target (e.g., the lungs), thus requiring only a fraction of the systemic dose (see, e.g., Goodson, in MEDICAL APPLICATIONS OF CONTROLLED RELEASE, supra, vol. 2, pp. 115-138 (1984)). In another embodiment, polymeric compositions useful as controlled release implants are used according to Dunn et al. (See U.S. Pat. No. 5,945,155). This particular method is based upon the therapeutic effect of the in situ-controlled release of the bioactive material from the polymer system. The implantation can generally occur anywhere within the body of the patient in need of therapeutic treatment. In another embodiment, a non-polymeric sustained delivery system is used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled release systems are discussed in the review by Langer (1990, “New Methods Of Drug Delivery,” Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al. (1996) “Intratumoral Radioimmunotheraphy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety.

In a specific embodiment where the composition of the invention is a nucleic acid encoding a Tri-Specific Binding Molecule of the present invention, the nucleic acid can be administered in vivo to promote expression of its encoded Tri-Specific Binding Molecule, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of the Tri-Specific Binding Molecules of the present invention can include a single treatment or, preferably, can include a series of treatments. In a preferred example, a subject is treated with molecules of the invention one time per week for between about 1 to 10 weeks, preferably between 2 to 8 weeks, more preferably between about 3 to 7 weeks, and even more preferably for about 4, 5, or 6 weeks. In other embodiments, the pharmaceutical compositions of the invention are administered once a day, twice a day, or three times a day. In other embodiments, the pharmaceutical compositions are administered once a week, twice a week, once every two weeks, once a month, once every six weeks, once every two months, twice a year or once per year. It will also be appreciated that the effective dosage of the molecules used for treatment may increase or decrease over the course of a particular treatment.

Having now generally described the invention, the same will be more readily understood through reference to the following Examples. Such Examples are provided by way of illustration and are not intended to be limiting of the present invention unless specified.

### Example 1

### Production and Properties of Exemplary Anti-CD3, Anti-CD8, Anti-B7-H3 Tri-Specific Binding Molecules

In order to develop a therapeutic molecule that would exhibit greater specificity to CD8+ T cells, and more potent re-directed killing, Tri-Specific Binding Molecules were constructed having the ability to coordinately bind to CD3, to CD8 and to a Disease-Associated Antigen. The produced Tri-Specific Binding Molecule additionally possessed an Fc Domain to enhance the half-life of the Tri-Specific Binding Molecule in vivo. The general structures of the Tri-Specific Binding Molecules are shown in FIGS. 4A-4D. An exemplary Tri-Specific Binding Molecule specific for the Disease-Associated Antigen B7-H3 was constructed. The Tri-Specific Binding Molecule is termed B7-H3 mAb 1/CD3 mAb 2/CD8 mAb 1 to denote the relative positions of the Binding Domains within the Tri-Specific Binding Molecule. The B7-H3 Binding Domain occupies the Site A position, the CD3 Binding Domain occupies the Site B position and the CD8 Binding Domain occupies the Site C position (FIG. 4A). The B7-H3 mAb 1/CD3 mAb 2/CD8 mAb 1 Tri-Specific Binding Molecule was composed of four different polypeptide chains (Table 5).

TABLE 5

| Poly- | | |
|---|---|---|
| peptide | | |
| Chain | Domains | Binding Affinity |
| 1 | VL(B7-H3 mAb 1)-VH(CD3 mAb 2)- | Light Chain: B7-H3 |
| | E-Coil-(CH2—CH3) | Heavy Chain: CD3 |
| 2 | VL(CD3 mAb 2)-VH(B7-H3 mAb 1)- | Light Chain: CD3 |
| | K-Coil | Heavy Chain: B7-H3 |
| 3 | Heavy Chain CD8 mAb 1 | CD8 |
| 4 | Light Chain CD8 mAb 1 | CD8 |

The amino acid sequence of the first polypeptide chain of the B7-H3 mAb 1/CD3 mAb 2/CD8 mAb 1 Tri-Specific Binding Molecule is (SEQ ID NO:59):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIKGGG SGGGGEVQLV
ESGGGLVQPG GSLRLSCAAS GFTFSTYAMN WVRQAPGKGL
EWVGRIRSKY NNYATYYADS VKDRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

In the first polypeptide chain, VL(B7-H3 mAb 1) has the amino acid sequence of SEQ ID NO:41, VH(CD3 mAb 2) has the amino acid sequence of SEQ ID NO:27, E-coil has the amino acid sequence of SEQ ID NO:3 and (CH2-CH3) has the amino acid sequence of the “knob-bearing” amino acid sequence of SEQ ID NO:7.

The amino acid sequence of the second polypeptide chain of the B7-H3 mAb 1/CD3 mAb 2/CD8 mAb 1 Tri-Specific Binding Molecule is (SEQ ID NO:60):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSY WMQWVRQAPG
QGLEWMGTIY PGDGDTRYTQ KFKGRVTITA DKSTSTAYME
LSSLRSEDTA VYYCARRGIP RLWYFDVWGQ GTTVTVSSGG
CGGGKVAALK EKVAALKEKV AALKEKVAAL KE

In the second polypeptide chain, VL(CD3 mAb 2) has the amino acid sequence of SEQ ID NO:26, VH(B7-H3 mAb 1) has the amino acid sequence of SEQ ID NO:42, and K-coil has the amino acid sequence of SEQ ID NO:4.

The amino acid sequence of the third polypeptide chain of the B7-H3 mAb 1/CD3 mAb 2/CD8 mAb 1 Tri-Specific Binding Molecule is (SEQ ID NO:61):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

In the third polypeptide chain, the amino acid sequence of the employed CD8 mAb 1 Heavy Chain Variable Domain has the amino acid sequence of SEQ ID NO:30, a Hinge Domain, a CH1 Domain and the “hole-bearing” CH2-CH3 Domain with an H435R substitution to remove the Protein A binding site (SEQ ID NO:8).

The amino acid sequence of the fourth polypeptide chain of the B7-H3 mAb 1/CD3 mAb 2/CD8 mAb 1 Tri-Specific Binding Molecule is (SEQ ID NO:62):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

In the fourth polypeptide chain, the amino acid sequence of the employed CD8 mAb 1 Light Chain Variable Domain has the amino acid sequence of SEQ ID NO:29 and a kappa Light Chain Constant Domain.

The expressed B7H3 mAb 1/CD3 mAb 2/CD8 mAb 1 Tri-Specific Binding Molecule was loaded onto an MSA resin, washed with 10 mM NaPO₄ (pH6); 10 mM NaPO₄, 1M NaCl (pH6) and 10 mM NaPO₄ (pH6). Polypeptides were eluted from the resin with 50 mM glycine (pH3) and neutralized with 1M Tris (pH8). Expression was found to be 1.7 mg/L; the Tri-Specific Binding Molecule preparation was 0.6 mg/ml, having a final yield of 0.42 mg.

The properties of the B7-H3 mAb 1/CD3 mAb 2/CD8 mAb 1 Tri-Specific Binding Molecule were compared with those of a B7-H3×CD3 DART and a B7-H3×CD3 DART with an Fc Domain. As shown in FIGS. 5A-5B, the B7-H3 mAb 1/CD3 mAb 2/CD8 mAb 1 Tri-Specific Binding Molecule showed similar target cell binding (A498 cells (FIG. 5A); JIMT-1 (FIG. 5B)) compared to the B7-H3×CD3 DART and a B7-H3×CD3 DART with an Fc Domain. However, as shown in FIGS. 5C-5D, the B7-H3 mAb 1/CD3 mAb 2/CD8 mAb 1 Tri-Specific Binding Molecule demonstrated greatly increased binding to CD8+ T cells compared to CD4+ T cells.

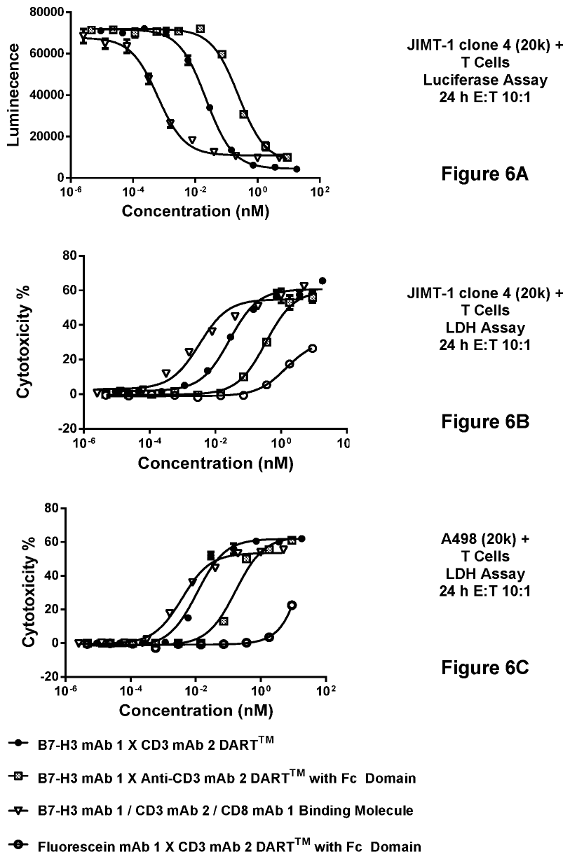

In order to demonstrate the ability of the B7-H3 mAb 1/CD3 mAb 2/CD8 mAb 1 Tri-Specific Binding Molecules of the present invention to mediate the re-directed killing of target cells, such molecules were incubated in the presence of T cells and either JIMT-1 or A498 target cells. JIMT-1 cells are a trastuzumab-resistant carcinoma line (Tanner, M. et al. (2004) “Characterization Of A Novel Cell Line Established From A Patient With Herceptin-Resistant Breast Cancer,” Mol. Cancer Ther. 3(12):1585-1592). The A498 cell line is a renal cell carcinoma cell line (Gogh, J. (1978) “Cultivation, Characterization, And Identification Of Human Tumor Cells With Emphasis On Kidney, Testis, And Bladder Tumors,” Natl. Cancer Inst. Monogr. 49:5-9). As shown in FIGS. 6A-6C, re-directed killing of the target cells was observed. Unexpectedly, such killing was substantially more potent than that observed for corresponding B7-H3×CD3 DART and a B7-H3×CD3 DART with an Fc Domain. The observed re-directed killing is summarized in Table 6.

TABLE 6

| | Re-Directed Killing | | | | |
|---|---|---|---|---|---|
| | JIMT-1 Cells | | | A498 Cells | |
| | LDH | LDH | Luciferase | LDH | LDH |
| Binding Molecule | Max Killing | EC50 | EC50 | Max | EC50 |
| Assay | (%) | (pM) | (pM) | Killing (%) | (pM) |
| B7-H3 X CD3 | 60.72 | 27 | 22 | 61.95 | 11 |
| DART ™ | | | | | |
| B7-H3 X CD3 | 59.95 | 343 | 245 | 63.2 | 168 |
| DART ™ with Fc | | | | | |
| Domain | | | | | |
| B7-H3 mAb 1/ | 54.88 | 0.4 | 0.6 | 53.5 | 4 |
| CD3 mAb 2/CD8 | | | | | |
| mAb 1 | | | | | |
| Tri-Specific | | | | | |
| Binding Molecule | | | | | |

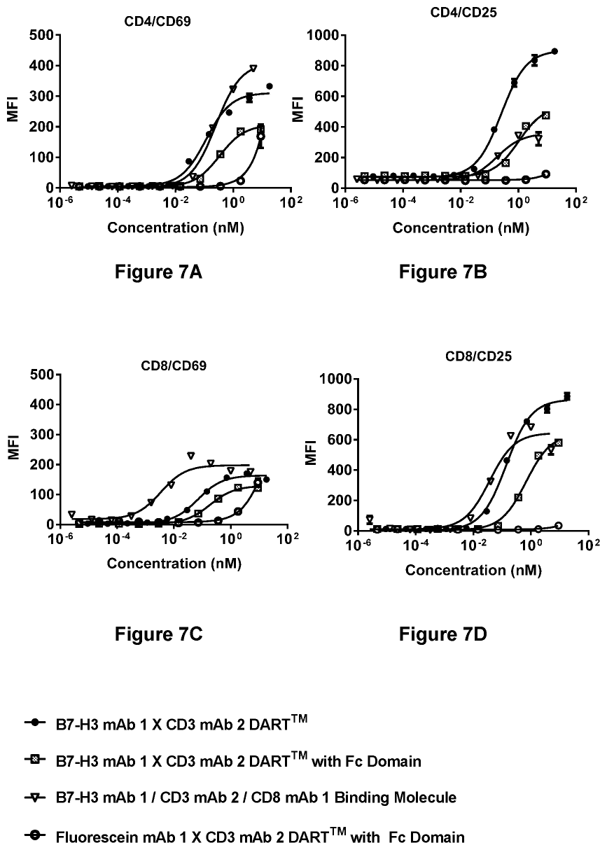

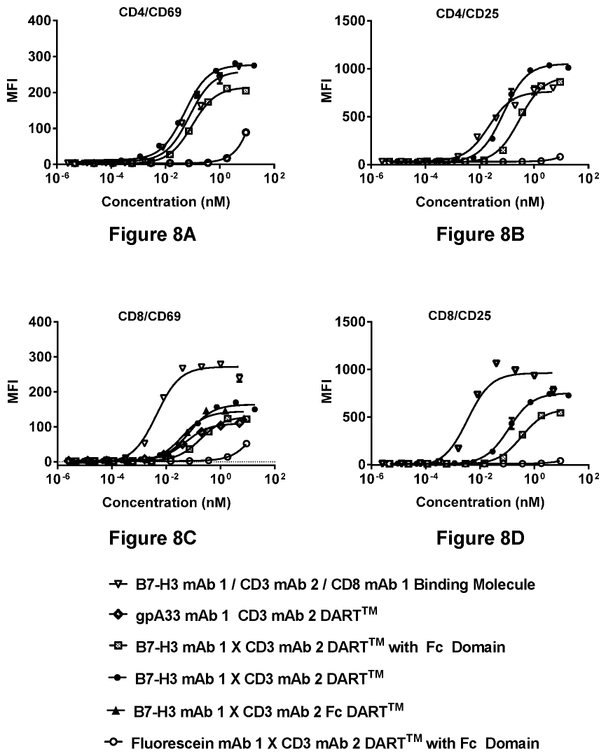

FIGS. 7A-7D show the ability of the B7-H3 mAb 1/CD3 mAb 2/CD8 mAb 1 Tri-Specific Binding Molecules to mediate T cell activation after incubation with JIMT-1 cells. FIGS. 8A-8D show the ability of the B7-H3 mAb 1/CD3 mAb 2/CD8 mAb 1 Tri-Specific Binding Molecules to mediate T cell activation after incubation with A498 cells. In both cases, such activation was unexpectedly superior to the activation observed with comparative B7-H3×CD3 DART™ and a B7-H3×CD3 DART™ with an Fc Domain. Table 7 summarizes the EC50 results.

TABLE 7

| | | EC50 Values of B7-H3 mAb 1/CD3 mAb | | |
|---|---|---|---|---|
| | | 2/CD8 mAb 1 Tri-Specific Binding | | |
| | | Molecule Compared to DARTs | | |
| | | | | Tri-Specific |
| Tumor | T Cell | | DART ™ with | Binding |
| Cells | Subset | DART ™ | Fc Domain | Molecule |
| A498 | CD4/CD69 | 51 | 95 | 75 |
| | CD4/CD25 | 75 | 291 | 22 |
| | CD8/CD69 | 70 | 185 | 4 |
| | CD8/CD25 | 115 | 339 | 4 |
| | CTL | 10 | 48 | 4 |
| JIMT-1 | CD4/CD69 | 116 | 339 | 253 |
| | CD4/CD25 | 257 | 1034 | 185 |
| | CD8/CD69 | 70 | 185 | 3 |
| | CD8/CD25 | 140 | 678 | 37 |
| | CTL | 7 | 68 | 1 |

The expressed B7-H3 mAb 1/CD3 mAb 2/CD8 mAb 1 Tri-Specific Binding Molecule exhibited much greater (13-fold) cytolytic activity using CD8+ effector cells compared to CD4+ effector cells. The B7-H3 mAb 1/CD3 mAb 2/CD8 mAb 1 Tri-Specific Binding Molecule also exhibited greatly increased (85-fold) overall potency using ‘pan’ T cells as effectors compared to the DART™.

### Example 2

### Effect of CD8 Binding Domain on Re-Directed Cytotoxicity

In order to assess the effect of CD8 specificity, a second Tri-Specific Binding Molecule specific for the Disease-Associated Antigen B7-H3 was constructed utilizing a different CD8 antibody Variable Domain sequence. The B7-H3 Variable Domain specificities and CD3 Variable Domain specificities were identical to those used to construct the B7-H3 mAb 1/CD3 mAb 2/CD8 mAb 1 Tri-Specific Binding Molecule. The Tri-Specific Binding Molecule is termed B7-H3 mAb 1/CD3 mAb 2/CD8 mAb 2 and was composed of four different polypeptide chains (Table 8).

TABLE 8

| Polypeptide | | |
|---|---|---|
| Chain | Domains | Binding Affinity |
| 1 | VL(B7-H3 mAb 1)-VH(CD3 | Light Chain: B7-H3 |
| | mAb 2)-E-Coil-(CH2—CH3) | Heavy Chain: CD3 |
| 2 | VL(CD3 mAb 2)- | Light Chain: CD3 |
| | VH(B7-H3 mAb 1)-K-Coil | Heavy Chain: B7-H3 |
| 3 | Heavy Chain CD8 mAb 2 | CD8 |
| 4 | Light Chain CD8 mAb 2 | CD8 |

The amino acid sequence of the first polypeptide chain of the B7-H3 mAb 1/CD3 mAb 2/CD8 mAb 2 Tri-Specific Binding Molecule is (SEQ ID NO:63):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIKGGG SGGGGEVQLV
ESGGGLVQPG GSLRLSCAAS GFTFSTYAMN WVRQAPGKGL
EWVGRIRSKY NNYATYYADS VKDRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

The amino acid sequence of the second polypeptide chain of the B7-H3 mAb 1/CD3 mAb 2/CD8 mAb 2 Tri-Specific Binding Molecule is (SEQ ID NO:64):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSY WMQWVRQAPG
QGLEWMGTIY PGDGDTRYTQ KFKGRVTITA DKSTSTAYME
LSSLRSEDTA VYYCARRGIP RLWYFDVWGQ GTTVTVSSGG
CGGGKVAALK EKVAALKEKV AALKEKVAAL KE

The amino acid sequence of the third polypeptide chain of the B7-H3 mAb 1/CD3 mAb 2/CD8 mAb 2 Tri-Specific Binding Molecule is (SEQ ID NO:65):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

The amino acid sequence of the fourth polypeptide chain of the B7-H3 mAb 1/CD3 mAb 2/CD8 mAb 2 Tri-Specific Binding Molecule is (SEQ ID NO:66):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

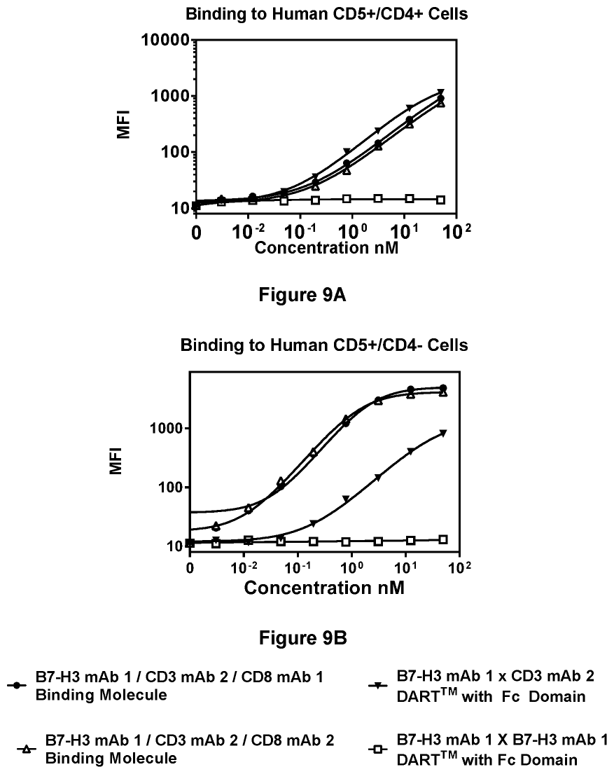

To compare the ability of the B7-H3 mAb 1/CD3 mAb 2/CD8 mAb 1 (construction and sequence described above) or B7-H3 mAb 1/CD3 mAb 2/CD8 mAb 2 Tri-Specific Binding Molecules to bind T cells, human PBMC of healthy donors were purified with Ficoll, wash twice with PBS and re-suspended in FACS buffer contained 10% Human AB serum and incubate at room temperature for 20 minutes, spin down the cell and re-suspended 4×10⁶ cells/mL cells in FACS buffer. 50 μl of serial titrated B7-H3 mAb 1/CD3 mAb 2/CD8 mAb 1 or B7-H3 mAb 1/CD3 mAb 2/CD8 mAb 2 Tri-Specific Binding Molecules or DART™ (B7-H3×CD3 or B7-H3×CD3 with Fc Domain) were added to the wells of a 96-well deep plate. 50 μL (4×10⁶ cells/mL) of well-mixed cells in FACS buffer containing 0.01% sodium azide were then added into corresponding wells and mixed thoroughly using a pipette. The plate was incubated in the dark for about 45 minutes at 2-8° C. At the end of the incubation, the cells were washed twice by adding 300 μL of FACS buffer to each well, centrifuging the plate at 1,200 rpm for 5 minutes, and discarding the supernatant. The cell pellets were re-suspended in 100 μL mixture of PE-conjugated goat anti-Human Fcγ 1:500 diluted, CD5-APC and CD4-PerCP5.5 in FACS buffer containing 0.01% sodium azide, and incubated in the dark for about 45 minutes at 2-8° C. At the end of the incubation, the cells were washed, re-suspended with FACS buffer, and analyzed with a BD Caliber flow cytometer. Cells were gated to CD5⁺ CD4⁺ (FIG. 9A) or CD5⁺ CD4⁻ (FIG. 9B). Differential staining was observed on the CD5⁺ CD4⁻ population comparing to binding molecules that possessed or lacked CD8 specificity.

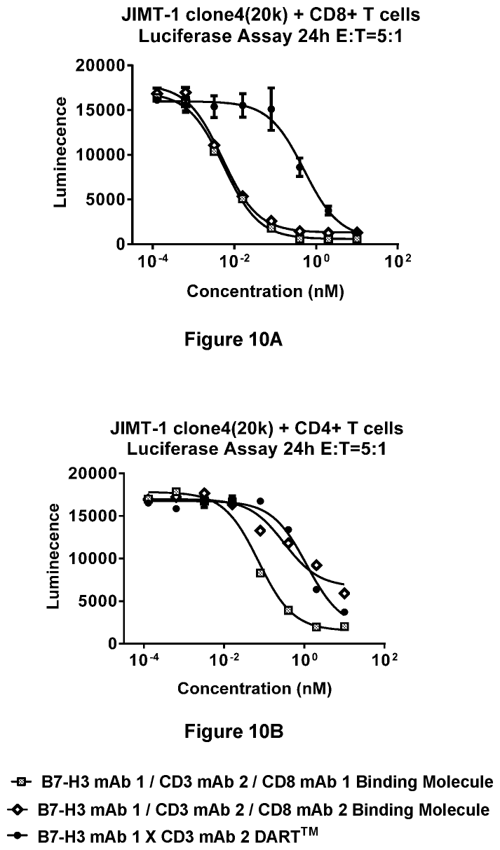

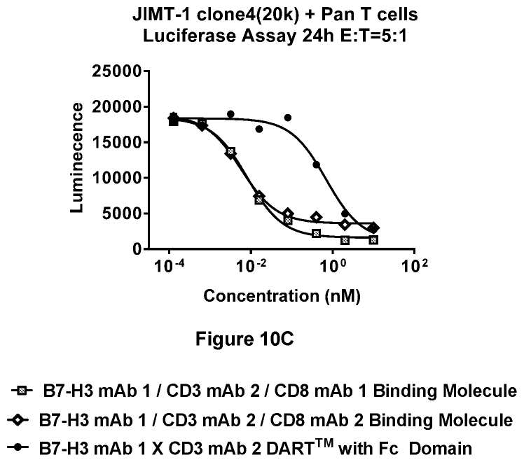

The cytotoxicity of the B7-H3 mAb 1/CD3 mAb 2/CD8 mAb 1 Tri-Specific Binding Molecule was compared to that of the B7-H3 mAb1/CD3 mAb 2/CD8 mAb 2 Tri-Specific Binding Molecule. Both a luciferase assay and an LDH assay were employed. The results of the two assays were in agreement. The two Tri-Specific Binding Molecules caused equivalent re-directed cytotoxicity in the presence of activating CD8+ T cell or pan T cell populations. The Tri-Specific Binding Molecule having a CD8 mAb 1 Binding Domain exhibited greater re-directed cytotoxicity in the presence of CD8+ cell populations or pan T cells compared to the B7H3×CD3 DART (FIG. 10A-10C).

An increase (60-fold) in EC50 for CD8+vs. CD4+ effector cells was also observed for the B7-H3 mAb 1/CD3 mAb 2/CD8 mAb 2 Tri-Specific Binding Molecule compared to the B7-H3 mAb 1/CD3 mAb 2/CD8 mAb 1 Tri-Specific Binding Molecule. For the B7-H3 mAb 1/CD3 mAb 2/CD8 mAb 2 Tri-Specific Binding Molecule, increased potency resulting in a decrease in the EC50 of greater that 100-fold was observed when pan T cells were used as effector cells.

### Example 3

### Effect Of Domain Positions On Re-Directed Cytotoxicity

In order to assess the effect of position for a given Binding Domain (CD3, CD8 and Disease-Associate Antigen) within the Tri-Specific Binding Molecule (Site A, Site B and Site C), several additional Tri-Specific Binding Molecules were constructed. Table 9 shows the Tri-Specific Binding Molecules and the location (Site A, Site B and Site C) of the various Binding Domains (CD3, CD8 and Disease-Associated Antigen).

TABLE 9

| Tri-Specific Binding Molecule | Site A | Site B | Site C |
|---|---|---|---|
| B7-H3 mAb 1/CD3 mAb 2/CD8 mAb 1 | B7-H3 | CD3 | CD8 |
| Tri-Specific Binding Molecule | mAb 1 | mAb 2 | mAb 1 |
| CD3 mAb 2/CD8 mAb 1/B7-H3 mAb 1 | CD3 | CD8 | B7-H3 |
| Tri-Specific Binding Molecule | mAb 2 | mAb 1 | mAb 1 |
| B7-H3 mAb 1/CD8 mAb 1/CD3 mAb 2 | B7-H3 | CD8 | CD3 |
| Tri-Specific Binding Molecule | mAb 1 | mAb 1 | mAb 2 |

The construction and sequence of the B7-H3 mAb 1/CD3 mAb 2/CD8 mAb 1 Tri-Specific Binding Molecule was described above. For the additional two Tri-Specific Binding Molecules (CD3 mAb 2/CD8 mAb 1/B7-H3 mAb 1 and B7-H3 mAb 1/CD8 mAb 1/CD3 mAb 2), the B7-H3 Variable Domain specificities, CD3 Variable Domain specificities and CD8 Variable Domain specificities were identical to those used to construct the B7-H3 mAb 1/CD3 mAb 2/CD8 mAb 1 Tri-Specific Binding Molecule. The CD3 mAb 2/CD8 mAb 1/B7-H3 mAb 1 Tri-Specific Binding Molecule was composed of four different polypeptide chains (Table 10).

TABLE 10

| Polypeptide | | |
|---|---|---|
| Chain | Domains | Binding Affinity |
| 1 | VL(CD3 mAb 2)-VH(CD8 mAb 1)- | Light Chain: CD3 |
| | E-Coil-(CH2—CH3) | Heavy Chain: CD8 |
| 2 | VL(CD8 mAb 1)-VH(CD3 mAb 2)- | Light Chain: CD8 |
| | K-Coil | Heavy Chain: CD3 |
| 3 | Heavy Chain B7-H3 mAb 1 | B7-H3 |
| 4 | Light Chain B7-H3 mAb 1 | B7-H3 |

The amino acid sequence of the first polypeptide chain of the CD3 mAb 2/CD8 mAb 1/B7-H3 mAb 1 Tri-Specific Binding Molecule is (SEQ ID NO:67):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

The amino acid sequence of the second polypeptide chain of the CD3 mAb 2/CD8 mAb 1/B7-H3 mAb 1 Tri-Specific Binding Molecule is (SEQ ID NO:68):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLQQSGAELV KPGASVKLSC TASGFNIKDT YIHFVRQRPE
QGLEWIGRID PANDNTLYAS KFQGKATITA DTSSNTAYMH
LCSLTSGDTA VYYCGRGYGY YVFDHWGQGT TLTVSSGGCG
GGKVAALKEK VAALKEKVAA LKEKVAALKE

The amino acid sequence of the third polypeptide chain of the CD3 mAb 2/CD8 mAb 1/B7-H3 mAb 1 Tri-Specific Binding Molecule is (SEQ ID NO:69):

QVQLVQSGAE VKKPGASVKV SCKASGYTFT SYWMQWVRQA
PGQGLEWMGT IYPGDGDTRY TQKFKGRVTI TADKSTSTAY
MELSSLRSED TAVYYCARRG IPRLWYFDVW GQGTTVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKRVEP KSCDKTHTCP PCPAPEAAGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSREE
MTKNQVSLSC AVKGFYPSDI AVEWESNGQP ENNYKTTPPV
LDSDGSFFLV SKLTVDKSRW QQGNVFSCSV MHEALHNRYT
QKSLSLSPGK

The amino acid sequence of the fourth polypeptide chain of the CD3 mAb 2/CD8 mAb 1/B7-H3 mAb 1 Tri-Specific Binding Molecule is (SEQ ID NO:70):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The B7-H3 mAb 1/CD8 mAb 1/CD3 mAb 2 Tri-Specific Binding Molecule was composed of four different polypeptide chains (Table 11).

TABLE 11

| Polypeptide | | |
|---|---|---|
| Chain | Domains | Binding Affinity |
| 1 | VL(B7-H3 mAb 1)-VH(CD8 | Light Chain: B7-H3 |
| | mAb 1)-E-Coil-(CH2—CH3) | Heavy Chain: CD8 |
| 2 | VL(CD8 mAb 1)-VH(B7-H3 | Light Chain: CD8 |
| | mAb1)-K-Coil | Heavy Chain: B7-H3 |
| 3 | Heavy Chain CD3 mAb 2 | CD3 |
| 4 | Light Chain CD3 mAb 2 | CD3 |

The amino acid sequence of the first polypeptide chain of the B7-H3 mAb 1/CD8 mAb 1/CD3 mAb 2 Tri-Specific Binding Molecule is (SEQ ID NO:71):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRGGG SGGGGQVQLV
QSGAEVKKPG ASVKVSCKAS GYTFTSYWMQ WVRQAPGQGL
EWMGTIYPGD GDTRYTQKFK GRVTITADKS TSTAYMELSS
LRSEDTAVYY CARRGIPRLW YFDVWGQGTT VTVSSGGCGG
GEVAALEKEV AALEKEVAAL EKEVAALEKG GGDKTHTCPP
CPAPEAAGGP SVFLFPPKPK DTLMISRTPE VTCVVVDVSH
EDPEVKFNWY VDGVEVHNAK TKPREEQYNS TYRVVSVLTV
LHQDWLNGKE YKCKVSNKAL PAPIEKTISK AKGQPREPQV
YTLPPSREEM TKNQVSLWCL VKGFYPSDIA VEWESNGQPE
NNYKTTPPVL DSDGSFFLYS KLTVDKSRWQ QGNVFSCSVM
HEALHNHYTQ KSLSLSPGK

The amino acid sequence of the second polypeptide chain of the B7-H3 mAb 1/CD8 mAb 1/CD3 mAb 2 Tri-Specific Binding Molecule is (SEQ ID NO:72):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIKGGG SGGGGEVQLQ
QSGAELVKPG ASVKLSCTAS GFNIKDTYIH FVRQRPEQGL
EWIGRIDPAN DNTLYASKFQ GKATITADTS SNTAYMHLCS
LTSGDTAVYY CGRGYGYYVF DHWGQGTTLT VSSGGCGGGK
VAALKEKVAA LKEKVAALKE KVAALKE

The amino acid sequence of the third polypeptide chain of the B7-H3 mAb 1/CD8 mAb 1/CD3 mAb 2 Tri-Specific Binding Molecule is (SEQ ID NO:73):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSSASTKG PSVFPLAPSS KSTSGGTAAL GCLVKDYFPE
PVTVSWNSGA LTSGVHTFPA VLQSSGLYSL SSVVTVPSSS
LGTQTYICNV NHKPSNTKVD KRVEPKSCDK THTCPPCPAP
EAAGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLSCAVKGF YPSDIAVEWE SNGQPENNYK
TTPPVLDSDG SFFLVSKLTV DKSRWQQGNV FSCSVMHEAL
HNRYTQKSLS LSPGK

The amino acid sequence of the fourth polypeptide chain of the B7-H3 mAb 1/CD8 mAb 1/CD3 mAb 2 Tri-Specific Binding Molecule is (SEQ ID NO:74):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG RTVAAPSVFI
FPPSDEQLKS GTASVVCLLN NFYPREAKVQ WKVDNALQSG
NSQESVTEQD SKDSTYSLSS TLTLSKADYE KHKVYACEVT
HQGLSSPVTK SFNRGEC

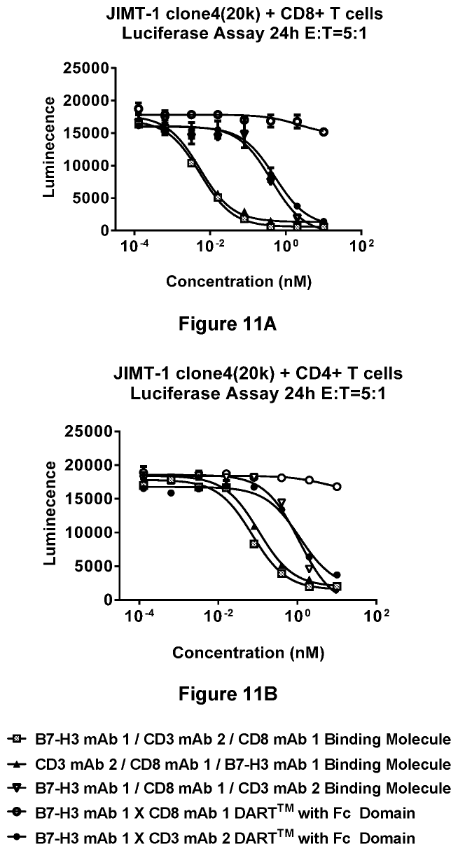

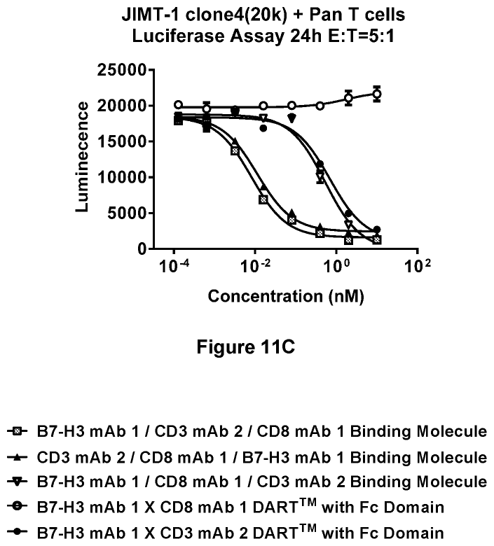

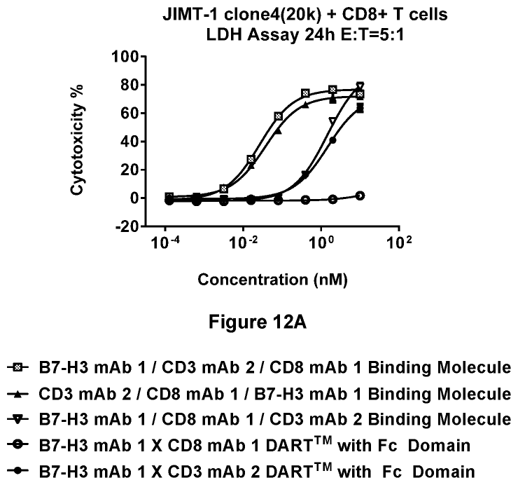

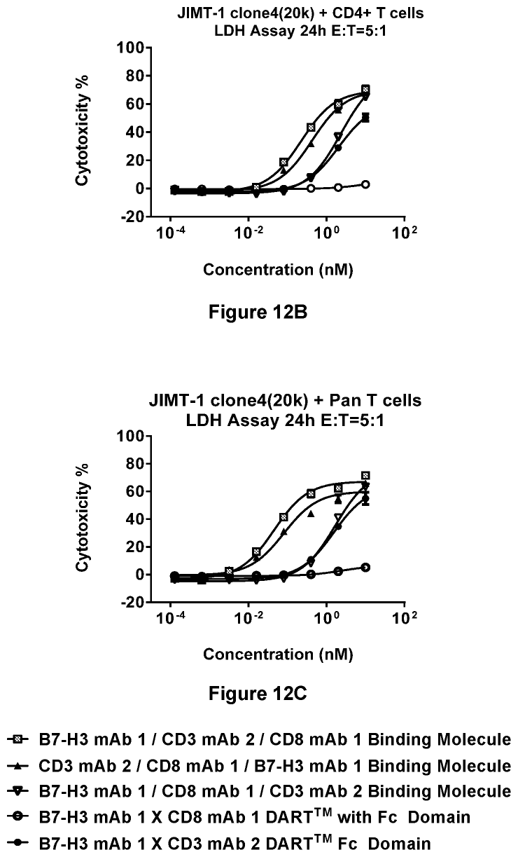

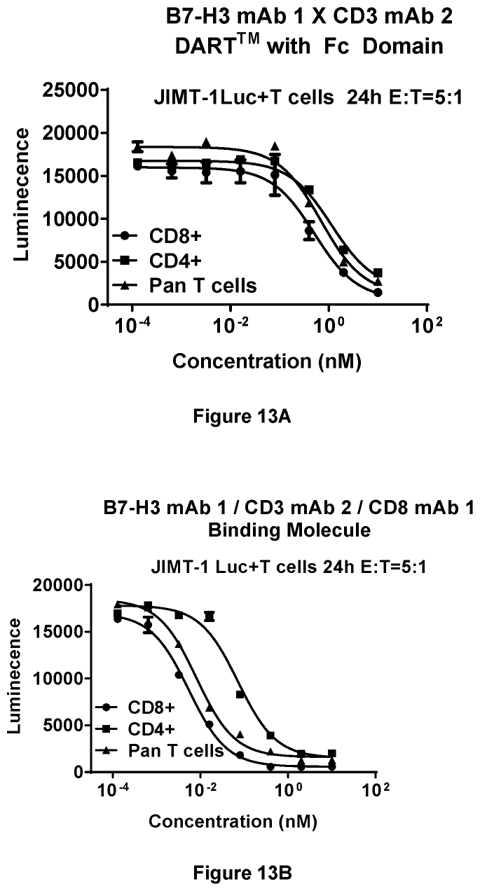

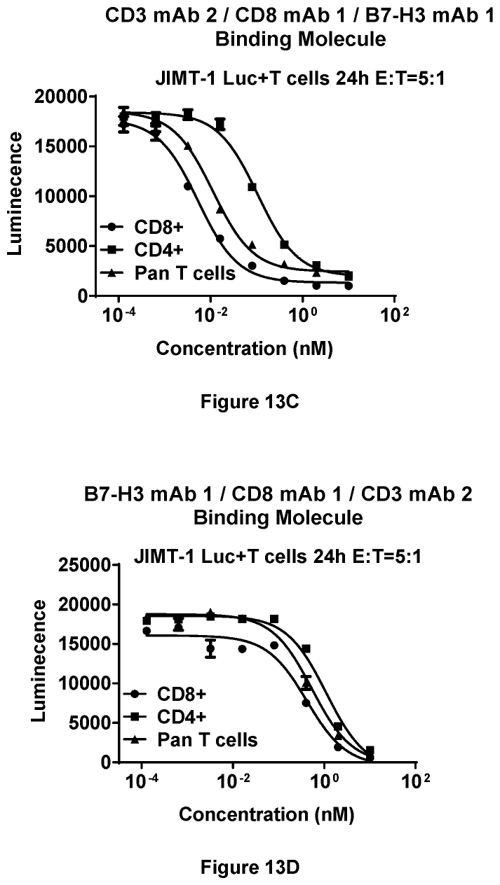

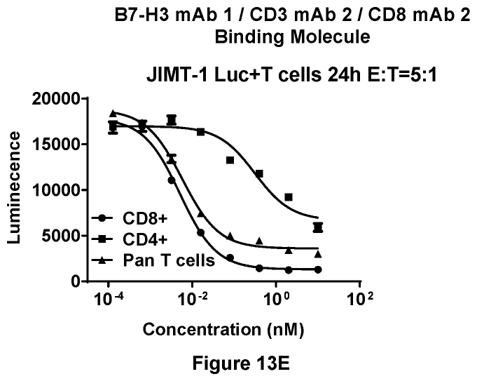

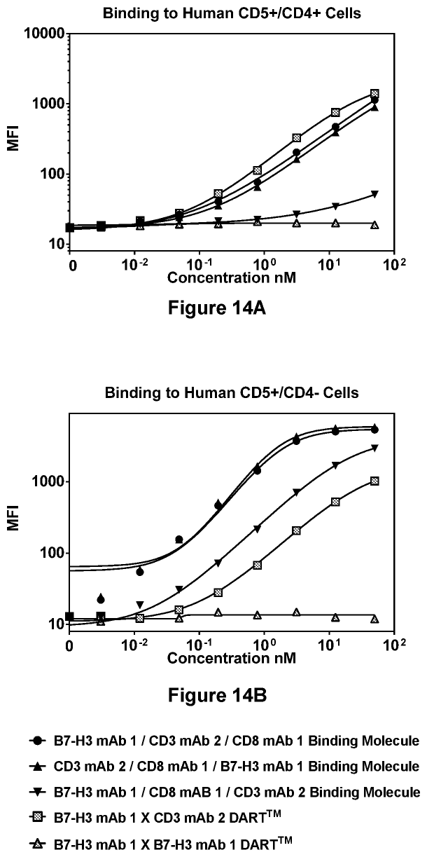

The results of this investigation are shown in FIGS. 11A-11C, FIGS. 12A-12C, FIGS. 13A-13E, and in Table 26. FIGS. 11A-11C and FIGS. 12A-12C independently demonstrate that placing a CD3 Binding Domain into Site C of the Tri-Specific Binding Molecules reduces the cytotoxicity of the molecule. FIGS. 13A-13E show that this decreased cytotoxicity is observed regardless of whether CD4+, CD8+ or pan T cells were employed.

As shown in FIGS. 14A-14B, placement of the CD3 Binding Domain at Site C, greatly diminished binding to both the CD5⁺ CD4⁺ cells (FIG. 14A) and the CD5⁺ CD4⁻ cells (FIG. 14B). Notably, however, regardless of the placement of the CD3 Binding Domain, all of the Tri-Specific Binding Molecules were capable of mediating re-directed cytotoxicity.

### Example 4

### Production and Properties of Exemplary Anti-CD3, Anti-CD8, Anti-5T4 Tri-Specific Binding Molecules

Additional exemplary Tri-Specific Binding Molecules specific for the Disease-Associated Antigen 5T4 were constructed. The 5T4 mAb 2/CD3 mAb 2/CD8 mAb 1 Tri-Specific Binding Molecule was composed of four different polypeptide chains (Table 12).

TABLE 12

| Polypeptide | | |
|---|---|---|
| Chain | Domains | Binding Affinity |
| 1 | VL(5T4 mAb 2)-VH(CD3 mAb 2)- | Light Chain: 5T4 |
| | E-Coil-(CH2—CH3) | Heavy Chain: CD3 |
| 2 | VL(CD3 mAb 2)-VH(5T4 mAb 2)- | Light Chain: CD3 |
| | K-Coil | Heavy Chain: 5T4 |
| 3 | Heavy Chain CD8 mAb 1 | CD8 |
| 4 | Light Chain CD8 mAb 1 | CD8 |

The amino acid sequence of the first polypeptide chain of the 5T4 mAb 2/CD3 mAb 2/CD8 mAb 1 Tri-Specific Binding Molecule is (SEQ ID NO:75):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IKGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSGGCGG GEVAALEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of the 5T4 mAb 2/CD3 mAb 2/CD8 mAb 1 Tri-Specific Binding Molecule is (SEQ ID NO:76):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLQQPGAELV KPGASVKMSC KASGYTFTSY WITWVKQRPG
QGLEWIGDIY PGSGRANYNE KFKSKATLTV DTSSSTAYMQ
LSSLTSEDSA VYNCARYGPL FTTVVDPNSY AMDYWGQGTS
VTVSSGGCGG GKVAALKEKV AALKEKVAAL KEKVAALKE

The amino acid sequence of the third polypeptide chain of the 5T4 mAb 2/CD3 mAb 2/CD8 mAb 1 Tri-Specific Binding Molecule is (SEQ ID NO:77):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The amino acid sequence of the fourth polypeptide chain of the 5T4 mAb 2/CD3 mAb 2/CD8 mAb 1 Tri-Specific Binding Molecule is (SEQ ID NO:78):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The 5T4 mAb 2/CD3 mAb 2/CD8 mAb 2 Tri-Specific Binding Molecule was composed of four different polypeptide chains (Table 13).

TABLE 13

| Polypeptide | | |
|---|---|---|
| Chain | Domains | Binding Affinity |
| 1 | VL(5T4 mAb 2)-VH(CD3 mAb 2)- | Light Chain: 5T4 |
| | E-Coil-(CH2—CH3) | Heavy Chain: CD3 |
| 2 | VL(CD3 mAb 2)-VH(5T4 mAb 2)- | Light Chain: CD3 |
| | K-Coil | Heavy Chain: 5T4 |
| 3 | Heavy Chain CD8 mAb 2 | CD8 |
| 4 | Light Chain CD8 mAb 2 | CD8 |

The amino acid sequence of the first polypeptide chain of the 5T4 mAb 2/CD3 mAb 2/CD8 mAb 2 Tri-Specific Binding Molecule is (SEQ ID NO:79):

DVLMTQTPLS LPVSLGDQAS ISCRSSQSIV YSNGNTYLEW
YLQKPGQSPK LLIYKVSNRF SGVPDRFSGS GSGTDFTLKI
SRVEAEDLGV YYCFQGSHVP FTFGSGTKLE IKGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSGGCGG GEVAALEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of the 5T4 mAb 2/CD3 mAb 2/CD8 mAb 2 Tri-Specific Binding Molecule is (SEQ ID NO:80):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLQQPGAELV KPGASVKMSC KASGYTFTSY WITWVKQRPG
QGLEWIGDIY PGSGRANYNE KFKSKATLTV DTSSSTAYMQ
LSSLTSEDSA VYNCARYGPL FTTVVDPNSY AMDYWGQGTS
VTVSSGGCGG GKVAALKEKV AALKEKVAAL KEKVAALKE

The amino acid sequence of the third polypeptide chain of the 5T4 mAb 2/CD3 mAb 2/CD8 mAb 2 Tri-Specific Binding Molecule is (SEQ ID NO:81):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

The amino acid sequence of the fourth polypeptide chain of the 5T4 mAb 2/CD3 mAb 2/CD8 mAb 2 Tri-Specific Binding Molecule is (SEQ ID NO:82):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

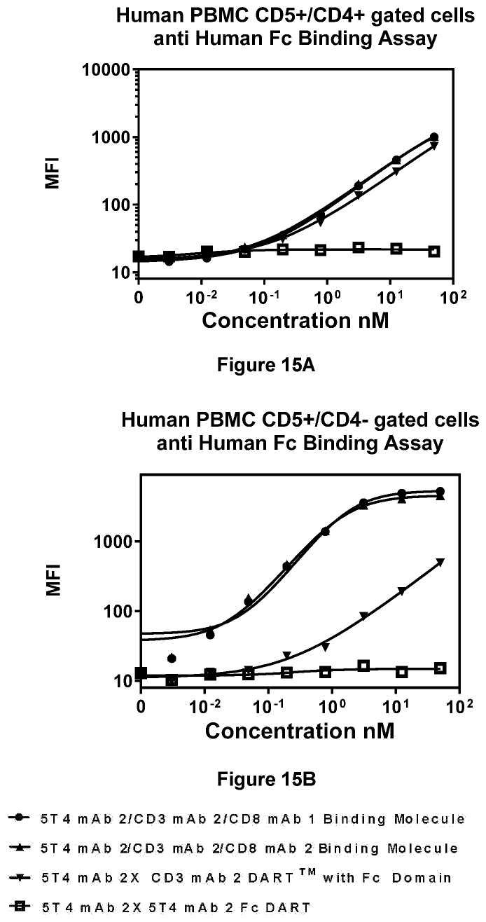

The 5T4 mAb 2/CD3 mAb 2/CD8 mAb 1 and 5T4 mAb 2/CD3 mAb 2/CD8 mAb 2 Tri-Specific Binding Molecules were expressed and purified as described above. The ability of these two Tri-Specific Binding Molecules to bind to CD5+/CD4+ gated and CD5+/CD4− gated human PBMCs were compared to those of a 5T4×CD3 DART with an Fc Domain. As shown in FIGS. 15A-15B, the 5T4/CD3 mAb 2/CD8 mAb 1 and 5T4/CD3 mAb 2/CD8 mAb 2 Tri-Specific Binding Molecules demonstrated greatly increased binding to CD8+ T cells (FIG. 15B) compared to CD4+ T cells (FIG. 15A).

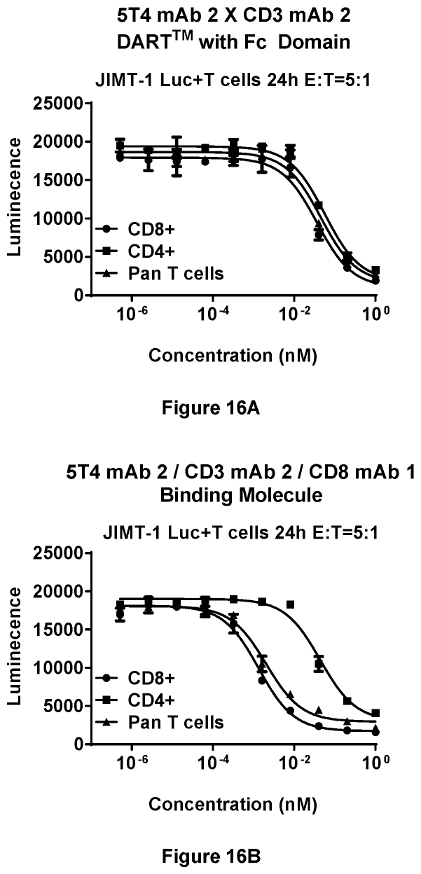

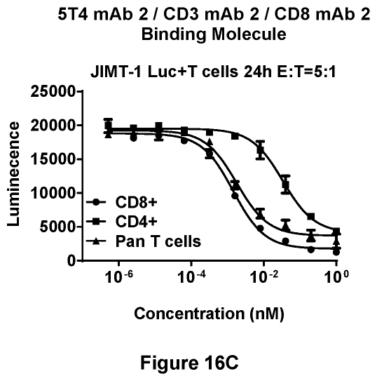

In order to demonstrate the ability of the 5T4 mAb 2/CD3 mAb 2/CD8 mAb 1 and 5T4 mAb 2/CD3 mAb 2/CD8 mAb 2 Tri-Specific Binding Molecules to mediate the redirected killing of target cells, such molecules were incubated in the presence of T cells and JIMT-1 target cells. As shown in FIGS. 16A-16C, re-directed killing of the target cells was observed. As observed for the B7-H3 Tri-Specific Binding Molecules described above, the killing was substantially more potent than that observed for the corresponding 5T4×CD3 DART containing an Fc Domain. Likewise, as observed for the B7-H3 Tri-Specific Binding Molecules, the use of different CD8 Variable Domains had no effect on the ability of the 5T4 Tri-Specific Binding Molecules to re-direct CD8+ T cells to the target cells. The 5T4 mAb 2/CD3 mAb 2/CD8 mAb 2 Tri-Specific Binding Molecule was also found to be highly active and demonstrated much greater CTL activity using CD8+vs. CD4+ effector cells (23-fold lower EC50). The 5T4 mAb 2/CD3 mAb 2/CD8 mAb 1 Tri-Specific Binding Molecule was 22-fold more potent using pan T cell effectors compared to a 5T4×CD3 DART™ and the 5T4 mAb 2/CD3 mAb 2/CD8 mAb 2 Tri-Specific Binding Molecule was 25-fold more potent.

### Example 5

### Properties of Exemplary Anti-CD3, Anti-CD8, Anti-ROR1 Tri-Specific Binding Molecules

Additional exemplary Tri-Specific Binding Molecules specific for the Disease-Associated Antigen ROR1 were constructed. The ROR1 mAb 1/CD3 mAb 2/CD8 mAb 1 Tri-Specific Binding Molecule was composed of four different polypeptide chains (Table 14).

TABLE 14

| Polypeptide | | |
|---|---|---|
| Chain | Domains | Binding Affinity |
| 1 | VL(ROR1 mAb 1)-VH(CD3 mAb 2)- | Light Chain: ROR1 |
| | E-Coil-(CH2—CH3) | Heavy Chain: CD3 |
| 2 | VL(CD3 mAb 2)-VH(ROR1 mAb 1)- | Light Chain: CD3 |
| | K-Coil | Heavy Chain: ROR1 |
| 3 | Heavy Chain CD8 mAb 1 | CD8 |
| 4 | Light Chain CD8 mAb 1 | CD8 |

The amino acid sequence of the first polypeptide chain of the ROR1 mAb 1/CD3 mAb 2/CD8 mAb 1 Tri-Specific Binding Molecule is (SEQ ID NO:83):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLGGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSGGCG GGEVAALEKE VAALEKEVAA LEKEVAALEK
GGGDKTHTCP PCPAPEAAGG PSVFLFPPKP KDTLMISRTP
EVTCVVVDVS HEDPEVKFNW YVDGVEVHNA KTKPREEQYN
STYRVVSVLT VLHQDWLNGK EYKCKVSNKA LPAPIEKTIS
KAKGQPREPQ VYTLPPSREE MTKNQVSLWC LVKGFYPSDI
AVEWESNGQP ENNYKTTPPV LDSDGSFFLY SKLTVDKSRW
QQGNVFSCSV MHEALHNHYT QKSLSLSPGK

The amino acid sequence of the second polypeptide chain of the ROR1 mAb 1/CD3 mAb 2/CD8 mAb 1 Tri-Specific Binding Molecule is (SEQ ID NO:84):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWVRQAPG
KGLEWVATIY PSSGKTYYAD SVKGRFTISS DNAKNSLYLQ
MNSLRAEDTA VYYCARDSYA DDAALFDIWG QGTTVTVSSG
GCGGGKVAAL KEKVAALKEK VAALKEKVAA LKE

The amino acid sequence of the third polypeptide chain of the ROR1 mAb 1/CD3 mAb 2/CD8 mAb 1 Tri-Specific Binding Molecule is (SEQ ID NO:85):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPG

The amino acid sequence of the fourth polypeptide chain of the ROR1 mAb 1/CD3 mAb 2/CD8 mAb 1 Tri-Specific Binding Molecule is (SEQ ID NO:86):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The ROR1 mAb 1/CD3 mAb 2/CD8 mAb 2 Tri-Specific Binding Molecule was composed of four different polypeptide chains (Table 15).

TABLE 15

| Polypeptide | | |
|---|---|---|
| Chain | Domains | Binding Affinity |
| 1 | VL(ROR1 mAb 1)-VH(CD3 mAb 2)- | Light Chain: ROR1 |
| | E-Coil-(CH2—CH3) | Heavy Chain: CD3 |
| 2 | VL(CD3 mAb 2)-VH(ROR1 mAb 1)- | Light Chain: CD3 |
| | K-Coil | Heavy Chain: ROR1 |
| 3 | Heavy Chain CD8 mAb 2 | CD8 |
| 4 | Light Chain CD8 mAb 2 | CD8 |

The amino acid sequence of the first polypeptide chain of the ROR1 mAb 1/CD3 mAb 2/CD8 mAb 2 Tri-Specific Binding Molecule is (SEQ ID NO:87):

QLVLTQSPSA SASLGSSVKL TCTLSSGHKT DTIDWYQQQP
GKAPRYLMKL EGSGSYNKGS GVPDRFGSGS SSGADRYLTI
SSLQSEDEAD YYCGTDYPGN YLFGGGTQLT VLGGGGSGGG
GEVQLVESGG GLVQPGGSLR LSCAASGFTF STYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSGGCG GGEVAALEKE VAALEKEVAA LEKEVAALEK
GGGDKTHTCP PCPAPEAAGG PSVFLFPPKP KDTLMISRTP
EVTCVVVDVS HEDPEVKFNW YVDGVEVHNA KTKPREEQYN
STYRVVSVLT VLHQDWLNGK EYKCKVSNKA LPAPIEKTIS
KAKGQPREPQ VYTLPPSREE MTKNQVSLWC LVKGFYPSDI
AVEWESNGQP ENNYKTTPPV LDSDGSFFLY SKLTVDKSRW
QQGNVFSCSV MHEALHNHYT QKSLSLSPGK

The amino acid sequence of the second polypeptide chain of the ROR1 mAb 1/CD3 mAb 2/CD8 mAb 2 Tri-Specific Binding Molecule is (SEQ ID NO:88):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQE
QLVESGGGLV QPGGSLRLSC AASGFTFSDY YMSWVRQAPG
KGLEWVATIY PSSGKTYYAD SVKGRFTISS DNAKNSLYLQ
MNSLRAEDTA VYYCARDSYA DDAALFDIWG QGTTVTVSSG
GCGGGKVAAL KEKVAALKEK VAALKEKVAA LKE

The amino acid sequence of the third polypeptide chain of the ROR1 mAb 1/CD3 mAb 2/CD8 mAb 2 Tri-Specific Binding Molecule is (SEQ ID NO:89):

QVQLVESGGG VVQPGRSLRL SCAASGFTFS DFGMNWVRQA
PGKGLEWVAL IYYDGSNKFY ADSVKGRFTI SRDNSKNTLY
LQMNSLRAED TAVYYCAKPH YDGYYHFFDS WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKRVE PKSCDKTHTC PPCPAPEAAG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRE
EMTKNQVSLS CAVKGFYPSD IAVEWESNGQ PENNYKTTPP
VLDSDGSFFL VSKLTVDKSR WQQGNVFSCS VMHEALHNRY
TQKSLSLSPG K

The amino acid sequence of the fourth polypeptide chain of the ROR1 mAb 1/CD3 mAb 2/CD8 mAb 2 Tri-Specific Binding Molecule is (SEQ ID NO:90):

DIQMTQSPSS LSASVGDRVT ITCKGSQDIN NYLAWYQQKP
GKAPKLLIYN TDILHTGVPS RFSGSGSGTD FTFTISSLQP
EDIATYYCYQ YNNGYTFGQG TKVEIKRTVA APSVFIFPPS
DEQLKSGTAS VVCLLNNFYP REAKVQWKVD NALQSGNSQE
SVTEQDSKDS TYSLSSTLTL SKADYEKHKV YACEVTHQGL
SSPVTKSFNR GEC

The properties of ROR1 mAb 1/CD3 mAb 2/CD8 mAb 1 and ROR1 mAb 1/CD3 mAb 2/CD8 mAb 2 Tri-Specific Binding Molecules were compared with those of a ROR1×CD3 DART™ containing an Fc Domain. The DART™ and ROR1 mAb 1/CD3 mAb 2/CD8 mAb 1 and ROR1 mAb 1/CD3 mAb 2/CD8 mAb 2 Tri-Specific Binding Molecules were constructed using the Fv sequences from an anti-ROR1 monoclonal antibody, ROR1 mAb 1, that binds to the ROR-1 antigen.

The two ROR1 mAb 1/CD3 mAb 2/CD8 mAb 1 and ROR1 mAb 1/CD3 mAb 2/CD8 mAb 2 Tri-Specific Binding Molecules and DART all were active in CTL assays against JIMT1-luc and A549 target cells. However, the ROR1 mAb 1/CD3 mAb 2/CD8 mAb 1 and ROR1 mAb 1/CD3 mAb 2/CD8 mAb 2 Tri-Specific Binding Molecules exhibited dramatically increased activity with CD8+ T cell and pan T cell effectors.

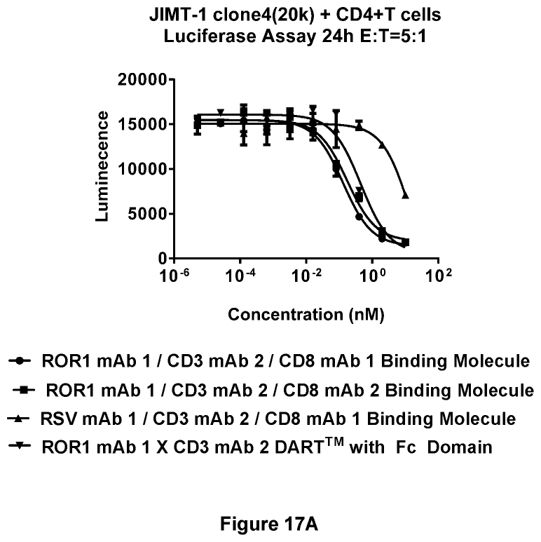

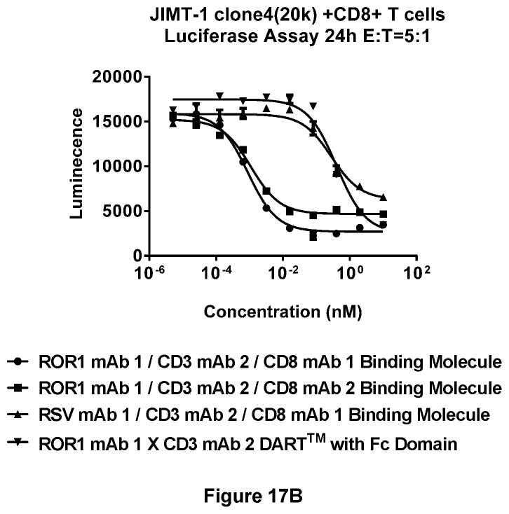

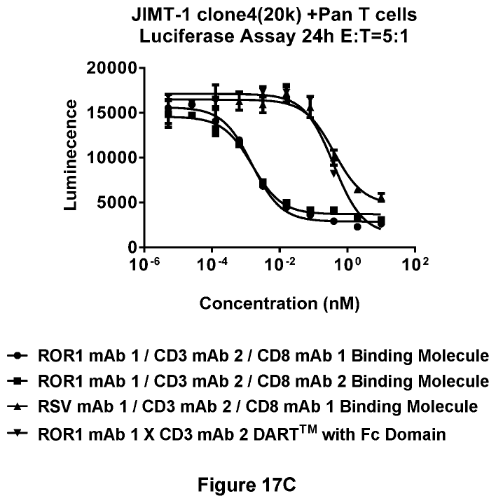

The ability of the ROR1 mAb 1/CD3 mAb 2/CD8 mAb 1 and ROR1 mAb 1/CD3 mAb 2/CD8 mAb 2 Tri-Specific Binding Molecules to mediate re-directed killing of target cells was measured using both a luciferase assay and an LDH assay. In both cases, re-directed killing was observed. FIGS. 17A-17C demonstrate the re-directed killing of target cells as measured using the luciferase assay. Results are summarized in Table 16.

TABLE 16

| | EC50 | | | Ratio | | |
|---|---|---|---|---|---|---|
| | Effector Cells | | | CD4 | CD4 | Pan |
| Tri-Specific Binding Molecule | CD4 | CD8 | Pan T | CD8 | CD8 | T |
| | JIMT-1 LUC Target Cells (Lucferase Assay) | | | | | |
| ROR1 X CD3 DART ™ | 0.45 | 0.4064 | 0.3931 | 1.1 | 1.0 | 1.0 |
| ROR 1 mAb 1/CD3 mAb 2/CD8 | 0.131 | 0.0009 | 0.0015 | 145.3 | 131.3 | 262.1 |
| mAb 1 | | | | | | |
| Tri-Specific Binding Molecule | | | | | | |
| ROR 1 mAb 1/CD3 mAb 2/CD8 | 0.174 | 0.0011 | 0.0016 | 158.0 | 142.7 | 245.7 |
| mAb 2 | | | | | | |
| Tri-Specific Binding Molecule | | | | | | |
| | JIMT-1 LUC Target Cells (LDH Assay) | | | | | |
| ROR1 X CD3 DART ™ | 0.291 | 0.7761 | 0.7643 | 0.4 | 1.0 | 1.0 |
| ROR 1 mAb 1/CD3 mAb 2/CD8 | 0.095 | 0.0017 | 0.0053 | 55.9 | 149.0 | 144.2 |
| mAb 1 | | | | | | |
| Tri-Specific Binding Molecule | | | | | | |
| ROR 1 mAb 1/CD3 mAb 2/CD8 | 0.277 | 0.0021 | 0.0057 | 131.8 | 351.3 | 134.1 |
| mAb 2 | | | | | | |
| Tri-Specific Binding Molecule | | | | | | |
| | A549 | | | | | |
| ROR1 X CD3 DART ™ | 0.762 | 0.468 | 0.5305 | 1.6 | 1.0 | 1.0 |
| ROR 1 mAb 1/CD3 mAb 2/CD8 | 0.879 | 0.0102 | 0.0231 | 86.2 | 53.0 | 23.0 |
| mAb 1 | | | | | | |
| Tri-Specific Binding Molecule | | | | | | |
| ROR 1 mAb 1/CD3 mAb 2/CD8 | 1.257 | 0.009 | 0.0126 | 139.7 | 85.8 | 42.1 |
| mAb 2 | | | | | | |
| Tri-Specific Binding Molecule | | | | | | |

### Example 6

### Properties of Exemplary Anti-CD3, Anti-CD8, Anti-Env Tri-Specific Binding Molecules

Additional exemplary Tri-Specific Binding Molecules specific for the Disease-Associated Antigen HIV (gp140 antigen) were constructed. The HIV mAb 1/CD3 mAb 2/CD8 mAb 1 Tri-Specific Binding Molecule was composed of four different polypeptide chains (Table 17).

TABLE 17

| Polypeptide | | |
|---|---|---|
| Chain | Domains | Binding Affinity |
| 1 | VL(HIV mAb 1)-VH(CD3 mAb 2)- | Light Chain: HIV |
| | E-Coil-(CH2—CH3) | Heavy Chain: CD3 |
| 2 | VL(CD3 mAb 2)-VH(HIV mAb 1)- | Light Chain: CD3 |
| | K-Coil | Heavy Chain: HIV |
| 3 | Heavy Chain CD8 mAb 1 | CD8 |
| 4 | Light Chain CD8 mAb 1 | CD8 |

The amino acid sequence of the first polypeptide chain of the HIV mAb 1/CD3 mAb 2/CD8 mAb 1 Tri-Specific Binding Molecule is (SEQ ID NO:91):

DIVMTQSPDS LAVSPGERAT IHCKSSQTLL YSSNNRHSIA
WYQQRPGQPP KLLLYWASMR LSGVPDRFSG SGSGTDFTLT
INNLQAEDVA IYYCHQYSSH PPTFGHGTRV EIKGGGSGGG
GEVQLVESGG GLVQPGGSLR LSCAASGFT FSTYAMNWVRQ
APGKGLEWVG RIRSKYNNYA TYYADSVKGR FTISRDDSKN
SLYLQMNSLK TEDTAVYYCV RHGNFGNSYV SWFAYWGQGT
LVTVSSASTK GEVAACEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The amino acid sequence of the second polypeptide chain of the HIV mAb 1/CD3 mAb 2/CD8 mAb 1 Tri-Specific Binding Molecule polypeptide is (SEQ ID NO:92):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGGGVF KPGGSLRLSC EASGFTFTEY YMTWVRQAPG
KGLEWLAYIS KNGEYSKYSP SSNGRFTISR DNAKNSVFLQ
LDRLSADDTA VYYCARADGL TYFSELLQYI FDLWGQGARV
TVSSASTKGK VAACKEKVAA LKEKVAALKE KVAALKE

The amino acid sequence of the third polypeptide chain of the HIV mAb 1/CD3 mAb 2/CD8 mAb 1 Tri-Specific Binding Molecule is (SEQ ID NO:93):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPG

The amino acid sequence of the fourth polypeptide chain of the HIV mAb 1/CD3 mAb 2/CD8 mAb 1 Tri-Specific Binding Molecule is (SEQ ID NO:94):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The HIV mAb 2/CD3 mAb 2/CD8 mAb 1 Tri-Specific Binding Molecule was composed of four different polypeptide chains (Table 18).

TABLE 18

| Polypeptide | | |
|---|---|---|
| Chain | Domains | Binding Affinity |
| 1 | VL(HIV mAb 2)-VH(CD3 mAb 2)- | Light Chain: HIV |
| | E-Coil-(CH2—CH3) | Heavy Chain: CD3 |
| 2 | VL(CD3 mAb 2)-VH(HIV mAb 2)- | Light Chain: CD3 |
| | K-Coil | Heavy Chain: HIV |
| 3 | Heavy Chain CD8 mAb 1 | CD8 |
| 4 | Light Chain CD8 mAb 1 | CD8 |

The amino acid sequence of the first polypeptide chain of the HIV mAb 2/CD3 mAb 2/CD8 mAb 1 Tri-Specific Binding Molecule is (SEQ ID NO:95):

QSALTQPPSA SGSPGQSVTI SCTGTSSDVG GYNYVSWYQH
HPGKAPKLII SEVNNRPSGV PDRFSGSKSG NTASLTVSGL
QAEDEAEYYC SSYTDIHNFV FGGGTKLTVL GGGSGGGGEV
QLVESGGGLV QPGGSLRLSC AASGFTFSTY AMNWVRQAPG
KGLEWVGRIR SKYNNYATYY ADSVKGRFTI SRDDSKNSLY
LQMNSLKTED TAVYYCVRHG NFGNSYVSWF AYWGQGTLVT
VSSASTKGEV AACEKEVAAL EKEVAALEKE VAALEKGGGD
KTHTCPPCPA PEAAGGPSVF LFPPKPKDTL MISRTPEVTC
VVVDVSHEDP EVKFNWYVDG VEVHNAKTKP REEQYNSTYR
VVSVLTVLHQ DWLNGKEYKC KVSNKALPAP IEKTISKAKG
QPREPQVYTL PPSREEMTKN QVSLWCLVKG FYPSDIAVEW
ESNGQPENNY KTTPPVLDSD GSFFLYSKLT VDKSRWQQGN
VFSCSVMHEA LHNHYTQKSL SLSPGK

The amino acid sequence of the second polypeptide chain of the HIV mAb 2/CD3 mAb 2/CD8 mAb 1 Tri-Specific Binding Molecule is (SEQ ID NO:96):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLQESGPGLV KPSQTLSLSC TVSGGSSSSG AHYWSWIRQY
PGKGLEWIGY IHYSGNTYYN PSLKSRITIS QHTSENQFSL
KLNSVTVADT AVYYCARGTR LRTLRNAFDI WGQGTLVTVS
SASTKGKVAA CKEKVAALKE KVAALKEKVA ALKE

The amino acid sequence of the third polypeptide chain of the HIV mAb 2/CD3 mAb 2/CD8 mAb 1 Tri-Specific Binding Molecule is (SEQ ID NO:97):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPG

The amino acid sequence of the fourth polypeptide chain of the HIV mAb 2/CD3 mAb 2/CD8 mAb 1 Tri-Specific Binding Molecule is (SEQ ID NO:98):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

Tri-Specific Binding Molecules were prepared having a Binding Domain capable of binding to the gp140 antigen of Human immunodeficiency Virus (HIV) (i.e., an HIV mAb 1/CD3 mAb 2/CD8 mAb 1 Tri-Specific Binding Molecule and an HIV mAb 2/CD3 mAb 2/CD8 mAb 1 Tri-Specific Binding Molecule).

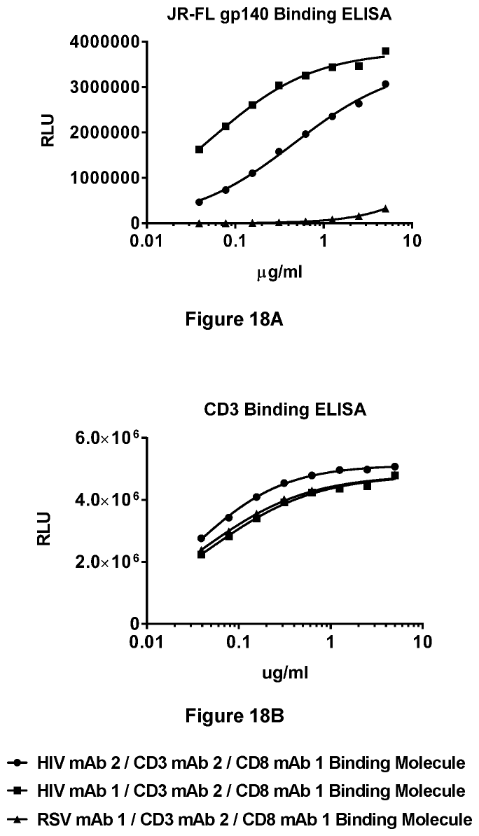

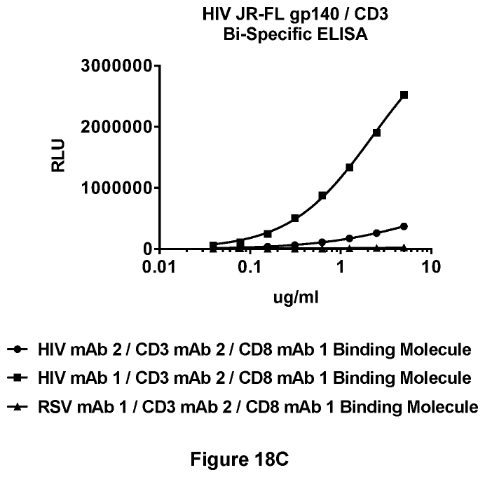

As a preliminary step, the ability of the HIV mAb 1/CD3 mAb 2/CD8 mAb 1 and HIV mAb 2/CD3 mAb 2/CD8 mAb 1 Tri-Specific Binding Molecules were assessed. Two g/ml of JR-FL strain gp140 protein in 0.2 M carbonate-bicarbonate buffer (pH 9.4) was coated onto a solid support, and then incubated with the Tri-Specific Binding Molecules (at a starting concentration of 5 μg/ml, followed by 1:2 serial dilutions). At the conclusion of the assay, binding was blocked with phosphate buffered saline (PBS) containing 3% bovine serum albumin (BSA). Binding was detected by ELISA (pico (ThermoScientific-Pierce) using anti-human IgG—that had been conjugated to horseradish peroxidase (HRP). The assay was also used with immobilized human CD3 (2 μg/ml) in order to assess the ability of the molecules to bind to CD3. Both Tri-Specific Binding Molecules were found to be able to bind to the soluble, immobilized gp140 protein (FIG. 18A) and to human CD3 (FIG. 18B). A sandwich ELISA was conducted to demonstrate that the Tri-Specific Binding Molecules were capable of coordinated binding to both gp140 and CD3. For this purpose, 2 μg/ml of the JR-FL strain gp140 proteins in 0.2 M carbonate-bocarbonate buffer (pH 9.4) was coated onto the solid support, and then incubated with the Tri-Specific Binding Molecules (at a starting concentration of 5 g/ml, followed by 1:2 serial dilutions). Human CD3, labeled with biotin was then added (0.5 μg/ml). Binding was detected by ELISA (pico (ThermoScientific-Pierce) using a streptavidin-HRP conjugate. FIG. 18C shows that the Tri-Specific Binding Molecules were capable of coordinated binding to both gp140 and CD3.

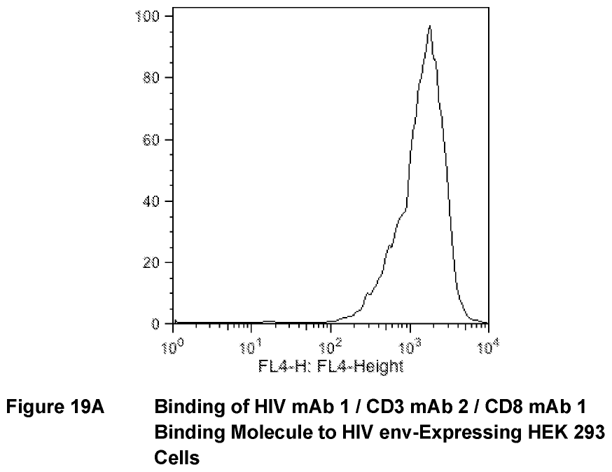

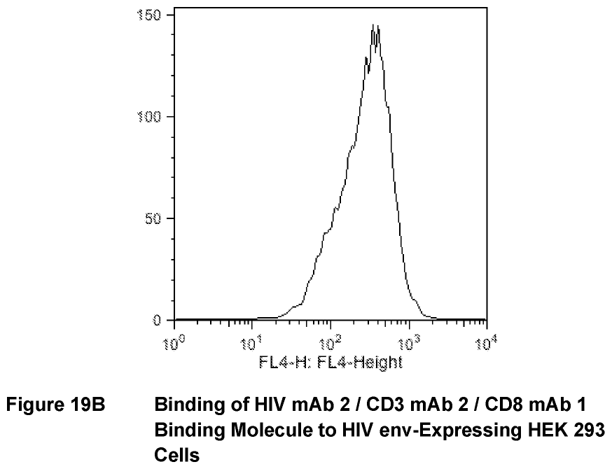

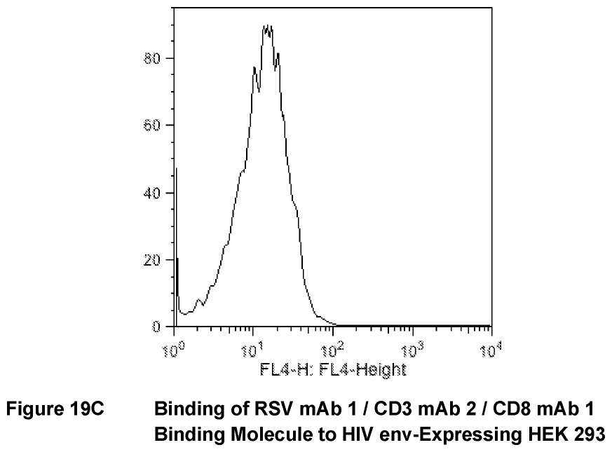

The Tri-Specific Binding Molecules were found to be capable of binding to the surface of cells that express the HIV env protein. To demonstrate this aspect of the invention, HIV env-expressing HEK293/D375 cells under doxycycline induction were incubated with the Tri-Specific Binding Molecules. Detection of binding was performed using biotinylated anti-human Fc antibody and Streptavidin-APC. As shown in FIGS. 19A-19C, both Tri-Specific Binding Molecules were found to be able to bind to the HEK293/D375 cells in contrast to a control Tri-Specific Binding Molecule (FIG. 19C).

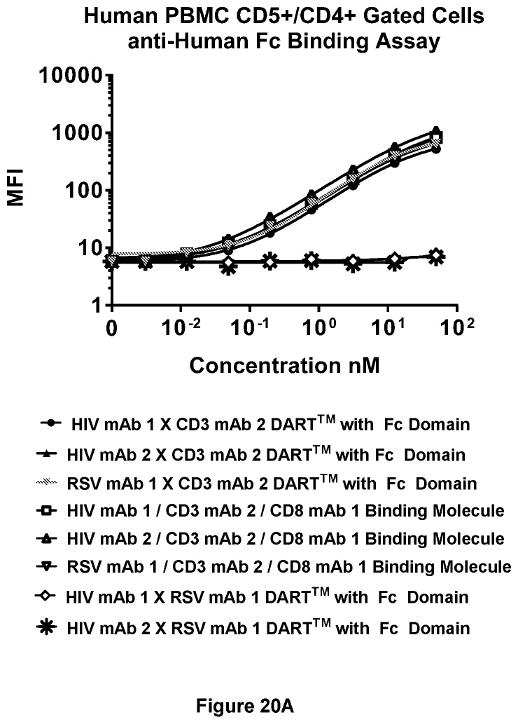

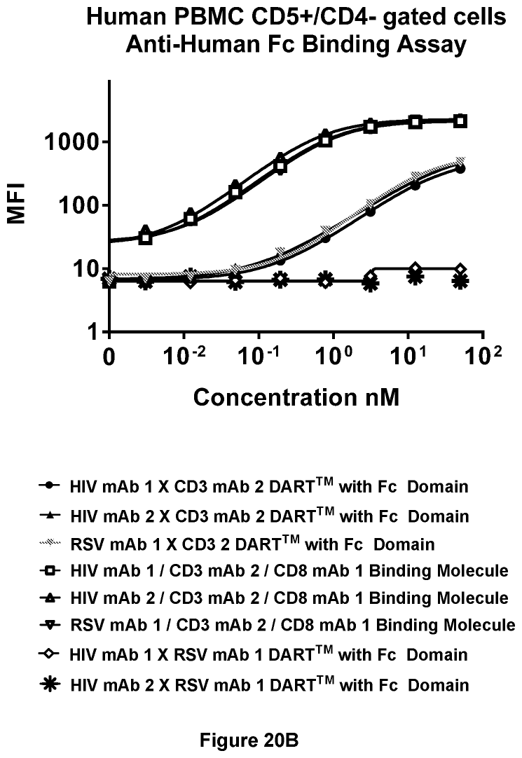

In order to demonstrate the ability of such Tri-Specific Binding Molecules to mediate re-directed killing of HIV-infected cells, the ability of such molecules to bind to PBMC was assessed. Human blood was lysed with ACK lysing buffer, washed 2× with PBS and re-suspended in FACS buffer contained 10% Human AB serum and incubated at room temperature for 20 minutes. Thereafter, the cells were pelleted by centrifugation and re-suspended (4×10⁶ cells/mL) in FACS buffer. 50 μL of serial titrated Tri-Specific Binding Molecules were added into wells of a 96-well deep plate. 50 μL of the cells (4×10⁶ cells/mL) well-mixed cells in FACS buffer containing 0.01% sodium azide were then added into corresponding wells and mixed thoroughly using a pipette. The plate was incubated in the dark for about 45 minutes at 2-8° C. At the end of incubation, the cells were washed twice by adding 300 μL of FACS buffer to each well, the plate was centrifuged at 1,200 rpm for 5 minutes, and the supernatant was discarded. The cell pellets were re-suspended in 100 μL mixture of goat anti-Human IgG Fc7-PE, CD5-APC and CD4-PerCP5.5 prepared in FACS buffer containing 0.01% sodium azide, and the cells were incubated in the dark for about 45 minutes at 2-8° C. At the end of the incubation, the cells were washed, re-suspended with FACS buffer, and analyzed with a BD Caliber flow cytometer. As shown in FIGS. 20A-20B, the Tri-Specific Binding Molecules were found to exhibit specific binding to the CD5+/CD4− cell population.

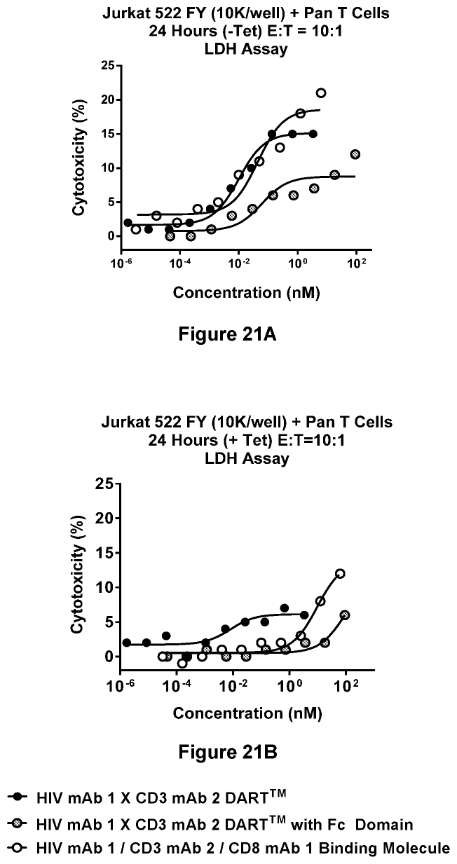

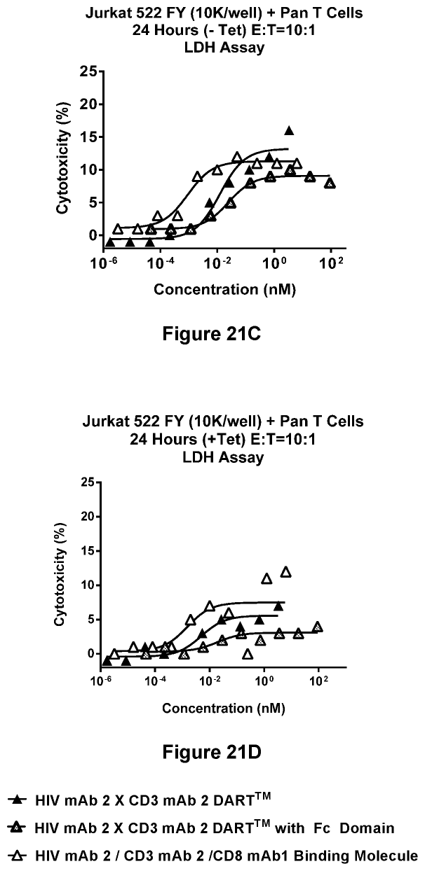

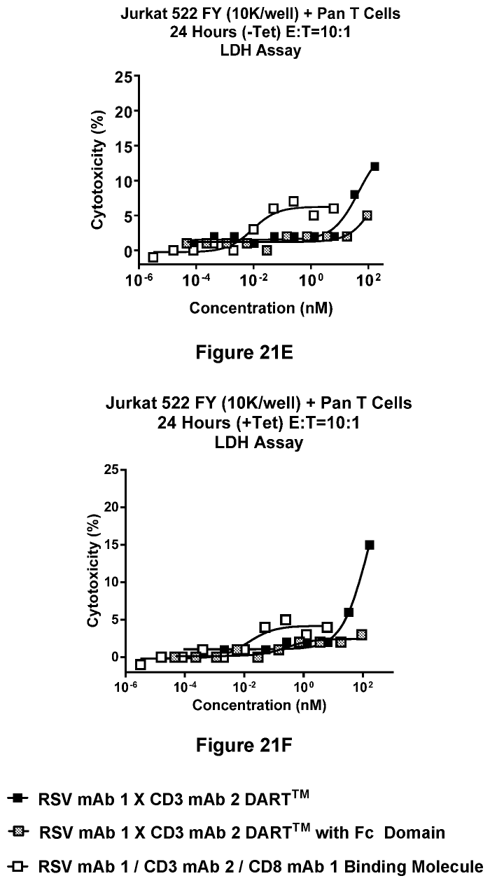

The Tri-Specific Binding Molecules were incubated at 37° C. for 24 hours in the presence of HIV env-expressing Jurkat 522 FY cells and pan T cells in the presence or absence of tetracycline, and cytotoxicity was measured using an LDH assay. As shown in FIGS. 21A-21F, the Tri-Specific Binding Molecules mediated substantial cytotoxicity.

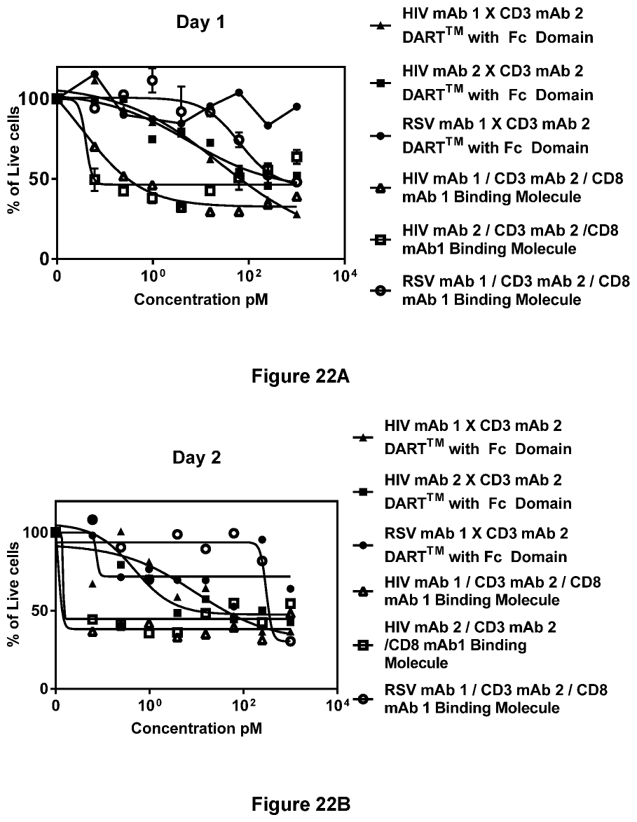

A cytotoxicity analysis was conducted using purified pan T cells and HIV env-expressing Jurkat 522 FY cells and the percent of live cells was measured. The results of this analysis are shown in FIGS. 22A-22B.

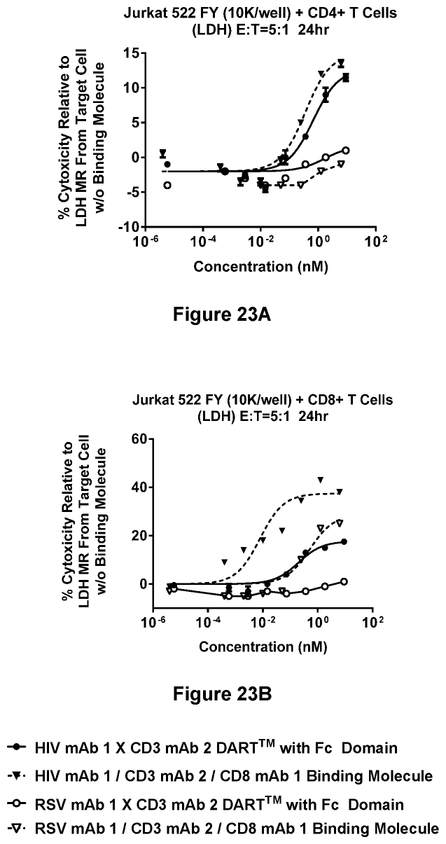

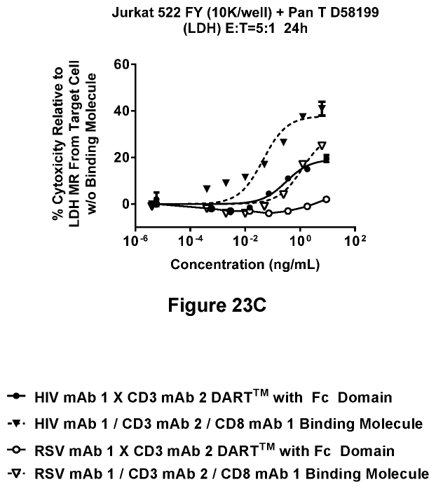

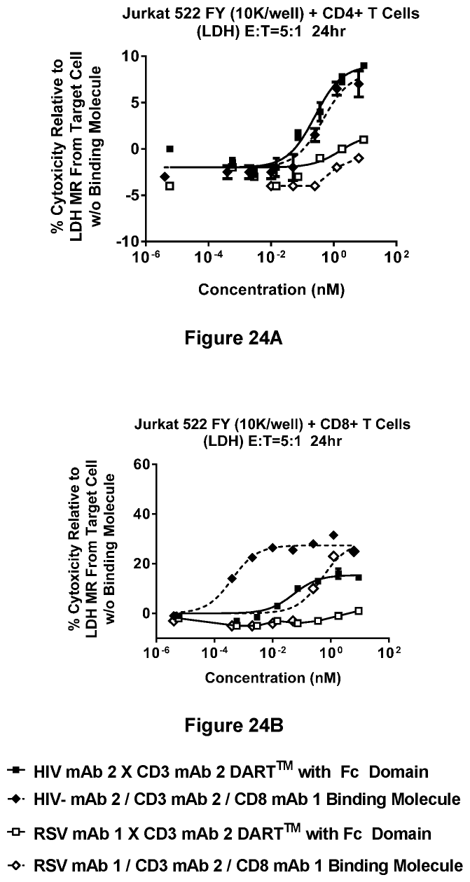

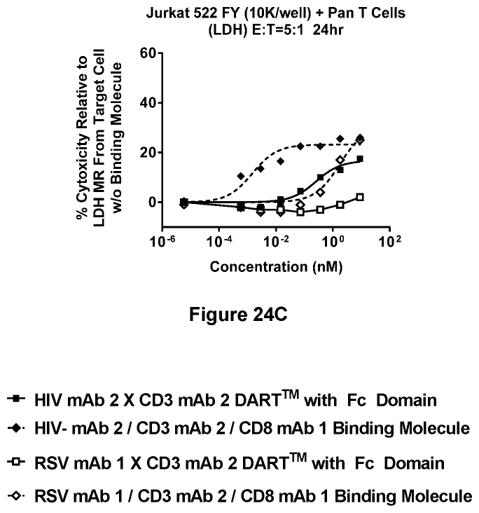

An assessment was made of the CTL activity of Tri-Specific Binding Molecules on HIV env-expressing Jurkat 522 FY cells using CD4+, CD8+ or pan T cells. The results of this assessment with respect to the HIV mAb 1/CD3 mAb 2/CD8 mAb 1 Tri-Specific Binding Molecule are shown in FIGS. 23A-23C. The results of this assessment with respect to the HIV mAb 2/CD3 mAb 2/CD8 mAb 1 Tri-Specific Binding Molecule are shown in FIGS. 24A-24C. Table 19 summarizes the results.

TABLE 19
Jurkat Cells 522FY
(% Cytotoxicity Relative to LDH MR from Target Cell w/o Binding Molecule)

| | | HIV mAb 1/CD3 mAb | | | | HIV mAb 2/CD3 mAb | |
|---|---|---|---|---|---|---|---|
| HIV mAb 1, | | 2/CD8 mAb 1 | | HIV mAb 2, | | 2/CD8 mAb 1 | |
| CD3 mAb 2 | | Tri-Specific Binding | | CD3 mAb 2 | | Tri-Specific Binding | |
| DART ™ | | Molecule | | DART ™ | | Molecule | |
| EC₅₀, nM | Eₘₐₓ, % | EC₅₀, nM | Eₘₐₓ, % | EC₅₀, nM | Eₘₐₓ, % | EC₅₀, nM | Eₘₐₓ, % |

CD4

| | | | | | | | |
|---|---|---|---|---|---|---|---|
| 0.67 | 13 | 0.36 | 15 | 0.25 | 9 | 0.45 | 8 |

CD8

| | | | | | | | |
|---|---|---|---|---|---|---|---|
| 0.20 | 18 | 0.00781 | 37 | 0.052 | 16 | 0.00040 | 27 |

Pan T

| | | | | | | | |
|---|---|---|---|---|---|---|---|
| 0.31 | 19 | 0.044 | 38 | 0.26 | 16 | 0.0015 | 23 |

The impact of varying the ratio or target:effector cells was evaluated. Table 20 summarizes the obtained results.

TABLE 20

| | Jurkat Cells 522FY | | | | | | | |
|---|---|---|---|---|---|---|---|---|
| | (% Cytotoxicity Relative to LDH MR from | | | | | | | |
| | Target Cell w/o Binding Molecule | | | | | | | |
| | HIV mAb 1 Binding Domain | | | | HIV mAb 2 Binding Domain | | | |
| | E/T = 5:1 | | E/T = 10:1 | | E/T = 5:1 | | E/T = 10:1 | |
| | EC₅₀ | Eₘₐₓ | EC₅₀ | Eₘₐₓ | EC₅₀ | Eₘₐₓ | EC₅₀ | Eₘₐₓ |
| | nM | % | nM | % | nM | % | nM | % |
| DART ™ | 0.076 | 26 | 0.066 | 27 | 0.00057 | 24 | 0.00061 | 25 |
| DART ™ | 0.0092 | 15 | 0.0066 | 15 | 0.0058 | 15 | 0.0054 | 15 |
| Tri-Specific | 0.00019 | 22 | 0.00029 | 24 | 0.00032 | 25 | 0.00026 | 24 |
| Binding | | | | | | | | |
| Molecule | | | | | | | | |

An assessment was also made of the CTL activity of Tri-Specific Binding Molecules on HIV env-expressing HEK293 cells using CD4+, CD8+ or pan T cells. The results of this assessment are summarized in Table 21.

TABLE 21
(% Cytotoxicity Relative to LDH MR from Target Cell w/o Tri-Specific
Binding Molecule)

| HEK293 D371 Cells | | | | HEK293 D375 Cells | | | |
|---|---|---|---|---|---|---|---|
| | | HIV mAb 1/CD3 | | | | HIV mAb 2/CD3 | |
| | | mAb 2/CD8 | | | | mAb 2/CD8 | |
| HIV mAb 1, | | mAb 1 | | HIV mAb 2, | | mAb 1 | |
| CD3 mAb 2 | | Tri-Specific | | CD3 mAb 2 | | Tri-Specific | |
| DART ™ | | Binding Molecule | | DART ™ | | Binding Molecule | |
| EC₅₀, nM | Eₘₐₓ, % | EC₅₀, nM | Eₘₐₓ, % | EC₅₀, nM | Eₘₐₓ, % | EC₅₀, nM | Eₘₐₓ, % |

CD4

| | | | | | | | |
|---|---|---|---|---|---|---|---|
| 0.45 | 0.64 | 3.16 | 1.12 | 0.15 | 0.14 | 0.84 | 0.18 |

CD8

| | | | | | | | |
|---|---|---|---|---|---|---|---|
| 0.029 | 0.0062 | 1.77 | 0.0043 | 0.018 | 0.0012 | 1.00 | 0.0020 |

Pan T

| | | | | | | | |
|---|---|---|---|---|---|---|---|
| 0.082 | 0.025 | 2.06 | N/A | 0.042 | 0.0085 | 1.08 | N/A |

### Example 7

### Comparison of CD3 Binding Domain Affinity Variants

As noted above, the CD3, CD8 or Disease-Associated Antigen-Binding Domains of the Tri-Specific Binding Molecules of the present invention may be mutated in order to isolate Binding Domains having more desired binding characteristics. The CD3 mAb 2 Binding Domain was subjected to such mutagenesis and two affinity variants (CD3 mAb 2 Low and CD3 mAb 2 Fast) were isolated.

The amino acid sequence of the Variable Light Chain Domain of anti-human CD3 mAb 2 Low is (SEQ ID NO:99):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The amino acid sequence of the Variable Heavy Chain Domain of anti-human CD3 mAb 2 Low is (SEQ ID NO:100):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVT WFAYWGQGTL
VTVSS

The amino acid sequence of the Variable Light Chain Domain of anti-human CD3 mAb 2 Fast is (SEQ ID NO:101):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The amino acid sequence of the Variable Heavy Chain Domain of anti-human CD3 mAb 2 Fast is (SEQ ID NO:102):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HKNFGNSYVT WFAYWGQGTL
VTVSS

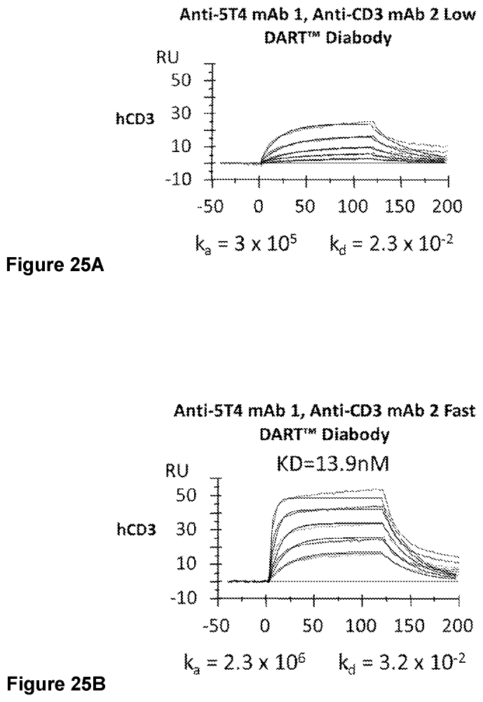

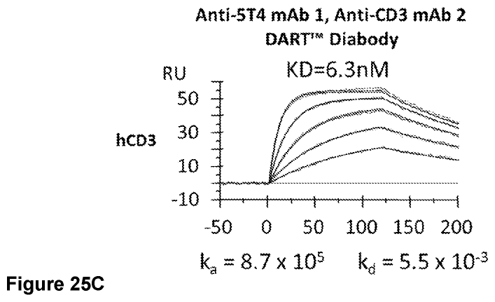

FIGS. 25A-25C show the kinetics of binding for these variants. CD3 mAb 2 Low (FIG. 25A) is a low affinity variant, while CD3 mAb 2 Fast (FIG. 25B) has a faster off rate relative to the wild-type CD3 mAb 2 (FIG. 25C).

In order to assess the effect of CD3 binding characteristics, two CD3 Binding Domain mutants were used. Three Tri-Specific Binding Molecule specific for the Disease-Associated Antigen 5T4 were constructed utilizing a wild-type CD3 mAb 2 Variable Domain sequence, a CD3 mAb 2 Variable Domain sequence with low affinity for CD3 and a CD3 mAb 2 Variable Domain sequence with wild-type affinity but a faster off rate. The 5T4 Variable Domain specificities and CD8 Variable Domain specificities were the same between the three Tri-Specific Binding Molecules. The first Tri-Specific Binding Molecule is termed 5T4 mAb 1/CD3 mAb 2/CD8 mAb 1 and was composed of four different polypeptide chains (Table 22).

TABLE 22

| Polypeptide | | |
|---|---|---|
| Chain | Domains | Binding Affinity |
| 1 | VL(5T4 mAb 1)-VH(CD3 mAb 2)-E- | Light Chain: 5T4 |
| | Coil-(CH2—CH3) | Heavy Chain: CD3 |
| 2 | VL(CD3 mAb 2)-VH(5T4 mAb 1)- | Light Chain: CD3 |
| | K-Coil | Heavy Chain: 5T4 |
| 3 | Heavy Chain CD8 mAb 1 | CD8 |
| 4 | Light Chain CD8 mAb 1 | CD8 |

The amino acid sequence of the first polypeptide chain of the 5T4 mAb 1/CD3 mAb 2/CD8 mAb 1 Tri-Specific Binding Molecule is (SEQ ID NO:103):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

The amino acid sequence of the second polypeptide chain of the 5T4 mAb 1/CD3 mAb 2/CD8 mAb 1 Tri-Specific Binding Molecule is (SEQ ID NO:104):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSF WMHWVRQAPG
QGLEWMGRID PNRGGTEYNE KAKSRVTMTA DKSTSTAYME
LSSLRSEDTA VYYCAGGNPY YPMDYWGQGT TVTVSSGGCG
GGKVAALKEK VAALKEKVAA LKEKVAALKE

The amino acid sequence of the third polypeptide chain of the 5T4 mAb 1/CD3 mAb 2/CD8 mAb 1 Tri-Specific Binding Molecule is (SEQ ID NO:105):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The amino acid sequence of the fourth polypeptide chain of the 5T4 mAb 1/CD3 mAb 2/CD8 mAb 1 Tri-Specific Binding Molecule is (SEQ ID NO:106):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The second Tri-Specific Binding Molecule is termed 5T4 mAb 1/CD3 mAb 2 Low/CD8 mAb 1 and was composed of four different polypeptide chains (Table 23).

TABLE 23

| Polypeptide | | |
|---|---|---|
| Chain | Domains | Binding Affinity |
| 1 | VL(5T4 mAb 1)-VH(CD3 mAb 2 | Light Chain: 5T4 |
| | Low)-E-coil-(CH2—CH3) | Heavy Chain: CD3 |
| 2 | VL(CD3 mAb 2 Low)-VH(5T4 | Light Chain: CD3 |
| | mAb 1)-K-Coil | Heavy Chain: 5T4 |
| 3 | Heavy Chain CD8 mAb 1 | CD8 |
| 4 | Light Chain CD8 mAb 1 | CD8 |

The amino acid sequence of the first polypeptide chain of the 5T4 mAb 1/CD3 mAb 2 Low/CD8 mAb 1 Tri-Specific Binding Molecule is (SEQ ID NO:107):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVTWFAYW GQGTLVTVSS
ASTKGEVAAC EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The amino acid sequence of the second polypeptide chain of the 5T4 mAb 1/CD3 mAb 2 Low/CD8 mAb 1 Tri-Specific Binding Molecule is (SEQ ID NO: 108):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSF WMHWVRQAPG
QGLEWMGRID PNRGGTEYNE KAKSRVTMTA DKSTSTAYME
LSSLRSEDTA VYYCAGGNPY YPMDYWGQGT TVTVSSASTK
GKVAACKEKV AALKEKVAAL KEKVAALKE

The amino acid sequence of the third polypeptide chain of the 5T4 mAb 1/CD3 mAb 2 Low/CD8 mAb 1 Tri-Specific Binding Molecule is (SEQ ID NO:109):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The amino acid sequence of the fourth polypeptide chain of the 5T4 mAb 1/CD3 mAb 2 Low/CD8 mAb 1 Tri-Specific Binding Molecule is (SEQ ID NO:110):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

The third Tri-Specific Binding Molecule is termed 5T4 mAb 1/CD3 mAb 2 Fast/CD8 mAb 1 and was composed of four different polypeptide chains (Table 24).

TABLE 24

| Polypeptide | | |
|---|---|---|
| Chain | Domains | Binding Affinity |
| 1 | VL(5T4 mAb 1)-VH(CD3 mAb 2 | Light Chain: 5T4 |
| | Fast)-E-coil-(CH2—CH3) | Heavy Chain: CD3 |
| 2 | VL(CD3 mAb 2 Fast)-VH(5T4 | Light Chain: CD3 |
| | mAb 1)-K-Coil | Heavy Chain: 5T4 |
| 3 | Heavy Chain CD8 mAb 1 | CD8 |
| 4 | Light Chain CD8 mAb 1 | CD8 |

The amino acid sequence of the first polypeptide chain of the 5T4 mAb 1/CD3 mAb 2 Fast/CD8 mAb 1 Tri-Specific Binding Molecule is (SEQ ID NO:111):

DIQMTQSPSS LSASVGDRVT ITCRASQGIS NYLAWFQQKP
GKAPKSLIYR ANRLQSGVPS RFSGSGSGTD FTLTISSLQP
EDVATYYCLQ YDDFPWTFGQ GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHKNFG NSYVTWFAYW GQGTLVTVSS
ASTKGEVAAC EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The amino acid sequence of the second polypeptide chain of the 5T4 mAb 1/CD3 mAb 2 Fast/CD8 mAb 1 Tri-Specific Binding Molecule is (SEQ ID NO:112):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSF WMHWVRQAPG
QGLEWMGRID PNRGGTEYNE KAKSRVTMTA DKSTSTAYME
LSSLRSEDTA VYYCAGGNPY YPMDYWGQGT TVTVSSASTK
GKVAACKEKV AALKEKVAAL KEKVAALKE

The amino acid sequence of the third polypeptide chain of the 5T4 mAb 1/CD3 mAb 2 Fast/CD8 mAb 1 Tri-Specific Binding Molecule is (SEQ ID NO:113):

EVQLQQSGAE LVKPGASVKL SCTASGFNIK DTYIHFVRQR
PEQGLEWIGR IDPANDNTLY ASKFQGKATI TADTSSNTAY
MHLCSLTSGD TAVYYCGRGY GYYVFDHWGQ GTTLTVSSAS
TKGPSVFPLA PSSKSTSGGT AALGCLVKDY FPEPVTVSWN
SGALTSGVHT FPAVLQSSGL YSLSSVVTVP SSSLGTQTYI
CNVNHKPSNT KVDKRVEPKS CDKTHTCPPC PAPEAAGGPS
VFLFPPKPKD TLMISRTPEV TCVVVDVSHE DPEVKFNWYV
DGVEVHNAKT KPREEQYNST YRVVSVLTVL HQDWLNGKEY
KCKVSNKALP APIEKTISKA KGQPREPQVY TLPPSREEMT
KNQVSLSCAV KGFYPSDIAV EWESNGQPEN NYKTTPPVLD
SDGSFFLVSK LTVDKSRWQQ GNVFSCSVMH EALHNRYTQK
SLSLSPGK

The amino acid sequence of the fourth polypeptide chain of the 5T4 mAb 1/CD3 mAb 2 Fast/CD8 mAb 1 Tri-Specific Binding Molecule is (SEQ ID NO:114):

DVQINQSPSF LAASPGETIT INCRTSRSIS QYLAWYQEKP
GKTNKLLIYS GSTLQSGIPS RFSGSGSGTD FTLTISGLEP
EDFAMYYCQQ HNENPLTFGA GTKLELRRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC

Human PBMC of healthy donors were treated as described above and incubated with the 5T4 mAb 1/CD3 mAb 2/CD8 mAb 1 Tri-Specific Binding Molecule, the 5T4 mAb 1/CD3 mAb 2 Low/CD8 mAb 1 Tri-Specific Binding Molecule and the 5T4 mAb 1/CD3 mAb 2 Fast/CD8 mAb 1 Tri-Specific Binding Molecule. 5T4×CD3 DART™ (with wild-type, Low and Fast CD3 specificities) were used as controls. The 5T4 mAb 1/CD3 mAb 2 Low/CD8 mAb 1 and 5T4 mAb 1/CD3 mAb 2 Fast/CD8 mAb 1 Tri-Specific Binding Molecules contain mutated CD3 Binding Domains that exhibit altered affinity and binding kinetics for CD3.

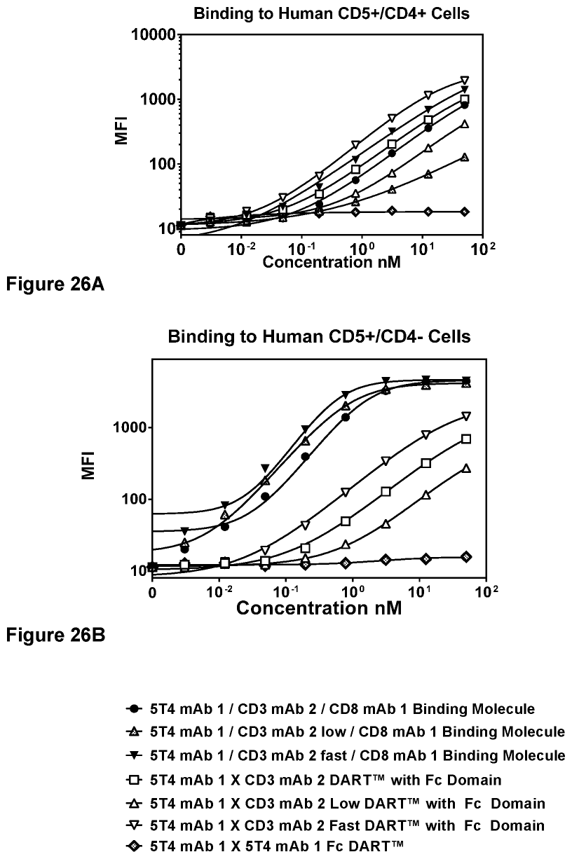

The Tri-Specific Binding Molecules were found to exhibit weaker binding to the CD5+CD4⁺ cells (FIG. 26A), but much stronger binding to the CD5+CD4 cells (FIG. 26B) than the DART™ controls.

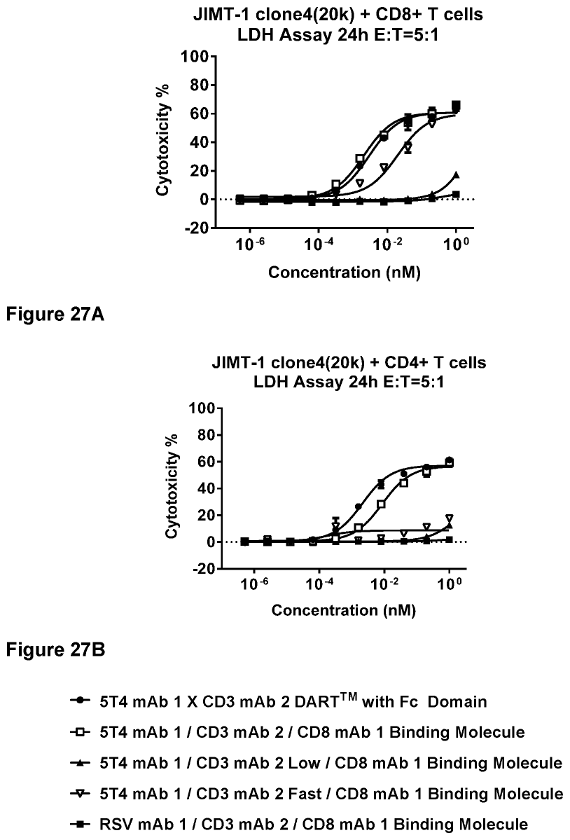

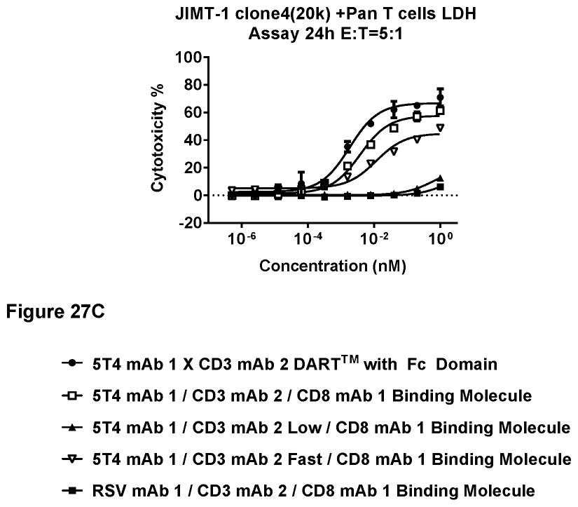

FIGS. 27A-27C show the effect of the CD3 mAb variants on the cytotoxicity of a 5T4 mAb 1/CD3 mAb 2/CD8 mAb 1 Tri-Specific Binding Molecule using an LDH assay. Similar results were obtained using a luciferase assay and with Tri-Specific Binding Molecules having a B7-H3 Binding Domain in lieu of the 5T4 mAb 1 Binding Domain. The results show that the CD3 mAb 2 Low Tri-Specific Binding Molecule exhibited minimal cytotoxicity, but that the CD3 mAb 2 Fast Tri-Specific Binding Molecule exhibited cytotoxicity with CD8+ T cells and pan T cells to a much greater extent than with CD4+ T cells.

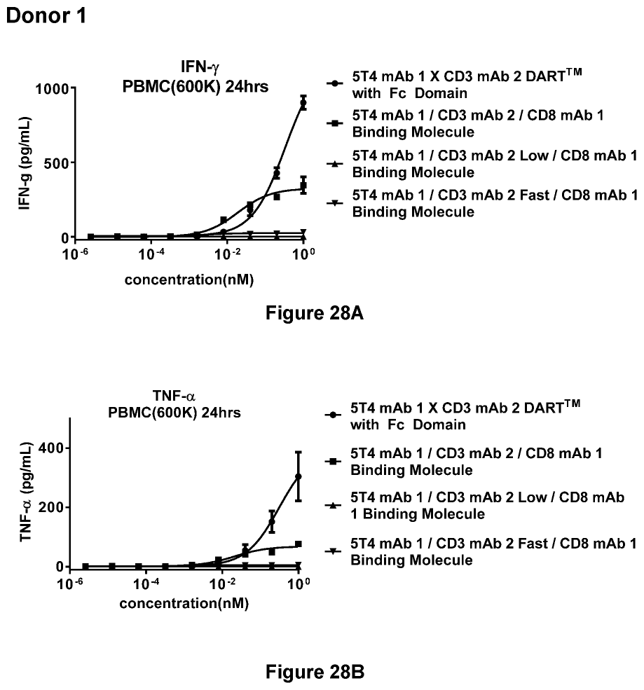

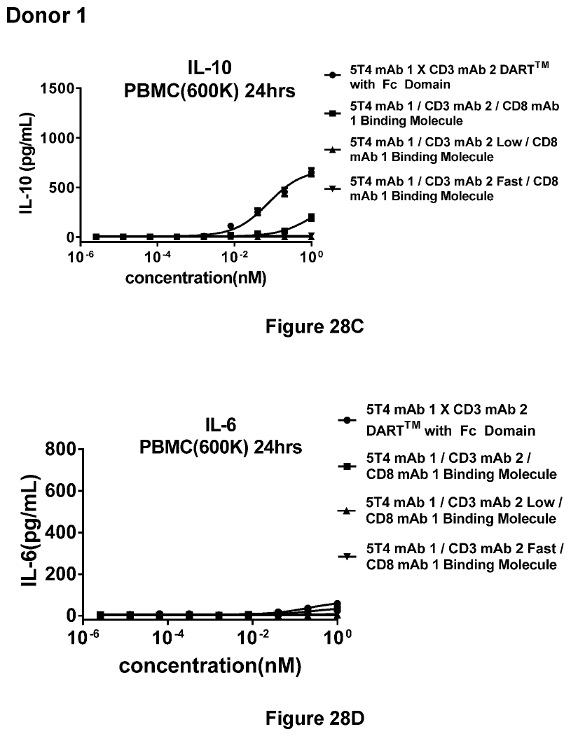

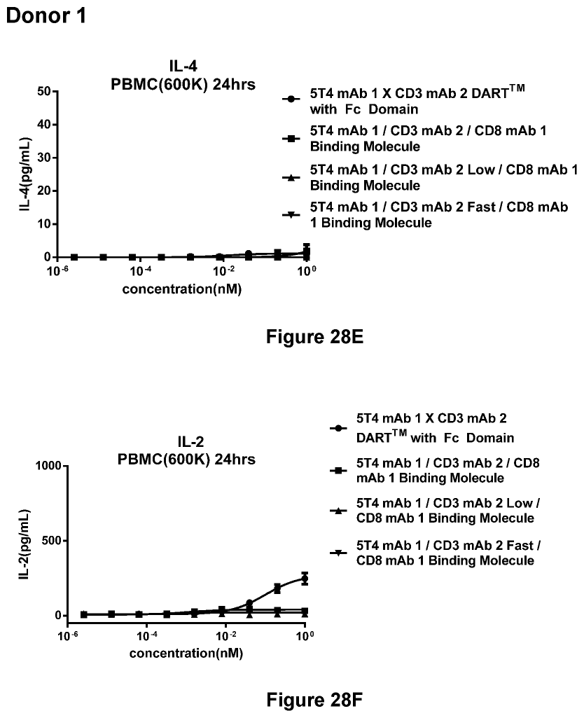

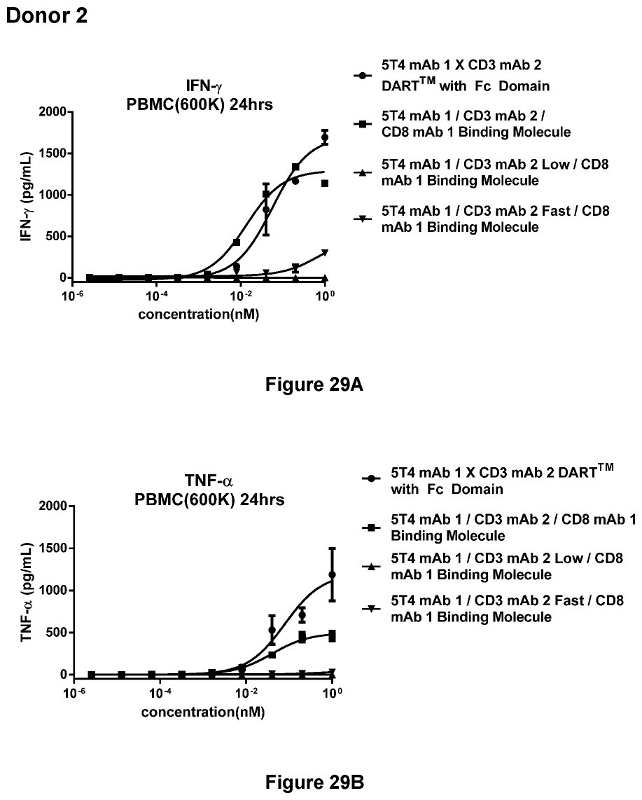

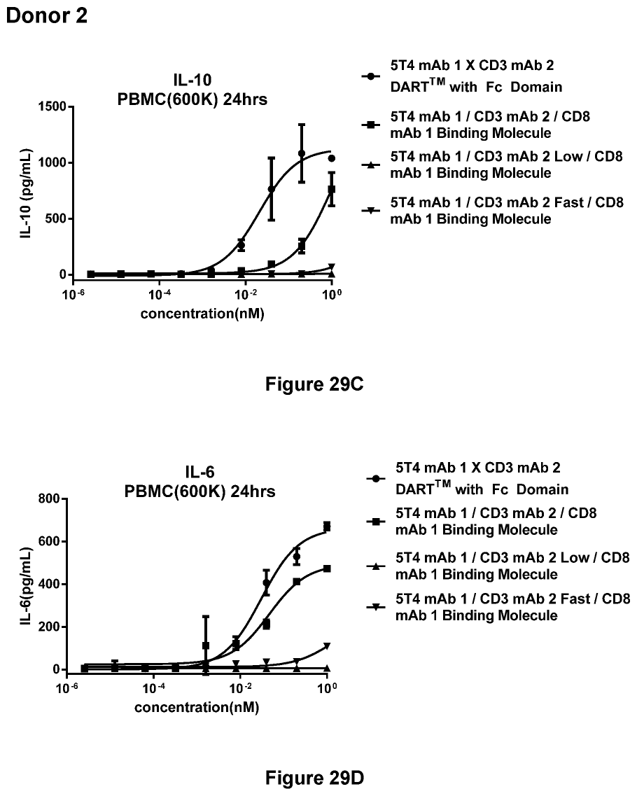

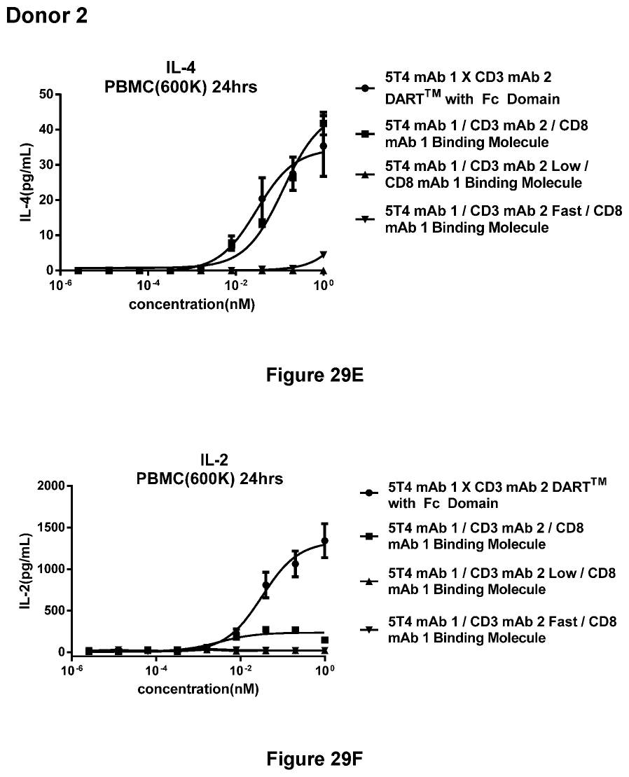

In order to assess the effects of the Tri-Specific Binding Molecules on cytokine profiles, PBMCs from two donors were incubated in the presence of increasing concentrations of the 5T4 mAb 1/CD3 mAb 2/CD8 mAb 1 Tri-Specific Binding Molecule, the 5T4 mAb 1/CD3 mAb 2 Low/CD8 mAb 1 Tri-Specific Binding Molecule and the 5T4 mAb 1/CD3 mAb 2 Fast/CD8 mAb 1 Tri-Specific Binding Molecule for 24 hours. The levels of six cytokines (IFN-γ, TNF-α, IL-10, IL-6, IL-4 and IL-2) were measured. The results are shown in FIGS. 28A-28F (Donor 1) and FIGS. 29A-29F (Donor 2). The results surprisingly show a dramatic decrease in the cytokines released from PBMCs compared to a DART™ that targets only CD3 and is not selective for CD8+ T cells.

The 5T4 mAb 1/CD3 mAb 2/CD8 mAb 1 Tri-Specific Binding Molecule exhibited similar potency to a DART™, 1 μM EC50 compared to 1.3 μM for the DART™, using pan T cells as effectors. The activity of the DART™ may already be maximal for both CD4+ and CD8+ T cells. The Tri-Specific Binding Molecule does shift the activity toward the CD8+ population and away from the CD4+ population which is beneficial in terms of cytokine release, particularly IL-2 and TNFα.

Although the 5T4 mAb 1/CD3 mAb 2 Low/CD8 mAb 1 Tri-Specific Binding Molecule was able to bind CD4+ and CD8+ T cells, its ability to redirect cytolysis of or by those cells was greatly reduced compared to the non-mutated CD3 mAb 2 Binding Domain. The 5T4 mAb 1/CD3 mAb 2 Fast/CD8 mAb 1 Tri-Specific Binding Molecule on the other hand retained CTL activity, particularly with CD8+ T cells compared/relative to CD4+ T cells (75-fold difference in EC50s) compared to the 5T4 mAb 1/CD3 mAb 2/CD8 mAb 1 Tri-Specific Binding Molecule (7-fold difference in EC50s). The EC50 for the 5T4 mAb 1/CD3 mAb 2 Fast/CD8 mAb 1 Tri-Specific Binding Molecule was similar to that of the 5T4 mAb 1/CD3 mAb 2/CD8 mAb 1 Tri-Specific Binding Molecule (2.8 vs 1.3 μM), but the dramatic difference in CD8+vs CD4+ T cell targeting virtually eliminated the cytokine release that was observed with a 5T4×CD3 DART™ in human PBMC cultures.

### Example 8

### Summary of EC50 Data of Exemplary Tri-Specific Binding Molecules

In summary, a series of 14 Tri-Specific Binding Molecules were constructed (Table 25), each having two Diabody-Type Binding Domains (Site A and Site B), and one Fab-Type Binding Domain (Sites C):

TABLE 25

| | Binding Domain | | |
|---|---|---|---|
| Name | Site A | Site B | Site C |
| B7-H3 mAb 1/CD3 mAb 2/CD8 mAb 1 | B7-H3 | CD3 | CD8 |
| CD3 mAb 2/CD8 mAb 1/B7-H3 mAb 1 | CD3 | CD8 | B7-H3 |
| B7-H3 mAb 1/CD8 mAb 1/CD3 mAb 2 | B7-H3 | CD8 | CD3 |
| B7-H3 mAb 1/CD3 mAb 2/CD8 mAb 1 | B7-H3 | CD3 | CD8 |
| 5T4 mAb 1/CD3 mAb 2/CD8 mAb 1 | 5T4 | CD3 | CD8 |
| 5T4 mAb 1/CD3 mAb 2 Low/CD8 mAb 1 | 5T4 | CD3 Low | CD8 |
| 5T4 mAb 1/CD3 mAb 2 Fast/CD8 mAb 1 | 5T4 | CD3 Fast | CD8 |
| 5T4 mAb 2/CD3 mAb 2/CD8 mAb 1 | 5T4 | CD3 | CD8 |
| 5T4 mAb 2/CD3 mAb 2/CD8 mAb 2 | 5T4 | CD3 | CD8 |
| Palivizumab/CD3 mAb 2/CD8 mAb 1 | RSV | CD3 | CD8 |
| ROR1 mAb 1/CD3 mAb 2/CD8 mAb 1 | ROR1 | CD3 | CD8 |
| ROR1 mAb 1/CD3 mAb 2/CD8 mAb 2 | ROR1 | CD3 | CD8 |
| HIV mAb 1/CD3 mAb 2/CD8 mAb 1 | HIV | CD3 | CD8 |
| HIV mAb 2/CD3 mAb 2/CD8 mAb 1 | HIV | CD3 | CD8 |

The EC50 data of such Tri-Specific Binding Molecules is summarized in Table 26 (Target cells: JIMT1-luc; Effector:Target Cell Ratio 5:1). In some cases (shown as NR in Table 26), the EC50 could not be calculated from the data because maximal killing was not achieved.

TABLE 26

| | | | | | | to | to |
|---|---|---|---|---|---|---|---|
| | | EC50 (nM) | | | | DART | DART |
| | | | | | CD4/ | CD4/ | EC50 |
| Target | Binding Molecule | CD8+ | CD4+ | PanT | CD8 | CD8 | w/PanT |
| B7H3 | B7-H3/CD3 mAb 2 | 0.5090 | 1.1370 | 0.6943 | 2.2 | 1.0 | 1.0 |
| | DART ™ | | | | | | |
| | B7-H3/CD8 mAb 1 | NR | NR | NR | — | — | — |
| | DART ™ | | | | | | |
| | B7-H3 mAb 1/CD3 mAb | 0.0054 | 0.0721 | 0.0082 | 13.3 | 5.9 | 85.1 |
| | 2/CD8 mAb 1 | | | | | | |
| | Tri-Specific Binding | | | | | | |
| | Molecule | | | | | | |
| | B7-H3 mAb 1/CD3 mAb | 0.0051 | 0.3138 | 0.0058 | 61.5 | 27.5 | 119.7 |
| | 2/CD8 mAb 2 | | | | | | |
| | Tri-Specific Binding | | | | | | |
| | Molecule | | | | | | |
| | CD3 mAb 2/CD8 mAb | 0.0053 | 0.1055 | 0.0113 | 19.8 | 8.9 | 61.4 |
| | 2/B7-H3 mAb 1 | | | | | | |
| | Tri-Specific Binding | | | | | | |
| | Molecule | | | | | | |
| | B7-H3 mAb 1/CD8 mAb | 0.4125 | 1.1030 | 0.5347 | 2.7 | 1.2 | 1.3 |
| | 2/CD3 mAb 2 | | | | | | |
| | Tri-Specific Binding | | | | | | |
| | Molecule | | | | | | |
| 5T4 | 5T4 mAb 1/CD3 mAb 2 | 0.0012 | 0.0013 | 0.0013 | 1.1 | 1.0 | 1.0 |
| | DART ™ | | | | | | |
| | 5T4 mAb 1/CD3 mAb | 0.0007 | 0.0045 | 0.0009 | 6.8 | 6.4 | 1.5 |
| | 2/CD8 mAb 1 | | | | | | |
| | Tri-Specific Binding | | | | | | |
| | Molecule | | | | | | |
| | 5T4 mAb 1/CD3 mAb 2 | NR | NR | NR | — | — | — |
| | Low/CD8 mAb 1 | | | | | | |
| | Tri-Specific Binding | | | | | | |
| | Molecule | | | | | | |
| | 5T4 mAb 1/CD3 mAb 2 | 0.0021 | 0.1598 | 0.0028 | 74.7 | 70.6 | 0.5 |
| | Fast/CD8 mAb 1 | | | | | | |
| | Tri-Specific Binding | | | | | | |
| | Molecule | | | | | | |
| | 5T4 mAb 2/CD3 mAb 2 | 0.0362 | 0.0561 | 0.0449 | 1.6 | 1.0 | 1.0 |
| | DART ™ | | | | | | |
| | 5T4 mAb 2/CD3 mAb | 0.0013 | 0.0435 | 0.0020 | 32.7 | 21.1 | 22.1 |
| | 2/CD8 mAb 1 | | | | | | |
| | Tri-Specific Binding | | | | | | |
| | Molecule | | | | | | |
| | 5T4 mAb 2/CD3 mAb | 0.0015 | 0.0336 | 0.0018 | 23.2 | 14.9 | 25.0 |
| | 2/CD8 mAb 2 | | | | | | |
| | Tri-Specific Binding | | | | | | |
| | Molecule | | | | | | |
| RSV | Palivizumab/CD3 mAb | NR | NR | NR | — | — | — |
| | 2/CD8 mAb 1 | | | | | | |
| | Tri-Specific Binding | | | | | | |
| | Molecule | | | | | | |
| ROR1 | ROR1/CD3 mAb 2 | 0.7761 | 0.2911 | 0.7643 | 0.4 | 1.0 | 1.0 |
| | DART ™ | | | | | | |
| | ROR1 mAb 1/CD3 mAb | 0.0017 | 0.095 | 0.0053 | 55.9 | 149.0 | 144.2 |
| | 2/CD8 mAb 1 | | | | | | |
| | Tri-Specific Binding | | | | | | |
| | Molecule | | | | | | |
| | ROR1 mAb 1/CD3 mAb | 0.0021 | 0.2767 | 0.0057 | 131.8 | 351.3 | 134.1 |
| | 2/CD8 mAb 2 | | | | | | |
| | Tri-Specific Binding | | | | | | |
| | Molecule | | | | | | |

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-23. (canceled)

24. A multi-chain polypeptide-containing Tri-Specific Binding Molecule that immunospecifically binds to three different epitopes, comprising four different polypeptide chains covalently complexed together; the Tri-Specific Binding Molecule Comprising:
(I) an Antigen-Binding Domain I that immunospecifically binds to an Epitope I present on a first antigen, and an Antigen-Binding Domain II that immunospecifically binds to an Epitope II present on a second antigen;
(II) a Receptor-Type Binding Domain; and
(III) a Fc Domain; wherein:
(A) a first polypeptide chain comprises, from the N-terminus to the C-terminus:
(VL_{I} Domain)-(Linker 1)-(VH_{II} Domain)-(Linker 2)-(Heterodimer-Promoting Domain)-(Linker 3)-(CH2-CH3 Domain); or
(CH3-CH2 Domain)-(Linker 4)-(VL_{I} Domain)-(Linker 1)-(VH_{II} Domain)-(Linker 2)-(Heterodimer-Promoting Domain); or
(Cysteine-Containing Domain)-(CH2-CH3 Domain)-(Linker 4)-(VL_{I} Domain)-(Linker 1)-(VH_{II} Domain)-(Linker 2)-(Heterodimer-Promoting Domain);
(B) a second polypeptide chain comprises, from the N-terminus to the C-terminus:
(VL_{II} Domain)-(Linker 1)-(VH_{I} Domain)-(Linker 2)-(Heterodimer-Promoting Domain);
(C) a third polypeptide chain comprises, from the N-terminus to the C-terminus:
(first polypeptide of a Receptor-Type Binding Domain)-(Cysteine-Containing Domain);
(D) a fourth polypeptide chain comprises, from the N-terminus to the C-terminus:
(second polypeptide of a Receptor-Type Binding Domain)-(Cysteine-Containing Domain)-(CH2-CH3 Domain);
(E) the VL_{I} Domain is a Light Chain Variable Domain of an immunoglobulin that binds Epitope I, the VH_{I} Domain is a Heavy Chain Variable Domain of an immunoglobulin that binds Epitope I, the VL_{II} Domain is a Light Chain Variable Domain of an immunoglobulin that binds Epitope II, the VH_{II} Domain is a Heavy Chain Variable Domain of an immunoglobulin that binds Epitope II;
(F) the VL_{I} Domain and the VH1 Domain associate to form the Antigen-Binding Domain I, the VL_{II} Domain and the VH_{II} Domain associate to form the Antigen-Binding Domain II, the first polypeptide of the Receptor-Type Binding Domain and the second polypeptide of the Receptor-Type Binding Domain associate to form a Receptor-Type Domain, and the CH2-CH3 Domain of the first polypeptide chain and the CH2-CH3 Domain of the third polypeptide chain associate to form the Fc Domain;
(G) the first, second and third antigens are the same antigen, or are independently the same or different from another of the antigens.

25. The Tri-Specific Binding Molecule of claim 24, wherein (i) the first polypeptide chain and the second polypeptide chain are covalently bonded to one another; and (ii) the third polypeptide chain and the fourth polypeptide chain are covalently bonded to one another.

26. The Tri-Specific Binding Molecule of claim 24, wherein the Antigen-Binding Domain I and the Antigen-Binding Domain II are Diabody-Type Binding Domains, and the Receptor-Type Binding Domain is a Non-Diabody-Type Binding Domain.

27. The Tri-Specific Binding Molecule of claim 24, wherein the Linker 1 comprises the sequence of SEQ ID NO: 1; the Linker 2 comprises the sequence of SEQ ID NO: 2 or 131; the Linker 3 comprises the sequence of SEQ ID NO: 5 or GGG; and the Linker 4 comprises the sequence of SEQ ID NO: 15, 16, 131 or 132, or GGC or GGG.

28. The Tri-Specific Binding Molecule of claim 24, wherein the Heterodimer-Promoting Domain on the first polypeptide chain is an E-coil Domain and the Heterodimer-Promoting Domain on the second polypeptide chain is a K-coil Domain, or the Heterodimer-Promoting Domain on the first polypeptide chain is a K-coil Domain and the Heterodimer-Promoting Domain on the second polypeptide chain is an E-coil Domain.

29. The Tri-Specific Binding Molecule of claim 28, wherein the E-coil Domain independently comprises the sequence of SEQ ID NO: 3 or 115, and the K-coil Domain independently comprises the sequence of SEQ ID NO: 4 or 116.

30. The Tri-Specific Binding Molecule of claim 24, wherein the Cysteine-Containing Domain independently comprises the sequence of SEQ ID NO: 2, 5, 10, 11, 12, 13, 14, 127, 128, 129, 130 or 133.

31. The Tri-Specific Binding Molecule of claim 24, wherein one of Epitope I or Epitope II is an epitope of:
an effector cell chosen from CD2, CD3, CD16, CD19, CD20, CD22, CD32B, CD64, B cell Receptor (BCR), T cell Receptor (TCR), or NKG2D Receptor; or
CD8; or
a Disease-Associated Antigen.

32. The Tri-Specific Binding Molecule of claim 24, wherein the Receptor-Type Binding Domain refers to an epitope-binding domain of a cellular receptor selected from:
T cell receptor, IL-2 receptor, IL-4 receptor, IL-7 receptor, IL-9 receptor, IL-15 receptor, IL-21 the insulin receptor, and thymic stromal lymphopoietin.

33. The Tri-Specific Binding Molecule of claim 32, wherein the cellular receptor is a T cell receptor, which is formed from the interaction of a Variable Domain of a T Cell Receptor alpha chain and a Variable Domain of a T Cell Receptor beta chain, that is capable of physiospecific binding to an antigen molecule displayed in the class I MHC complex arrayed on the surface of a Cell.

34. The Tri-Specific Binding Molecule of claim 31, wherein the Disease-Associated Antigen is a cancer antigen selected from the group consisting of: colon cancer antigen 19.9; gastric cancer mucin antigen 4.2; colorectal carcinoma antigen A33; ADAM-9; AFP oncofetal antigen-alpha-fetoprotein; ALCAM; B7-H3; BAGE; beta-catenin; CA125; Carboxypeptidase M; B1; CD5; CD19; CD20; CD22; CD23; CD25; CD27; CD28; CD30; CD33; CD36; CD40/CD154; CD45; CD56; CD46; CD52; CD79a/CD79b; CD103; CD317; CDK4; CEA carcinoembryonic antigen; CEACAM5 and CEACAM6; CO-43 (blood group Le^{b}) and CO-514 (blood group Le^{a}); CTLA-1 and CTLA-4; Cytokeratin 8; DR5; E1 series (blood group B); EGF-R epidermal growth factor receptor; Ephrin receptors (EphA2); Erb (ErbB1; ErbB3; ErbB4); lung adenocarcinoma antigen F3; antigen FC10.2; GAGE (GAGE-1; GAGE-2); GD2/GD3/GD49/GM2/GM3; GICA 19-9; gp37 (human leukemia T cell antigen); gp75 (melanoma antigen); gp100; HER-2/neu; human B-lymphoma antigen-CD20; human milk fat globule antigen; human papillomavirus-E6/human papillomavirus-E7; HMW-MAA (high molecular weight melanoma antigen); I antigen (differentiation antigen); I(Ma) as found in gastric adenocarcinomas; Integrin Alpha-V-Beta-6 Integrinβ6 (ITGB6); Interleukin-13 Receptor α2 (IL13Rα2); JAM-3; KID3; KID31; KS 1/4 pan-carcinoma antigen; KSA (17-1A); human lung carcinoma antigens L6 and L20; LEA; LUCA-2; M1:22:25:8, M18, M39; MAGE (MAGE-1; MAGE-3); MART; Myl, MUC-1; MUM-1; N-acetylglucosaminyltransferase; neoglycoprotein; NS-10; OFA-1 and OFA-2; Oncostatin M (Oncostatin Receptor Beta); ρ15; PSA (prostate specific antigen); PSMA; PEMA (polymorphic epithelial mucin antigen); PIPA; prostatic acid phosphate; R24; ROR1; SSEA-1, SSEA-3 and SSEA-4; sTn; T cell receptor derived peptide; T5A7; Tissue Antigens 37; TAG-72; TL5 (blood group A); TNF-receptor (TNF-α receptor, TNF-β receptor; or TNF-γ receptor); TRA-1-85 (blood group H); Transferrin Receptor; TSTA tumor-specific transplantation antigen; VEGF-R; Y hapten, Le^{y} or 5T4.

35. The Tri-Specific Binding Molecule of claim 24, wherein one of Epitope I or Epitope II is an epitope of CD3 and the Antigen-Binding Domain that immunospecifically binds to the epitope of CD3 comprises the six CDRs of SEQ ID NO: 17 and 18; 19 and 20; 21 and 22; 23 and 25; 24 and 25; 26 and 27; 26 and 28; or 101 and 102.

36. The Tri-Specific Binding Molecule of claim 24, wherein one of Epitope I or Epitope II is an epitope of CD8 and the Antigen-Binding Domain that immunospecifically binds to the epitope of CD8 comprises the six CDRs of SEQ ID NO: 29 and 30; or 31 and 32.

37. The Tri-Specific Binding Molecule of claim 24, wherein one of Epitope I or Epitope II is an epitope of B7-H3, A33, 5T4 or ROR1 and the Antigen-Binding Domain that immunospecifically binds to the epitope of B7-H3, A33, 5T4 or ROR1 comprises the six CDRs of SEQ ID NO: 39 and 40; 41 and 42; 43 and 44; 45 and 46; 47 and 48; 49 and 50; 51 and 52; 53 and 54; 55 and 56; or 57 and 58.

38. The Tri-Specific Binding Molecule of claim 24, wherein the CH2-CH3 Domain of the first polypeptide chain and the third polypeptide chain comprise:
(A) one substitution selected from the group consisting of:
F243L, R292P, Y300L, V305I, and P396L;
(B) two substitutions selected from the group consisting of:
(1) F243L and P396L;
(2) F243L and R292P; and
(3) R292P and V305I;
(C) three substitutions selected from the group consisting of:
(1) F243L, R292P and Y300L;
(2) F243L, R292P and V305I;
(3) F243L, R292P and P396L; and
(4) R292P, V305I and P396L;
(D) four substitutions selected from the group consisting of:
(1) F243L, R292P, Y300L and P396L; and
(2) F243L, R292P, V305I and P396L; or
(E) five substitutions selected from the group consisting of:
(1) F243L, R292P, Y300L, V305I and P396L; and
(2) L235V, F243L, R292P, Y300L and P396L.

39. The Tri-Specific Binding Molecule of claim 24, wherein the third polypeptide chain comprises a CH1 Domain.

40. The Tri-Specific Binding Molecule of claim 39, wherein the CH1 Domain comprises the sequence of SEQ ID NO: 9.

41. The Tri-Specific Binding Molecule of claim 39, wherein the fourth polypeptide chain comprises a CL Domain.

42. The Tri-Specific Binding Molecule of claim 41, wherein the CL Domain comprises the sequence of SEQ ID NO: 13 or 14.

43. A pharmaceutical composition comprising the Tri-Specific Binding Molecule of claim 24 and a pharmaceutically acceptable carrier, excipient or diluent.
